(19) United States
(12) Patent Application Publication
(10) Pub. No.: US 2024/0140926 A1
(43) Pub. Date: May 2, 2024

(54) ANTHRACENE-BASED COMPOUND, MIXTURE, AND ORGANIC ELECTRONIC DEVICE

(71) Applicant: Shenzhen China Star Optoelectronics Semiconductor Display Technology Co., Ltd., Shenzhen (CN)

(72) Inventors: Ruifeng HE, Shenzhen (CN); Canjie WU, Shenzhen (CN); Yan LI, Shenzhen (CN); Jingyao SONG, Shenzhen (CN)

(21) Appl. No.: 17/993,911

(22) Filed: Nov. 24, 2022

(30) Foreign Application Priority Data
Sep. 30, 2022 (CN) 202211210477.3

(51) Int. Cl. C07D 307/91; C07D 333/76; H10K 50/11; H10K 50/15; H10K 50/16; H10K 50/17; H10K 59/00; H10K 85/60

(52) U.S. Cl. CPC C07D 307/91; C07D 333/76; H01L 27/32; H01L 51/0052; H01L 51/0058; H01L 51/0059; H01L 51/0072; H01L 51/0073; H01L 51/0074; H01L 51/5004; H01L 51/5056; H01L 51/5072; H01L 51/5092

(57) ABSTRACT

An organic electronic device includes an anthracene-based compound represented by formula (I)

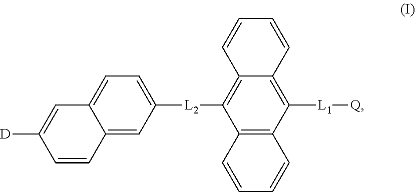

wherein Q is selected from groups represented by formula (A-1) and formula (A-2):

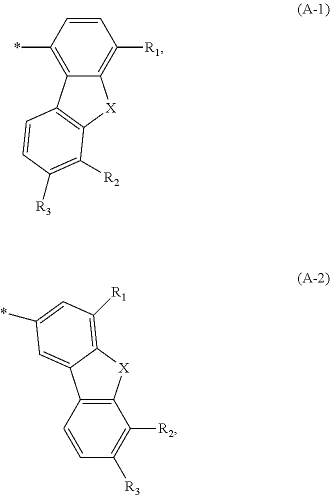

X is O or S;
each of R₁, R₂, and R₃ is independently selected from —H, -D, a C₁-C₆ alkyl group, a phenyl group, a biphenyl group, a phenyl group substituted by a C₁-C₆ alkyl group, and a biphenyl group substituted by a C₁-C₆ alkyl group;
each of L₁ and L₂ is independently selected from a single bond, a phenyl group, a biphenyl group, a phenyl group substituted by a C₁-C₆ alkyl group, and a biphenyl group substituted by a C₁-C₆ alkyl group; and
* indicates a binding site.

### FIELD

The present application relates to a field of organic light-emitting, in particularly to an anthracene-based compound, a mixture, and an organic electronic device.

### BACKGROUND

Due to diversity in synthesis, relatively low manufacturing cost, and excellent optical and electrical performances of organic semiconductor materials, organic light-emitting diodes (OLEDs) have great potential in application of optoelectronic devices (such as flat panel displays and illumination).

An organic electroluminescence phenomenon refers to a phenomenon of using organic substances to convert electrical energy into light energy. Organic electroluminescence elements, occur the organic electroluminescence phenomenon, usually include positive electrodes, negative electrodes, and organic layers arranged between the positive electrodes and the negative electrodes. In order to improve efficiency and lifespan of the organic electroluminescence elements, an organic layer may include a multi-layered structure, and each layer contains different organic substances. In detail, the organic layer may include a hole injection layer, a hole transport layer, a light-emitting layer, an electron transport layer, an electron injection layer, etc. In this organic electroluminescence elements, when voltages are applied to a positive electrode and a negative electrode, holes are injected into the organic layer from the positive electrode, and electrons are injected into the organic layer from the negative electrode. When the injected holes meet with the injected electrons, excitons will be formed and emit light when they transition back to a ground state. This kind of organic electroluminescent elements has performances of self-emission, high brightness, great efficiency, low driving voltages, wide viewing angles, high contrast ratios, good responsiveness, etc.

In order to improve light-emitting efficiency of the organic electroluminescence elements, various light-emitting material systems based on fluorescence and phosphorescence have been developed. However, development of excellent blue light materials is a huge challenge whether it is fluorescent materials or phosphorescent materials. In general, efficiency of most blue light materials is lower and not conducive to high-end display. At the same time, stability and lifespan of the OLEDs made of blue light-emitting materials need to be further improved. Therefore, it is feasible to develop new blue light-emitting materials with great efficiency and long lifespan to effectively improve an overall performance of display devices.

### SUMMARY

The present application aims to provide an anthracene-based compound, a mixture, and an organic electronic device.

The anthracene-based compound is represented by formula (I):

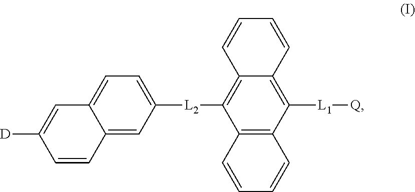

wherein Q is selected from groups represented by formula (A-1) and formula (A-2):

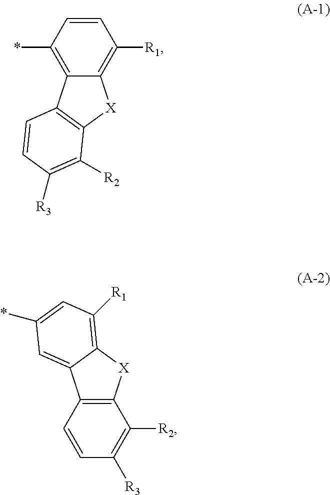

X is O or S;
R₁, R₂, and R₃ are each independently selected from —H, -D, a C₁-C₆ alkyl group, a phenyl group, a biphenyl group, a phenyl group substituted by a C₁-C₆ alkyl group, and a biphenyl group substituted by a C₁-C₆ alkyl group;
L₁ and L₂ are both independently selected from a single bond, a phenyl group, a biphenyl group, a phenyl group substituted by a C₁-C₆ alkyl group, and a biphenyl group substituted by a C₁-C₆ alkyl group; and
* indicates a binding site.

In the anthracene-based compound provided by the present application, an end of a conjugated naphthalene group is deuterated to improve stability of the anthracene-based compound. An end of an anthracene group is regulated by the Q group with better hole transport ability, at the same time, stability and charge transport ability of the anthracene-based compound can be improved. By taking the anthracene-based compound of the present application as a blue light material, stability and lifespan of devices can be effectively improved.

### BRIEF DESCRIPTION OF DRAWINGS

In order to illustrate technical solutions in embodiments of the present application more clearly, the following briefly introduces drawings needed to be used in description of the embodiments. Obviously, the drawings in the following description are only some embodiments of the present application. For those skilled in the art, other drawings can be obtained from these drawings without paying creative effort.

FIG. 1 is a schematic structural diagram of an organic electronic device provided by one embodiment of the present application.

### DETAILED DESCRIPTION

Technical solutions in the present application will be illustrated clearly and completely below in combination drawings in the embodiments of the present application. Obviously, the described embodiments are only a part of the embodiments of the present application, not all of them. Based on the embodiments in the present application, all other embodiments obtained by those skilled in the art without pay creative effort belong to a scope of the present application.

In order to make a purpose, technical solutions and effects of the present application more clearly and definitely, the present application will be further described in detail below. It should be understood that specific embodiments described herein are only used to explain the present application, not to define the present application.

Terms “and/or”, “or/and” used herein include any one of two or more related listed items, as well as any and all combinations of related listed items. All combinations include any two related listed items, any more related listed items, or combinations of all related listed items. It should be noted that when connecting at least three projects containing at least two conjunctions selected from “and/or”, “or/and”, it should be understood that in the present application, technical solutions undoubtedly include both technical solutions connected containing “logical AND” and technical solutions connected containing “logical OR”. For example, “A and/or B” includes three parallel solutions: A, B, and A+B. Another example is, technical solutions of “A, and/or, B, and/or, C, and/or, D” includes any one of A, B, C, and D (that is, the technical solutions connected containing “logical OR”), and any and all combinations of A, B, C, and D, that is, any two or any three combinations of A, B, C, and D, as well as four combinations of A, B, C, and D (that is, the technical solutions connected containing “logical AND”).

In the present application, an aromatic group and an aromatic ring system have a same meaning and may be interchanged.

In the present application, a heteroaromatic group and a heteroaromatic ring system have a same meaning and may be interchanged.

In the present application, “substituted” means that one or more hydrogen atoms in one substituted group are substituted by a substituent group.

In the present application, a same substituent group at different substituent site may be independently selected from different groups. If a formula includes a plurality of R, each R can be independently selected from different groups.

In the present application, “substituted or unsubstituted” means that a defined group may be substituted or not be substituted. When the defined group is substituted, it should be understood that the defined group may be substituted by one substituent R or more substituents R. The substituent R is selected from, but not limited thereto: -D, a cyano group, an isocyano group, a nitro group, a halogen group, a C₁-C₂₀ alkyl group, a heterocyclic group containing 3-20 ring atoms, an aromatic group containing 6-20 ring atoms, a heteroaromatic group containing 5-20 ring atoms, —NRR″, a silane group, a carbonyl group, an alkoxycarbonyl group, an aryloxycarbonyl group, a carbamoyl group, a haloformyl group, a formyl group, an isocyanate group, a thiocyanate group, an isothiocyanate group, a hydroxyl group, and a trifluoromethyl group. In some embodiments, the above groups may further be substituted by acceptable substituents in the art. Understandably, R′ and R′ in the —NR′R″ are both independently selected from, but not limited thereto: —H, -D, a cyanogen group, an isocyano group, a nitro group, a halogen group, a C₁-C₁₀ alkyl group, a heterocyclic group containing 3-20 ring atoms, an aromatic group containing 6-20 ring atoms, and a heteroaromatic group containing 5-20 ring atoms. In some embodiments, the R is selected from, but not limited thereto: -D, a cyano group, an isocyano group, a nitro group, a halogen group, a C₁-C₁₀ alkyl group, a heterocyclic group containing 3-10 ring atoms, an aromatic group containing 6-20 ring atoms, a heteroaromatic group containing 5-20 ring atoms, a silane group, a carbonyl group, an alkoxycarbonyl group, an aryloxycarbonyl group, a carbamoyl group, a haloformyl group, a formyl group, an isocyanate group, a thiocyanate group, an isothiocyanate group, a hydroxyl group, and a trifluoromethyl group, and the above groups may further be substituted by acceptable substituents in the art.

In the present application, “a ring atom number” refers to a number of atoms constituting a ring of structural compounds (such as a monocyclic compound, a fused ring compound, a cross-linked compound, a carbon ring compound, and a heterocyclic compound) obtained by atomic bonding. In a ring substituted by a substituent group, atoms contained in the substituent group is not included in the atoms forming the ring. The same applies to the “number of ring atoms” described below unless otherwise specified. For example, a ring atom number of a benzene ring is 6, a ring atom number of a naphthalene ring is 10, and a ring atom number of a thiophene group is 5.

“An aryl group or an aromatic group” refers to an aromatic hydrocarbon group derived from a basis of an aromatic ring compound removing an H. The aromatic ring compound may be a single ring aromatic group, a fused ring aromatic group, or a polycyclic aromatic group. For a polycyclic ring type, at least one ring is an aromatic ring system. For example, in some embodiments, “a substituted or unsubstituted aryl group containing 6 to 40 ring atoms” refers to an aryl group containing 6 to 40 ring atoms. In some embodiments, “the substituted or unsubstituted aryl group containing 6 to 40 ring atoms” refers to a substituted or unsubstituted aryl group containing 5 to 30 ring atoms. In some embodiments, “the substituted or unsubstituted aryl group containing 6 to 40 ring atoms” refers to a substituted or unsubstituted aryl group containing 6 to 18 ring atoms. In some embodiments, “the substituted or unsubstituted aryl group containing 6 to 40 ring atoms” refers to a substituted or unsubstituted aryl group containing 6 to 14 ring atoms, and the aryl group is optionally further substituted. Suitable examples include, but are not limited thereto: a phenyl group, a biphenyl group, a triphenyl group, a naphthyl group, an anthracyl group, a phenanthryl group, a fluoranthenyl group, a triphenylene group, a pyrenyl group, a perylene group, a tetraphenyl group, a fluorenyl group, a diphenyl group, an acenaphthenyl group, and derivatives of these groups. Understandably, multiple aryl groups may further be disconnected by short non-aromatic units (for example, a non hydrogenium atoms contenting less than 10%, such as C, N, or O). In detail, an acenaphthene group, a fluorene group, a 9,9-diarylfluorene group, a triarylamine group, and a diaryl ether system should be further included in a definition of the aryl groups.

“A heteroaryl group or a heteroaromatic group” refers a basis of an aryl group with at least one carbon atom substituted by a non-carbon atom, and the non-carbon atom may be N, O, S, etc. For example, in some embodiments, “a substituted or unsubstituted heteroaryl group containing 5 to 40 ring atoms” refers to a heteroaryl group containing 5 to 40 ring atoms. In some embodiments, “the substituted or unsubstituted heteroaryl group containing 5 to 40 ring atoms” refers to a substituted or unsubstituted heteroaryl group containing 5 to 30 ring atoms. In some embodiments, “the substituted or unsubstituted heteroaryl group containing 5 to 40 ring atoms” refers to a substituted or unsubstituted heteroaryl group containing 6 to 18 ring atoms. In some embodiments, “the substituted or unsubstituted heteroaryl group containing 5 to 40 ring atoms” refers to a substituted or unsubstituted heteroaryl group containing 6 to 14 ring atoms, and the heteroaryl group are optionally further substituted. Suitable examples include but are not limited thereto: a thiophene group, a furan group, a pyrrolyl group, a diazo group, a triazole group, an imidazolyl group, a pyridinyl group, a bipyridyl group, a pyrimidinyl group, a triazinyl group, an acridine group, a pyridazinyl group, a pyrazinyl group, a quinolinyl group, an isoquinolinyl group, a quinazolinyl group, a quinoxalinyl group, a phthalazinyl group, a pyridino pyrimidinyl group, a pyridino pyrazinyl group, a benzo thienyl group, a benzofuranyl group, an indolyl group, a pyrrolo imidazolyl group, a pyrrolo pyrrolyl group, a thiophenopyrrolyl group, a thiophenothiophenyl group, a furanopyrrolyl group, a furanofuranyl group, a thiophenofuranyl group, a benzoisoxazolyl group, a benzoisothiazolyl group, a benzimidazolyl group, an o-diaznaphthyl group, a phenanthryl group, a pyridinyl group, a quinazolinketone group, a dibenzothiophenyl group, a dibenzofuranyl group, a carbazolyl group, and derivatives of these groups.

“An amine group” refers to a derivative of amine, and has a structural characteristic of the formula —NR′R″, and the R′ and the R″ have same meanings as described above.

In the present application, the “*” connected to a single bond indicates a binding site or a fused site.

In the present application, when a binding site of a group is not specified, it means that any of connectable sites in the group may be selected as the binding site.

In the present application, when a same group contains a plurality of substituents having a same symbol, the substituents may be the same or be different, such as

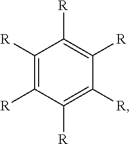

wherein six R groups of a benzene ring may be the same or different.

In the present application, a single bond connected to a substituent group and penetrated a corresponding ring indicates that the substituent group may be connected to any site of the ring. For example,

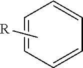

means that R is connected to any substituent site of the benzene ring.

“Combination” used in the present application include all appropriate combination methods of any two or more items in listed groups.

In the present application, the word “further” is used for a purpose of description to indicate differences in contents, but should not be construed as restrictions on a scope of the present application.

In the present application, “optionally” and “optional” refer to be chosen but not obligatory, that is, to select from two parallel solutions: “yes” and “no”. If there are multiple “optional” items in a technical solution, and if there is no special description, contradiction, or mutual restriction, each “optional” item is independent.

In the present application, scope of technical features described in an open form include both closed technical solutions consisting of listed features and open technical solutions containing the listed features.

The present application provides an anthracene-based compound, a mixture, a composition, and organic electronic device.

An anthracene-based compound is represented by formula (I):

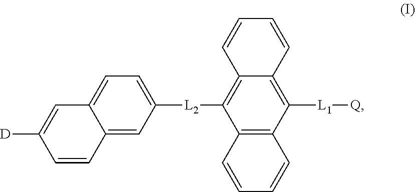

wherein Q is selected from groups represented by formula (A-1) and formula (A-2):

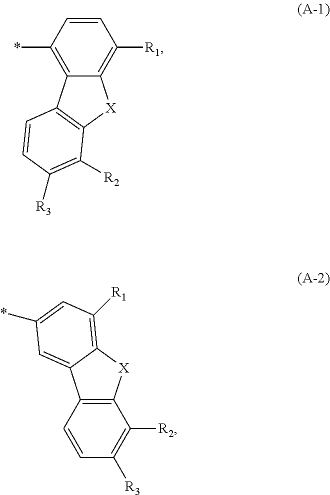

X is O or S;
each of R₁, R₂, and R₃ is independently selected from —H, -D, a C₁-C₆ alkyl group, a phenyl group, a biphenyl group, a phenyl group substituted by a C₁-C₆ alkyl group, and a biphenyl group substituted by a C₁-C₆ alkyl group;
each of L₁ and L₂ is independently selected from a single bond, a phenyl group, a biphenyl group, a phenyl group substituted by a C₁-C₆ alkyl group, and a biphenyl group substituted by a C₁-C₆ alkyl group; and
* indicates a binding site.

In the anthracene-based compound provided by the present application, an end of a conjugated naphthalene group is deuterated to improve stability of the anthracene-based compound. An end of an anthracene group is regulated by the Q group with better hole transport ability, at the same time, stability and charge transport ability of the anthracene-based compound can be improved. By taking the anthracene-based compound of the present application as a blue light material, stability and lifespan of devices can be effectively improved.

In one embodiment, “the C₁-C₆ alkyl group” is a methyl group, an ethyl group, a n-propyl group, an isopropyl group, a n-butyl group, a sec butyl group, a tert-butyl group, an isobutyl group, a 3,3-dimethylbutyl group, a n-amyl group, an isopentyl group, a neopentyl group, a tert amyl group, a cyclopentyl group, a 1-methylamyl group, a 3-methylamyl group, a n-hexyl group, or a cyclohexyl group.

In one embodiment, each of R₁, R₂, and R₃ is independently selected from —H, -D, a methyl group, an ethyl group, a n-propyl group, an isopropyl group, a n-butyl group, a tert-butyl group, and a phenyl group.

In one embodiment, R₁, R₂, and R₃ are all H, and the Q is selected from groups represented by formula (B-1) to (B-4):

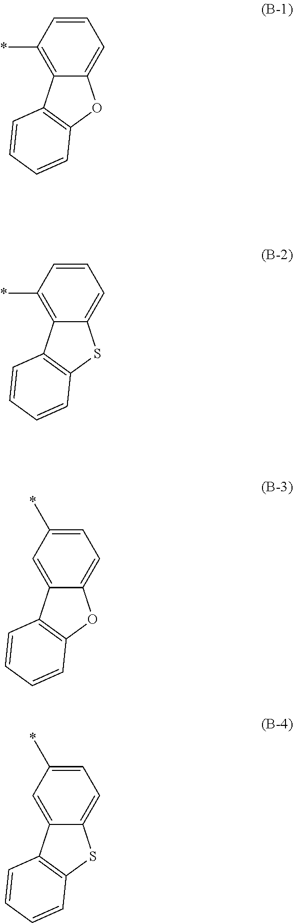 [SMILES: *c1cccc2oc3ccccc3c12 ; *c1cccc2sc3ccccc3c12 ; *c1ccc2oc3ccccc3c2c1 ; *c1ccc2sc3ccccc3c2c1]

In one embodiment, R₁ is not H. R₁ is selected from -D, a methyl group, an ethyl group, a n-propyl group, an isopropyl group, a n-butyl group, a tert-butyl group, and a phenyl group. R₂ and R₃ are both H. In detail, the Q is selected from groups represented by formula (B-5) to (B-23):

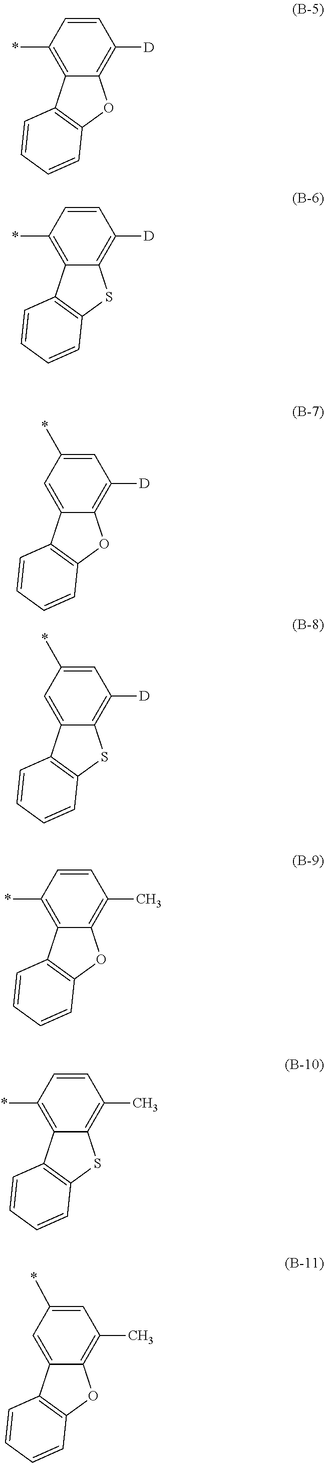

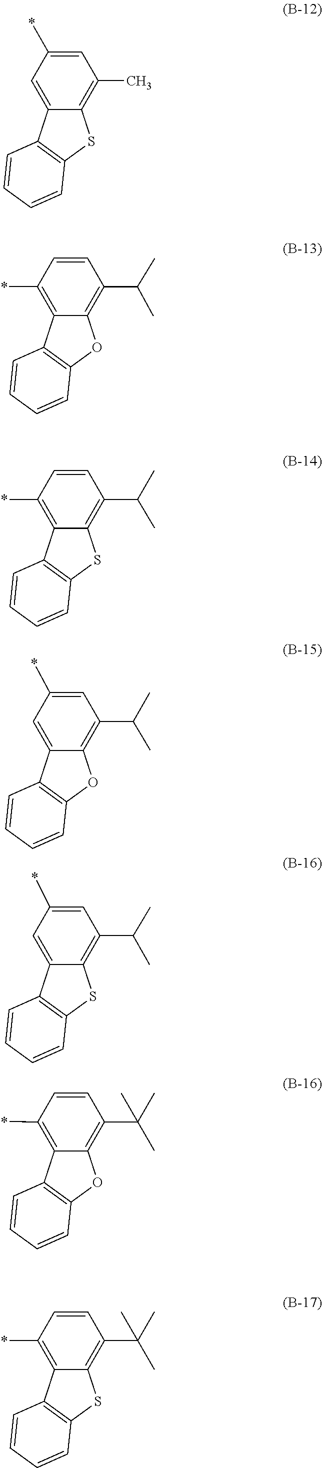

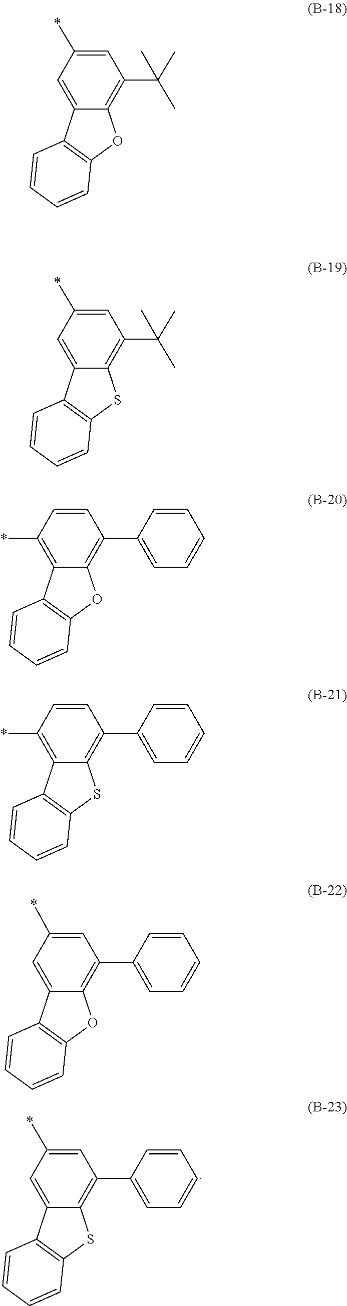

In one embodiment, R₂ is not H. R₂ is selected from -D, a methyl group, an ethyl group, a n-propyl group, an isopropyl group, a n-butyl group, a tert-butyl group, and a phenyl group. R₁ and R₃ are both H. In detail, the Q is selected from groups represented by formula (B-24) to (B-43):

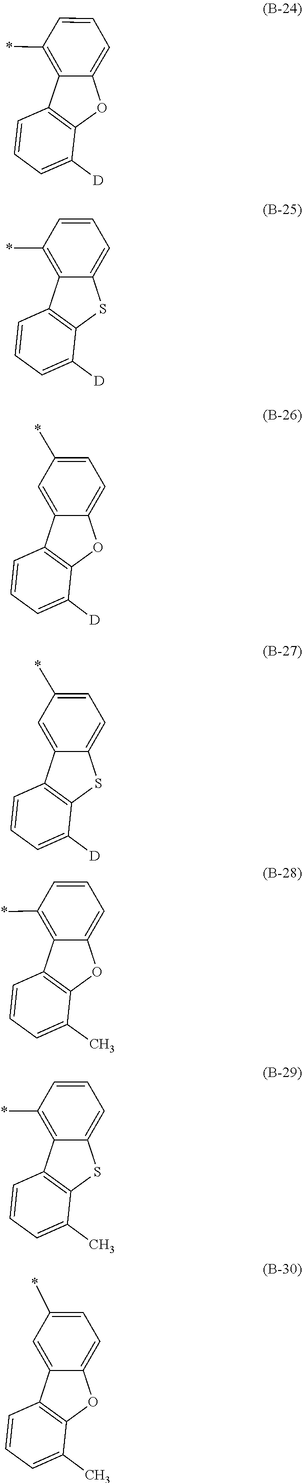

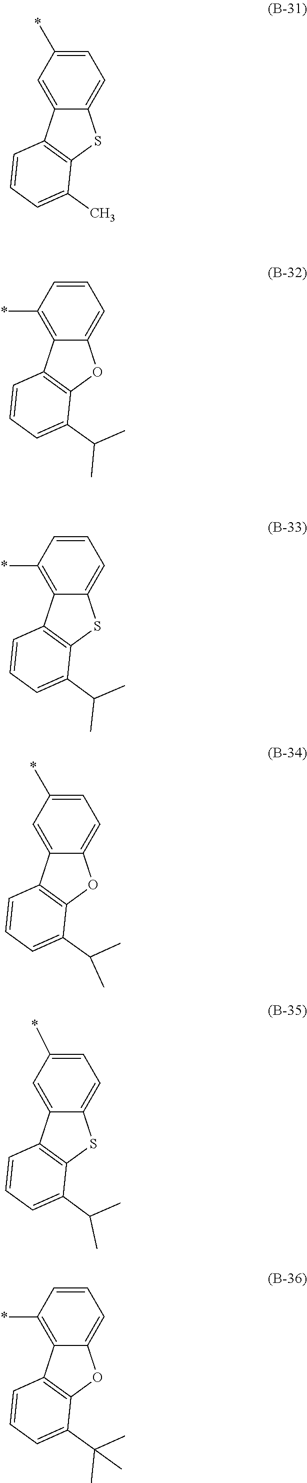

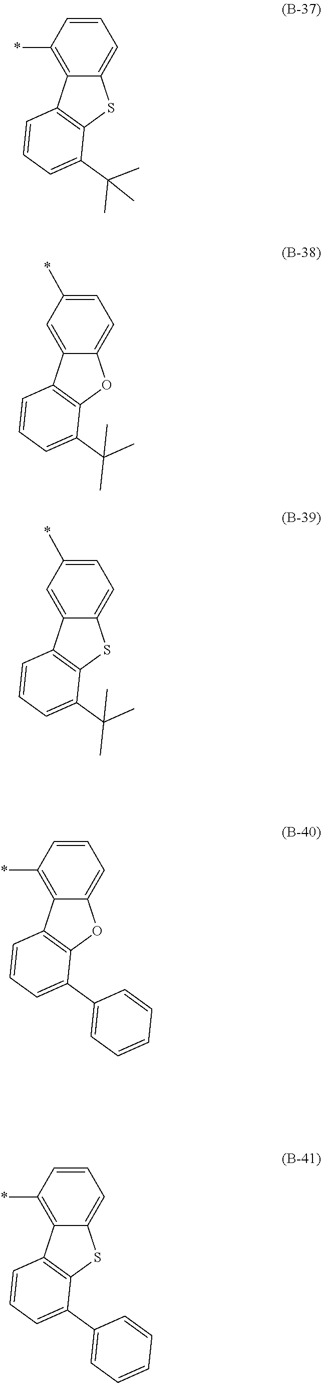 [SMILES: *c1cccc2sc3c(C(C)(C)C)cccc3c12 ; *c1ccc2oc3c(C(C)(C)C)cccc3c2c1 ; *c1ccc2sc3c(C(C)(C)C)cccc3c2c1 ; *c1cccc2oc3c(-c4ccccc4)cccc3c12 ; *c1cccc2sc3c(-c4ccccc4)cccc3c12]

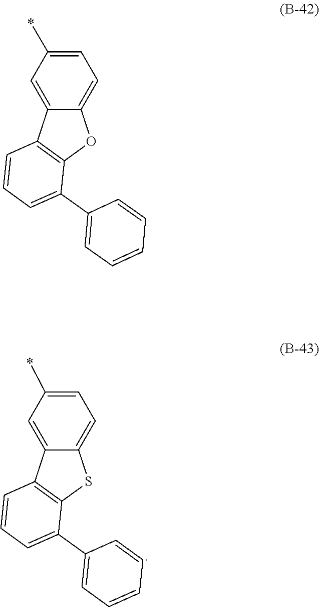 [SMILES: *c1ccc2oc3c(-c4ccccc4)cccc3c2c1 ; *c1ccc2sc3c(-c4ccccc4)cccc3c2c1]

In one embodiment, R₃ is not H. R₃ is selected from -D, a methyl group, an ethyl group, a n-propyl group, an isopropyl group, a n-butyl group, a tert-butyl group, and a phenyl group. R₁ and R₂ are both H. In detail, the Q is selected from groups represented by formula (B-44) to (B-63):

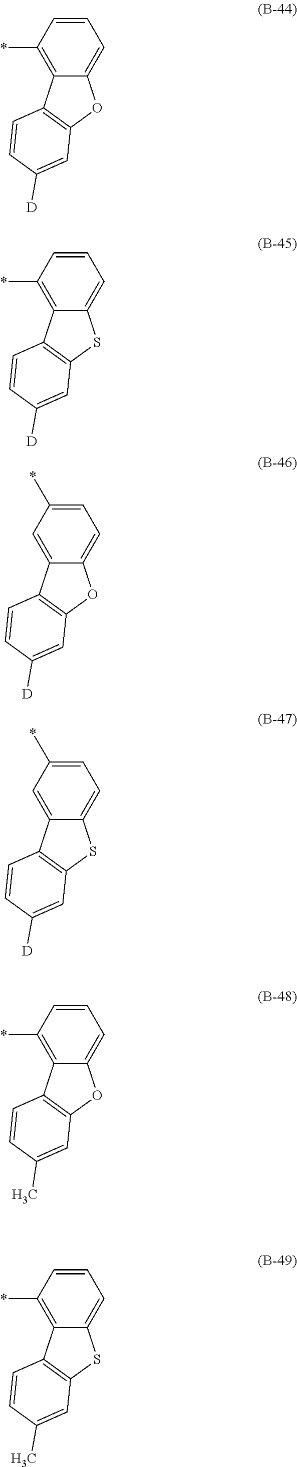

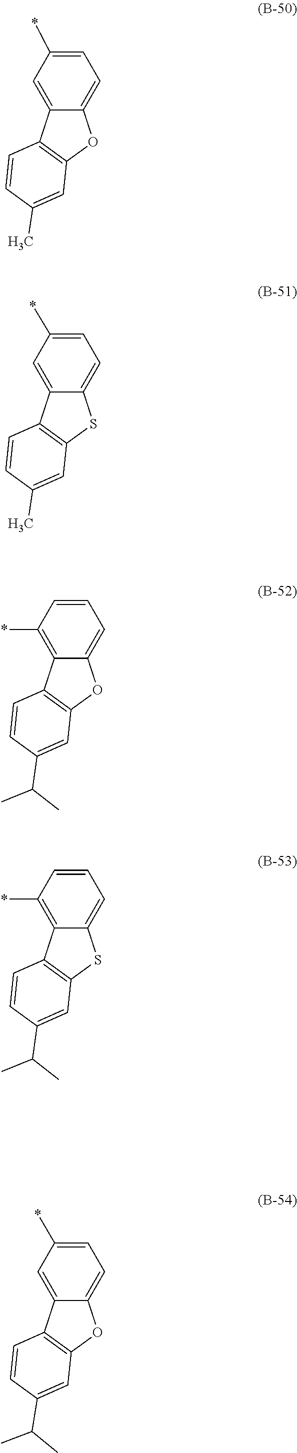 [SMILES: *c1ccc2oc3cc(C)ccc3c2c1 ; *c1ccc2sc3cc(C)ccc3c2c1 ; *c1cccc2oc3cc(C(C)C)ccc3c12 ; *c1cccc2sc3cc(C(C)C)ccc3c12 ; *c1ccc2oc3cc(C(C)C)ccc3c2c1]

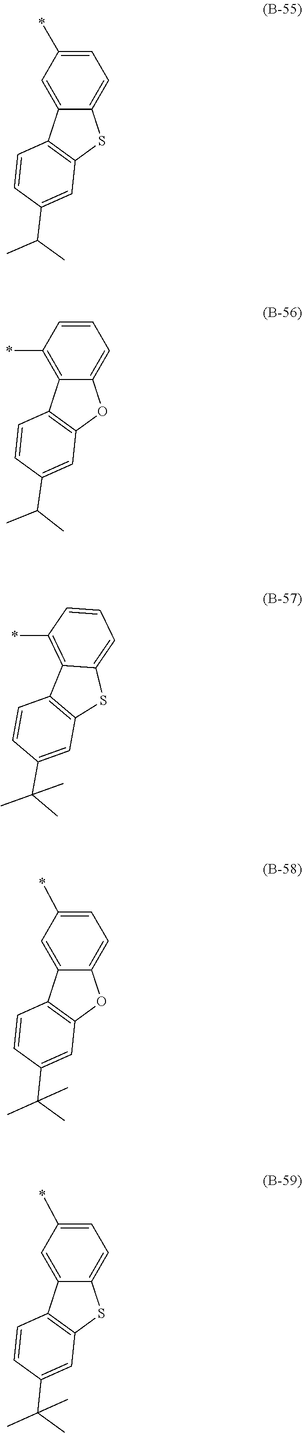 [SMILES: *c1ccc2sc3cc(C(C)C)ccc3c2c1 ; *c1cccc2oc3cc(C(C)C)ccc3c12 ; *c1cccc2sc3cc(C(C)(C)C)ccc3c12 ; *c1ccc2oc3cc(C(C)(C)C)ccc3c2c1 ; *c1ccc2sc3cc(C(C)(C)C)ccc3c2c1]

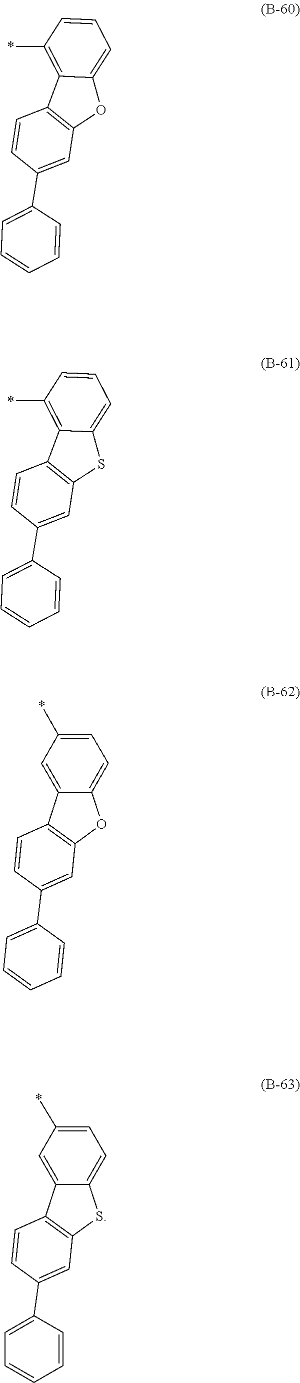 [SMILES: *c1cccc2oc3cc(-c4ccccc4)ccc3c12 ; *c1cccc2sc3cc(-c4ccccc4)ccc3c12 ; *c1ccc2oc3cc(-c4ccccc4)ccc3c2c1 ; *c1ccc2sc3cc(-c4ccccc4)ccc3c2c1]

In one embodiment, each of L₁ and L₂ is independently selected from a single bond and a phenyl group. In detail, each of L₁ and L₂ is independently selected from the single bond and groups represented by formula (C-1) to (C-3):

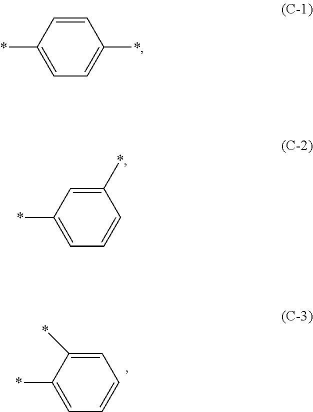

* indicates a binding site.

In one embodiment, the present application provides an anthracene-based compound, which is selected from compounds represented by formula (II-1) and formula (II-2):

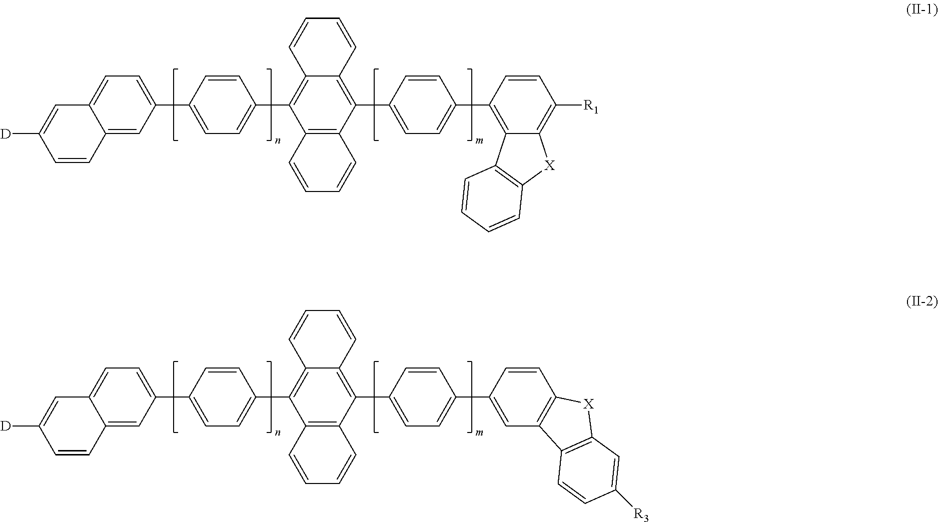

wherein n is selected from 0, 1, and 2, and m is selected from 0, 1, and 2.

In some embodiments, in the formula (II-1), R₁ is selected from —H and -D.

In some embodiments, in the formula (II-2), R₃ is selected from —H and -D. Deuterization can further prolong a lifespan of OLEDs in light-emitting.

In one embodiment, m+n=0, 1, or 2. In some embodiments, m+n=1. When m+n=1, light-emitting materials have good performances.

In one embodiment, the anthracene-based compound represented by the formula (I) are selected from, but not limited thereto:

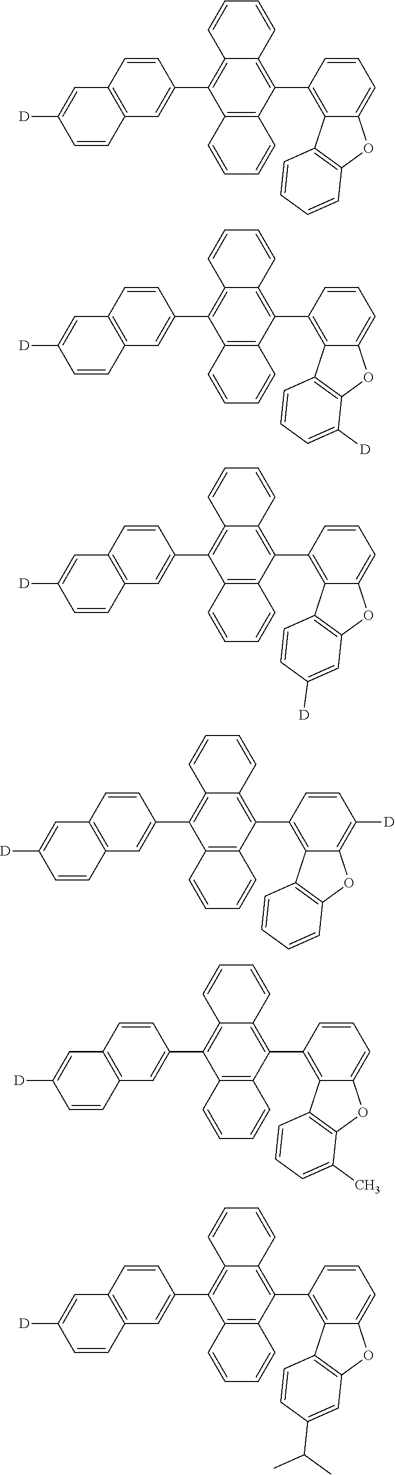

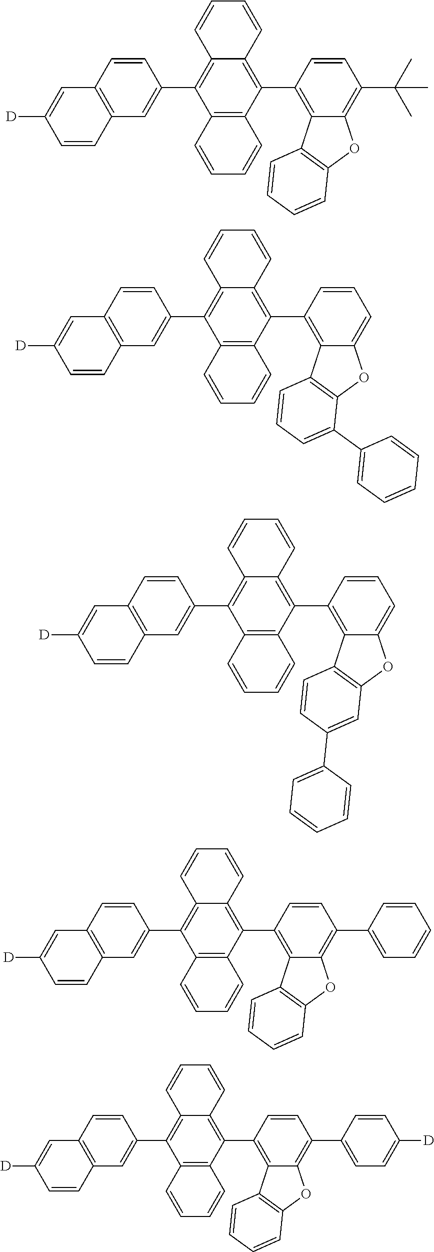

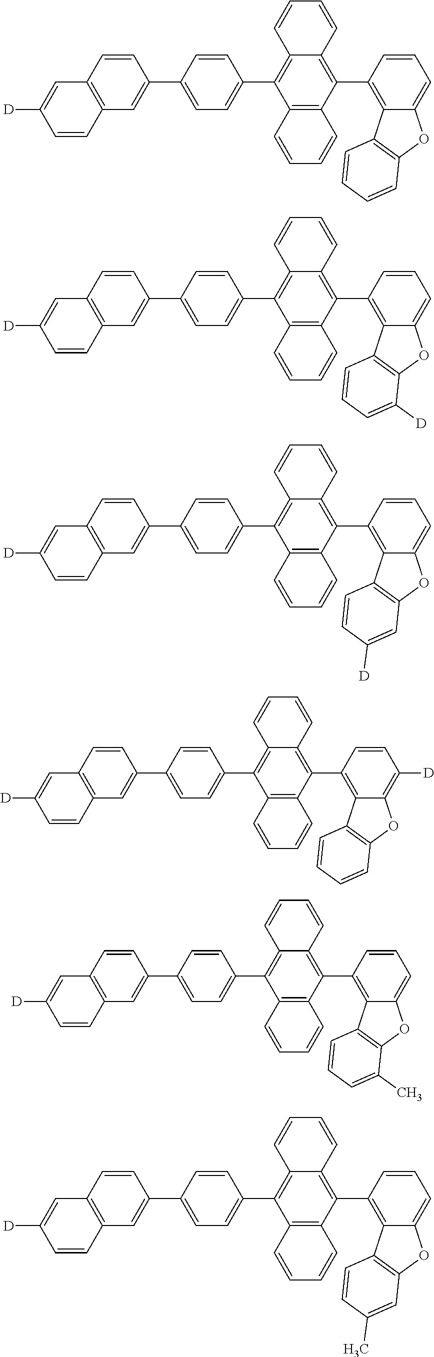

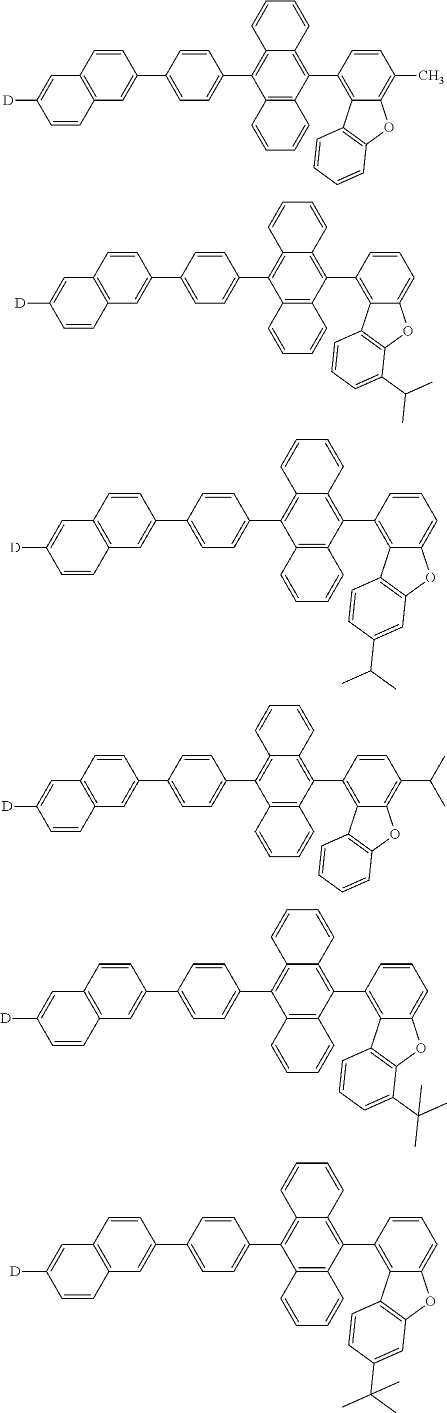

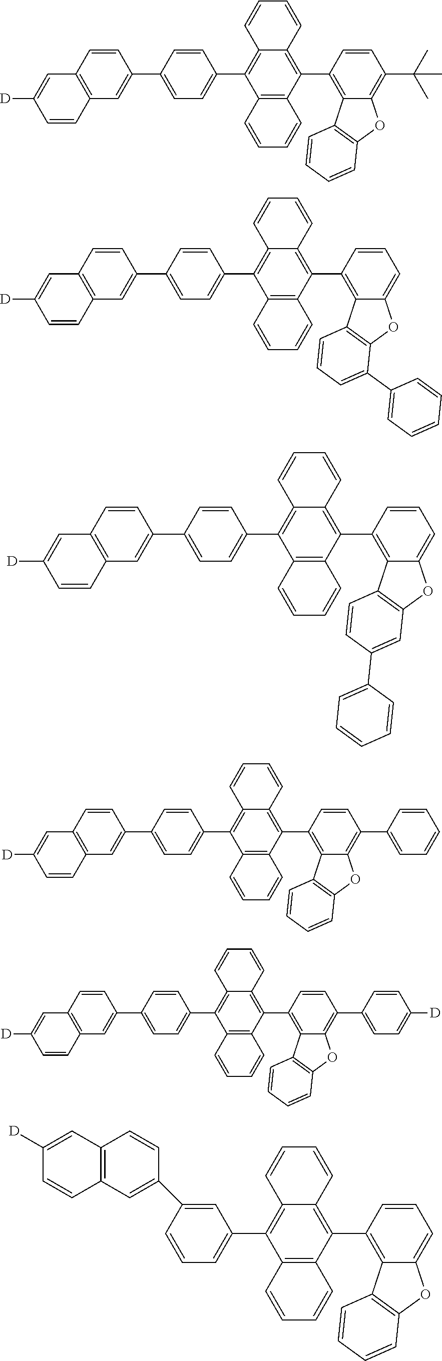

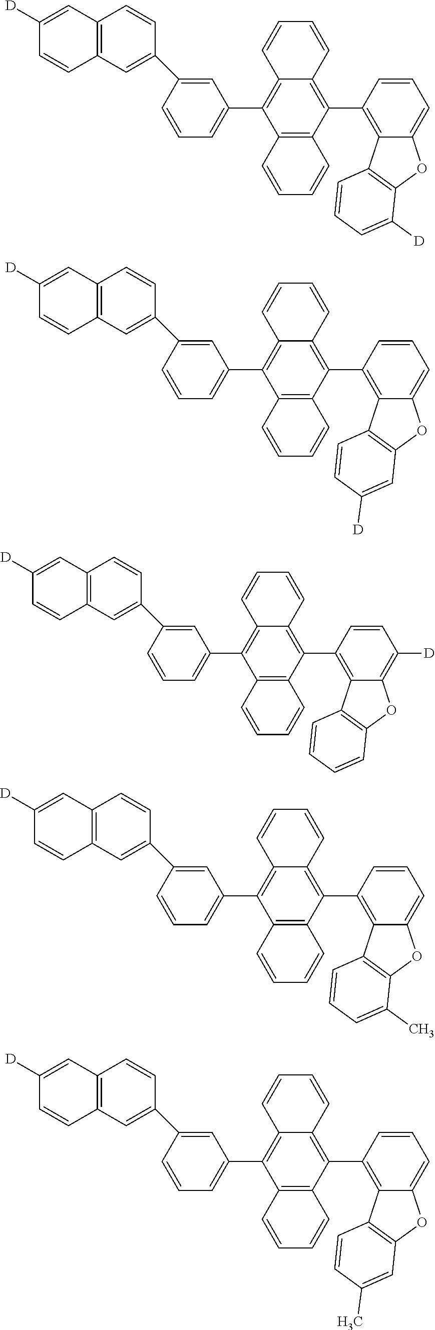

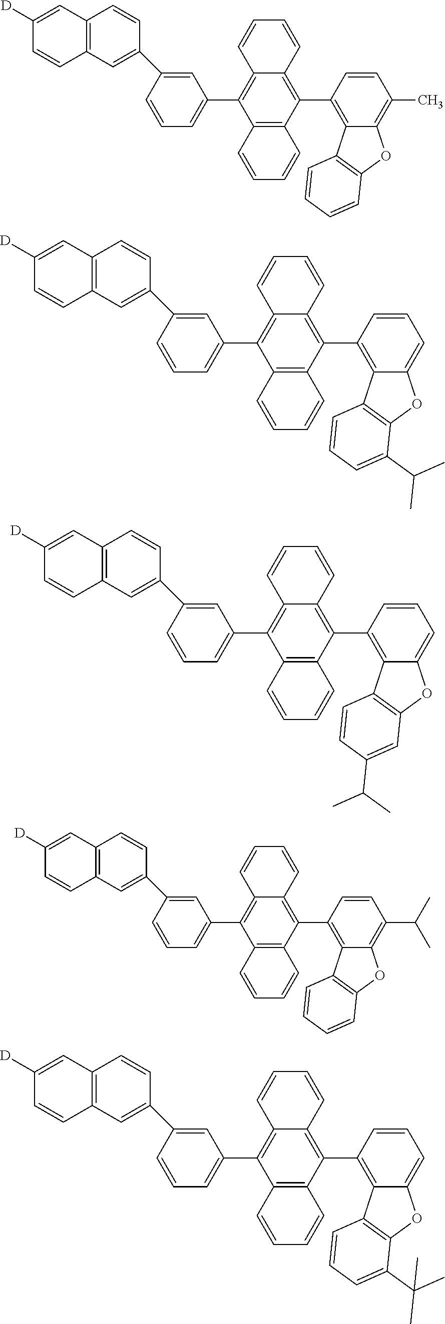

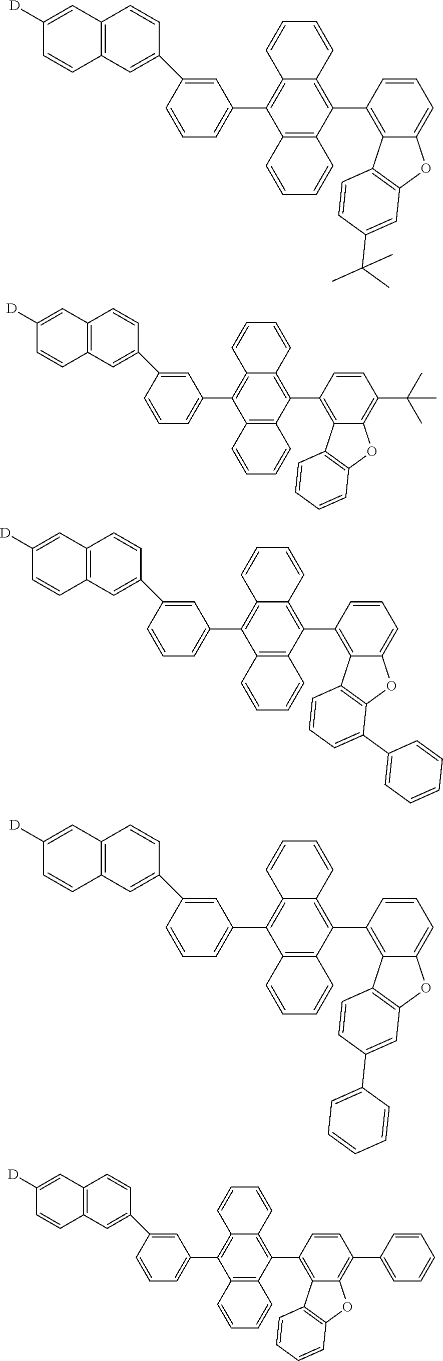 [SMILES: [2H]c1ccc2cc(-c3cccc(-c4c5ccccc5c(-c5cccc6oc7cc(C(C)(C)C)ccc7c56)c5ccccc45)c3)ccc2c1 ; [2H]c1ccc2cc(-c3cccc(-c4c5ccccc5c(-c5ccc(C(C)(C)C)c6oc7ccccc7c56)c5ccccc45)c3)ccc2c1 ; [2H]c1ccc2cc(-c3cccc(-c4c5ccccc5c(-c5cccc6oc7c(-c8ccccc8)cccc7c56)c5ccccc45)c3)ccc2c1 ; [2H]c1ccc2cc(-c3cccc(-c4c5ccccc5c(-c5cccc6oc7cc(-c8ccccc8)ccc7c56)c5ccccc45)c3)ccc2c1 ; [2H]c1ccc2cc(-c3cccc(-c4c5ccccc5c(-c5ccc(-c6ccccc6)c6oc7ccccc7c56)c5ccccc45)c3)ccc2c1]

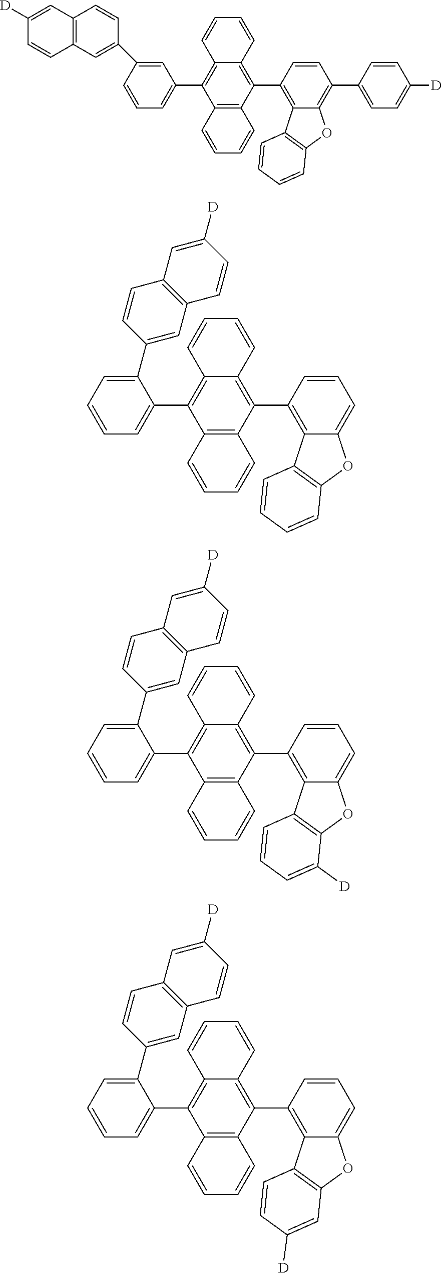 [SMILES: [2H]c1ccc(-c2ccc(-c3c4ccccc4c(-c4cccc(-c5ccc6cc([2H])ccc6c5)c4)c4ccccc34)c3c2oc2ccccc23)cc1 ; [2H]c1ccc2cc(-c3ccccc3-c3c4ccccc4c(-c4cccc5oc6ccccc6c45)c4ccccc34)ccc2c1 ; [2H]c1ccc2cc(-c3ccccc3-c3c4ccccc4c(-c4cccc5oc6c([2H])cccc6c45)c4ccccc34)ccc2c1 ; [2H]c1ccc2cc(-c3ccccc3-c3c4ccccc4c(-c4cccc5oc6cc([2H])ccc6c45)c4ccccc34)ccc2c1]

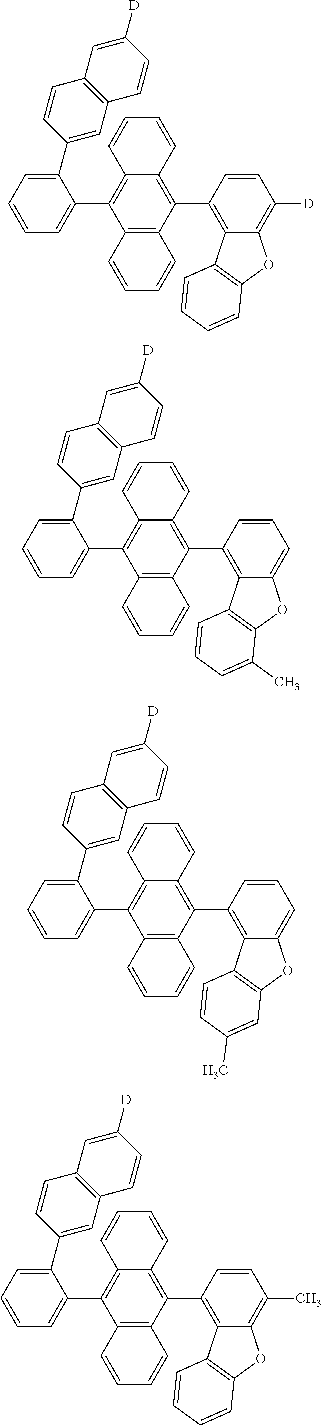 [SMILES: [2H]c1ccc2cc(-c3ccccc3-c3c4ccccc4c(-c4ccc([2H])c5oc6ccccc6c45)c4ccccc34)ccc2c1 ; [2H]c1ccc2cc(-c3ccccc3-c3c4ccccc4c(-c4cccc5oc6c(C)cccc6c45)c4ccccc34)ccc2c1 ; [2H]c1ccc2cc(-c3ccccc3-c3c4ccccc4c(-c4cccc5oc6cc(C)ccc6c45)c4ccccc34)ccc2c1 ; [2H]c1ccc2cc(-c3ccccc3-c3c4ccccc4c(-c4ccc(C)c5oc6ccccc6c45)c4ccccc34)ccc2c1]

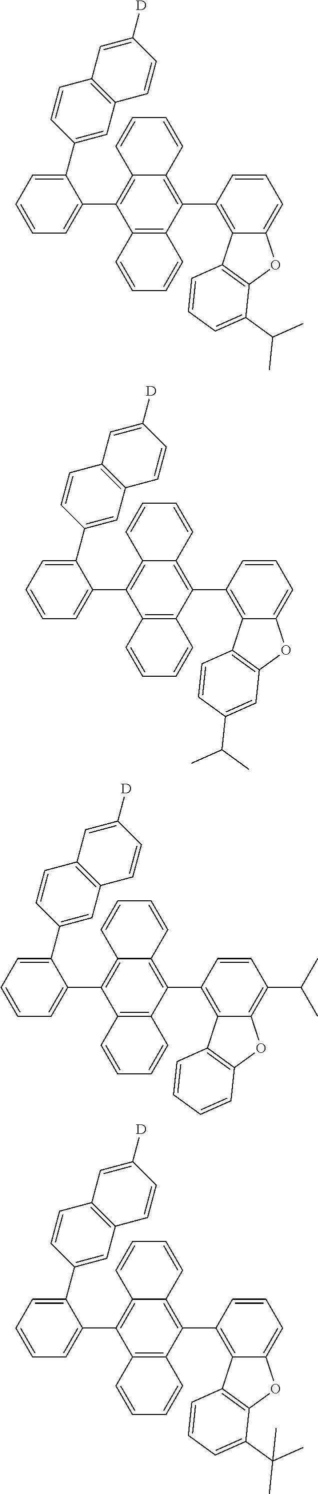 [SMILES: [2H]c1ccc2cc(-c3ccccc3-c3c4ccccc4c(-c4cccc5oc6c(C(C)C)cccc6c45)c4ccccc34)ccc2c1 ; [2H]c1ccc2cc(-c3ccccc3-c3c4ccccc4c(-c4cccc5oc6cc(C(C)C)ccc6c45)c4ccccc34)ccc2c1 ; [2H]c1ccc2cc(-c3ccccc3-c3c4ccccc4c(-c4ccc(C(C)C)c5oc6ccccc6c45)c4ccccc34)ccc2c1 ; [2H]c1ccc2cc(-c3ccccc3-c3c4ccccc4c(-c4cccc5oc6c(C(C)(C)C)cccc6c45)c4ccccc34)ccc2c1]

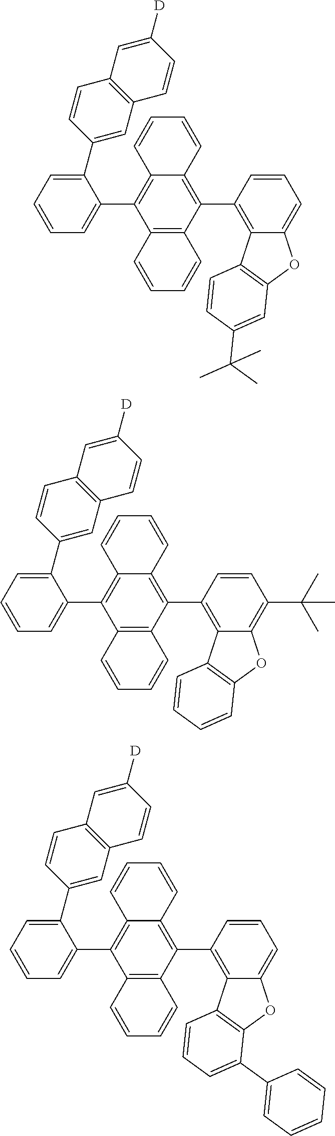 [SMILES: [2H]c1ccc2cc(-c3ccccc3-c3c4ccccc4c(-c4cccc5oc6cc(C(C)(C)C)ccc6c45)c4ccccc34)ccc2c1 ; [2H]c1ccc2cc(-c3ccccc3-c3c4ccccc4c(-c4ccc(C(C)(C)C)c5oc6ccccc6c45)c4ccccc34)ccc2c1 ; [2H]c1ccc2cc(-c3ccccc3-c3c4ccccc4c(-c4cccc5oc6c(-c7ccccc7)cccc6c45)c4ccccc34)ccc2c1]

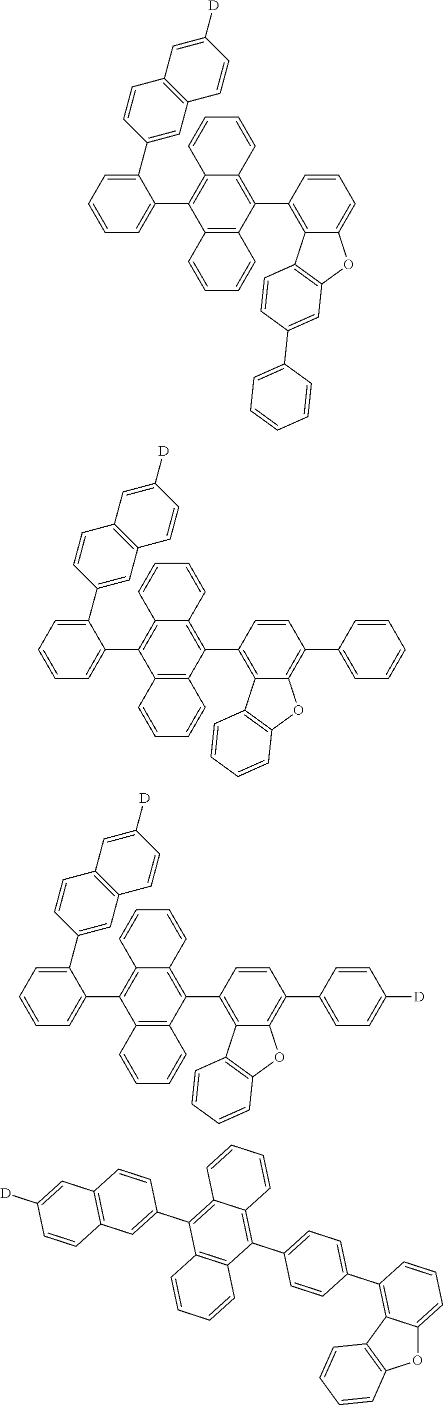

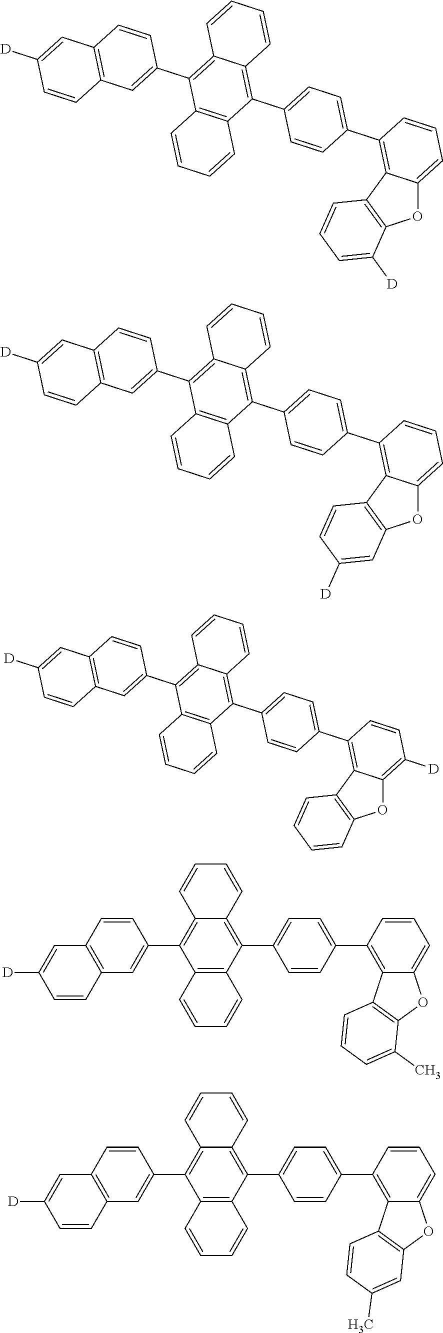 [SMILES: [2H]c1ccc2cc(-c3c4ccccc4c(-c4ccc(-c5cccc6oc7c([2H])cccc7c56)cc4)c4ccccc34)ccc2c1 ; [2H]c1ccc2cc(-c3c4ccccc4c(-c4ccc(-c5cccc6oc7cc([2H])ccc7c56)cc4)c4ccccc34)ccc2c1 ; [2H]c1ccc2cc(-c3c4ccccc4c(-c4ccc(-c5ccc([2H])c6oc7ccccc7c56)cc4)c4ccccc34)ccc2c1 ; [2H]c1ccc2cc(-c3c4ccccc4c(-c4ccc(-c5cccc6oc7c(C)cccc7c56)cc4)c4ccccc34)ccc2c1 ; [2H]c1ccc2cc(-c3c4ccccc4c(-c4ccc(-c5cccc6oc7cc(C)ccc7c56)cc4)c4ccccc34)ccc2c1]

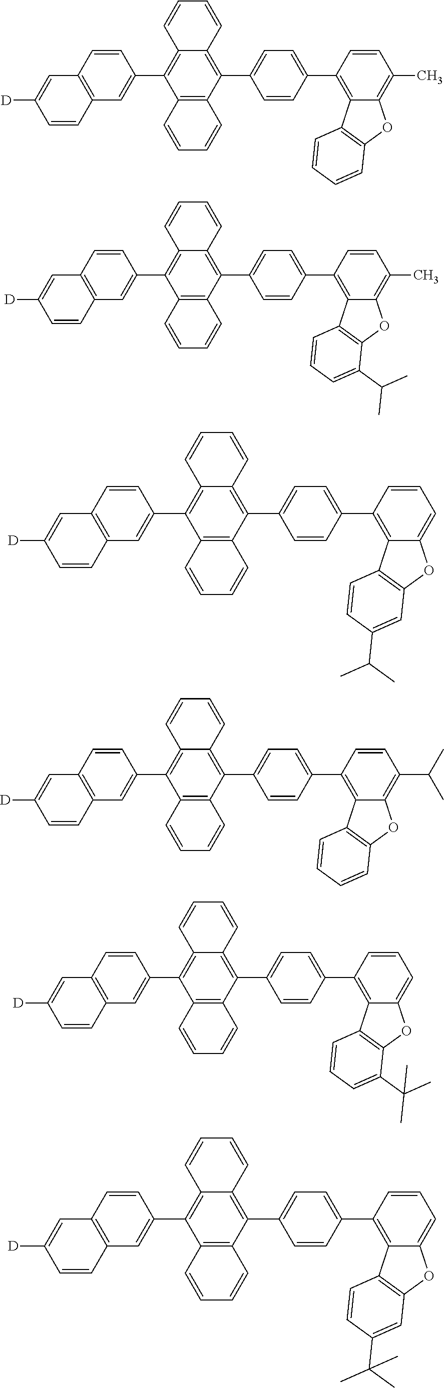

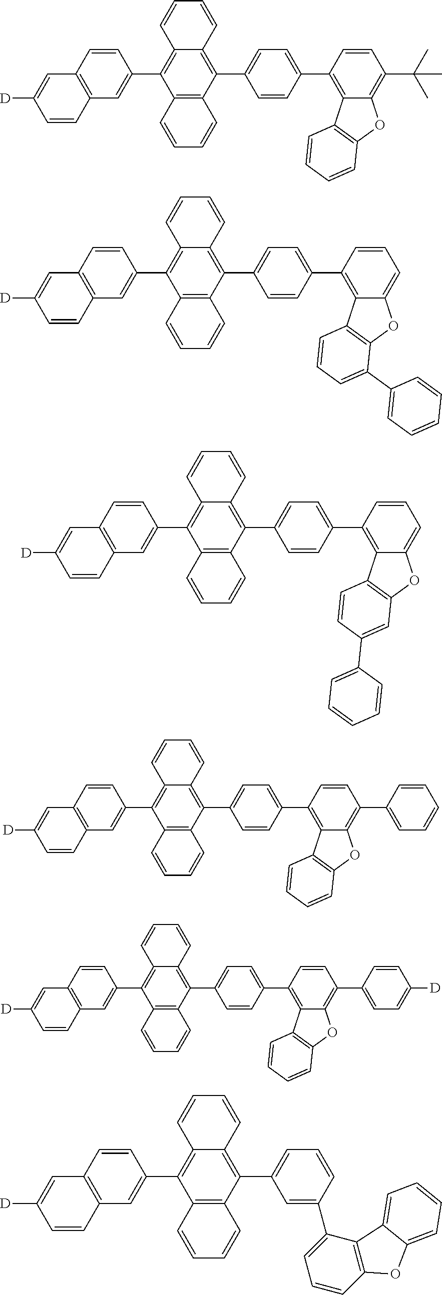

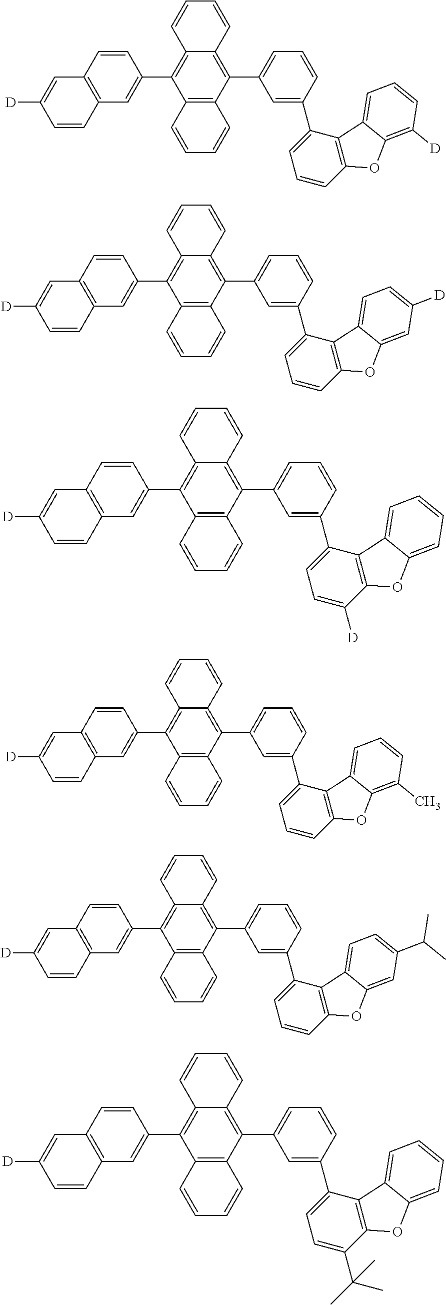 [SMILES: [2H]c1ccc2cc(-c3c4ccccc4c(-c4cccc(-c5cccc6oc7c([2H])cccc7c56)c4)c4ccccc34)ccc2c1 ; [2H]c1ccc2cc(-c3c4ccccc4c(-c4cccc(-c5cccc6oc7cc([2H])ccc7c56)c4)c4ccccc34)ccc2c1 ; [2H]c1ccc2cc(-c3c4ccccc4c(-c4cccc(-c5ccc([2H])c6oc7ccccc7c56)c4)c4ccccc34)ccc2c1 ; [2H]c1ccc2cc(-c3c4ccccc4c(-c4cccc(-c5cccc6oc7c(C)cccc7c56)c4)c4ccccc34)ccc2c1 ; [2H]c1ccc2cc(-c3c4ccccc4c(-c4cccc(-c5cccc6oc7cc(C(C)C)ccc7c56)c4)c4ccccc34)ccc2c1 ; [2H]c1ccc2cc(-c3c4ccccc4c(-c4cccc(-c5ccc(C(C)(C)C)c6oc7ccccc7c56)c4)c4ccccc34)ccc2c1]

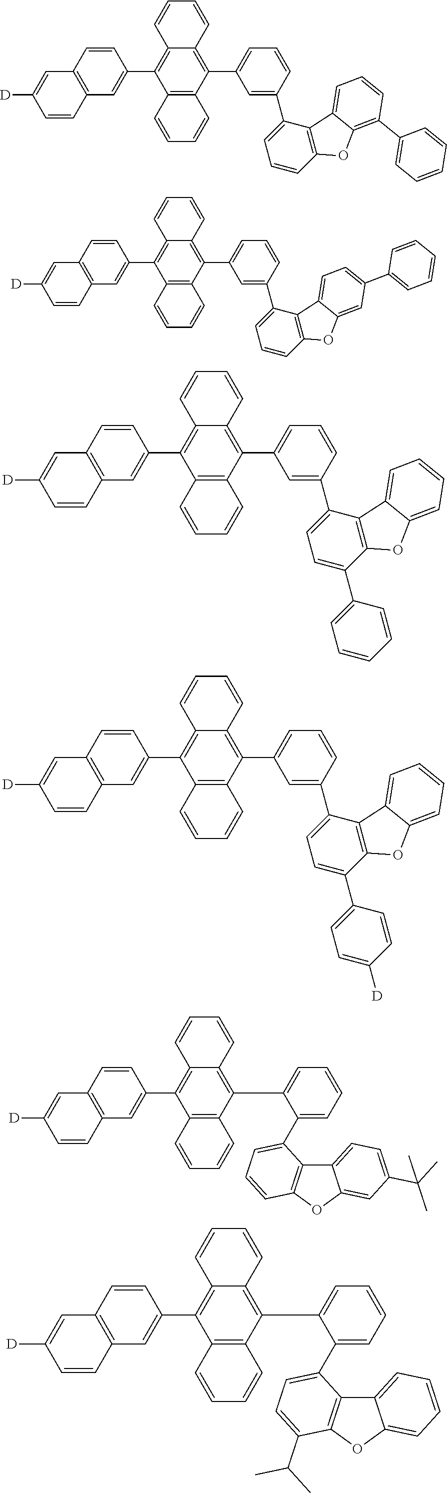

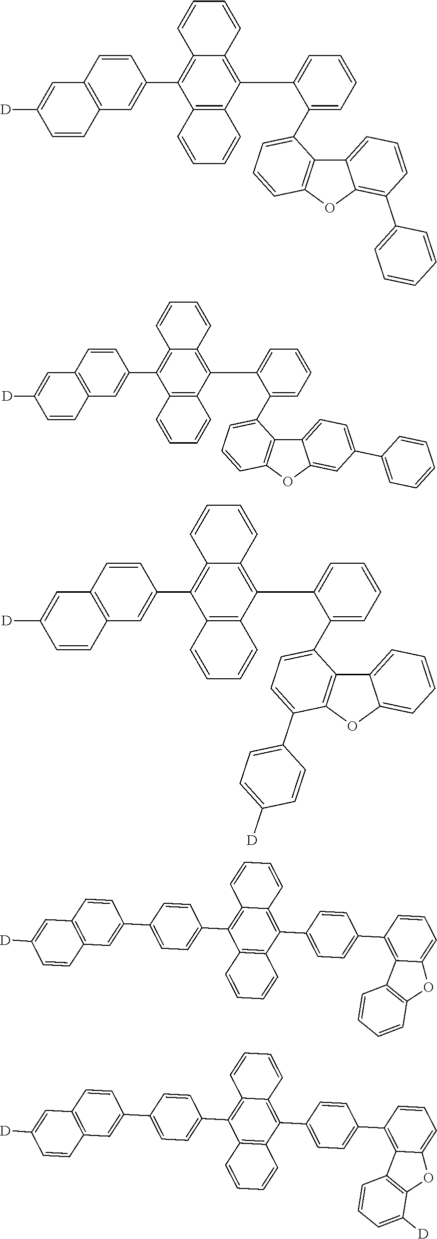

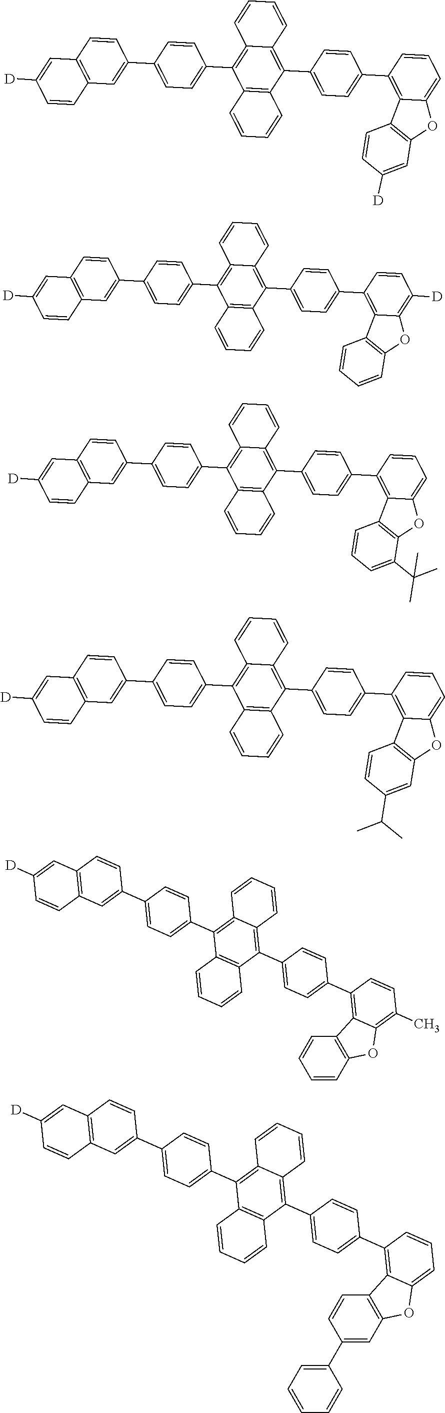

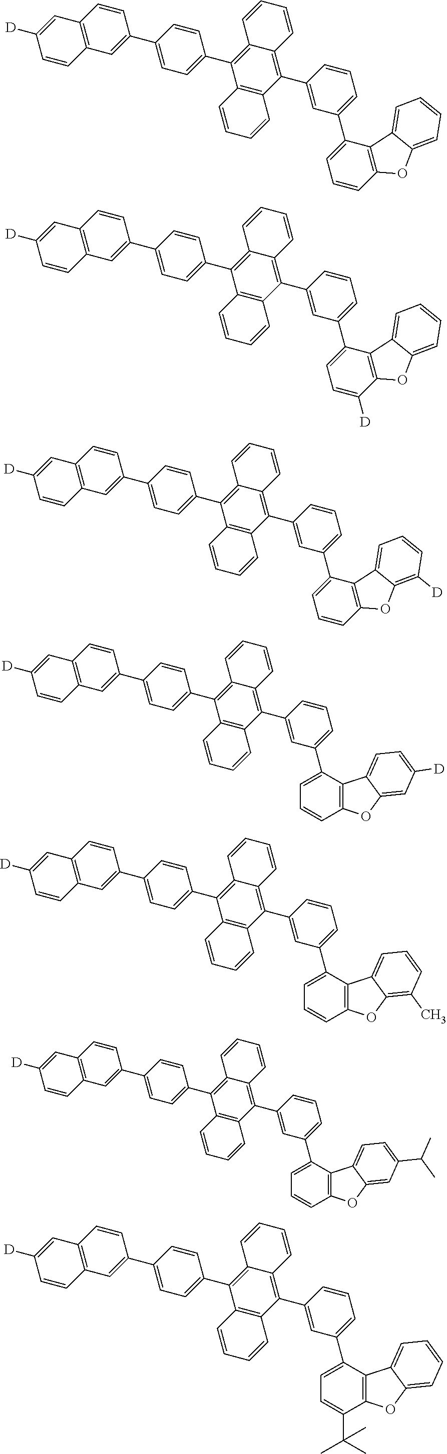

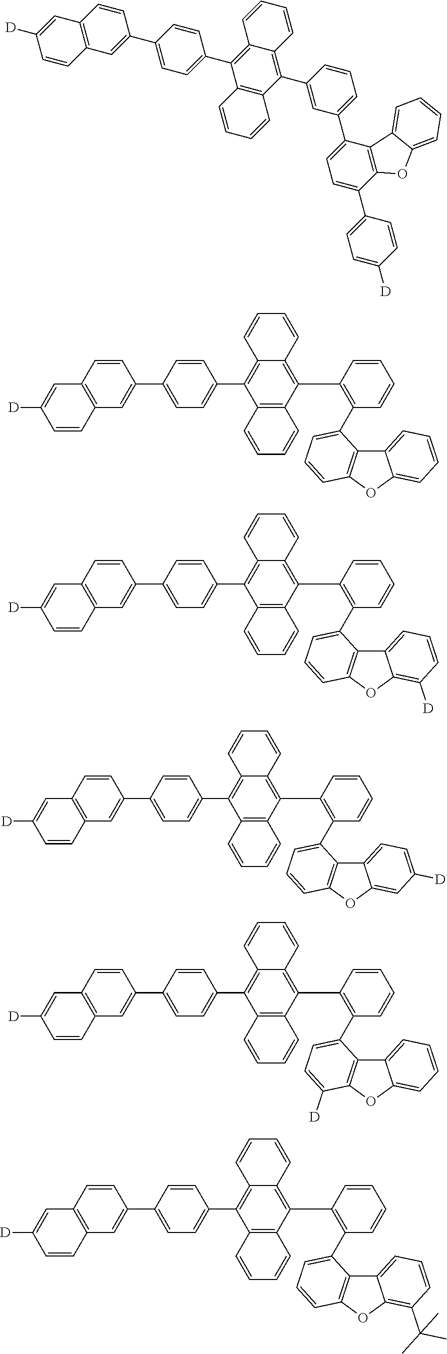

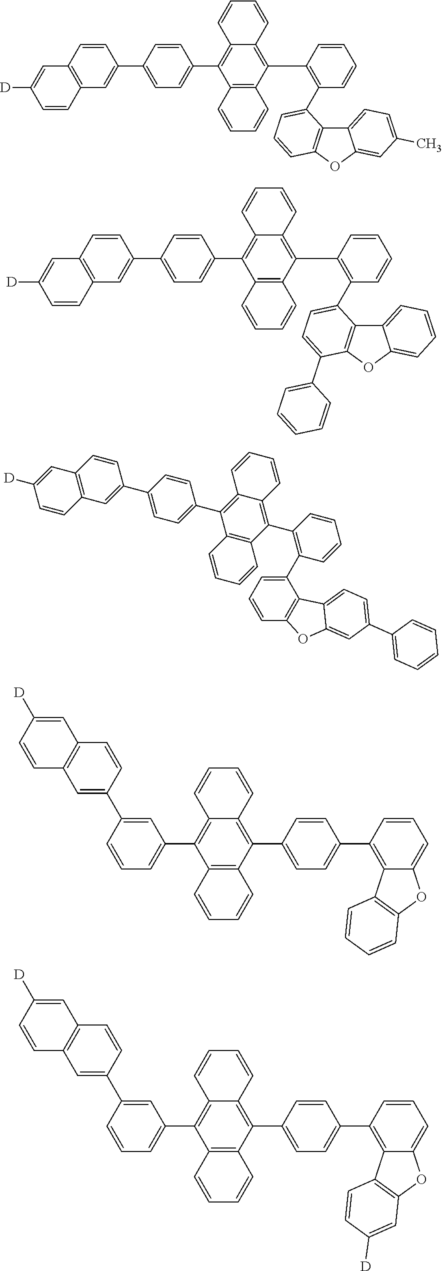

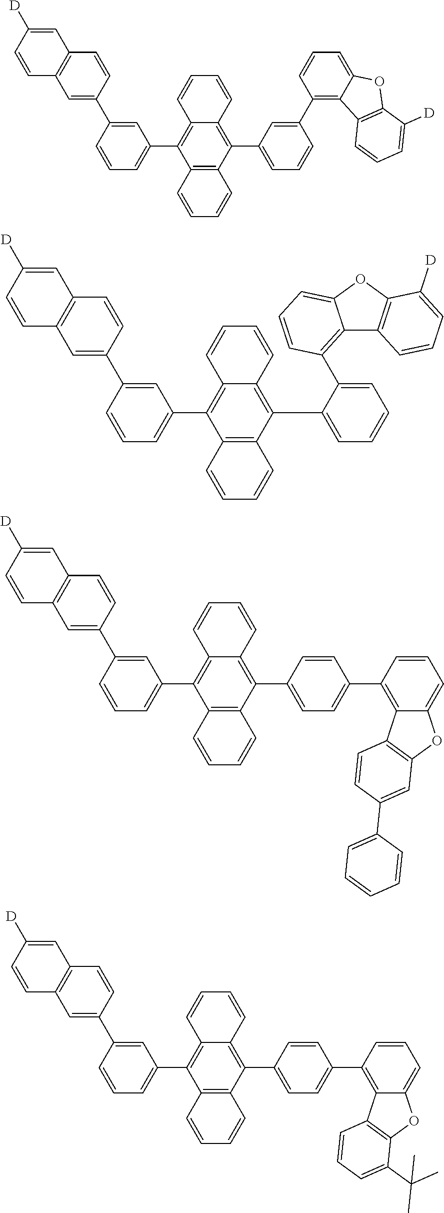 [SMILES: [2H]c1ccc2cc(-c3cccc(-c4c5ccccc5c(-c5cccc(-c6cccc7oc8c([2H])cccc8c67)c5)c5ccccc45)c3)ccc2c1 ; [2H]c1ccc2cc(-c3cccc(-c4c5ccccc5c(-c5ccccc5-c5cccc6oc7c([2H])cccc7c56)c5ccccc45)c3)ccc2c1 ; [2H]c1ccc2cc(-c3cccc(-c4c5ccccc5c(-c5ccc(-c6cccc7oc8cc(-c9ccccc9)ccc8c67)cc5)c5ccccc45)c3)ccc2c1 ; [2H]c1ccc2cc(-c3cccc(-c4c5ccccc5c(-c5ccc(-c6cccc7oc8c(C(C)(C)C)cccc8c67)cc5)c5ccccc45)c3)ccc2c1]

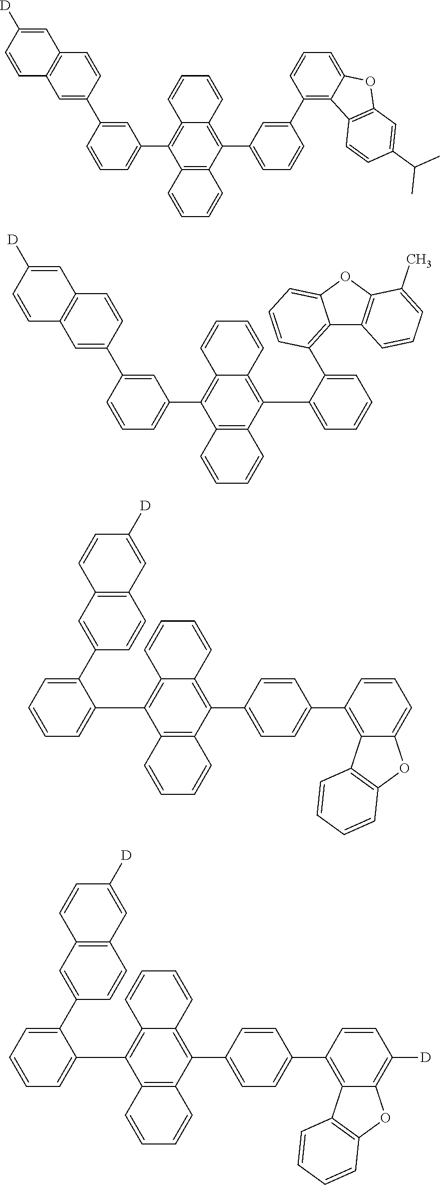 [SMILES: [2H]c1ccc2cc(-c3cccc(-c4c5ccccc5c(-c5cccc(-c6cccc7oc8cc(C(C)C)ccc8c67)c5)c5ccccc45)c3)ccc2c1 ; [2H]c1ccc2cc(-c3cccc(-c4c5ccccc5c(-c5ccccc5-c5cccc6oc7c(C)cccc7c56)c5ccccc45)c3)ccc2c1 ; [2H]c1ccc2cc(-c3ccccc3-c3c4ccccc4c(-c4ccc(-c5cccc6oc7ccccc7c56)cc4)c4ccccc34)ccc2c1 ; [2H]c1ccc2cc(-c3ccccc3-c3c4ccccc4c(-c4ccc(-c5ccc([2H])c6oc7ccccc7c56)cc4)c4ccccc34)ccc2c1]

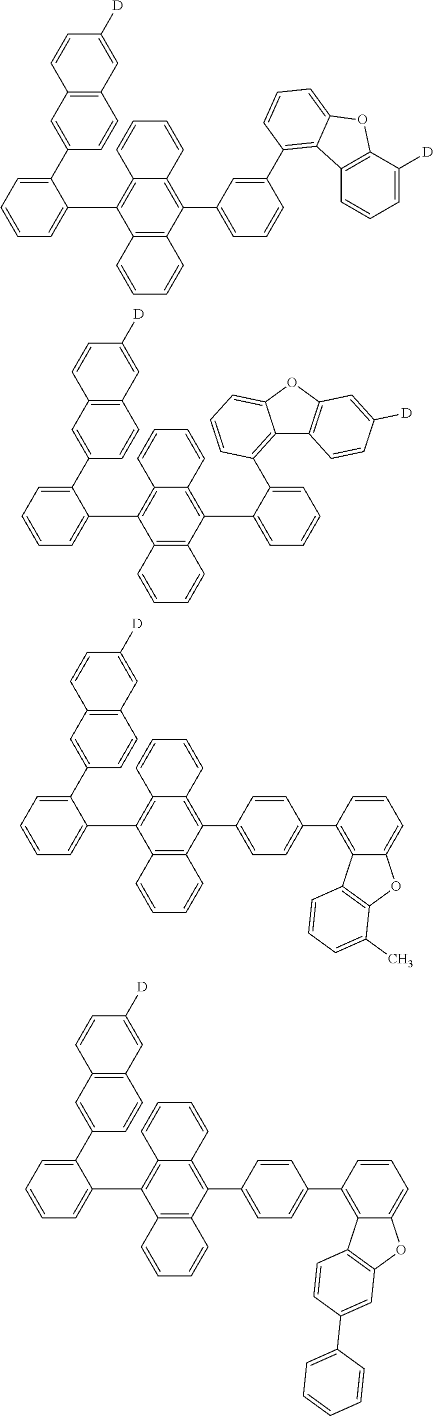

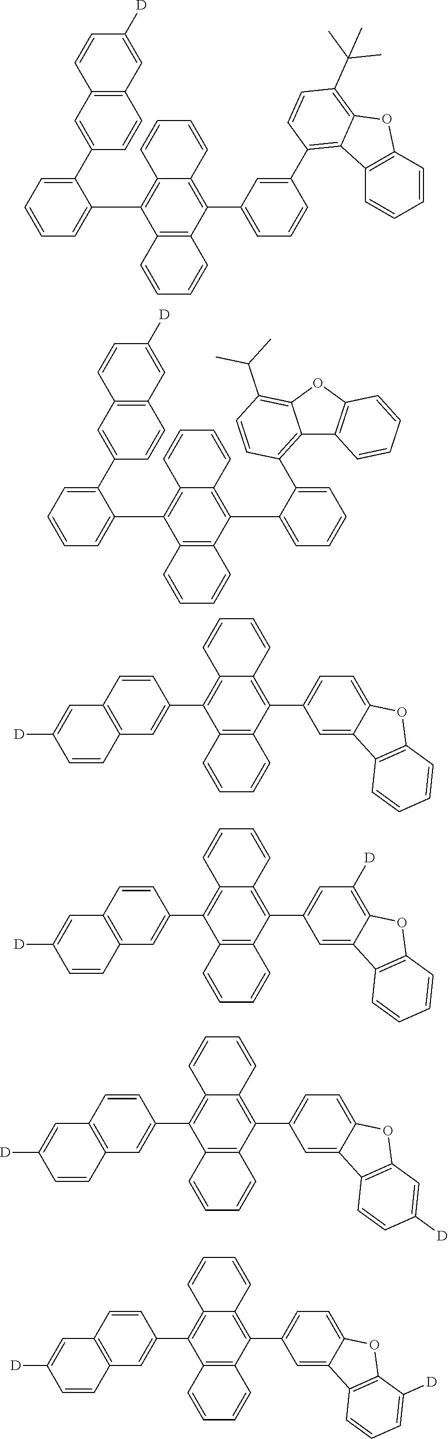

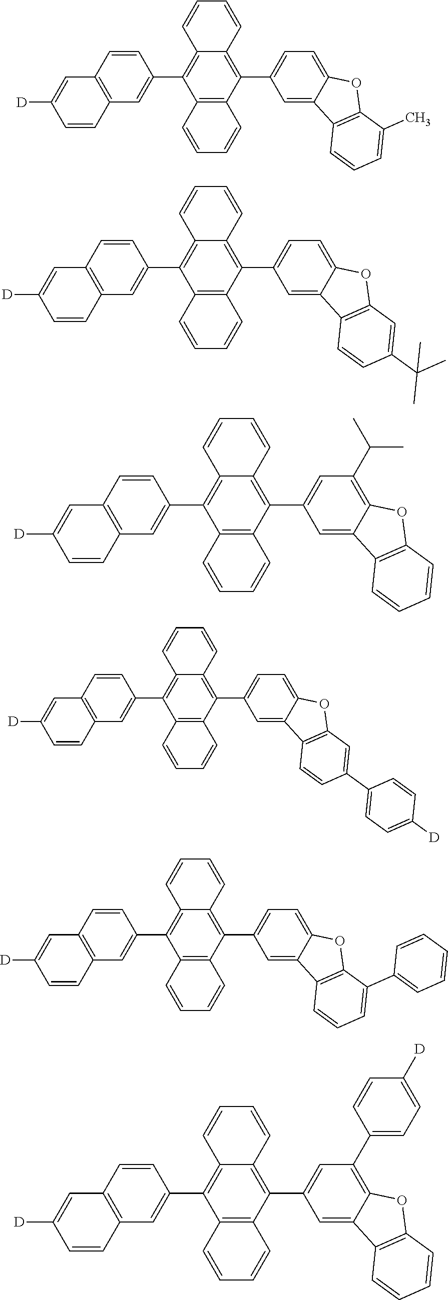

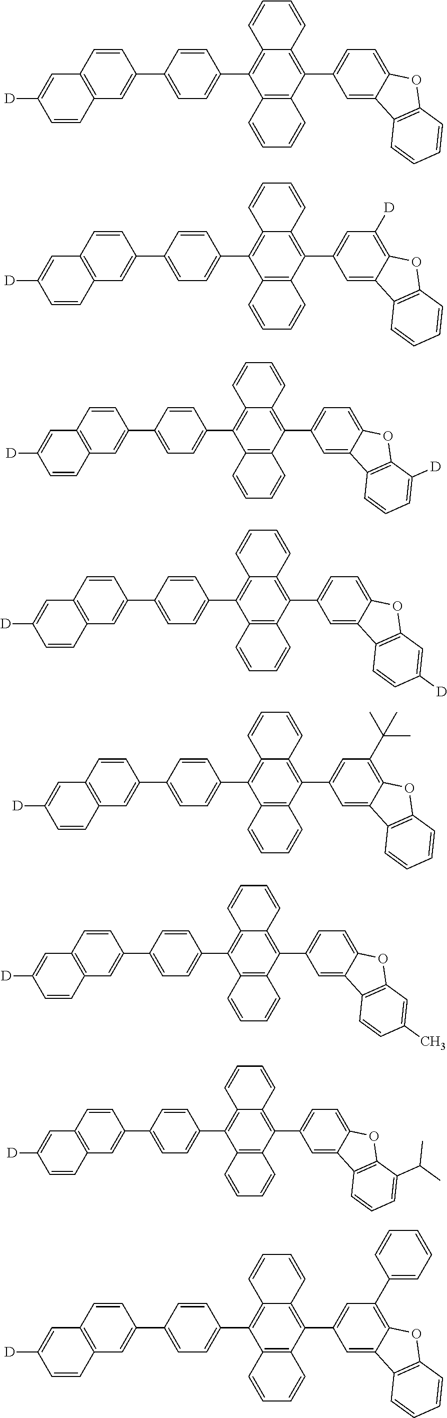

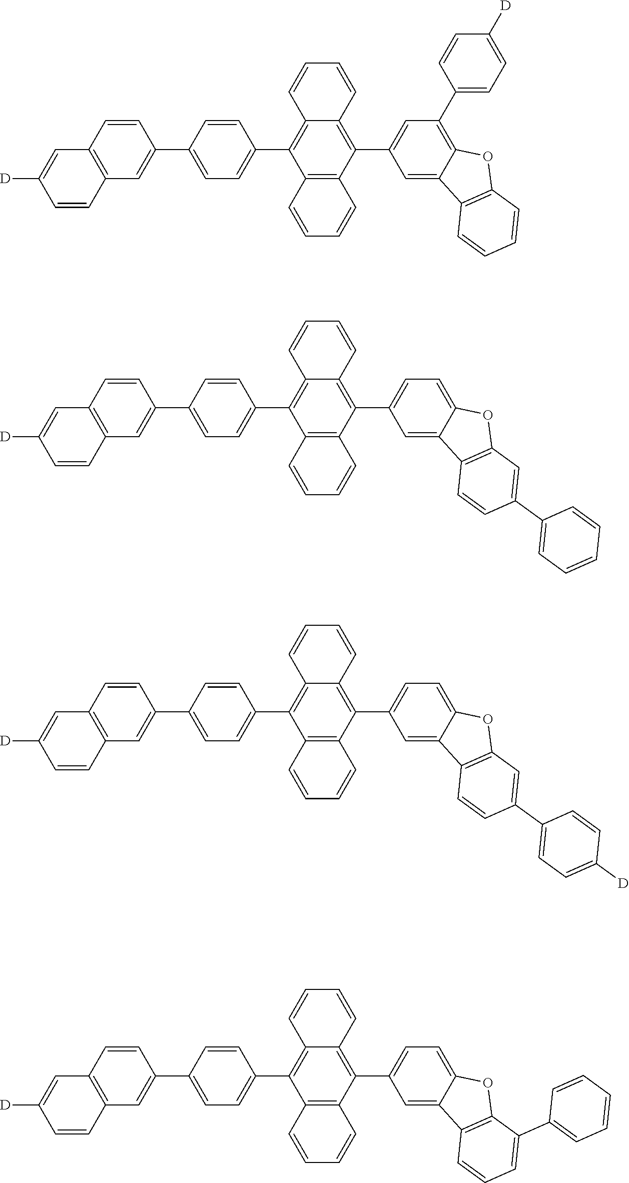 [SMILES: [2H]c1ccc(-c2cc(-c3c4ccccc4c(-c4ccc(-c5ccc6cc([2H])ccc6c5)cc4)c4ccccc34)cc3c2oc2ccccc23)cc1 ; [2H]c1ccc2cc(-c3ccc(-c4c5ccccc5c(-c5ccc6oc7cc(-c8ccccc8)ccc7c6c5)c5ccccc45)cc3)ccc2c1 ; [2H]c1ccc(-c2ccc3c(c2)oc2ccc(-c4c5ccccc5c(-c5ccc(-c6ccc7cc([2H])ccc7c6)cc5)c5ccccc45)cc23)cc1 ; [2H]c1ccc2cc(-c3ccc(-c4c5ccccc5c(-c5ccc6oc7c(-c8ccccc8)cccc7c6c5)c5ccccc45)cc3)ccc2c1]

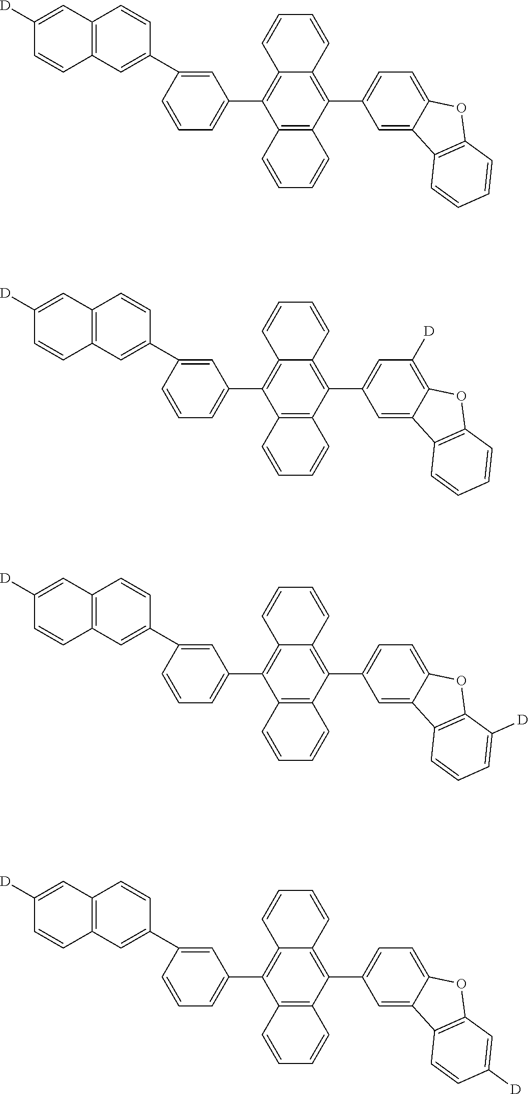 [SMILES: [2H]c1ccc2cc(-c3cccc(-c4c5ccccc5c(-c5ccc6oc7ccccc7c6c5)c5ccccc45)c3)ccc2c1 ; [2H]c1ccc2cc(-c3cccc(-c4c5ccccc5c(-c5cc([2H])c6oc7ccccc7c6c5)c5ccccc45)c3)ccc2c1 ; [2H]c1ccc2cc(-c3cccc(-c4c5ccccc5c(-c5ccc6oc7c([2H])cccc7c6c5)c5ccccc45)c3)ccc2c1 ; [2H]c1ccc2cc(-c3cccc(-c4c5ccccc5c(-c5ccc6oc7cc([2H])ccc7c6c5)c5ccccc45)c3)ccc2c1]

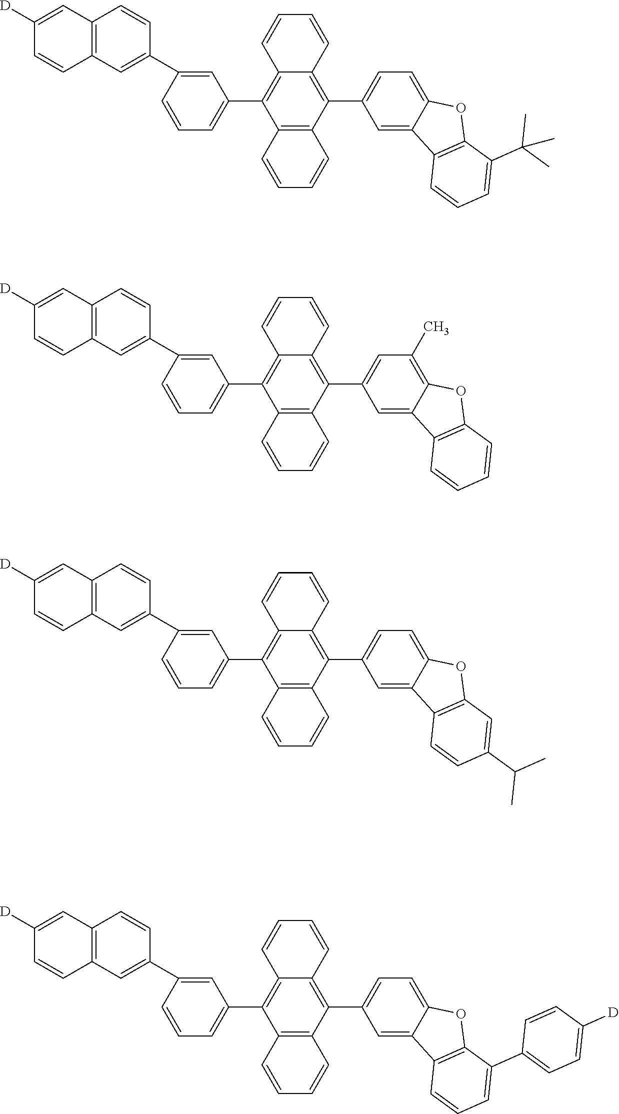 [SMILES: [2H]c1ccc2cc(-c3cccc(-c4c5ccccc5c(-c5ccc6oc7c(C(C)(C)C)cccc7c6c5)c5ccccc45)c3)ccc2c1 ; [2H]c1ccc2cc(-c3cccc(-c4c5ccccc5c(-c5cc(C)c6oc7ccccc7c6c5)c5ccccc45)c3)ccc2c1 ; [2H]c1ccc2cc(-c3cccc(-c4c5ccccc5c(-c5ccc6oc7cc(C(C)C)ccc7c6c5)c5ccccc45)c3)ccc2c1 ; [2H]c1ccc(-c2cccc3c2oc2ccc(-c4c5ccccc5c(-c5cccc(-c6ccc7cc([2H])ccc7c6)c5)c5ccccc45)cc23)cc1]

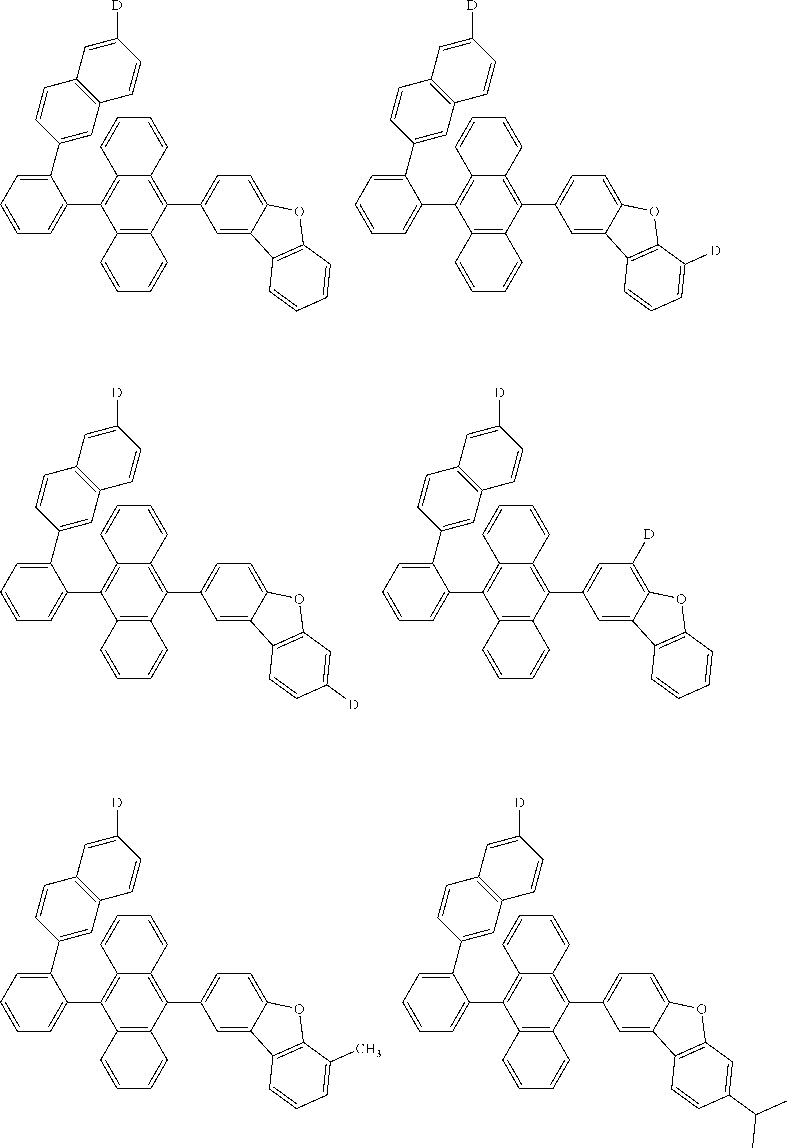 [SMILES: [2H]c1ccc2cc(-c3ccccc3-c3c4ccccc4c(-c4ccc5oc6ccccc6c5c4)c4ccccc34)ccc2c1 ; [2H]c1ccc2cc(-c3ccccc3-c3c4ccccc4c(-c4ccc5oc6c([2H])cccc6c5c4)c4ccccc34)ccc2c1 ; [2H]c1ccc2cc(-c3ccccc3-c3c4ccccc4c(-c4ccc5oc6cc([2H])ccc6c5c4)c4ccccc34)ccc2c1 ; [2H]c1ccc2cc(-c3ccccc3-c3c4ccccc4c(-c4cc([2H])c5oc6ccccc6c5c4)c4ccccc34)ccc2c1 ; [2H]c1ccc2cc(-c3ccccc3-c3c4ccccc4c(-c4ccc5oc6c(C)cccc6c5c4)c4ccccc34)ccc2c1 ; [2H]c1ccc2cc(-c3ccccc3-c3c4ccccc4c(-c4ccc5oc6cc(C(C)C)ccc6c5c4)c4ccccc34)ccc2c1]

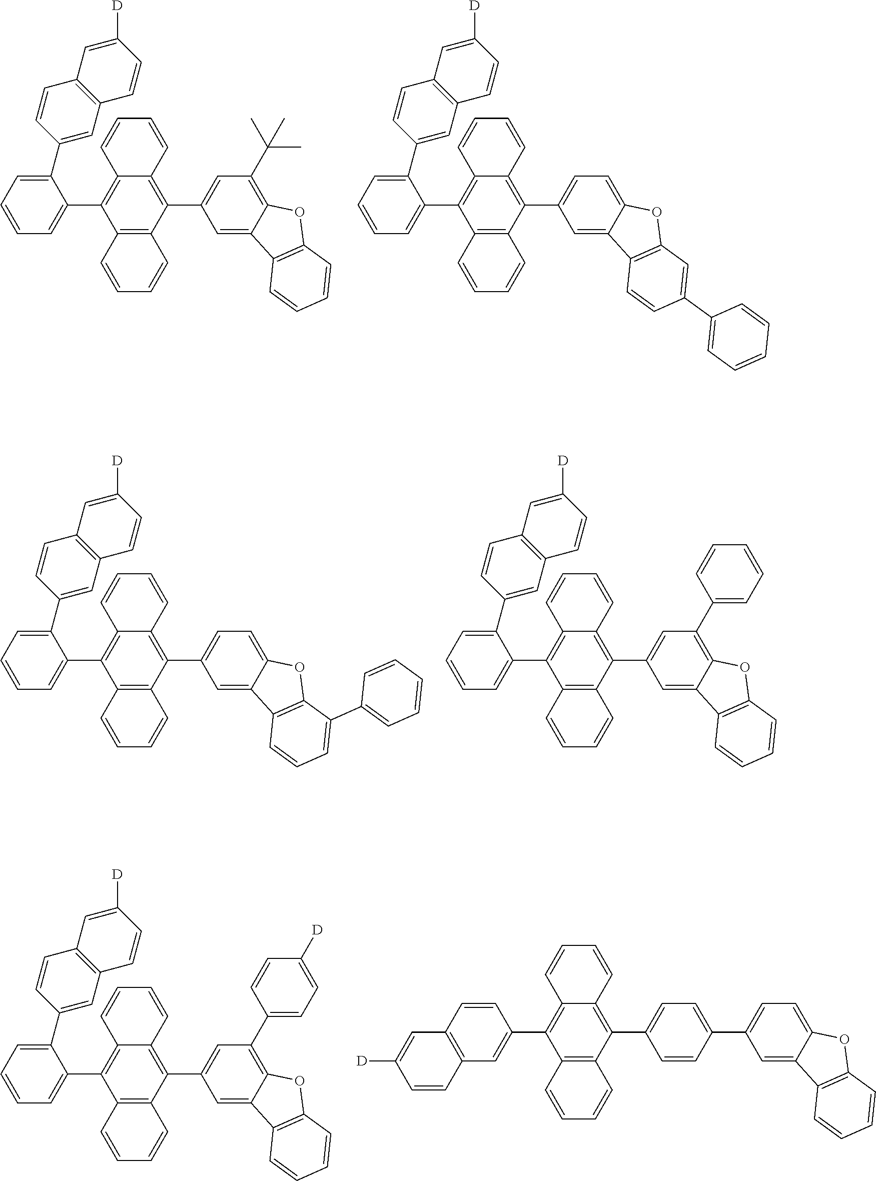

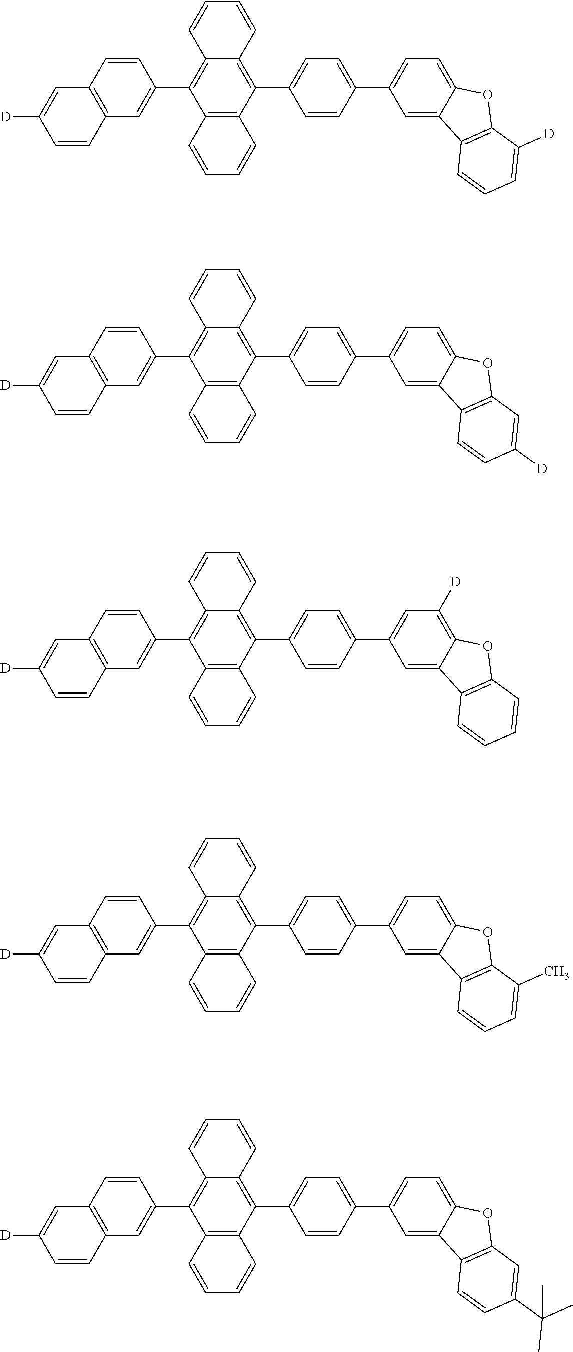 [SMILES: [2H]c1ccc2cc(-c3c4ccccc4c(-c4ccc(-c5ccc6oc7c([2H])cccc7c6c5)cc4)c4ccccc34)ccc2c1 ; [2H]c1ccc2cc(-c3c4ccccc4c(-c4ccc(-c5ccc6oc7cc([2H])ccc7c6c5)cc4)c4ccccc34)ccc2c1 ; [2H]c1ccc2cc(-c3c4ccccc4c(-c4ccc(-c5cc([2H])c6oc7ccccc7c6c5)cc4)c4ccccc34)ccc2c1 ; [2H]c1ccc2cc(-c3c4ccccc4c(-c4ccc(-c5ccc6oc7c(C)cccc7c6c5)cc4)c4ccccc34)ccc2c1 ; [2H]c1ccc2cc(-c3c4ccccc4c(-c4ccc(-c5ccc6oc7cc(C(C)(C)C)ccc7c6c5)cc4)c4ccccc34)ccc2c1]

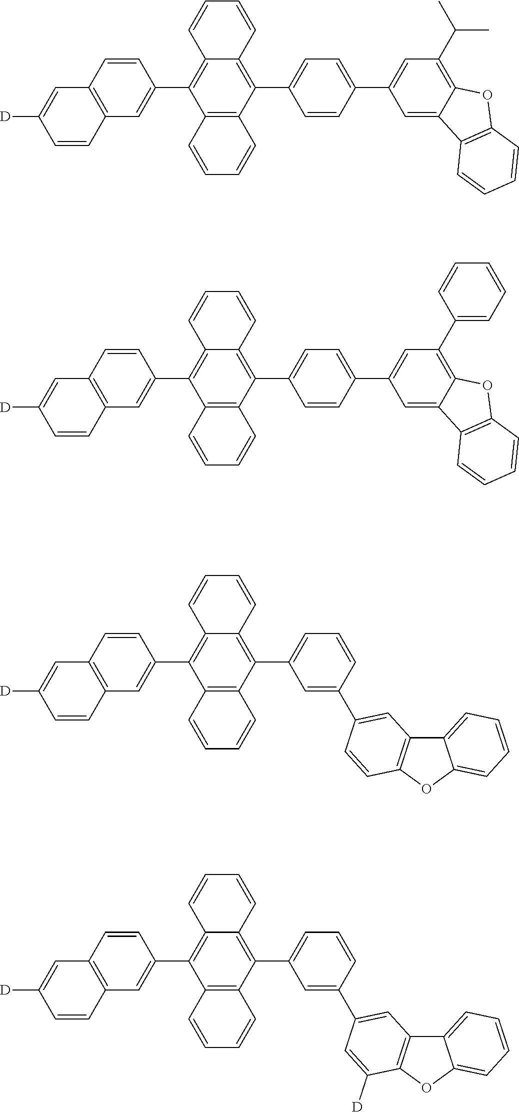 [SMILES: [2H]c1ccc2cc(-c3c4ccccc4c(-c4ccc(-c5cc(C(C)C)c6oc7ccccc7c6c5)cc4)c4ccccc34)ccc2c1 ; [2H]c1ccc2cc(-c3c4ccccc4c(-c4ccc(-c5cc(-c6ccccc6)c6oc7ccccc7c6c5)cc4)c4ccccc34)ccc2c1 ; [2H]c1ccc2cc(-c3c4ccccc4c(-c4cccc(-c5ccc6oc7ccccc7c6c5)c4)c4ccccc34)ccc2c1 ; [2H]c1ccc2cc(-c3c4ccccc4c(-c4cccc(-c5cc([2H])c6oc7ccccc7c6c5)c4)c4ccccc34)ccc2c1]

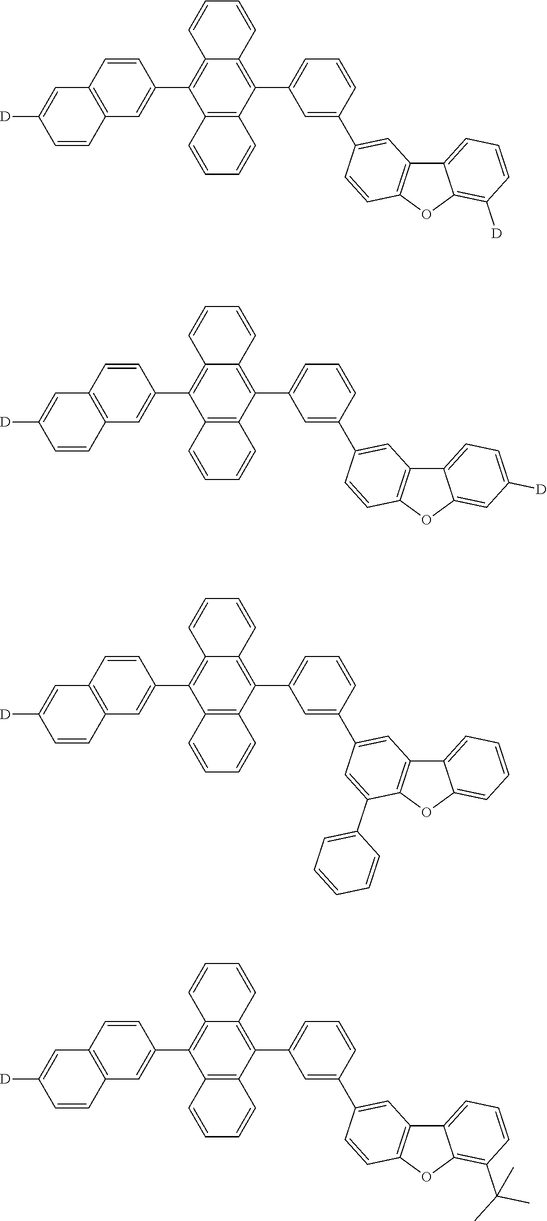 [SMILES: [2H]c1ccc2cc(-c3c4ccccc4c(-c4cccc(-c5ccc6oc7c([2H])cccc7c6c5)c4)c4ccccc34)ccc2c1 ; [2H]c1ccc2cc(-c3c4ccccc4c(-c4cccc(-c5ccc6oc7cc([2H])ccc7c6c5)c4)c4ccccc34)ccc2c1 ; [2H]c1ccc2cc(-c3c4ccccc4c(-c4cccc(-c5cc(-c6ccccc6)c6oc7ccccc7c6c5)c4)c4ccccc34)ccc2c1 ; [2H]c1ccc2cc(-c3c4ccccc4c(-c4cccc(-c5ccc6oc7c(C(C)(C)C)cccc7c6c5)c4)c4ccccc34)ccc2c1]

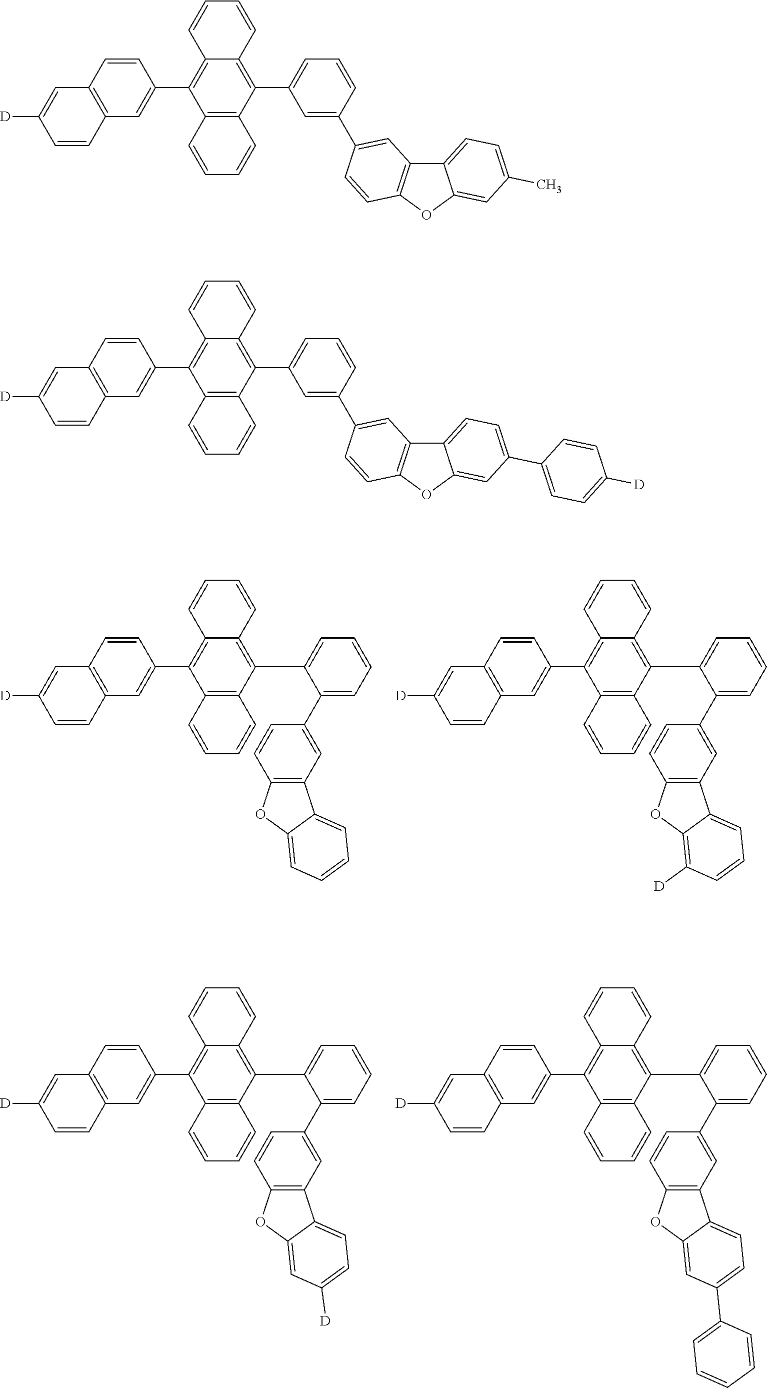

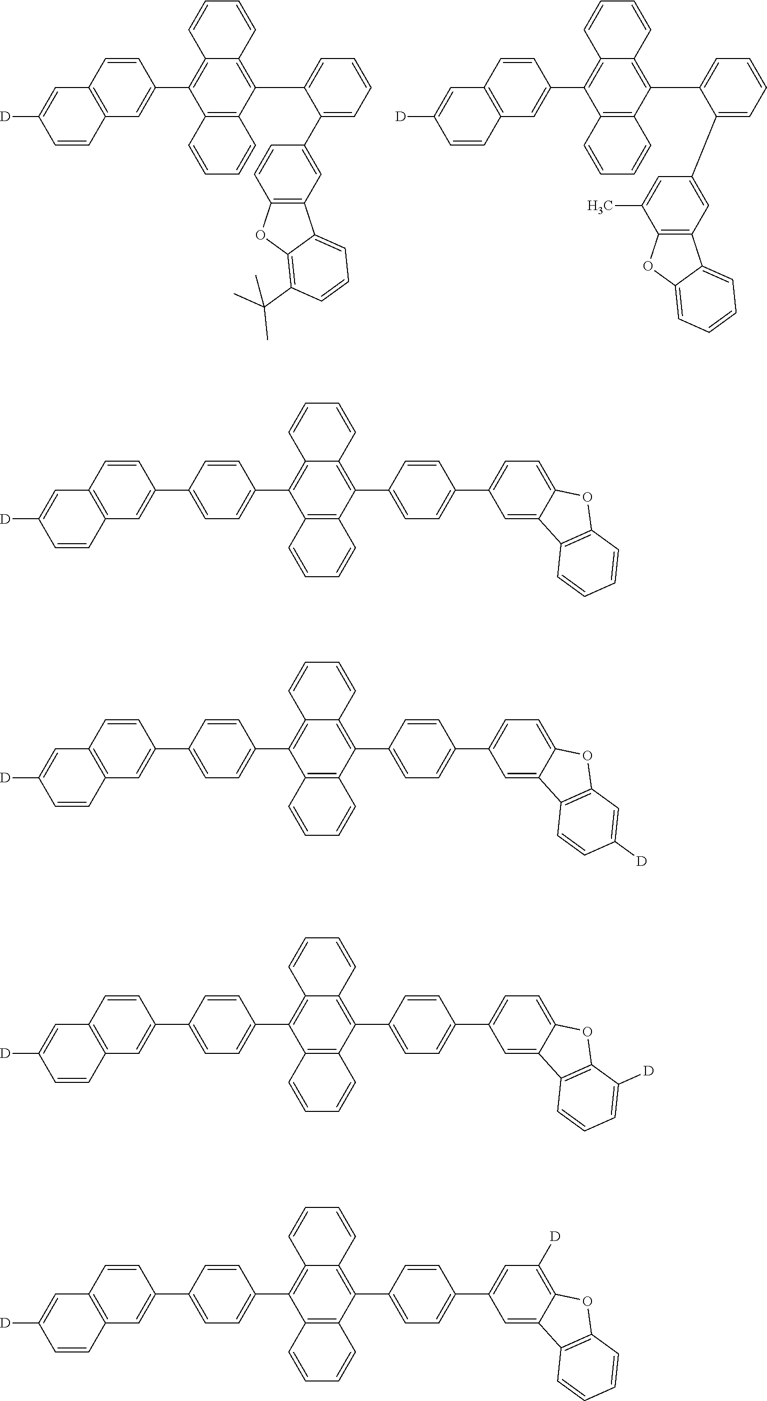

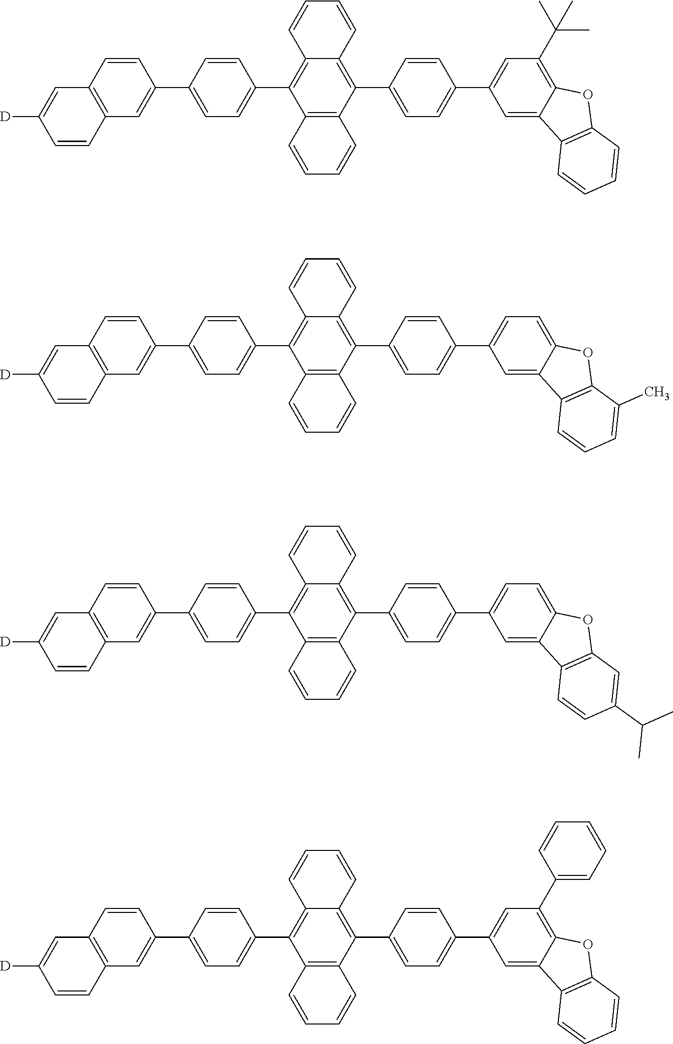 [SMILES: [2H]c1ccc2cc(-c3ccc(-c4c5ccccc5c(-c5ccc(-c6cc(C(C)(C)C)c7oc8ccccc8c7c6)cc5)c5ccccc45)cc3)ccc2c1 ; [2H]c1ccc2cc(-c3ccc(-c4c5ccccc5c(-c5ccc(-c6ccc7oc8c(C)cccc8c7c6)cc5)c5ccccc45)cc3)ccc2c1 ; [2H]c1ccc2cc(-c3ccc(-c4c5ccccc5c(-c5ccc(-c6ccc7oc8cc(C(C)C)ccc8c7c6)cc5)c5ccccc45)cc3)ccc2c1 ; [2H]c1ccc2cc(-c3ccc(-c4c5ccccc5c(-c5ccc(-c6cc(-c7ccccc7)c7oc8ccccc8c7c6)cc5)c5ccccc45)cc3)ccc2c1]

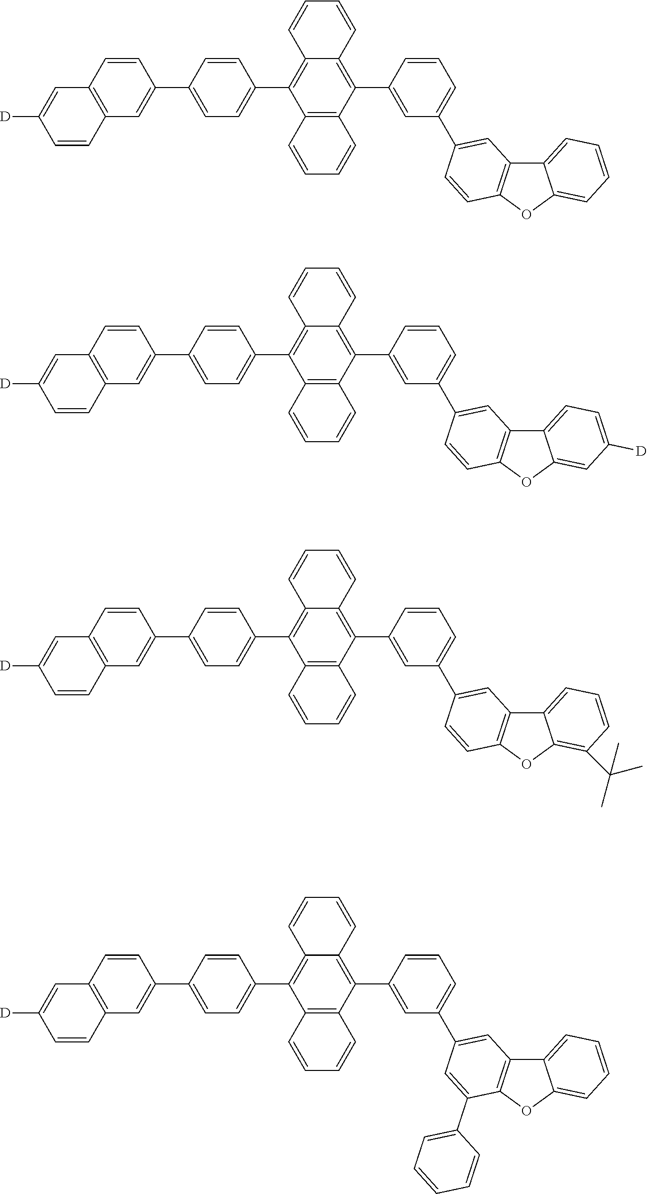 [SMILES: [2H]c1ccc2cc(-c3ccc(-c4c5ccccc5c(-c5cccc(-c6ccc7oc8ccccc8c7c6)c5)c5ccccc45)cc3)ccc2c1 ; [2H]c1ccc2cc(-c3ccc(-c4c5ccccc5c(-c5cccc(-c6ccc7oc8cc([2H])ccc8c7c6)c5)c5ccccc45)cc3)ccc2c1 ; [2H]c1ccc2cc(-c3ccc(-c4c5ccccc5c(-c5cccc(-c6ccc7oc8c(C(C)(C)C)cccc8c7c6)c5)c5ccccc45)cc3)ccc2c1 ; [2H]c1ccc2cc(-c3ccc(-c4c5ccccc5c(-c5cccc(-c6cc(-c7ccccc7)c7oc8ccccc8c7c6)c5)c5ccccc45)cc3)ccc2c1]

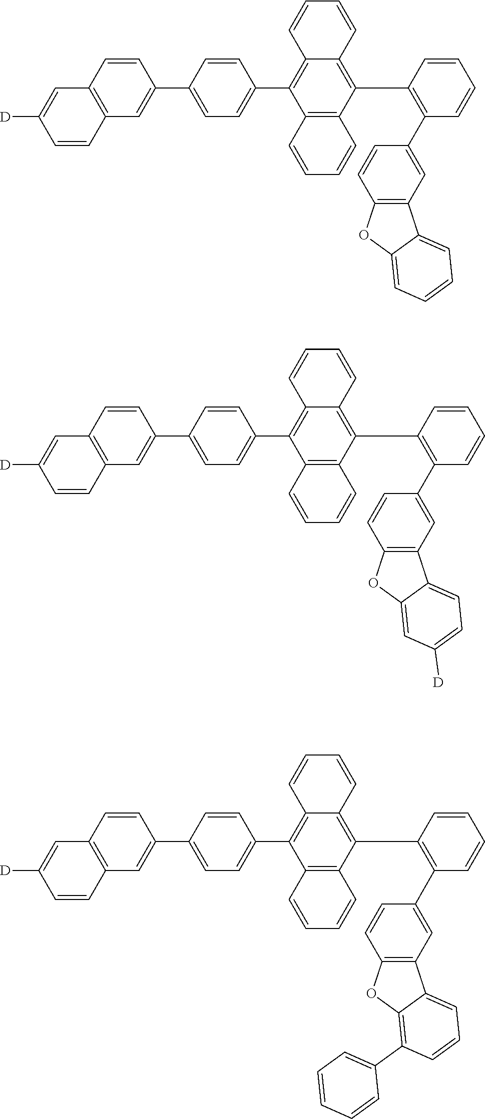 [SMILES: [2H]c1ccc2cc(-c3ccc(-c4c5ccccc5c(-c5ccccc5-c5ccc6oc7ccccc7c6c5)c5ccccc45)cc3)ccc2c1 ; [2H]c1ccc2cc(-c3ccc(-c4c5ccccc5c(-c5ccccc5-c5ccc6oc7cc([2H])ccc7c6c5)c5ccccc45)cc3)ccc2c1 ; [2H]c1ccc2cc(-c3ccc(-c4c5ccccc5c(-c5ccccc5-c5ccc6oc7c(-c8ccccc8)cccc7c6c5)c5ccccc45)cc3)ccc2c1]

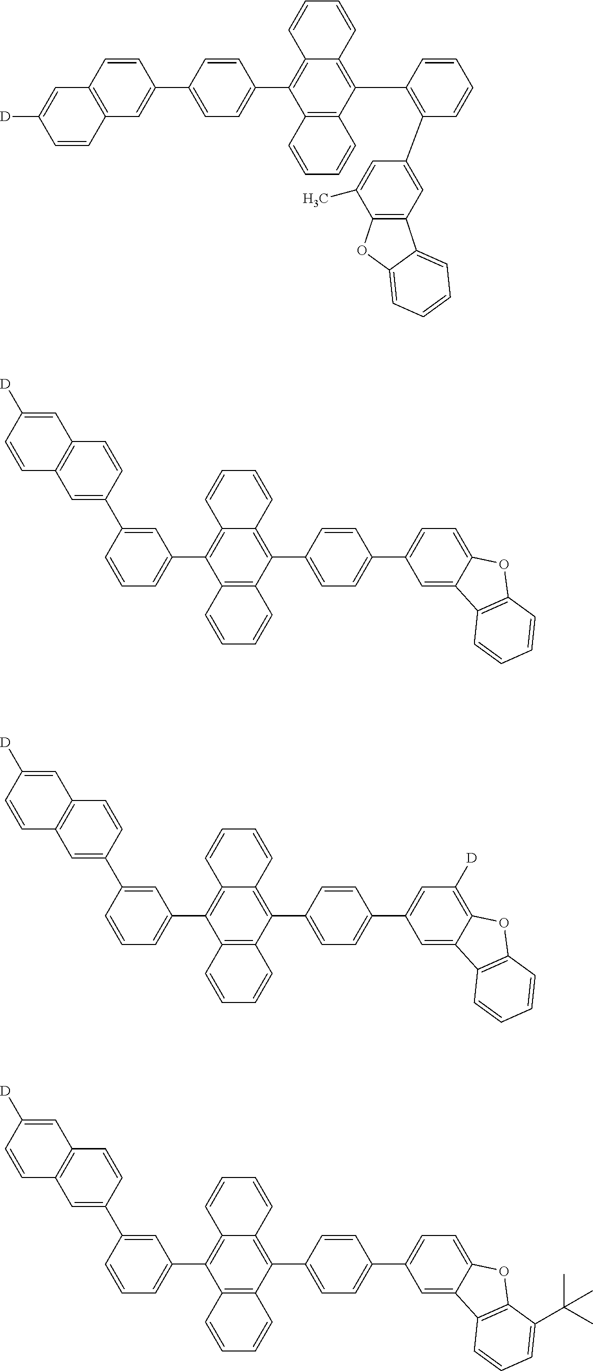 [SMILES: [2H]c1ccc2cc(-c3ccc(-c4c5ccccc5c(-c5ccccc5-c5cc(C)c6oc7ccccc7c6c5)c5ccccc45)cc3)ccc2c1 ; [2H]c1ccc2cc(-c3cccc(-c4c5ccccc5c(-c5ccc(-c6ccc7oc8ccccc8c7c6)cc5)c5ccccc45)c3)ccc2c1 ; [2H]c1ccc2cc(-c3cccc(-c4c5ccccc5c(-c5ccc(-c6cc([2H])c7oc8ccccc8c7c6)cc5)c5ccccc45)c3)ccc2c1 ; [2H]c1ccc2cc(-c3cccc(-c4c5ccccc5c(-c5ccc(-c6ccc7oc8c(C(C)(C)C)cccc8c7c6)cc5)c5ccccc45)c3)ccc2c1]

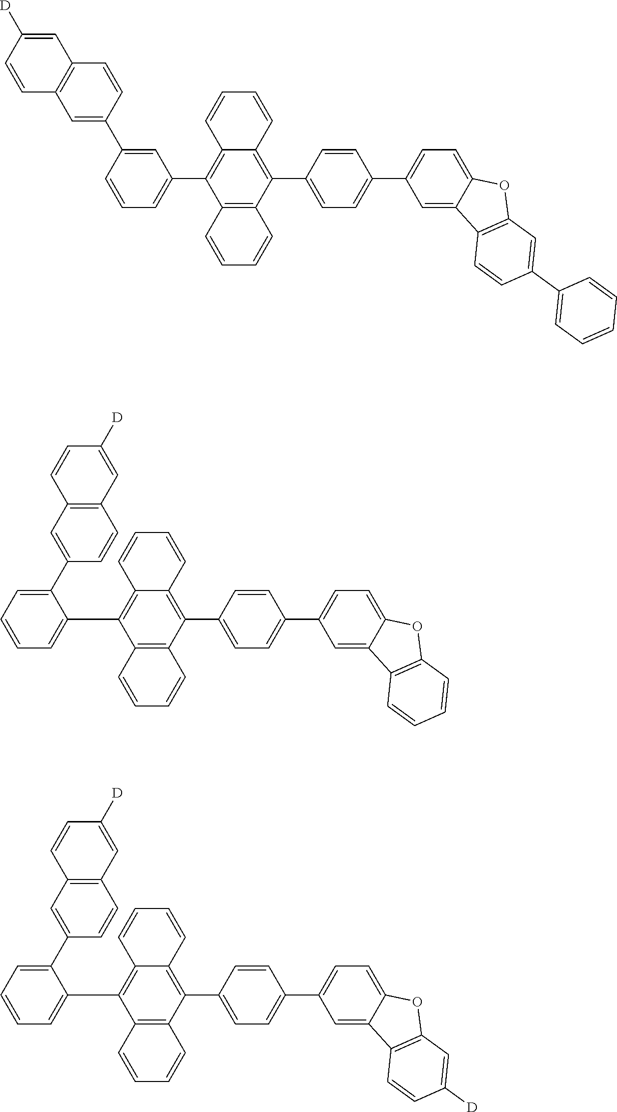 [SMILES: [2H]c1ccc2cc(-c3cccc(-c4c5ccccc5c(-c5ccc(-c6ccc7oc8cc(-c9ccccc9)ccc8c7c6)cc5)c5ccccc45)c3)ccc2c1 ; [2H]c1ccc2cc(-c3ccccc3-c3c4ccccc4c(-c4ccc(-c5ccc6oc7ccccc7c6c5)cc4)c4ccccc34)ccc2c1 ; [2H]c1ccc2cc(-c3ccccc3-c3c4ccccc4c(-c4ccc(-c5ccc6oc7cc([2H])ccc7c6c5)cc4)c4ccccc34)ccc2c1]

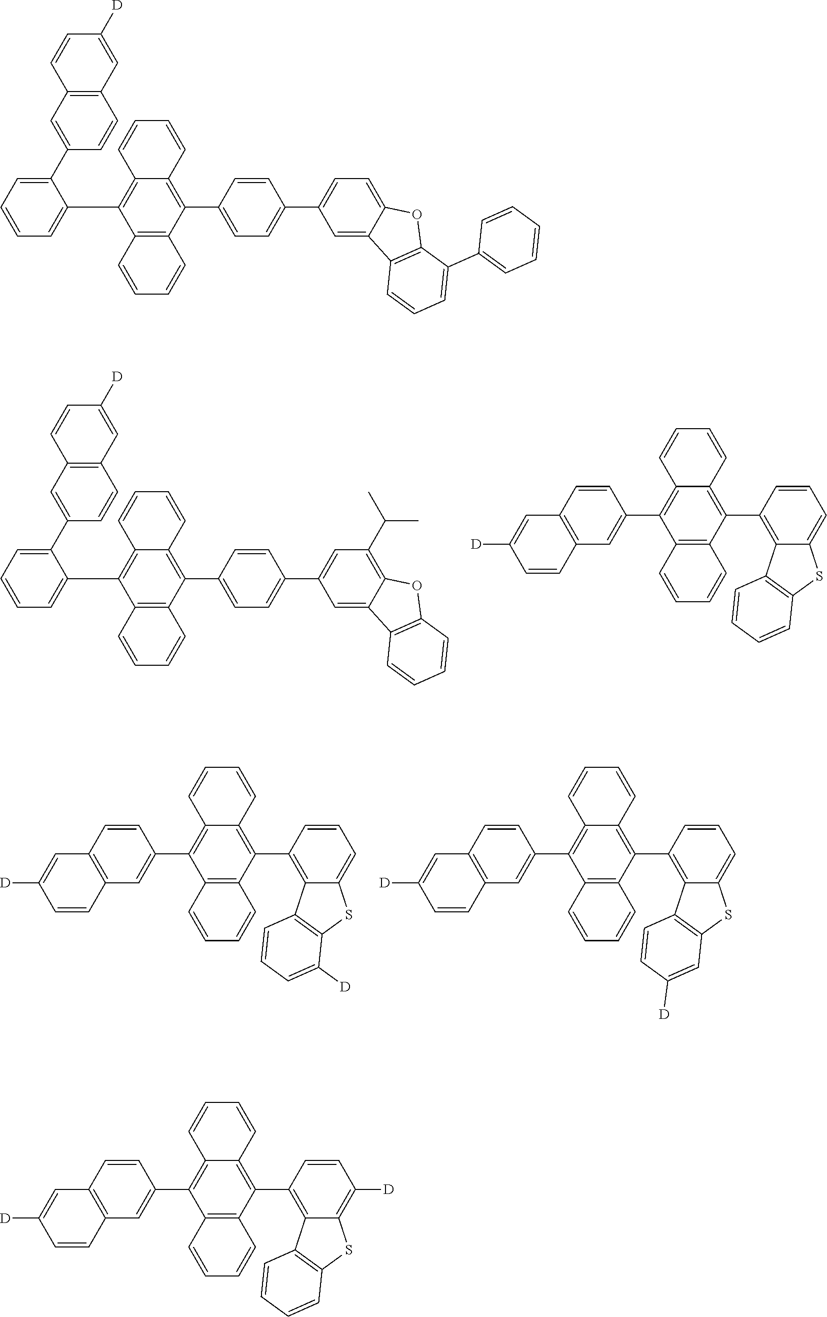

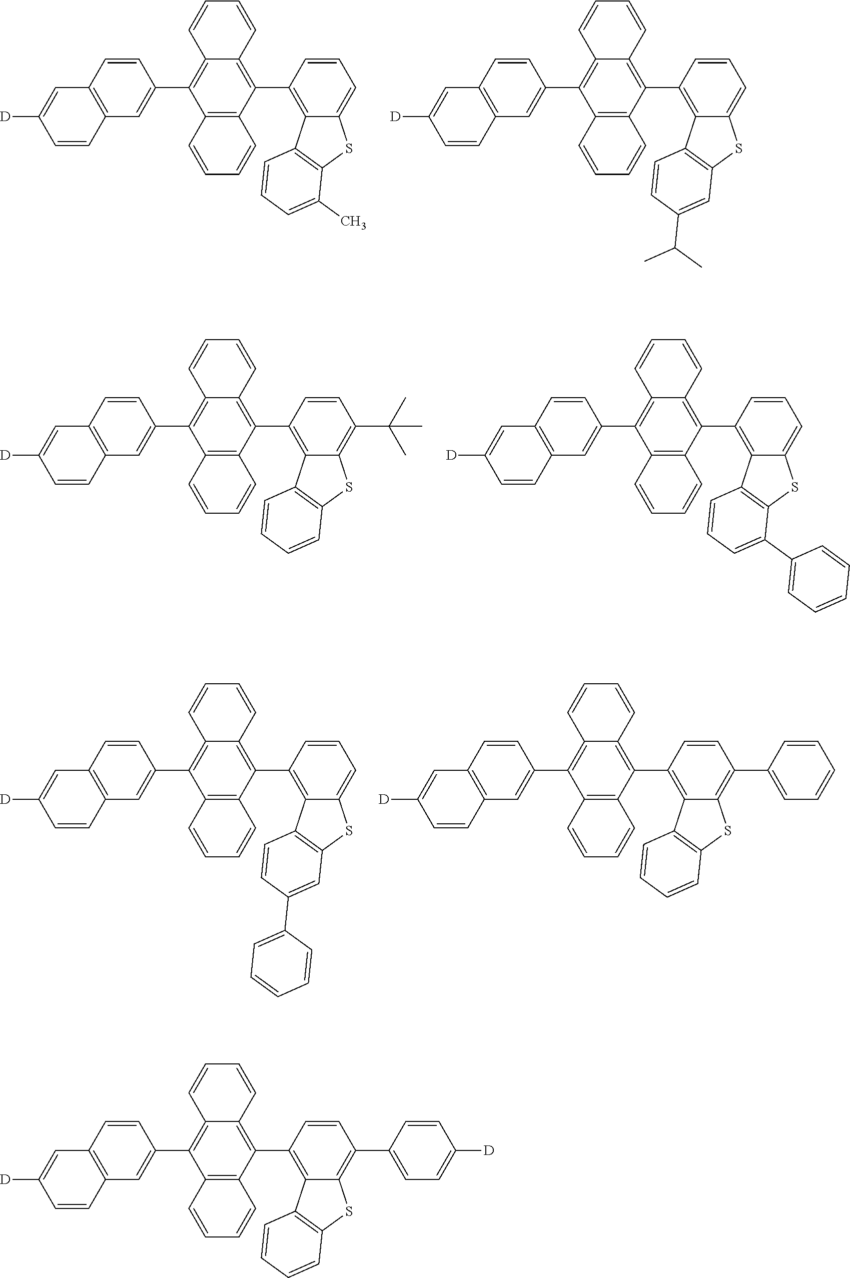

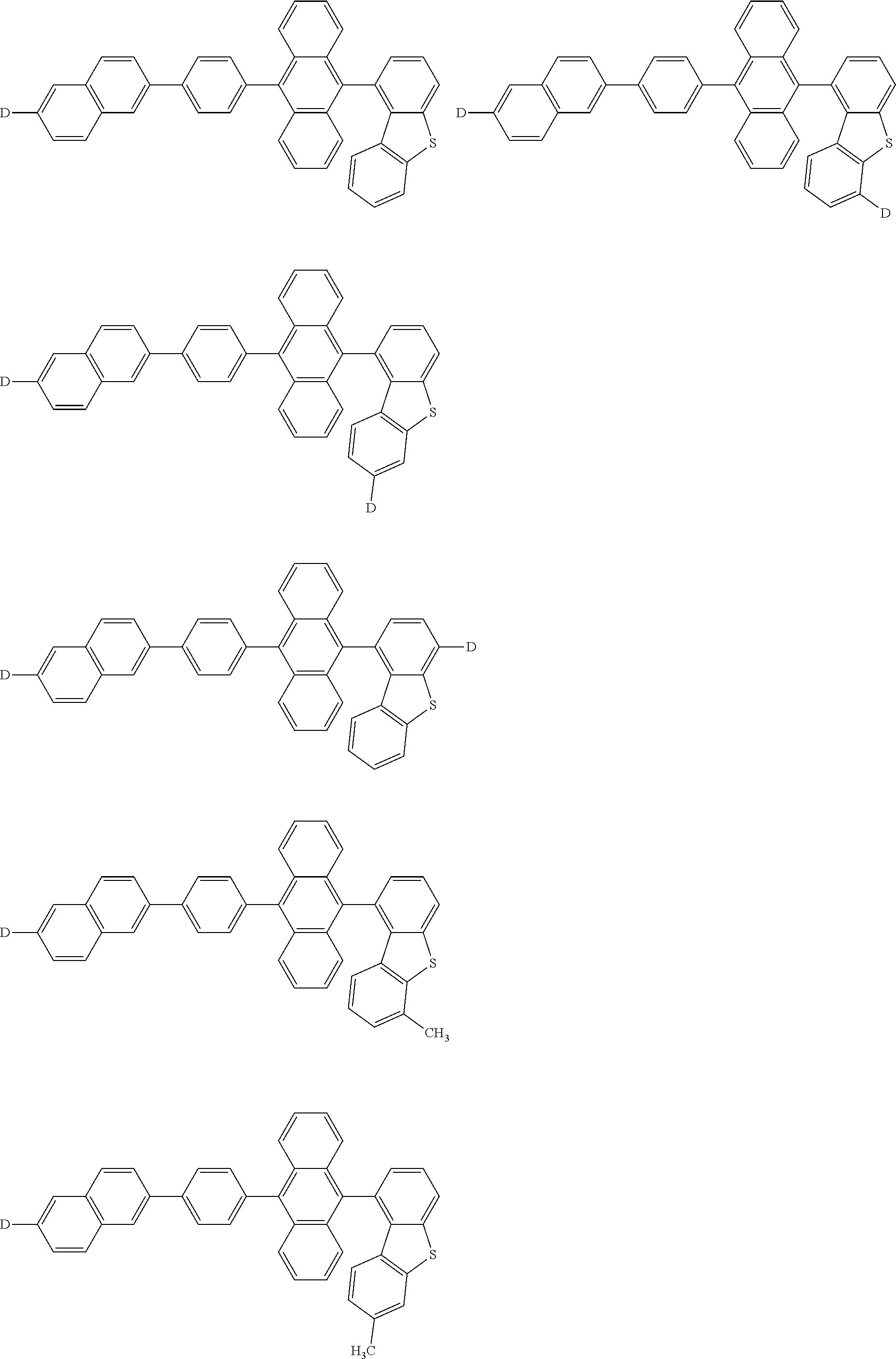

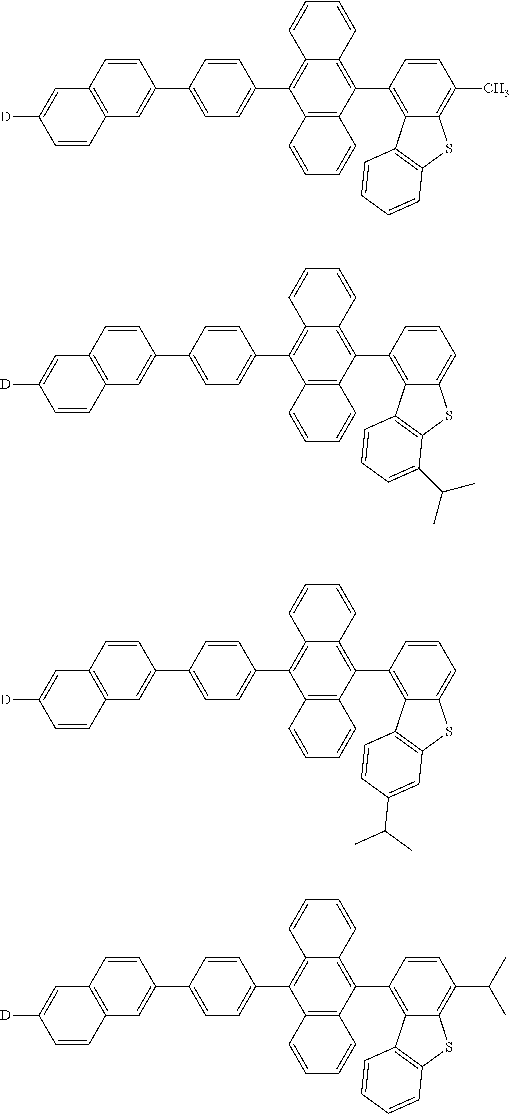 [SMILES: [2H]c1ccc2cc(-c3ccc(-c4c5ccccc5c(-c5ccc(C)c6sc7ccccc7c56)c5ccccc45)cc3)ccc2c1 ; [2H]c1ccc2cc(-c3ccc(-c4c5ccccc5c(-c5cccc6sc7c(C(C)C)cccc7c56)c5ccccc45)cc3)ccc2c1 ; [2H]c1ccc2cc(-c3ccc(-c4c5ccccc5c(-c5cccc6sc7cc(C(C)C)ccc7c56)c5ccccc45)cc3)ccc2c1 ; [2H]c1ccc2cc(-c3ccc(-c4c5ccccc5c(-c5ccc(C(C)C)c6sc7ccccc7c56)c5ccccc45)cc3)ccc2c1]

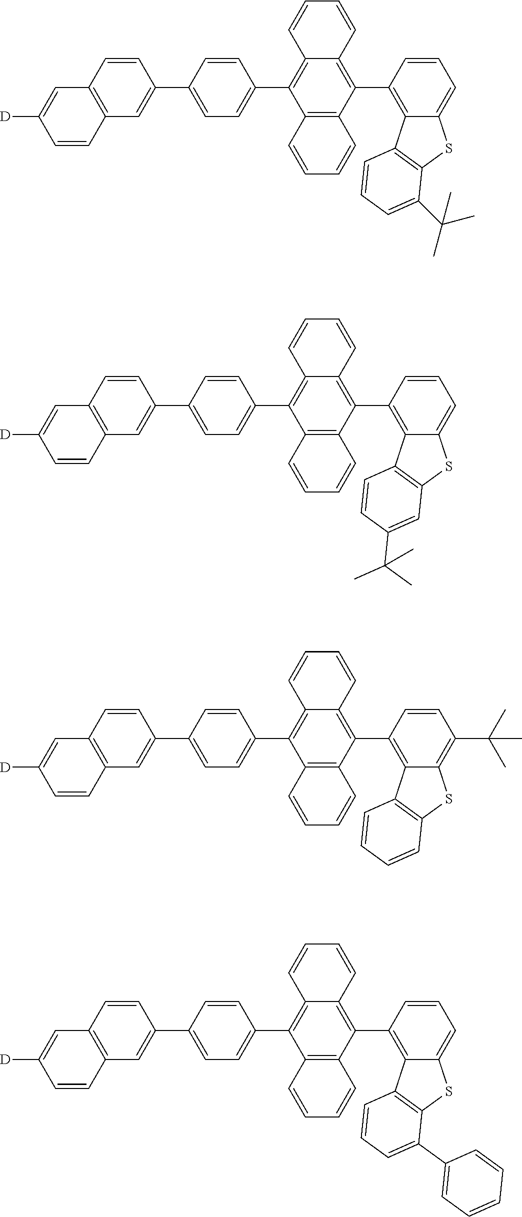 [SMILES: [2H]c1ccc2cc(-c3ccc(-c4c5ccccc5c(-c5cccc6sc7c(C(C)(C)C)cccc7c56)c5ccccc45)cc3)ccc2c1 ; [2H]c1ccc2cc(-c3ccc(-c4c5ccccc5c(-c5cccc6sc7cc(C(C)(C)C)ccc7c56)c5ccccc45)cc3)ccc2c1 ; [2H]c1ccc2cc(-c3ccc(-c4c5ccccc5c(-c5ccc(C(C)(C)C)c6sc7ccccc7c56)c5ccccc45)cc3)ccc2c1 ; [2H]c1ccc2cc(-c3ccc(-c4c5ccccc5c(-c5cccc6sc7c(-c8ccccc8)cccc7c56)c5ccccc45)cc3)ccc2c1]

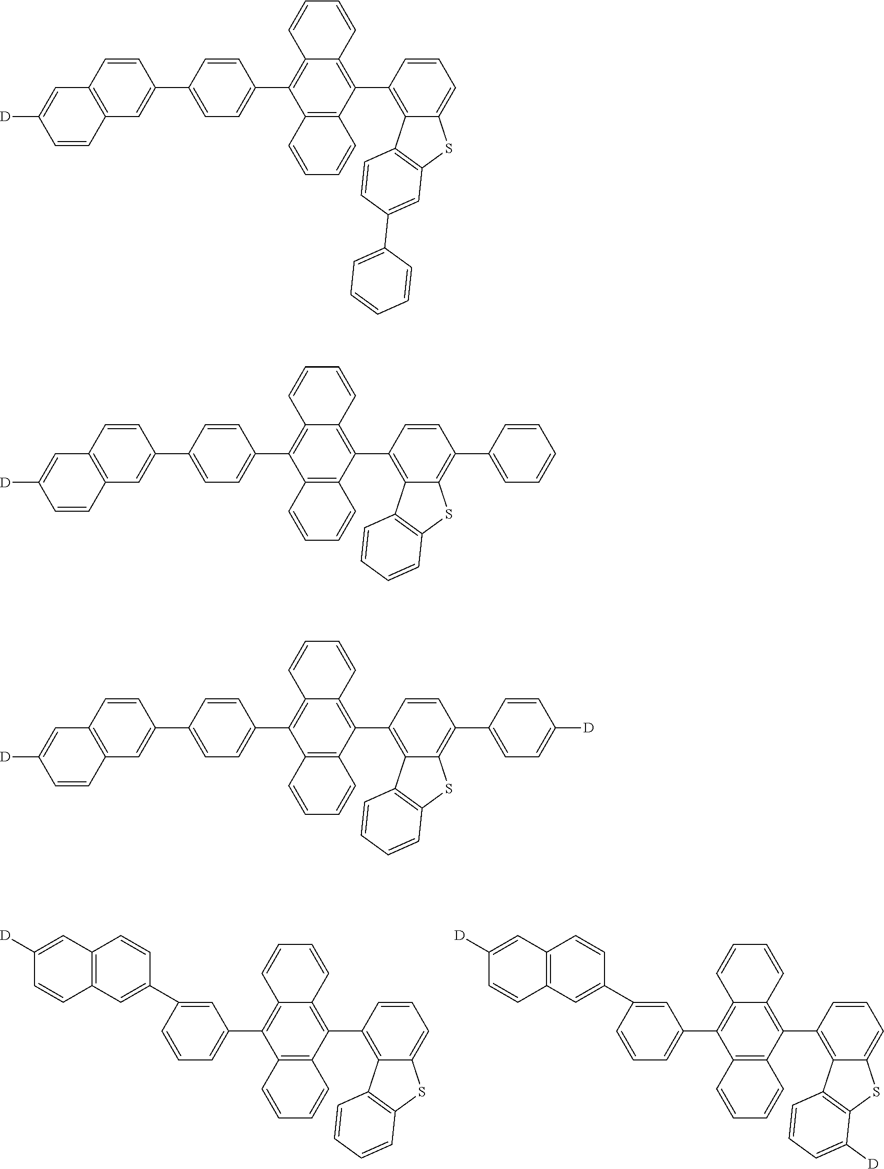 [SMILES: [2H]c1ccc2cc(-c3ccc(-c4c5ccccc5c(-c5cccc6sc7cc(-c8ccccc8)ccc7c56)c5ccccc45)cc3)ccc2c1 ; [2H]c1ccc2cc(-c3ccc(-c4c5ccccc5c(-c5ccc(-c6ccccc6)c6sc7ccccc7c56)c5ccccc45)cc3)ccc2c1 ; [2H]c1ccc(-c2ccc(-c3c4ccccc4c(-c4ccc(-c5ccc6cc([2H])ccc6c5)cc4)c4ccccc34)c3c2sc2ccccc23)cc1 ; [2H]c1ccc2cc(-c3cccc(-c4c5ccccc5c(-c5cccc6sc7ccccc7c56)c5ccccc45)c3)ccc2c1 ; [2H]c1ccc2cc(-c3cccc(-c4c5ccccc5c(-c5cccc6sc7c([2H])cccc7c56)c5ccccc45)c3)ccc2c1]

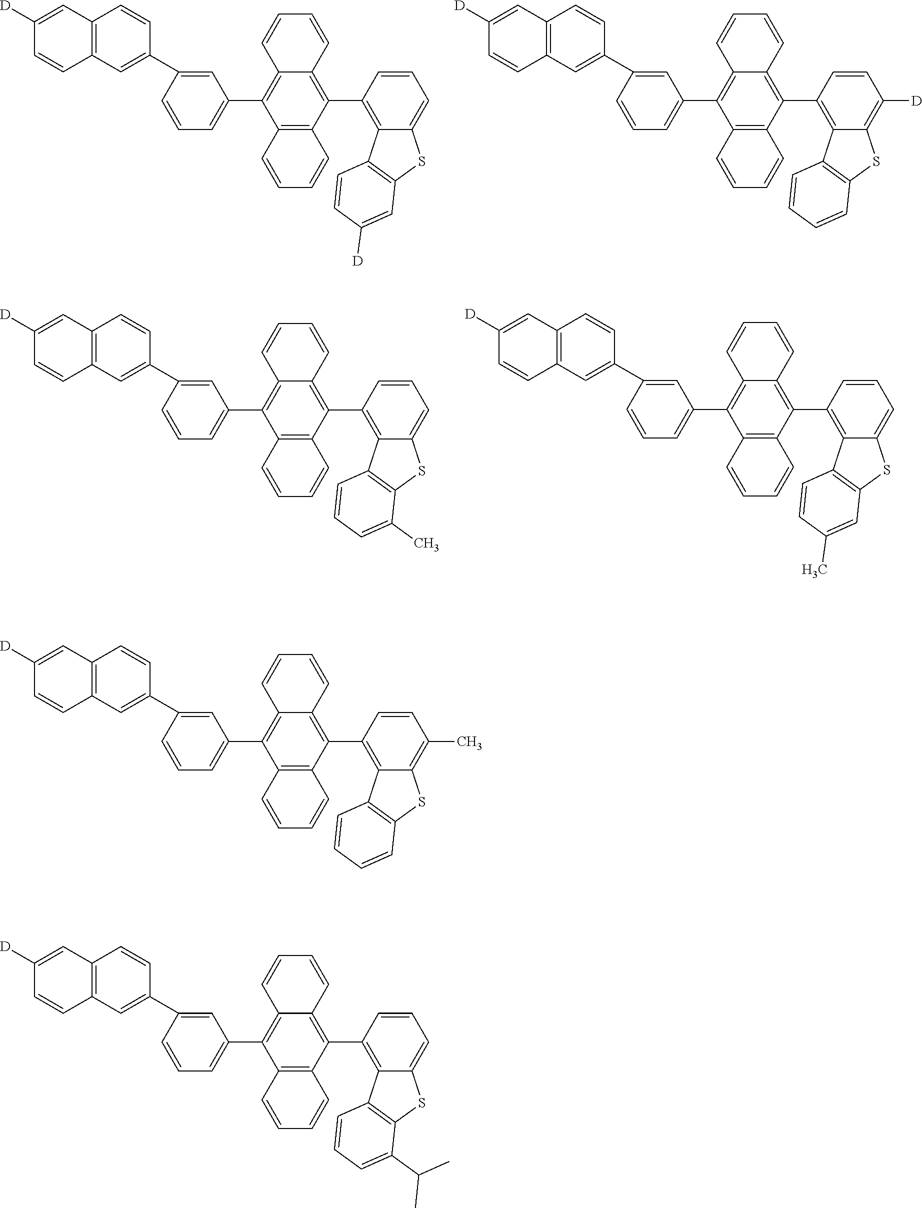 [SMILES: [2H]c1ccc2cc(-c3cccc(-c4c5ccccc5c(-c5cccc6sc7cc([2H])ccc7c56)c5ccccc45)c3)ccc2c1 ; [2H]c1ccc2cc(-c3cccc(-c4c5ccccc5c(-c5ccc([2H])c6sc7ccccc7c56)c5ccccc45)c3)ccc2c1 ; [2H]c1ccc2cc(-c3cccc(-c4c5ccccc5c(-c5cccc6sc7c(C)cccc7c56)c5ccccc45)c3)ccc2c1 ; [2H]c1ccc2cc(-c3cccc(-c4c5ccccc5c(-c5cccc6sc7cc(C)ccc7c56)c5ccccc45)c3)ccc2c1 ; [2H]c1ccc2cc(-c3cccc(-c4c5ccccc5c(-c5ccc(C)c6sc7ccccc7c56)c5ccccc45)c3)ccc2c1 ; [2H]c1ccc2cc(-c3cccc(-c4c5ccccc5c(-c5cccc6sc7c(C(C)C)cccc7c56)c5ccccc45)c3)ccc2c1]

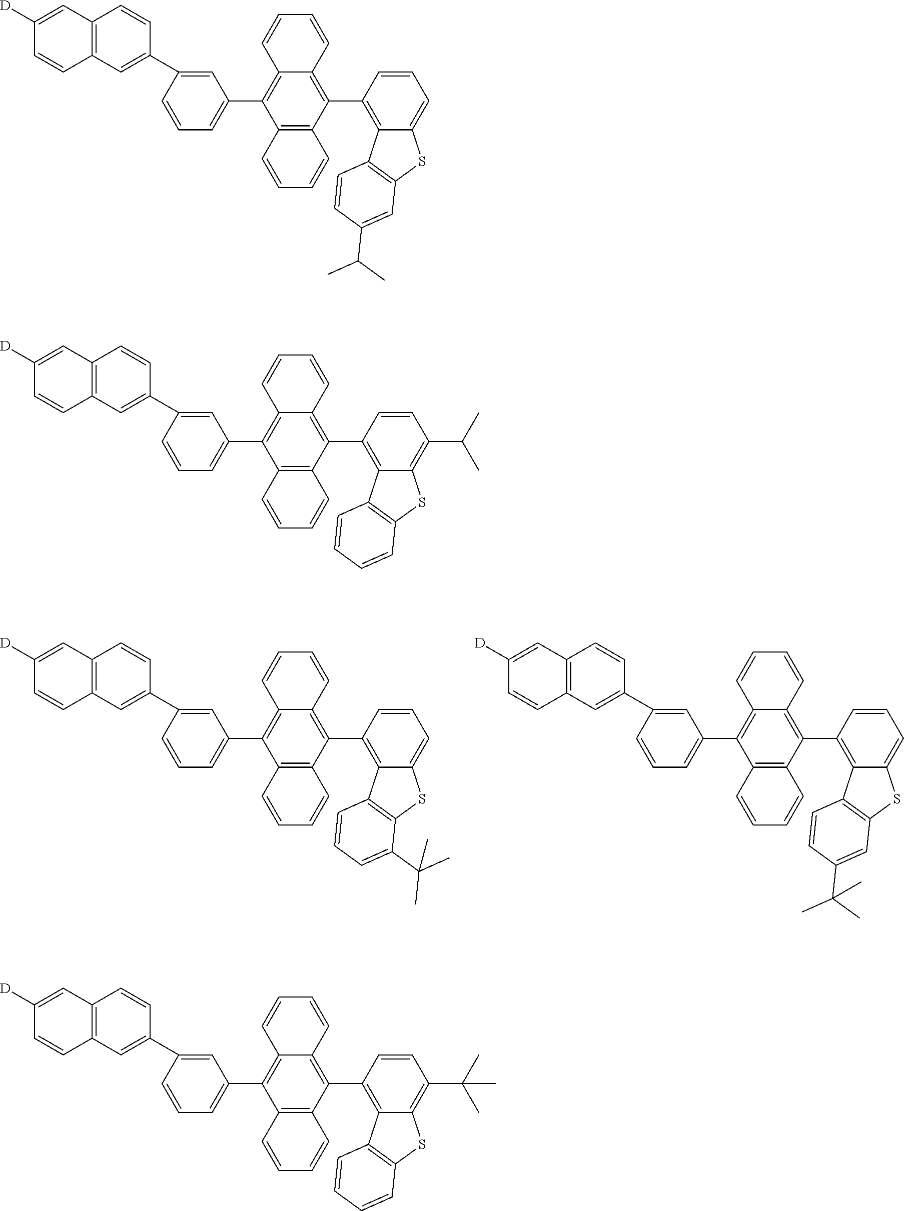 [SMILES: [2H]c1ccc2cc(-c3cccc(-c4c5ccccc5c(-c5cccc6sc7cc(C(C)C)ccc7c56)c5ccccc45)c3)ccc2c1 ; [2H]c1ccc2cc(-c3cccc(-c4c5ccccc5c(-c5ccc(C(C)C)c6sc7ccccc7c56)c5ccccc45)c3)ccc2c1 ; [2H]c1ccc2cc(-c3cccc(-c4c5ccccc5c(-c5cccc6sc7c(C(C)(C)C)cccc7c56)c5ccccc45)c3)ccc2c1 ; [2H]c1ccc2cc(-c3cccc(-c4c5ccccc5c(-c5cccc6sc7cc(C(C)(C)C)ccc7c56)c5ccccc45)c3)ccc2c1 ; [2H]c1ccc2cc(-c3cccc(-c4c5ccccc5c(-c5ccc(C(C)(C)C)c6sc7ccccc7c56)c5ccccc45)c3)ccc2c1]

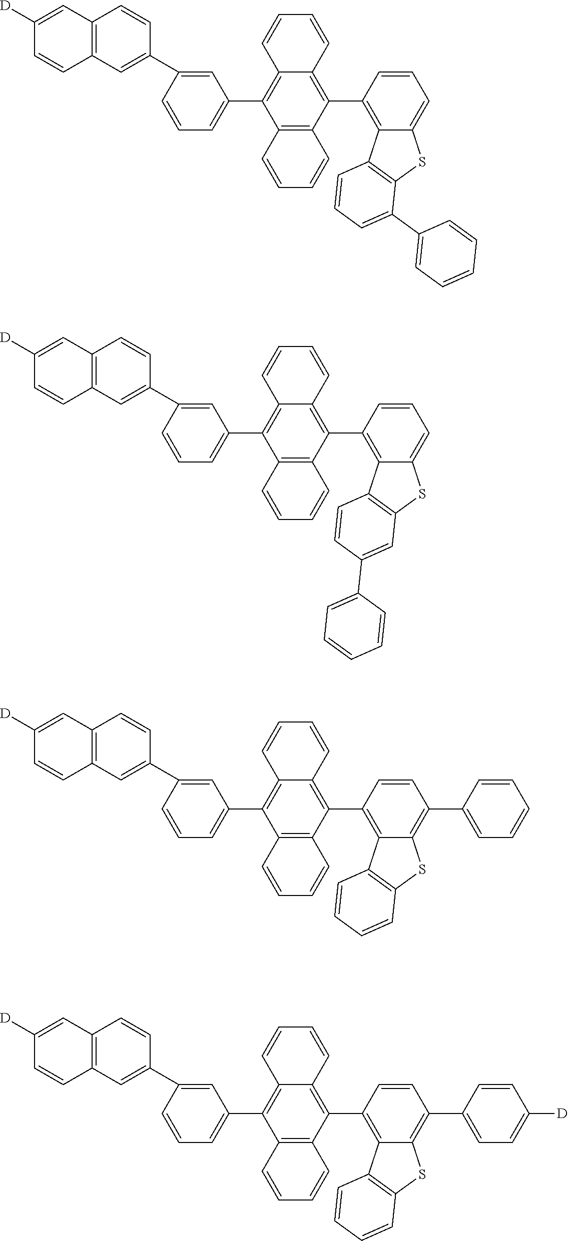 [SMILES: [2H]c1ccc2cc(-c3cccc(-c4c5ccccc5c(-c5cccc6sc7c(-c8ccccc8)cccc7c56)c5ccccc45)c3)ccc2c1 ; [2H]c1ccc2cc(-c3cccc(-c4c5ccccc5c(-c5cccc6sc7cc(-c8ccccc8)ccc7c56)c5ccccc45)c3)ccc2c1 ; [2H]c1ccc2cc(-c3cccc(-c4c5ccccc5c(-c5ccc(-c6ccccc6)c6sc7ccccc7c56)c5ccccc45)c3)ccc2c1 ; [2H]c1ccc(-c2ccc(-c3c4ccccc4c(-c4cccc(-c5ccc6cc([2H])ccc6c5)c4)c4ccccc34)c3c2sc2ccccc23)cc1]

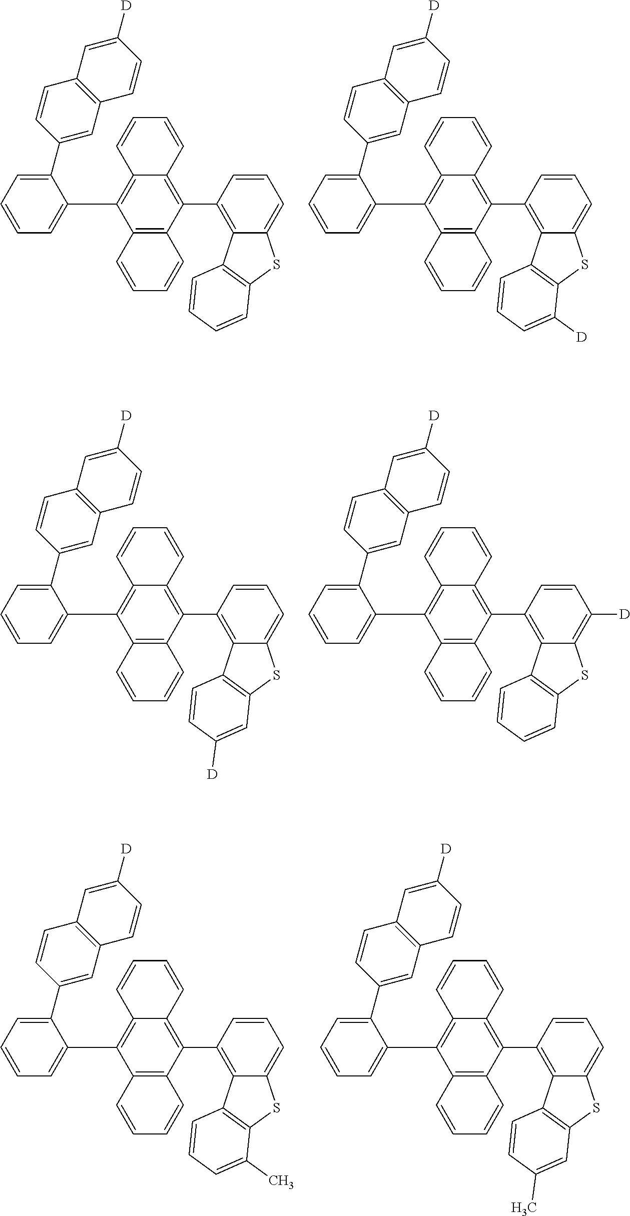

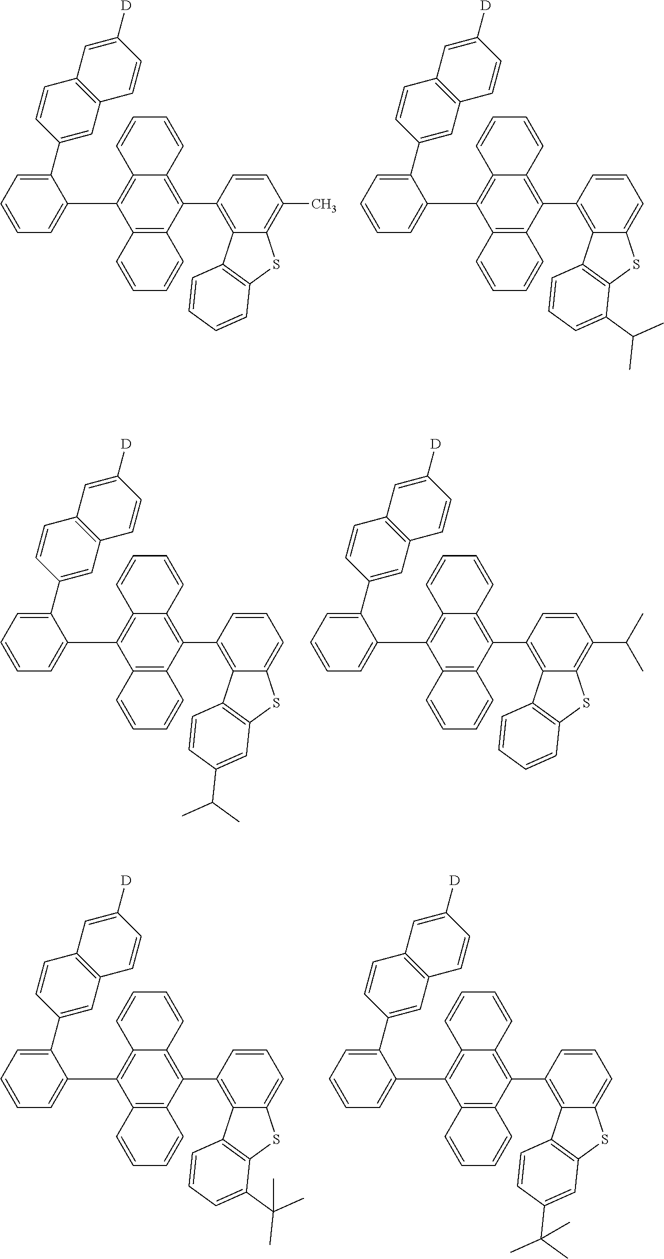

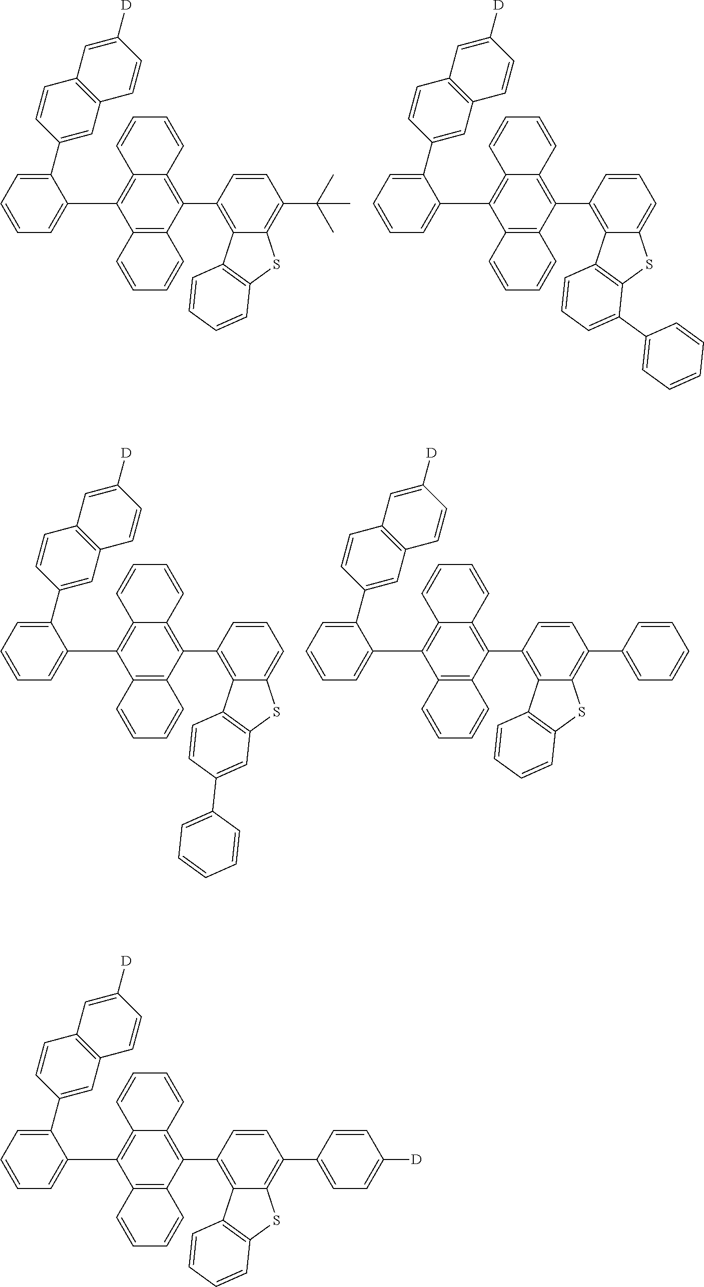

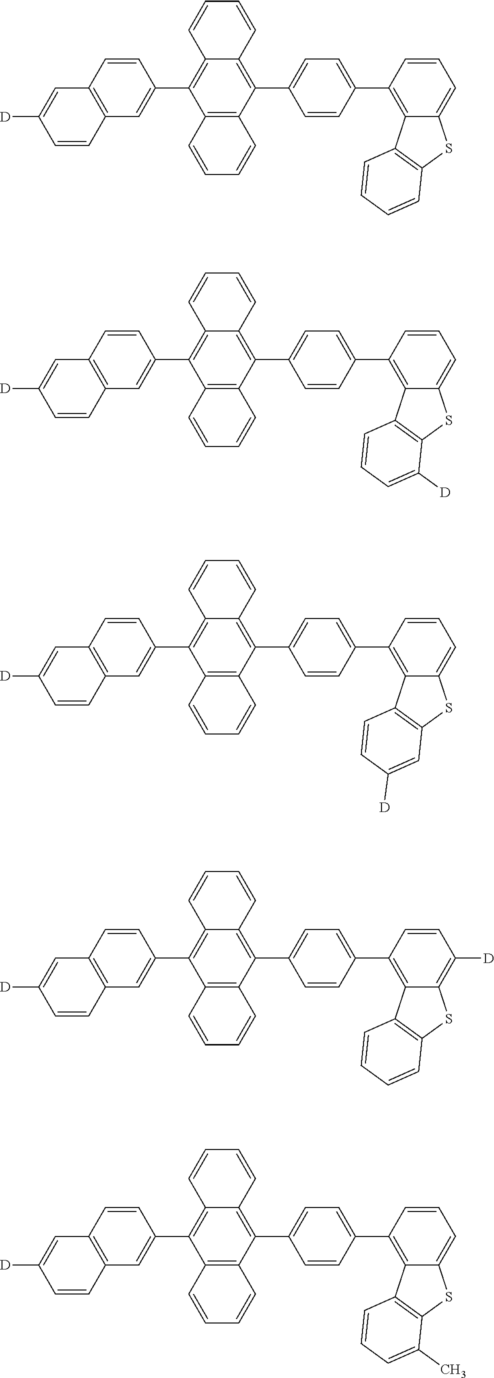 [SMILES: [2H]c1ccc2cc(-c3c4ccccc4c(-c4ccc(-c5cccc6sc7ccccc7c56)cc4)c4ccccc34)ccc2c1 ; [2H]c1ccc2cc(-c3c4ccccc4c(-c4ccc(-c5cccc6sc7c([2H])cccc7c56)cc4)c4ccccc34)ccc2c1 ; [2H]c1ccc2cc(-c3c4ccccc4c(-c4ccc(-c5cccc6sc7cc([2H])ccc7c56)cc4)c4ccccc34)ccc2c1 ; [2H]c1ccc2cc(-c3c4ccccc4c(-c4ccc(-c5ccc([2H])c6sc7ccccc7c56)cc4)c4ccccc34)ccc2c1 ; [2H]c1ccc2cc(-c3c4ccccc4c(-c4ccc(-c5cccc6sc7c(C)cccc7c56)cc4)c4ccccc34)ccc2c1]

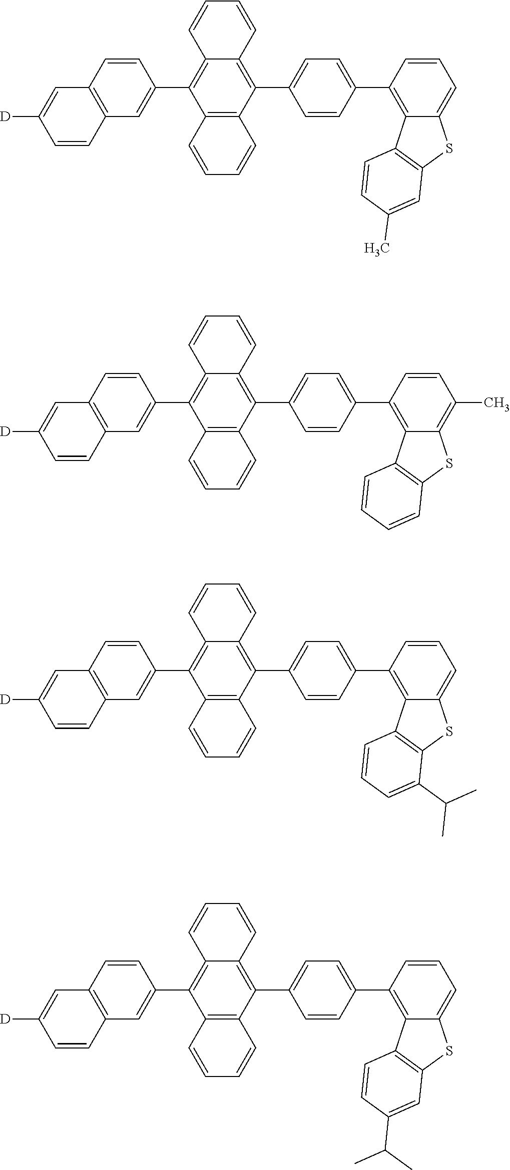 [SMILES: [2H]c1ccc2cc(-c3c4ccccc4c(-c4ccc(-c5cccc6sc7cc(C)ccc7c56)cc4)c4ccccc34)ccc2c1 ; [2H]c1ccc2cc(-c3c4ccccc4c(-c4ccc(-c5ccc(C)c6sc7ccccc7c56)cc4)c4ccccc34)ccc2c1 ; [2H]c1ccc2cc(-c3c4ccccc4c(-c4ccc(-c5cccc6sc7c(C(C)C)cccc7c56)cc4)c4ccccc34)ccc2c1 ; [2H]c1ccc2cc(-c3c4ccccc4c(-c4ccc(-c5cccc6sc7cc(C(C)C)ccc7c56)cc4)c4ccccc34)ccc2c1]

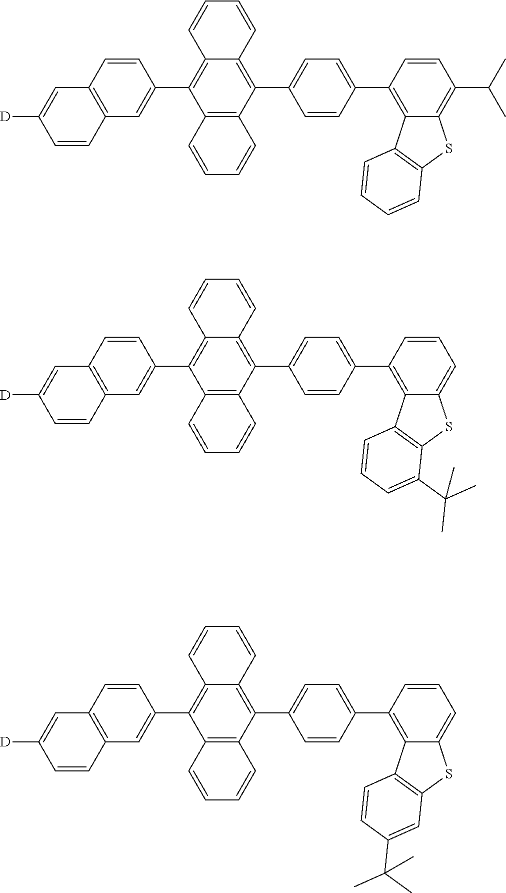 [SMILES: [2H]c1ccc2cc(-c3c4ccccc4c(-c4ccc(-c5ccc(C(C)C)c6sc7ccccc7c56)cc4)c4ccccc34)ccc2c1 ; [2H]c1ccc2cc(-c3c4ccccc4c(-c4ccc(-c5cccc6sc7c(C(C)(C)C)cccc7c56)cc4)c4ccccc34)ccc2c1 ; [2H]c1ccc2cc(-c3c4ccccc4c(-c4ccc(-c5cccc6sc7cc(C(C)(C)C)ccc7c56)cc4)c4ccccc34)ccc2c1]

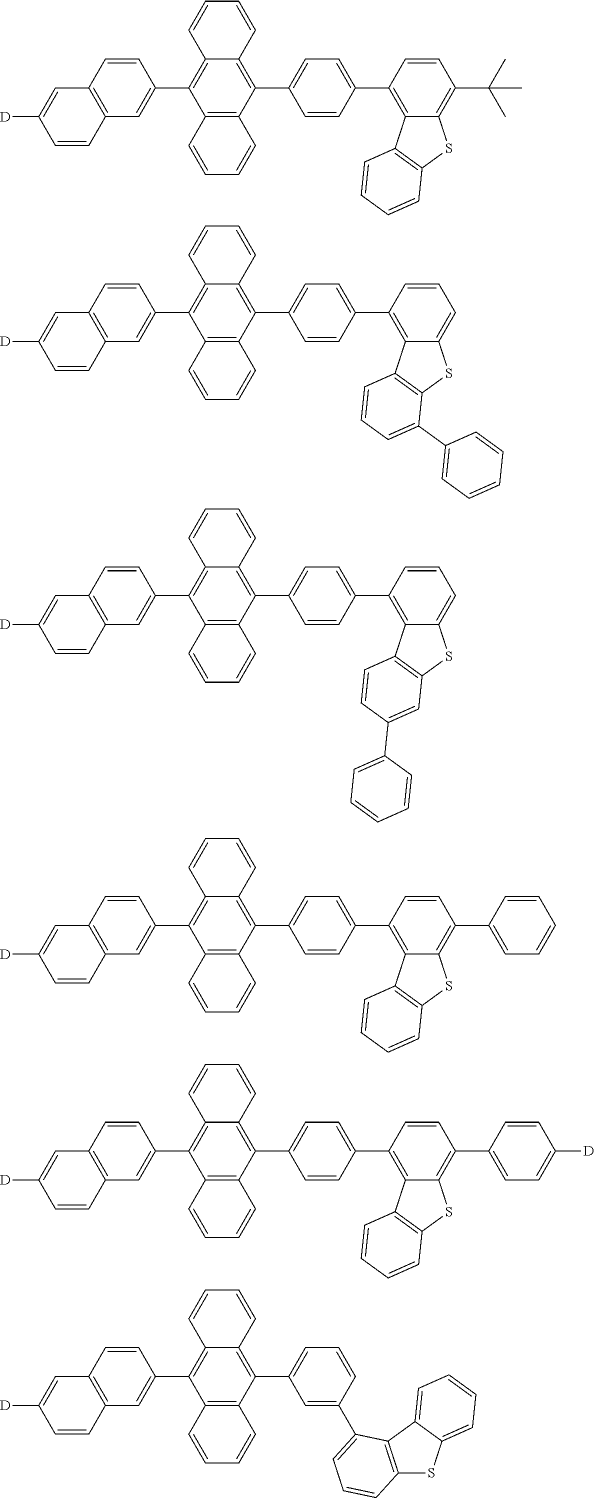

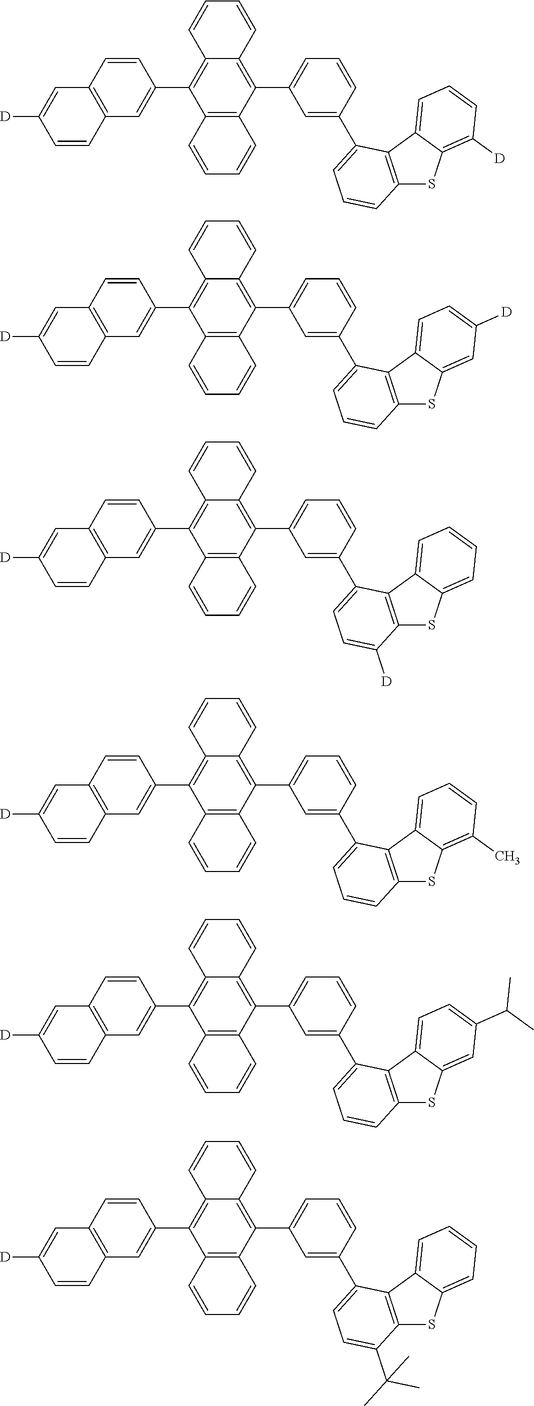 [SMILES: [2H]c1ccc2cc(-c3c4ccccc4c(-c4cccc(-c5cccc6sc7c([2H])cccc7c56)c4)c4ccccc34)ccc2c1 ; [2H]c1ccc2cc(-c3c4ccccc4c(-c4cccc(-c5cccc6sc7cc([2H])ccc7c56)c4)c4ccccc34)ccc2c1 ; [2H]c1ccc2cc(-c3c4ccccc4c(-c4cccc(-c5ccc([2H])c6sc7ccccc7c56)c4)c4ccccc34)ccc2c1 ; [2H]c1ccc2cc(-c3c4ccccc4c(-c4cccc(-c5cccc6sc7c(C)cccc7c56)c4)c4ccccc34)ccc2c1 ; [2H]c1ccc2cc(-c3c4ccccc4c(-c4cccc(-c5cccc6sc7cc(C(C)C)ccc7c56)c4)c4ccccc34)ccc2c1 ; [2H]c1ccc2cc(-c3c4ccccc4c(-c4cccc(-c5ccc(C(C)(C)C)c6sc7ccccc7c56)c4)c4ccccc34)ccc2c1]

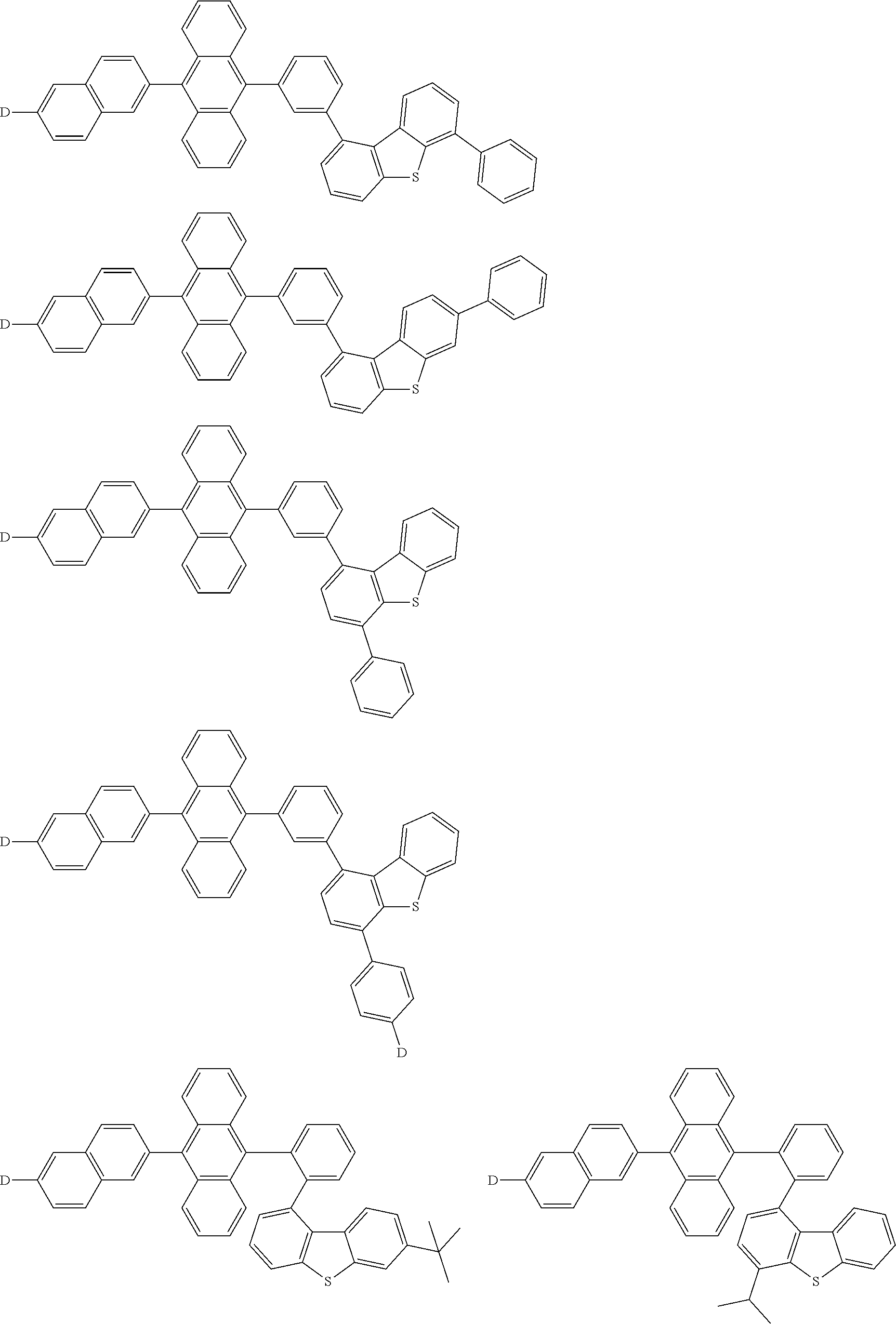

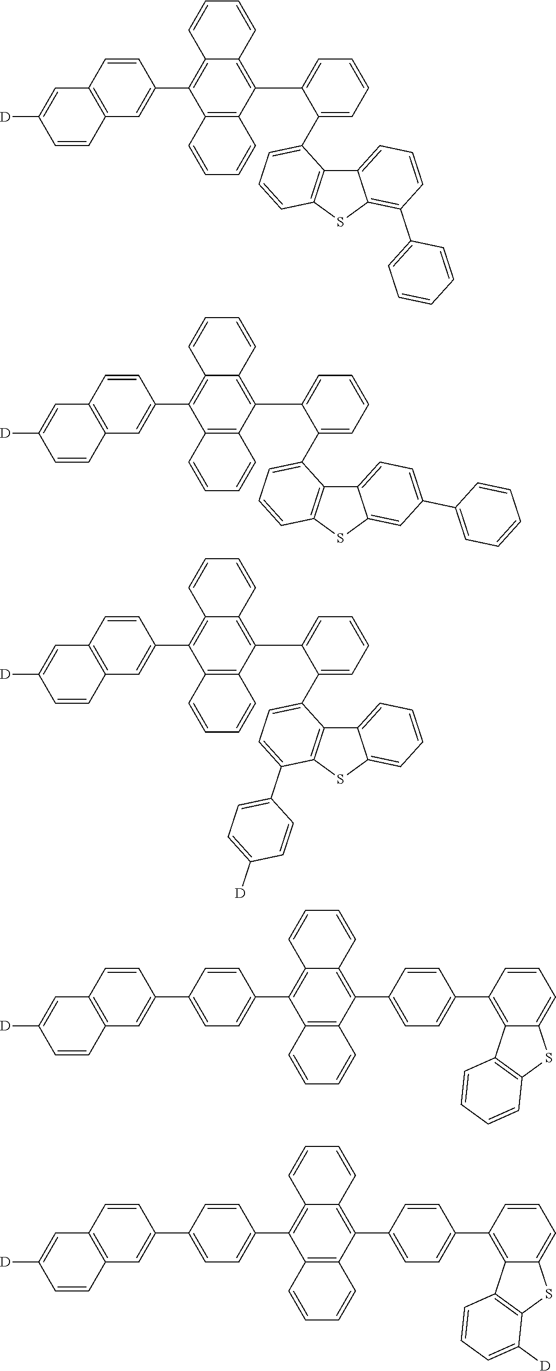 [SMILES: [2H]c1ccc2cc(-c3c4ccccc4c(-c4ccccc4-c4cccc5sc6c(-c7ccccc7)cccc6c45)c4ccccc34)ccc2c1 ; [2H]c1ccc2cc(-c3c4ccccc4c(-c4ccccc4-c4cccc5sc6cc(-c7ccccc7)ccc6c45)c4ccccc34)ccc2c1 ; [2H]c1ccc(-c2ccc(-c3ccccc3-c3c4ccccc4c(-c4ccc5cc([2H])ccc5c4)c4ccccc34)c3c2sc2ccccc23)cc1 ; [2H]c1ccc2cc(-c3ccc(-c4c5ccccc5c(-c5ccc(-c6cccc7sc8ccccc8c67)cc5)c5ccccc45)cc3)ccc2c1 ; [2H]c1ccc2cc(-c3ccc(-c4c5ccccc5c(-c5ccc(-c6cccc7sc8c([2H])cccc8c67)cc5)c5ccccc45)cc3)ccc2c1]

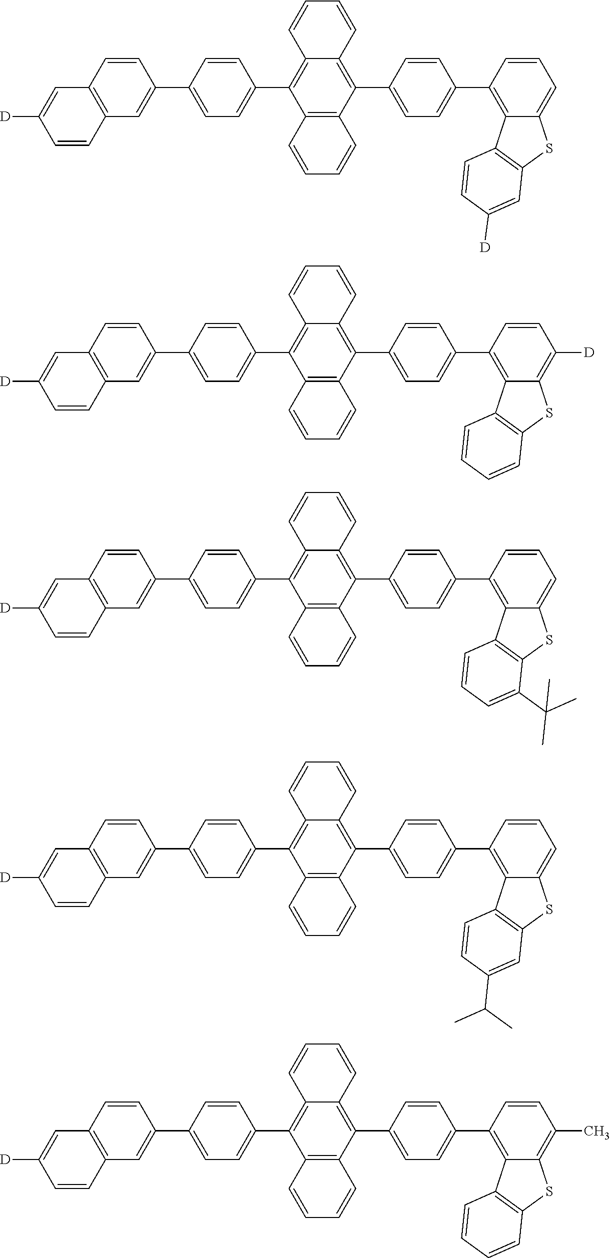 [SMILES: [2H]c1ccc2cc(-c3ccc(-c4c5ccccc5c(-c5ccc(-c6cccc7sc8cc([2H])ccc8c67)cc5)c5ccccc45)cc3)ccc2c1 ; [2H]c1ccc2cc(-c3ccc(-c4c5ccccc5c(-c5ccc(-c6ccc([2H])c7sc8ccccc8c67)cc5)c5ccccc45)cc3)ccc2c1 ; [2H]c1ccc2cc(-c3ccc(-c4c5ccccc5c(-c5ccc(-c6cccc7sc8c(C(C)(C)C)cccc8c67)cc5)c5ccccc45)cc3)ccc2c1 ; [2H]c1ccc2cc(-c3ccc(-c4c5ccccc5c(-c5ccc(-c6cccc7sc8cc(C(C)C)ccc8c67)cc5)c5ccccc45)cc3)ccc2c1 ; [2H]c1ccc2cc(-c3ccc(-c4c5ccccc5c(-c5ccc(-c6ccc(C)c7sc8ccccc8c67)cc5)c5ccccc45)cc3)ccc2c1]

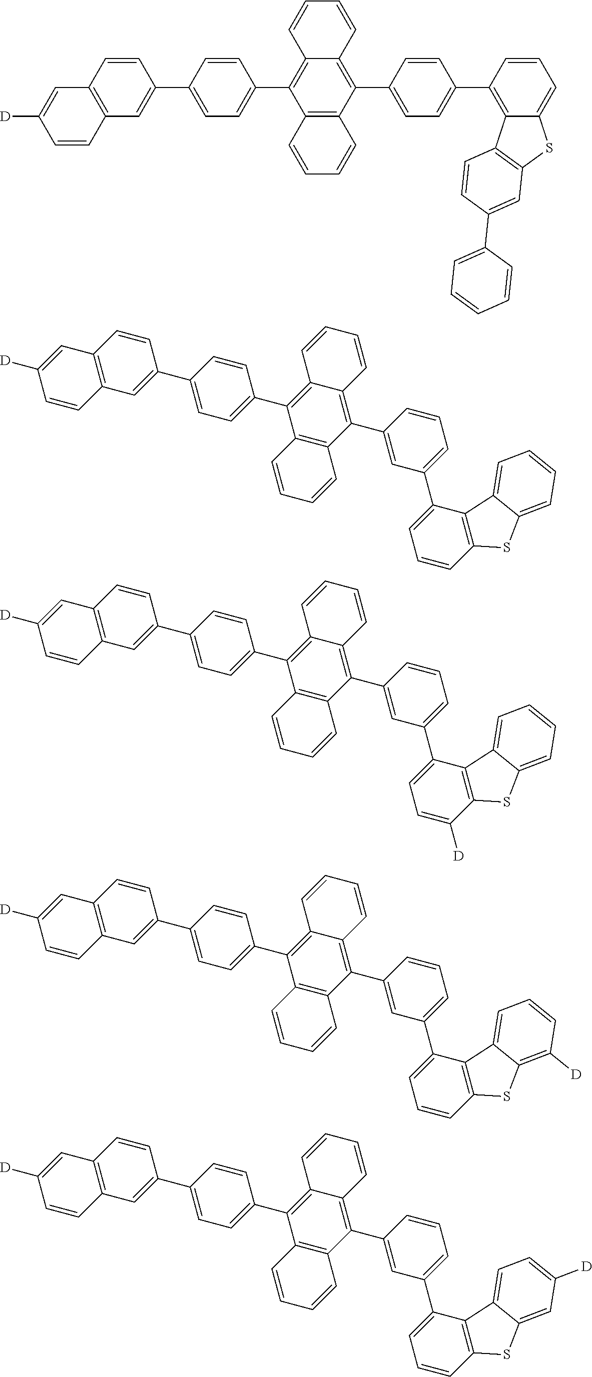 [SMILES: [2H]c1ccc2cc(-c3ccc(-c4c5ccccc5c(-c5ccc(-c6cccc7sc8cc(-c9ccccc9)ccc8c67)cc5)c5ccccc45)cc3)ccc2c1 ; [2H]c1ccc2cc(-c3ccc(-c4c5ccccc5c(-c5cccc(-c6cccc7sc8ccccc8c67)c5)c5ccccc45)cc3)ccc2c1 ; [2H]c1ccc2cc(-c3ccc(-c4c5ccccc5c(-c5cccc(-c6ccc([2H])c7sc8ccccc8c67)c5)c5ccccc45)cc3)ccc2c1 ; [2H]c1ccc2cc(-c3ccc(-c4c5ccccc5c(-c5cccc(-c6cccc7sc8c([2H])cccc8c67)c5)c5ccccc45)cc3)ccc2c1 ; [2H]c1ccc2cc(-c3ccc(-c4c5ccccc5c(-c5cccc(-c6cccc7sc8cc([2H])ccc8c67)c5)c5ccccc45)cc3)ccc2c1]

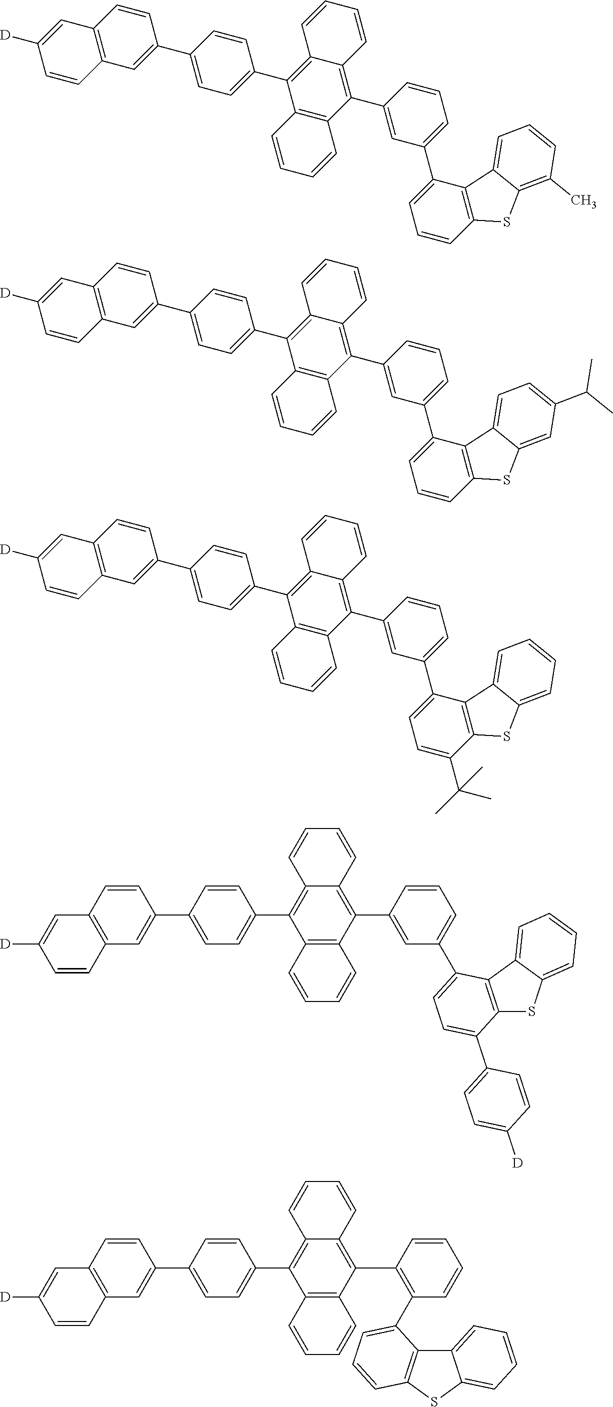 [SMILES: [2H]c1ccc2cc(-c3ccc(-c4c5ccccc5c(-c5cccc(-c6cccc7sc8c(C)cccc8c67)c5)c5ccccc45)cc3)ccc2c1 ; [2H]c1ccc2cc(-c3ccc(-c4c5ccccc5c(-c5cccc(-c6cccc7sc8cc(C(C)C)ccc8c67)c5)c5ccccc45)cc3)ccc2c1 ; [2H]c1ccc2cc(-c3ccc(-c4c5ccccc5c(-c5cccc(-c6ccc(C(C)(C)C)c7sc8ccccc8c67)c5)c5ccccc45)cc3)ccc2c1 ; [2H]c1ccc(-c2ccc(-c3cccc(-c4c5ccccc5c(-c5ccc(-c6ccc7cc([2H])ccc7c6)cc5)c5ccccc45)c3)c3c2sc2ccccc23)cc1 ; [2H]c1ccc2cc(-c3ccc(-c4c5ccccc5c(-c5ccccc5-c5cccc6sc7ccccc7c56)c5ccccc45)cc3)ccc2c1]

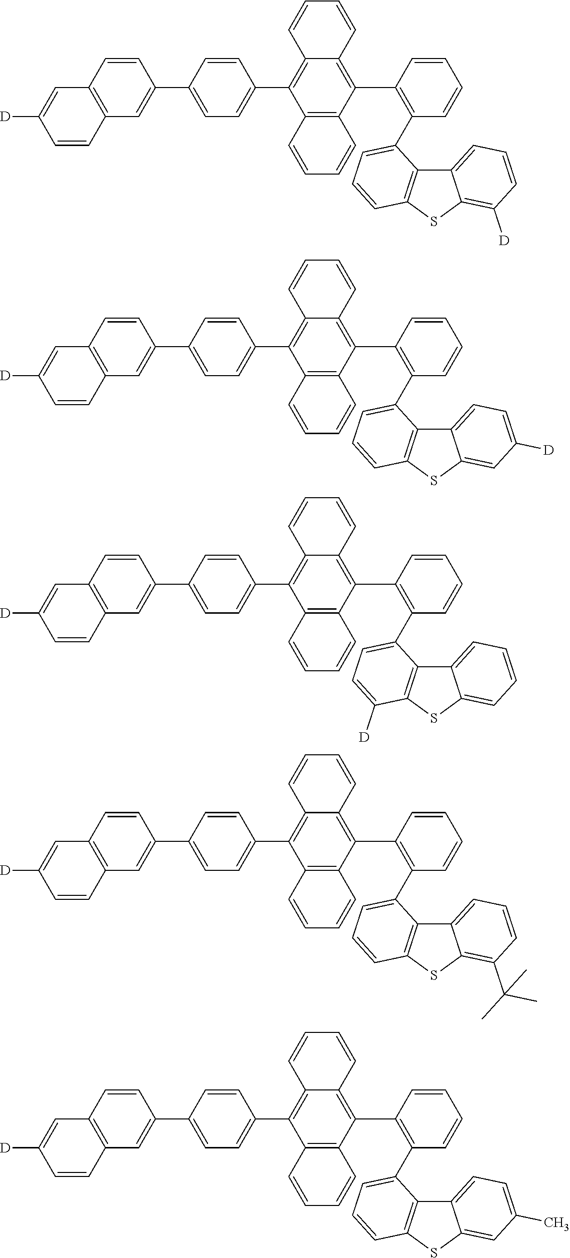

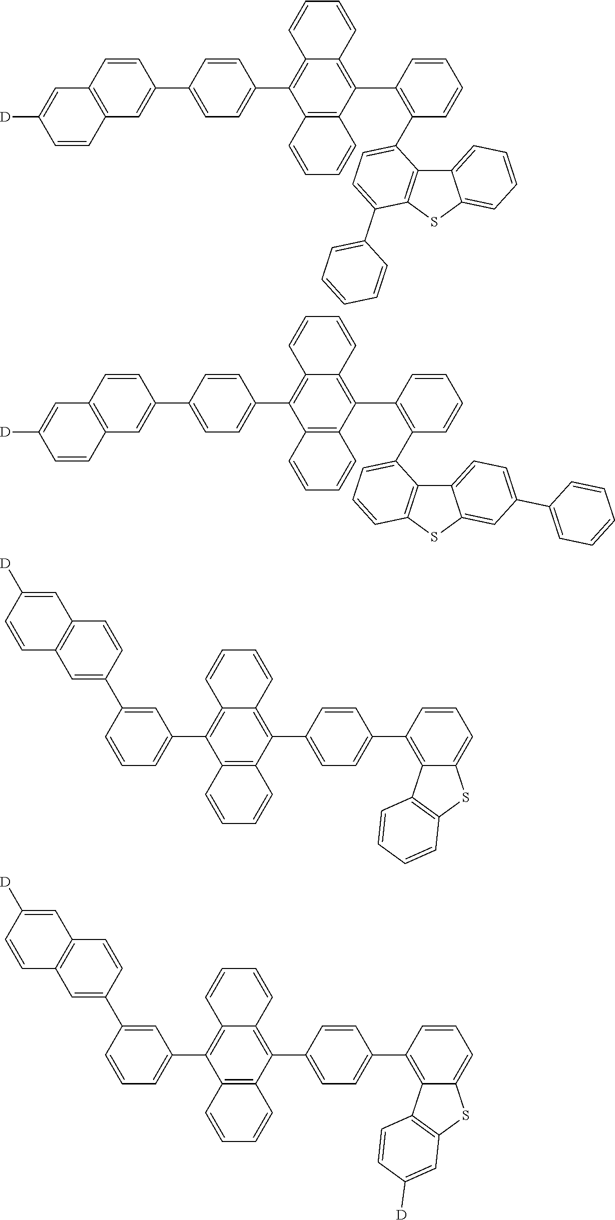

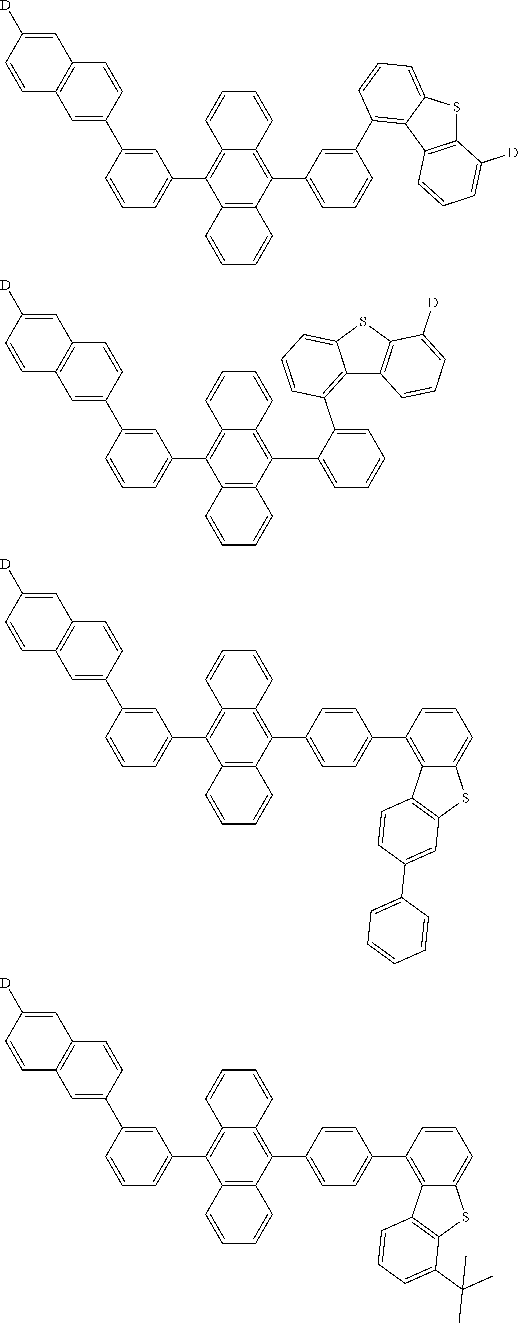 [SMILES: [2H]c1ccc2cc(-c3cccc(-c4c5ccccc5c(-c5cccc(-c6cccc7sc8c([2H])cccc8c67)c5)c5ccccc45)c3)ccc2c1 ; [2H]c1ccc2cc(-c3cccc(-c4c5ccccc5c(-c5ccccc5-c5cccc6sc7c([2H])cccc7c56)c5ccccc45)c3)ccc2c1 ; [2H]c1ccc2cc(-c3cccc(-c4c5ccccc5c(-c5ccc(-c6cccc7sc8cc(-c9ccccc9)ccc8c67)cc5)c5ccccc45)c3)ccc2c1 ; [2H]c1ccc2cc(-c3cccc(-c4c5ccccc5c(-c5ccc(-c6cccc7sc8c(C(C)(C)C)cccc8c67)cc5)c5ccccc45)c3)ccc2c1]

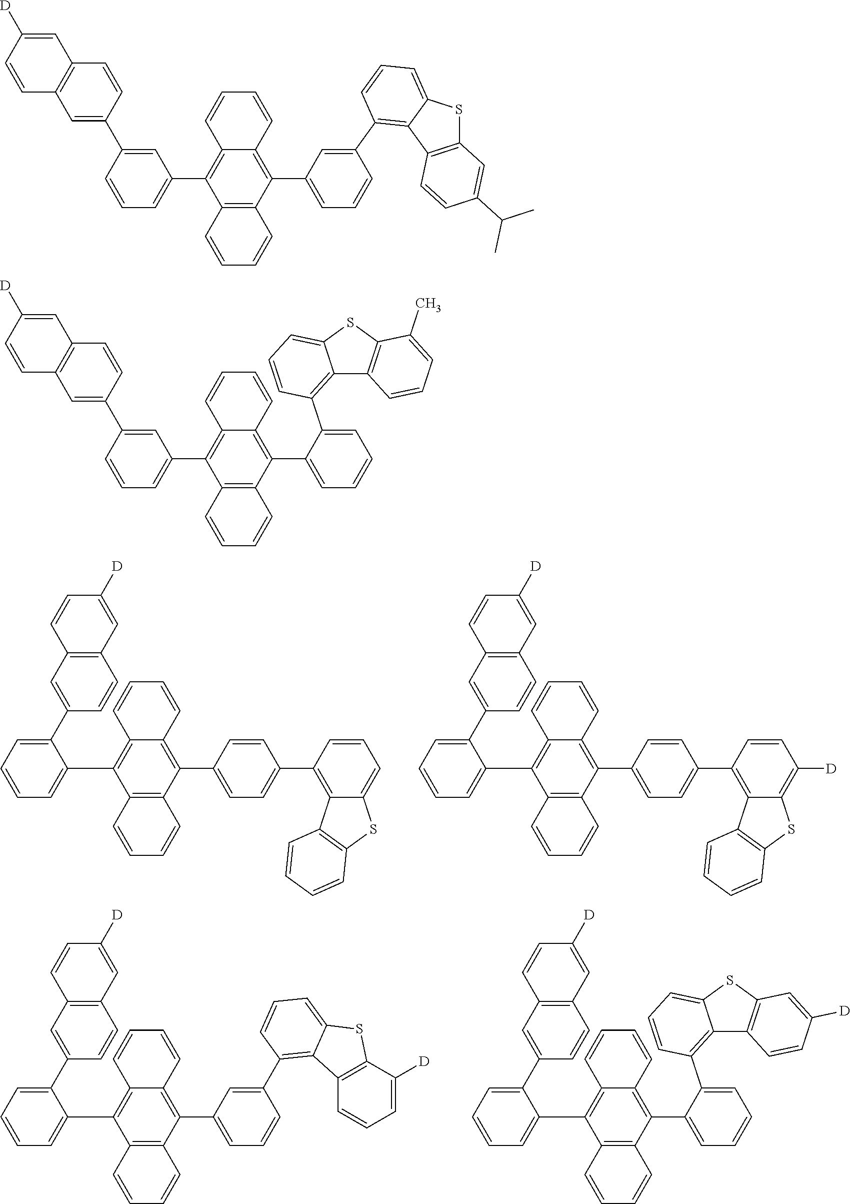

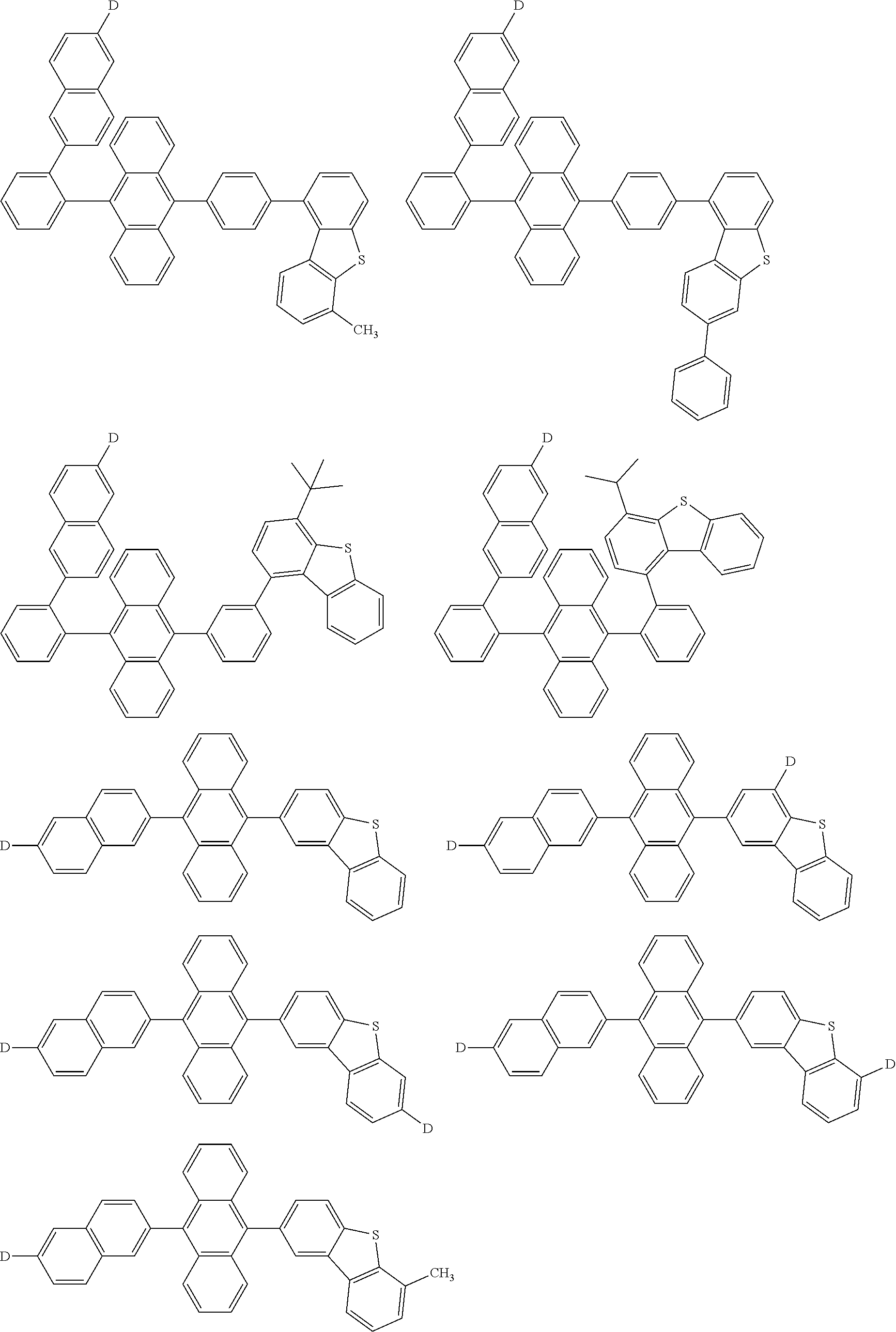

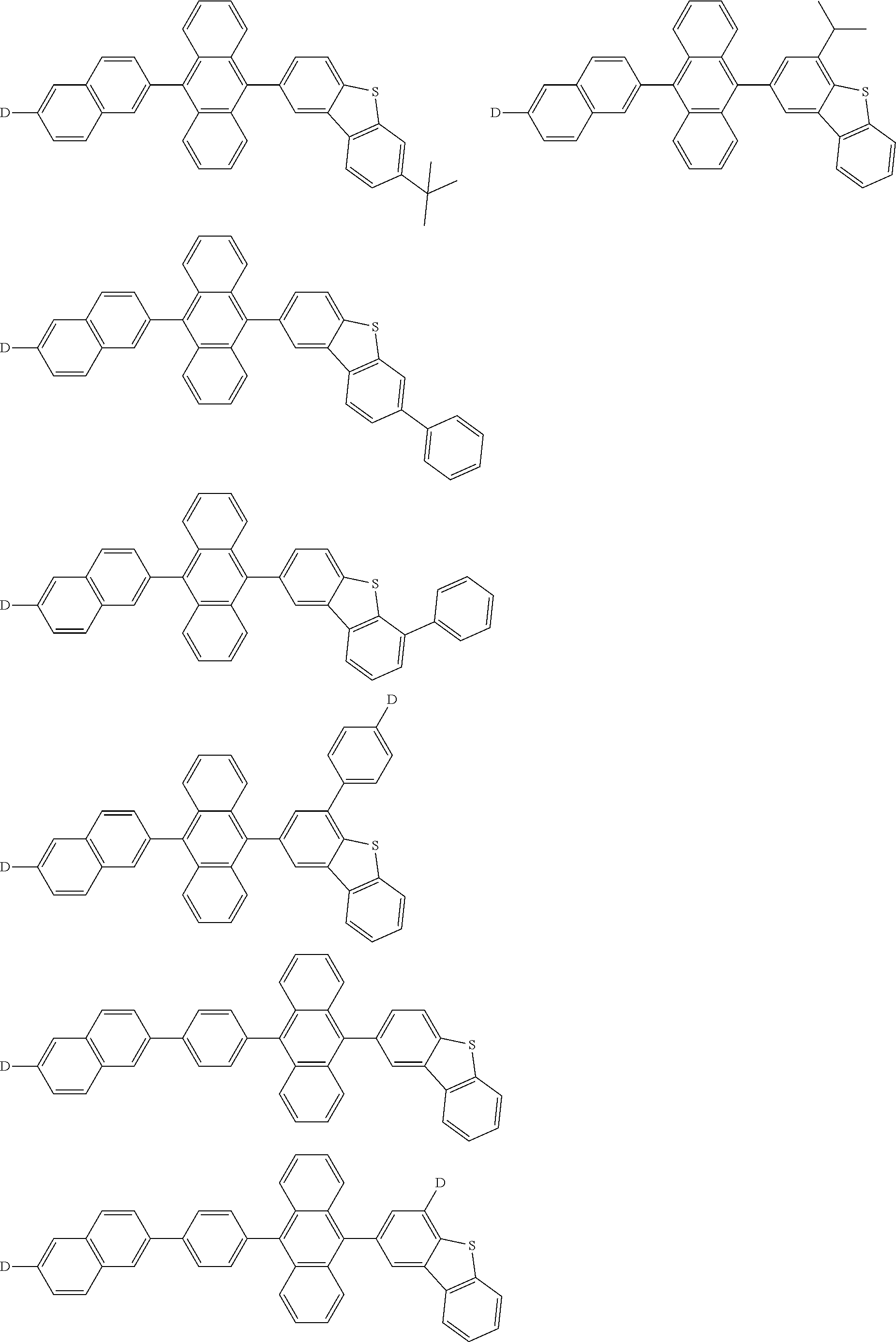

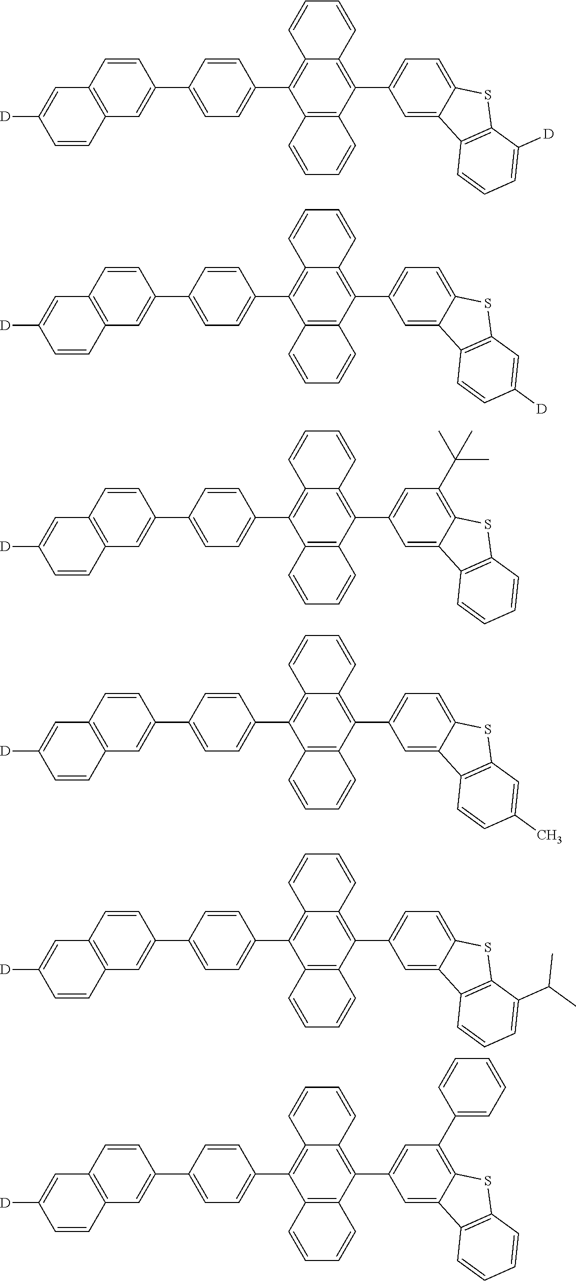

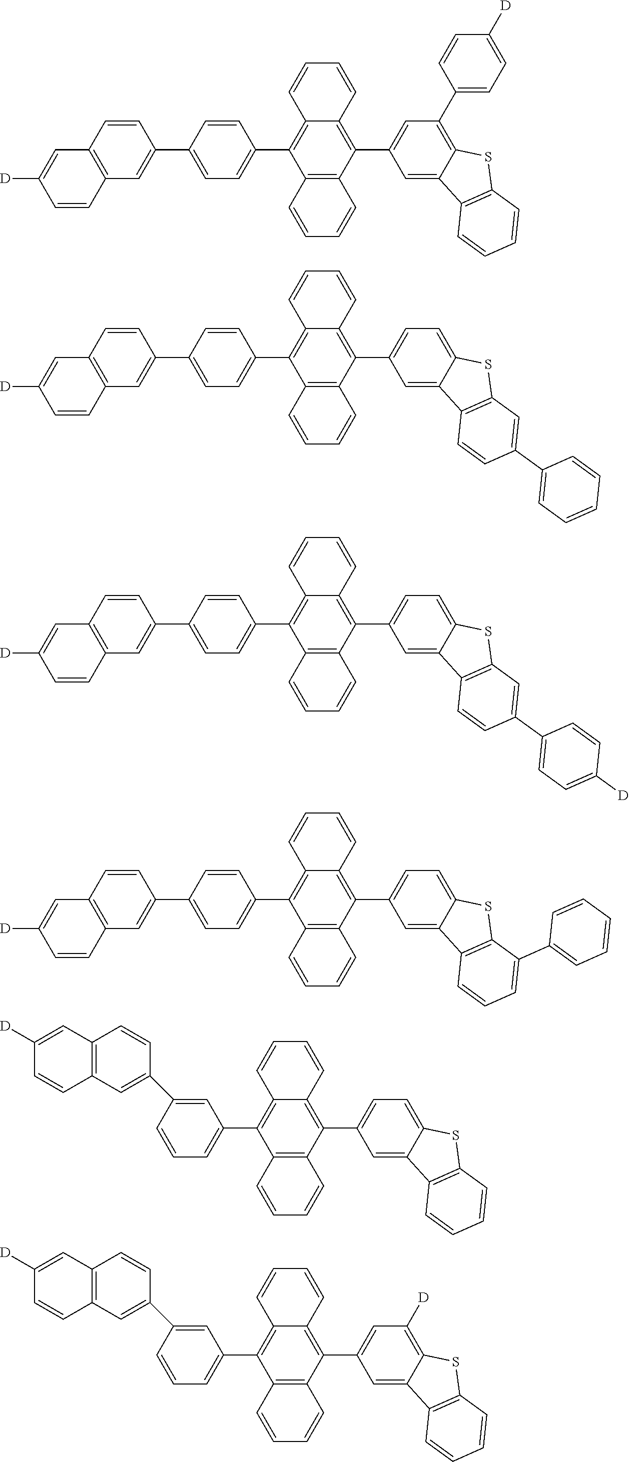

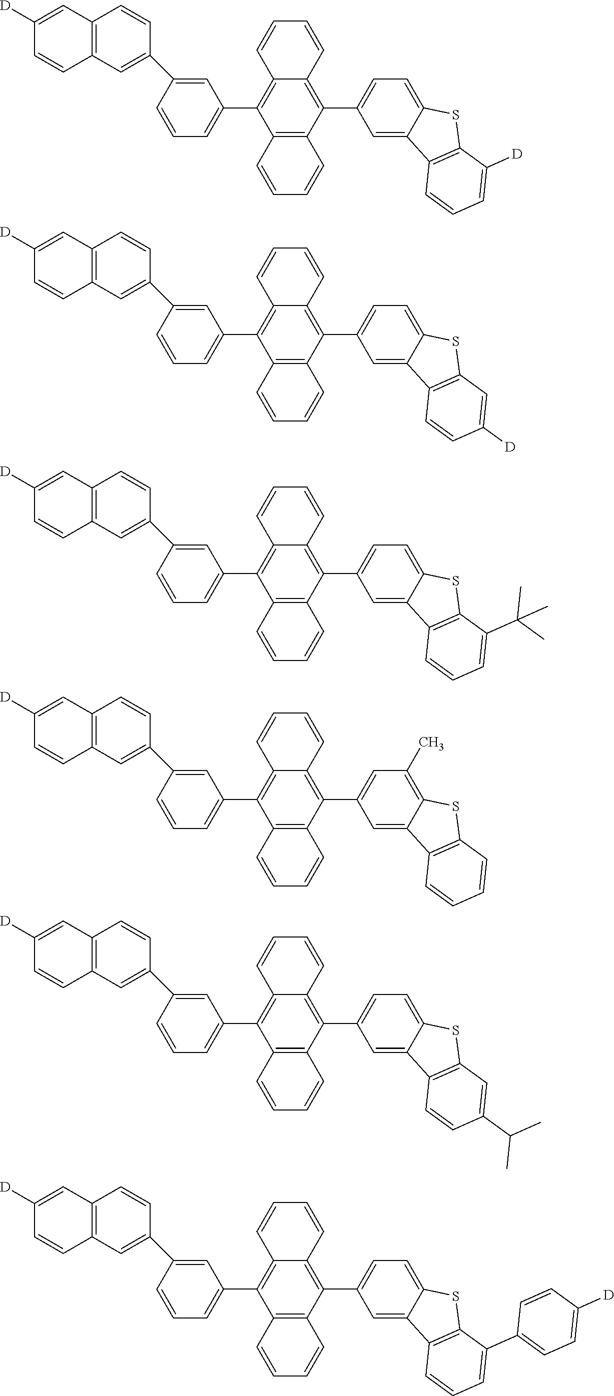

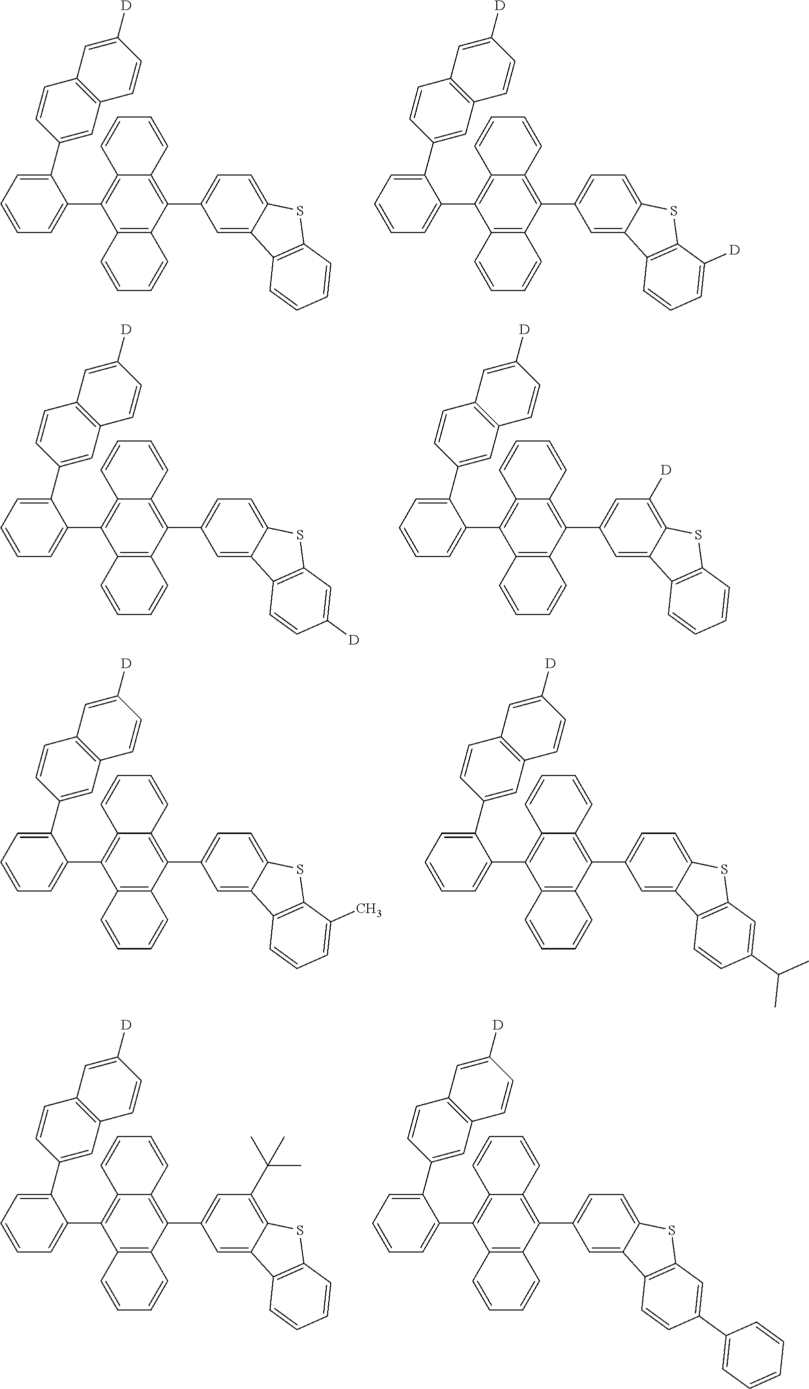

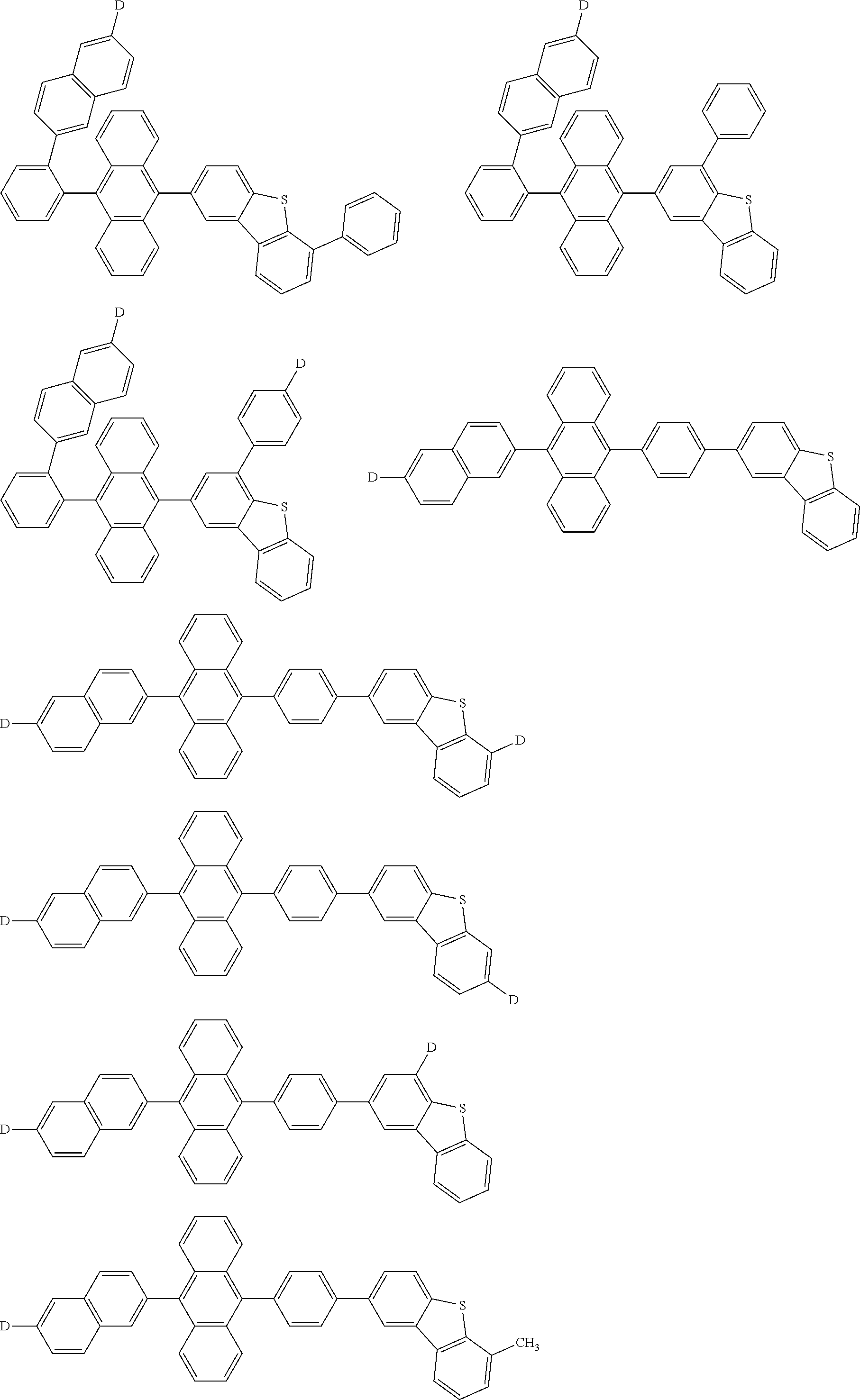

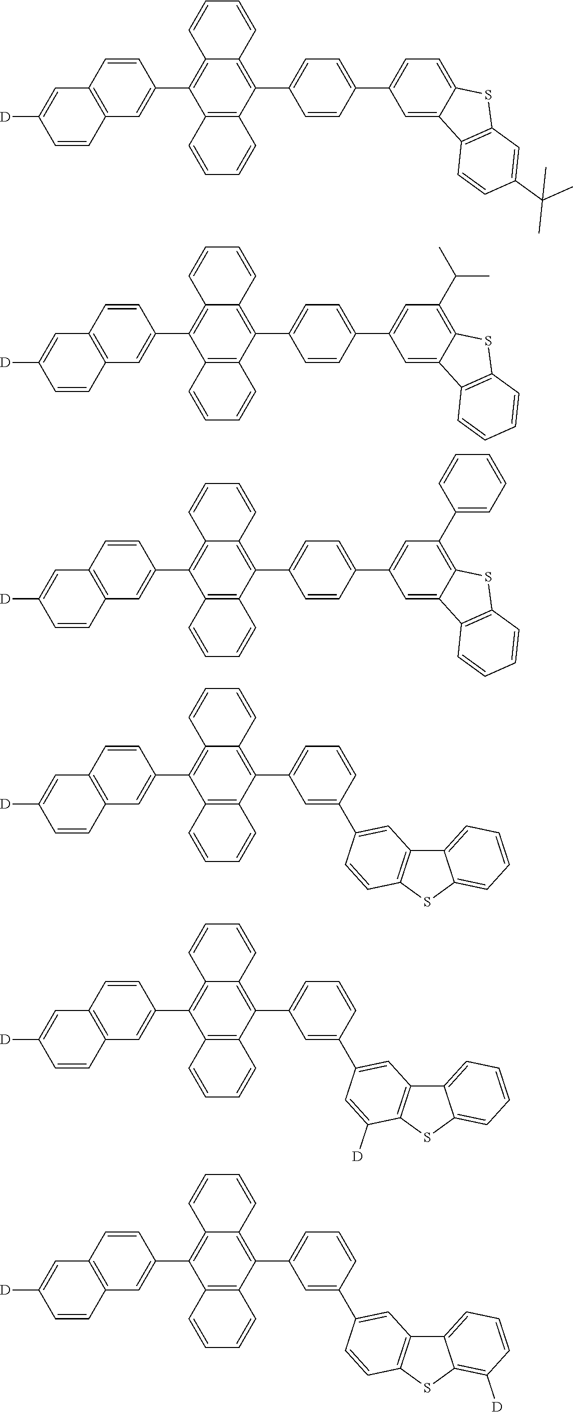

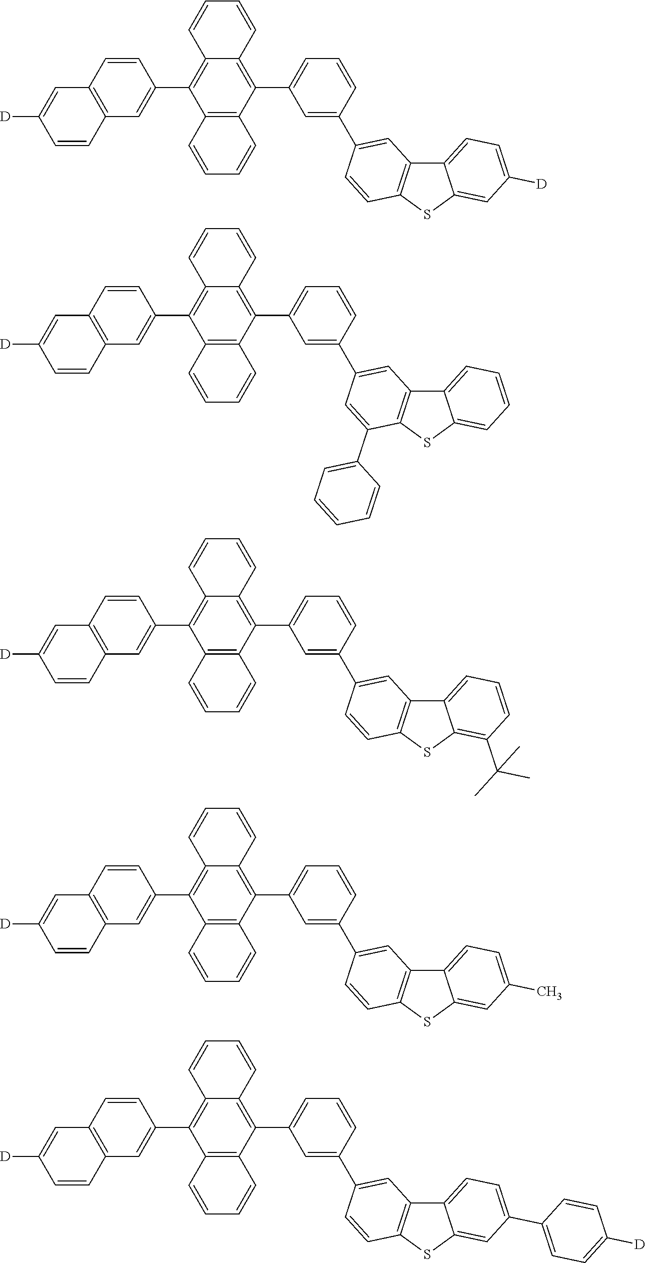 [SMILES: [2H]c1ccc2cc(-c3c4ccccc4c(-c4cccc(-c5ccc6sc7cc([2H])ccc7c6c5)c4)c4ccccc34)ccc2c1 ; [2H]c1ccc2cc(-c3c4ccccc4c(-c4cccc(-c5cc(-c6ccccc6)c6sc7ccccc7c6c5)c4)c4ccccc34)ccc2c1 ; [2H]c1ccc2cc(-c3c4ccccc4c(-c4cccc(-c5ccc6sc7c(C(C)(C)C)cccc7c6c5)c4)c4ccccc34)ccc2c1 ; [2H]c1ccc2cc(-c3c4ccccc4c(-c4cccc(-c5ccc6sc7cc(C)ccc7c6c5)c4)c4ccccc34)ccc2c1 ; [2H]c1ccc(-c2ccc3c(c2)sc2ccc(-c4cccc(-c5c6ccccc6c(-c6ccc7cc([2H])ccc7c6)c6ccccc56)c4)cc23)cc1]

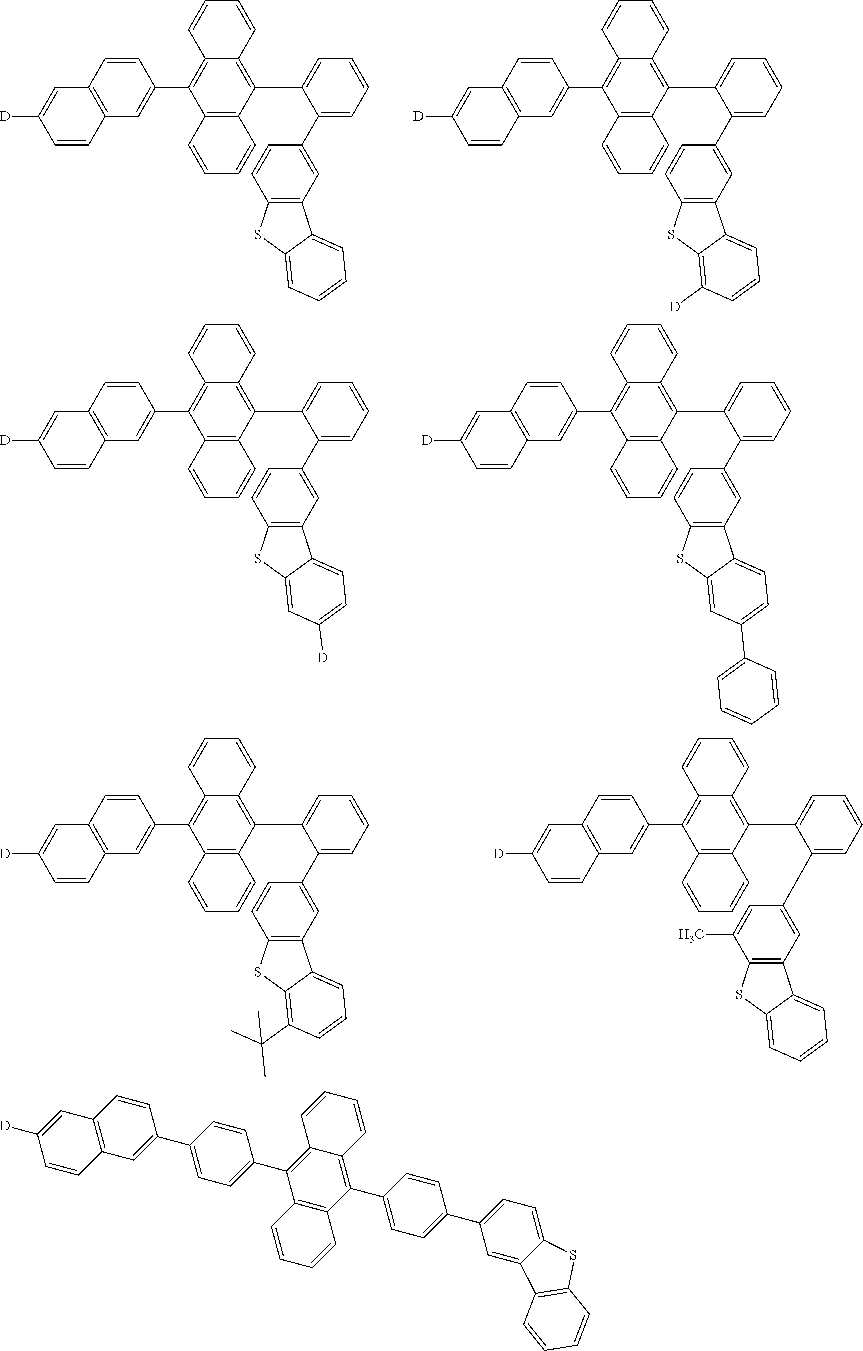

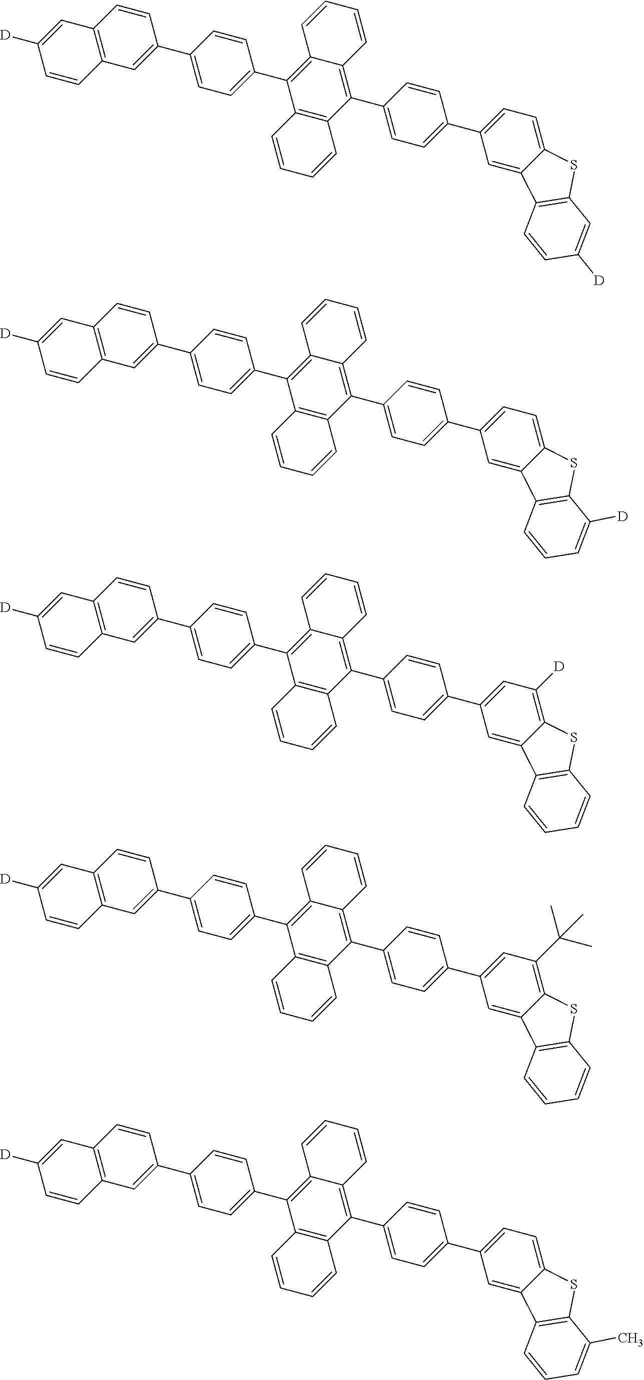 [SMILES: [2H]c1ccc2cc(-c3ccc(-c4c5ccccc5c(-c5ccc(-c6ccc7sc8cc([2H])ccc8c7c6)cc5)c5ccccc45)cc3)ccc2c1 ; [2H]c1ccc2cc(-c3ccc(-c4c5ccccc5c(-c5ccc(-c6ccc7sc8c([2H])cccc8c7c6)cc5)c5ccccc45)cc3)ccc2c1 ; [2H]c1ccc2cc(-c3ccc(-c4c5ccccc5c(-c5ccc(-c6cc([2H])c7sc8ccccc8c7c6)cc5)c5ccccc45)cc3)ccc2c1 ; [2H]c1ccc2cc(-c3ccc(-c4c5ccccc5c(-c5ccc(-c6cc(C(C)(C)C)c7sc8ccccc8c7c6)cc5)c5ccccc45)cc3)ccc2c1 ; [2H]c1ccc2cc(-c3ccc(-c4c5ccccc5c(-c5ccc(-c6ccc7sc8c(C)cccc8c7c6)cc5)c5ccccc45)cc3)ccc2c1]

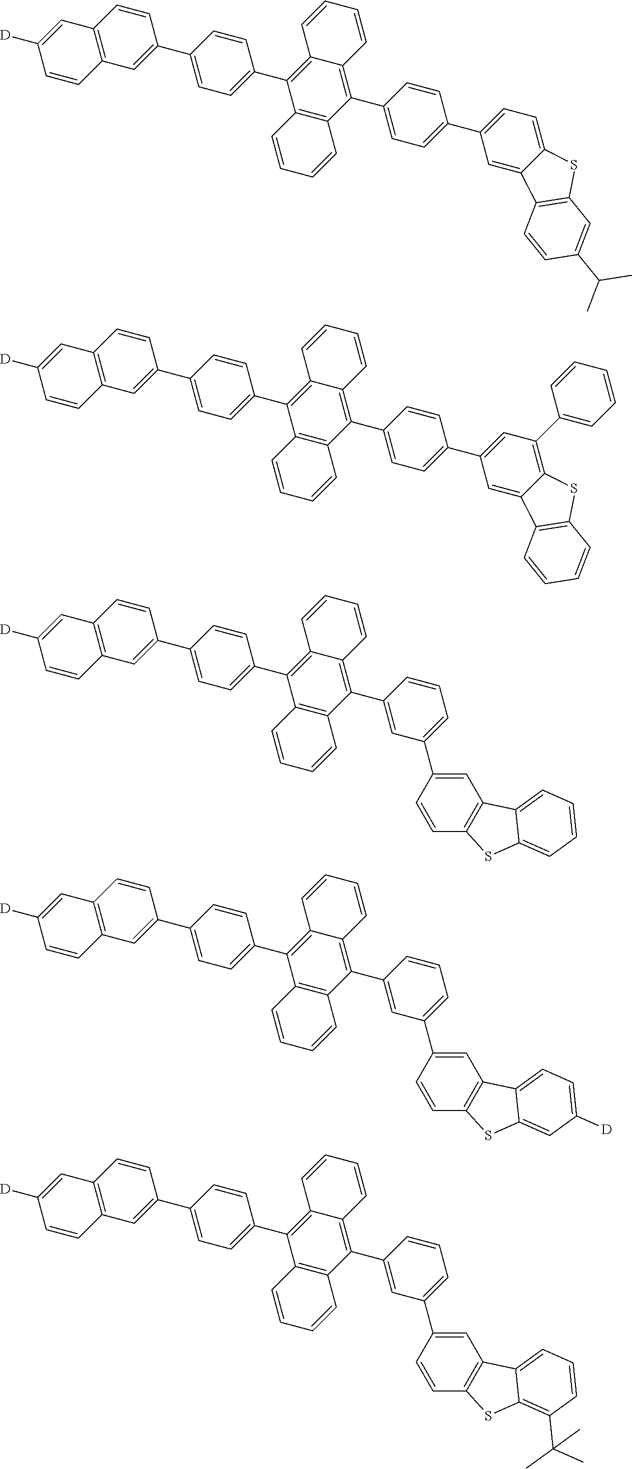 [SMILES: [2H]c1ccc2cc(-c3ccc(-c4c5ccccc5c(-c5ccc(-c6ccc7sc8cc(C(C)C)ccc8c7c6)cc5)c5ccccc45)cc3)ccc2c1 ; [2H]c1ccc2cc(-c3ccc(-c4c5ccccc5c(-c5ccc(-c6cc(-c7ccccc7)c7sc8ccccc8c7c6)cc5)c5ccccc45)cc3)ccc2c1 ; [2H]c1ccc2cc(-c3ccc(-c4c5ccccc5c(-c5cccc(-c6ccc7sc8ccccc8c7c6)c5)c5ccccc45)cc3)ccc2c1 ; [2H]c1ccc2cc(-c3ccc(-c4c5ccccc5c(-c5cccc(-c6ccc7sc8cc([2H])ccc8c7c6)c5)c5ccccc45)cc3)ccc2c1 ; [2H]c1ccc2cc(-c3ccc(-c4c5ccccc5c(-c5cccc(-c6ccc7sc8c(C(C)(C)C)cccc8c7c6)c5)c5ccccc45)cc3)ccc2c1]

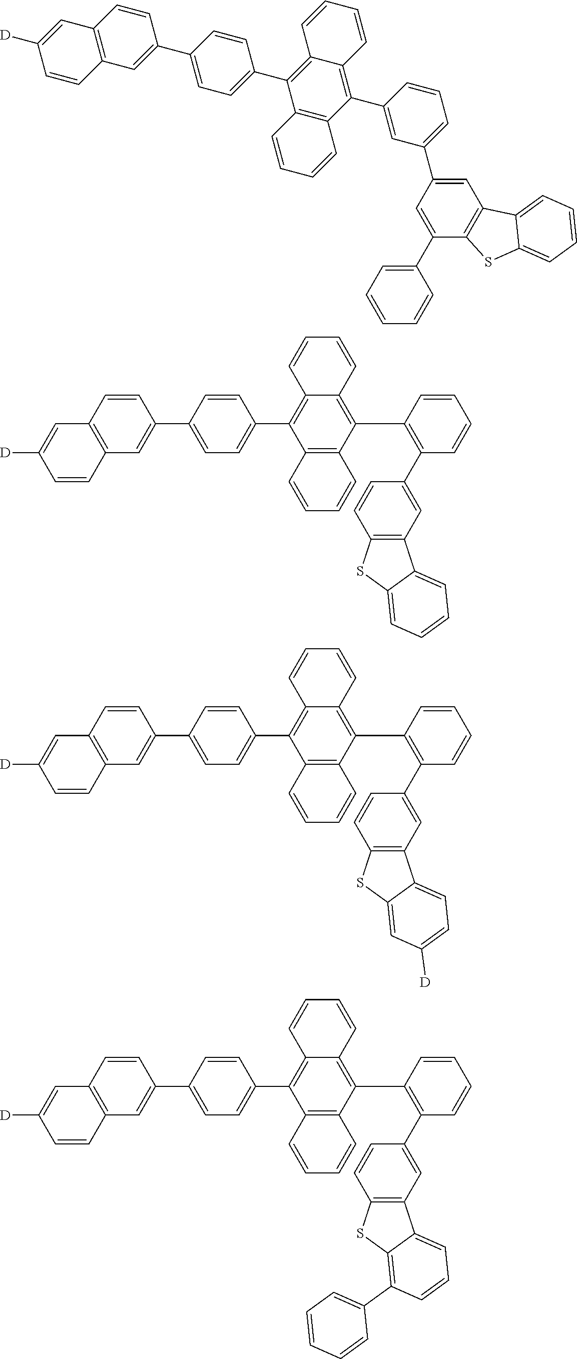

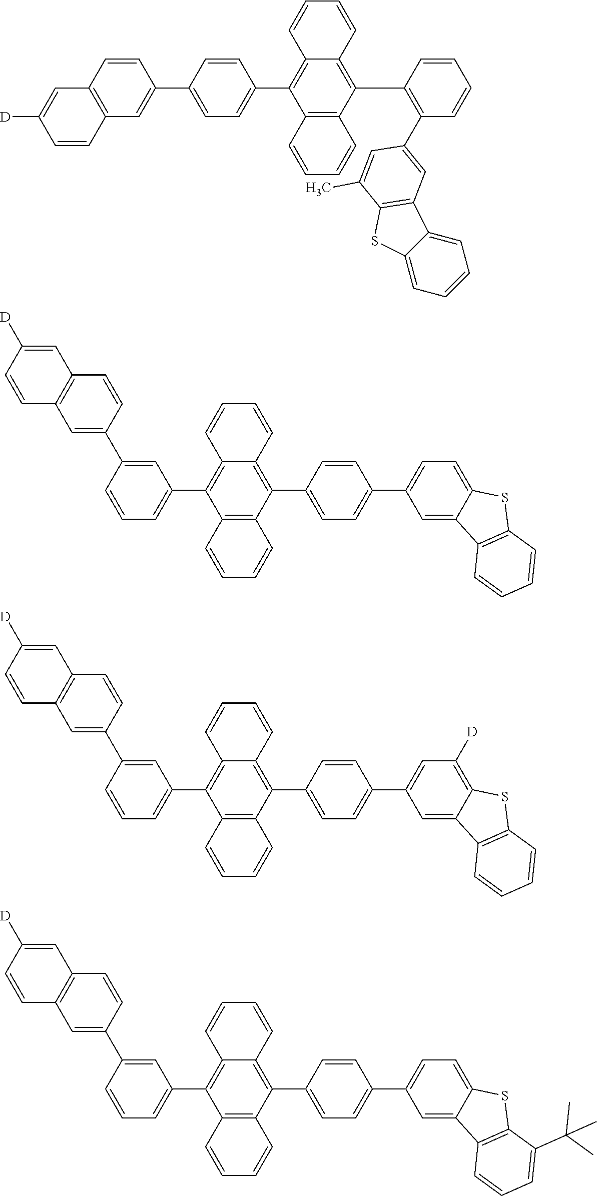 [SMILES: [2H]c1ccc2cc(-c3ccc(-c4c5ccccc5c(-c5ccccc5-c5cc(C)c6sc7ccccc7c6c5)c5ccccc45)cc3)ccc2c1 ; [2H]c1ccc2cc(-c3cccc(-c4c5ccccc5c(-c5ccc(-c6ccc7sc8ccccc8c7c6)cc5)c5ccccc45)c3)ccc2c1 ; [2H]c1ccc2cc(-c3cccc(-c4c5ccccc5c(-c5ccc(-c6cc([2H])c7sc8ccccc8c7c6)cc5)c5ccccc45)c3)ccc2c1 ; [2H]c1ccc2cc(-c3cccc(-c4c5ccccc5c(-c5ccc(-c6ccc7sc8c(C(C)(C)C)cccc8c7c6)cc5)c5ccccc45)c3)ccc2c1]

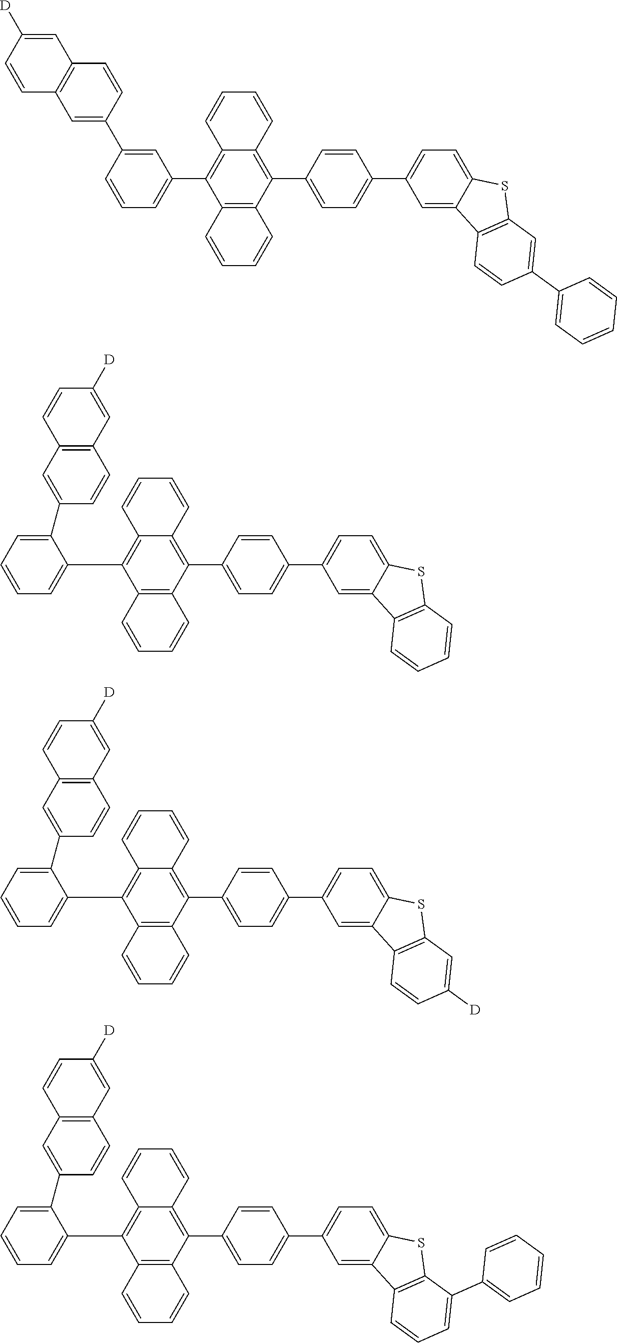 [SMILES: [2H]c1ccc2cc(-c3cccc(-c4c5ccccc5c(-c5ccc(-c6ccc7sc8cc(-c9ccccc9)ccc8c7c6)cc5)c5ccccc45)c3)ccc2c1 ; [2H]c1ccc2cc(-c3ccccc3-c3c4ccccc4c(-c4ccc(-c5ccc6sc7ccccc7c6c5)cc4)c4ccccc34)ccc2c1 ; [2H]c1ccc2cc(-c3ccccc3-c3c4ccccc4c(-c4ccc(-c5ccc6sc7cc([2H])ccc7c6c5)cc4)c4ccccc34)ccc2c1 ; [2H]c1ccc2cc(-c3ccccc3-c3c4ccccc4c(-c4ccc(-c5ccc6sc7c(-c8ccccc8)cccc7c6c5)cc4)c4ccccc34)ccc2c1]

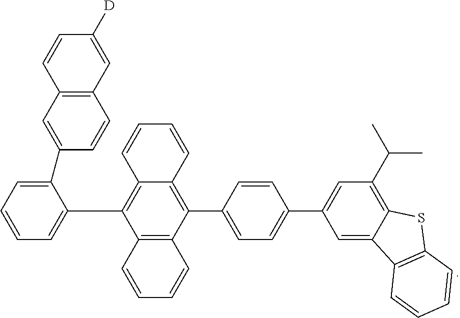 [SMILES: [2H]c1ccc2cc(-c3ccccc3-c3c4ccccc4c(-c4ccc(-c5cc(C(C)C)c6sc7ccccc7c6c5)cc4)c4ccccc34)ccc2c1]

The present application further provides a mixture. The mixture includes a first organic compound H1 and a second organic compound H2. The first organic compound H1 is the anthracene-based compound described above. The second organic compound H2 is at least one material selected from a hole injection material (HIM), a hole transport material (HTM), an electron transport material (ETM), an electron injection material (EIM), an electron blocking material (EBM), a hole blocking material (HBM), a light-emitting guest material, a light-emitting host material, and an organic dye.

In one embodiment, the second organic compound H2 is selected from an organic compound represented by formula (III-1) or formula (III-2):

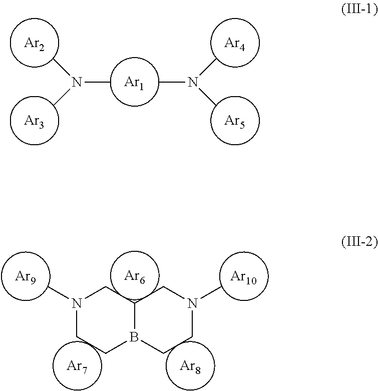

Each of Ar₁ to Ar₁₀ is independently selected from a substituted or unsubstituted aromatic group containing 6-40 ring atoms, and a substituted or unsubstituted heteroaromatic group containing 5-40 ring atoms. One or more of the groups may form a single ring or a multi ring of an aliphatic group or an aromatic ring system by combining each other and/or combining a ring bonded with the group.

In one embodiment, the Ar₁ is selected from formula (D-1), formula (D-2), and formula (D-3);

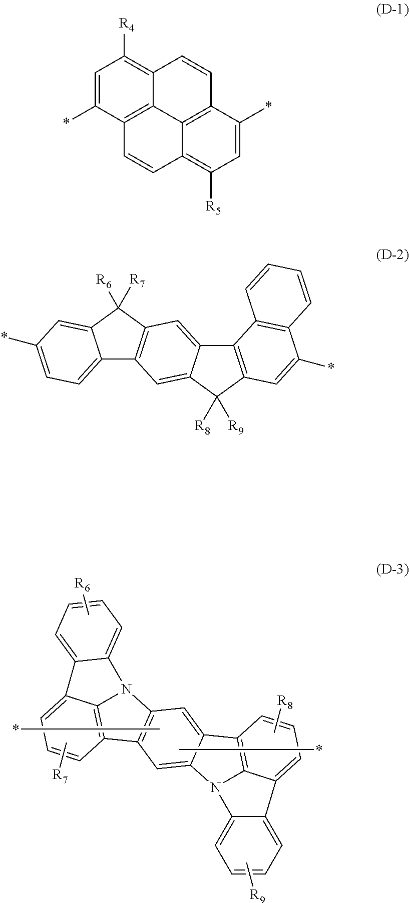

Each of R₄ to R₈ is independently selected from —H, -D, a C₁-C₂₀ linear alkyl group, a C₁-C₂₀ alkoxy group, a C₁-C₂₀ thioalkoxy group, a C₃-C₂₀ branched or cyclic alkyl group, a C₃-C₂₀ alkoxy group, a C₃-C₂₀ thioalkoxy group, a silyl group, a C₁-C₂₀ ketone group, a C₂-C₂₀ alkoxycarbonyl group, a C₇-C₂₀ aroxycarbonyl group, a cyano group, a carbamoyl group, a haloformyl group, a formyl group, an isocyano group, an isocyanate group, a thiocyanate ester group, an isothiocyanate ester group, a hydroxyl group, a nitro group, an amino group, —CF₃, —Cl, —Br, —F, —I, a substituted or unsubstituted C₂-C₂₀ alkenyl group, a substituted or unsubstituted aromatic group containing 5-30 ring atoms, a substituted or unsubstituted heteroaromatic group containing 5-30 ring atoms, an aryloxy group containing 5-30 ring atoms, a heteroaryloxy group containing 5-30 ring atoms, and combinations of these groups.

* indicates a binding site.

In one embodiment, each of R₄ to R₈ is independently selected from —H, -D, a C₁-C₁₀ linear alkyl group, a C₃-C₁₀ branched or cyclic alkyl group, a phenyl group, and a phenyl group substituted by -D, a C₁-C₁₀ linear alkyl group, or a C₃-C₁₀ branched or cyclic alkyl group.

In one embodiment, each of Ar₂ to Ar₈ is independently selected from a substituted or unsubstituted aromatic group containing 5-25 ring atoms, and a substituted or unsubstituted heteroaromatic group containing 5-25 ring atoms.

In some embodiments, each of Ar₂ to Ar₈ is independently selected from following groups:

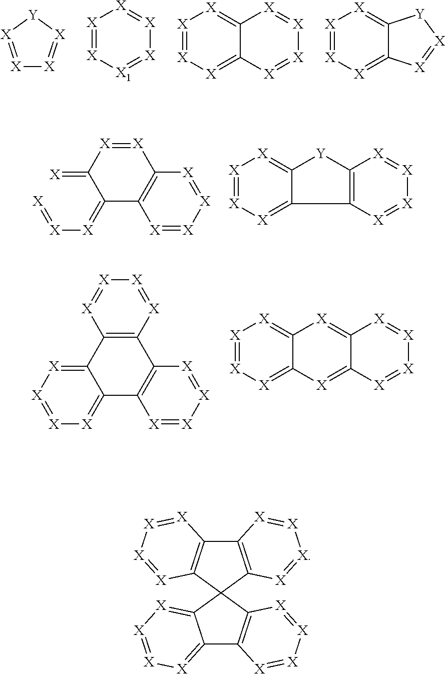

Each X is independently selected from CR₉ and N;

Each Y is independently selected from NR₁₀, PR₁₀, CR₁₁R₁₂, SiR₁₁R₁₂, O, S, S(═O)₂, and S(═O);

each of R₉, R₁₀, R₁₁, and R₁₂ is independently selected from —H, -D, a C₁-C₂₉ linear alkyl group, a C₁-C₂₉ alkoxy group, a C₁-C₂₉ thioalkoxy group, a C₃-C₂₀ branched or cyclic alkyl group, a C₃-C₂₀ alkoxy group, a C₃-C₂₀ thioalkoxy group, a silyl group, a C₁-C₂₉ ketone group, a C₂-C₂₀ alkoxycarbonyl group, an aroxycarbonyl group, a cyano group, a carbamoyl group, a haloformyl group, a formyl group, an isocyano group, an isocyanate group, a thiocyanate ester group, an isothiocyanate ester group, a hydroxyl group, a nitro group, an amino group, —CF₃, —Cl, —Br, —F, —I, a substituted or unsubstituted C₂-C₂₀ alkenyl group, a substituted or unsubstituted aromatic group containing 5-30 ring atoms, a substituted or unsubstituted heteroaromatic group containing 5-30 ring atoms, an aryloxy group containing 5-30 ring atoms, a heteroaryloxy group containing 5-30 ring atoms, and combinations of these groups.

In the present application, when the X is the binding site, X is C, and when the Y is the binding site, Y is N.

In one embodiment, the second organic compound represented by the formula (III-1) is selected from, but not limited thereto:

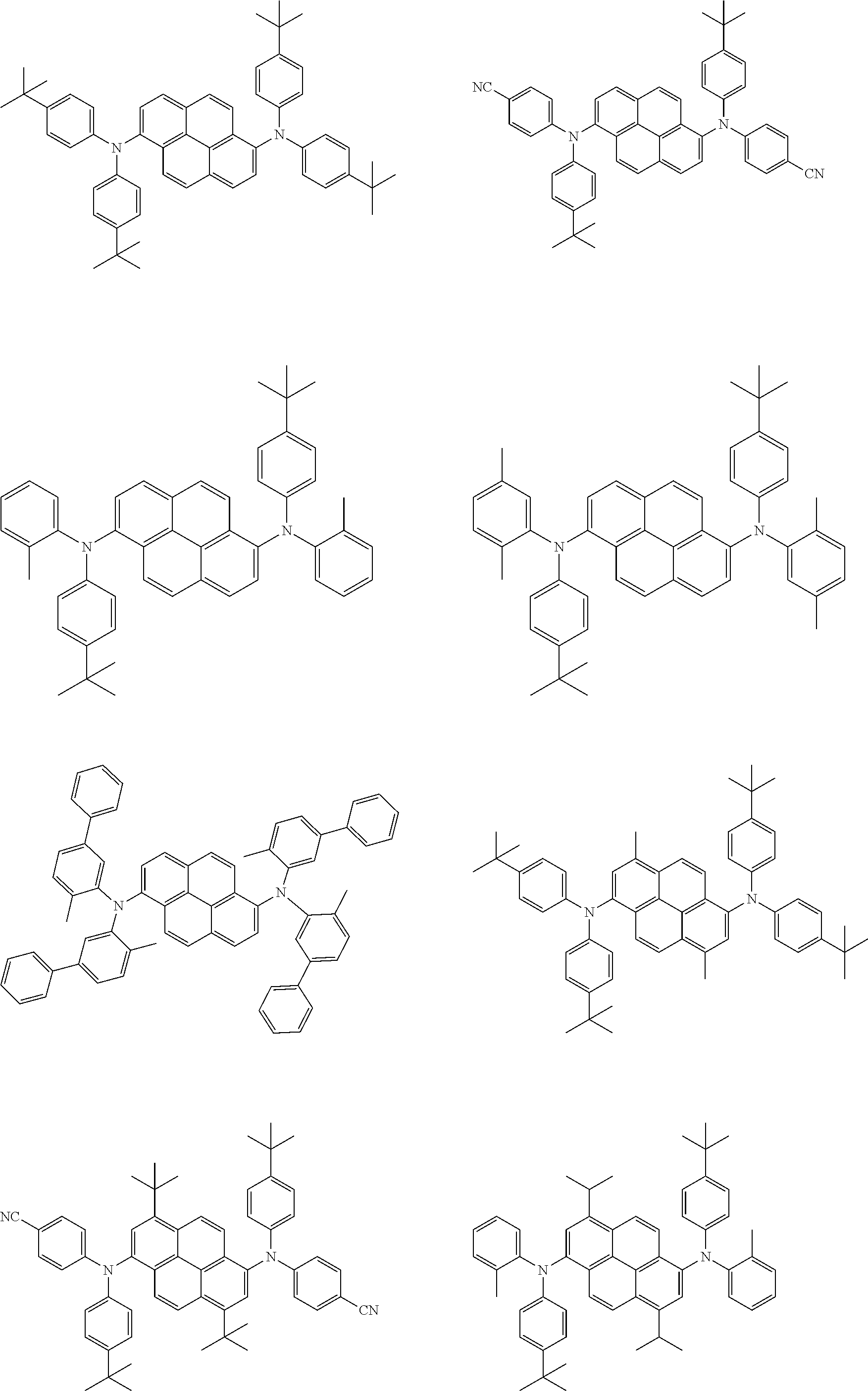

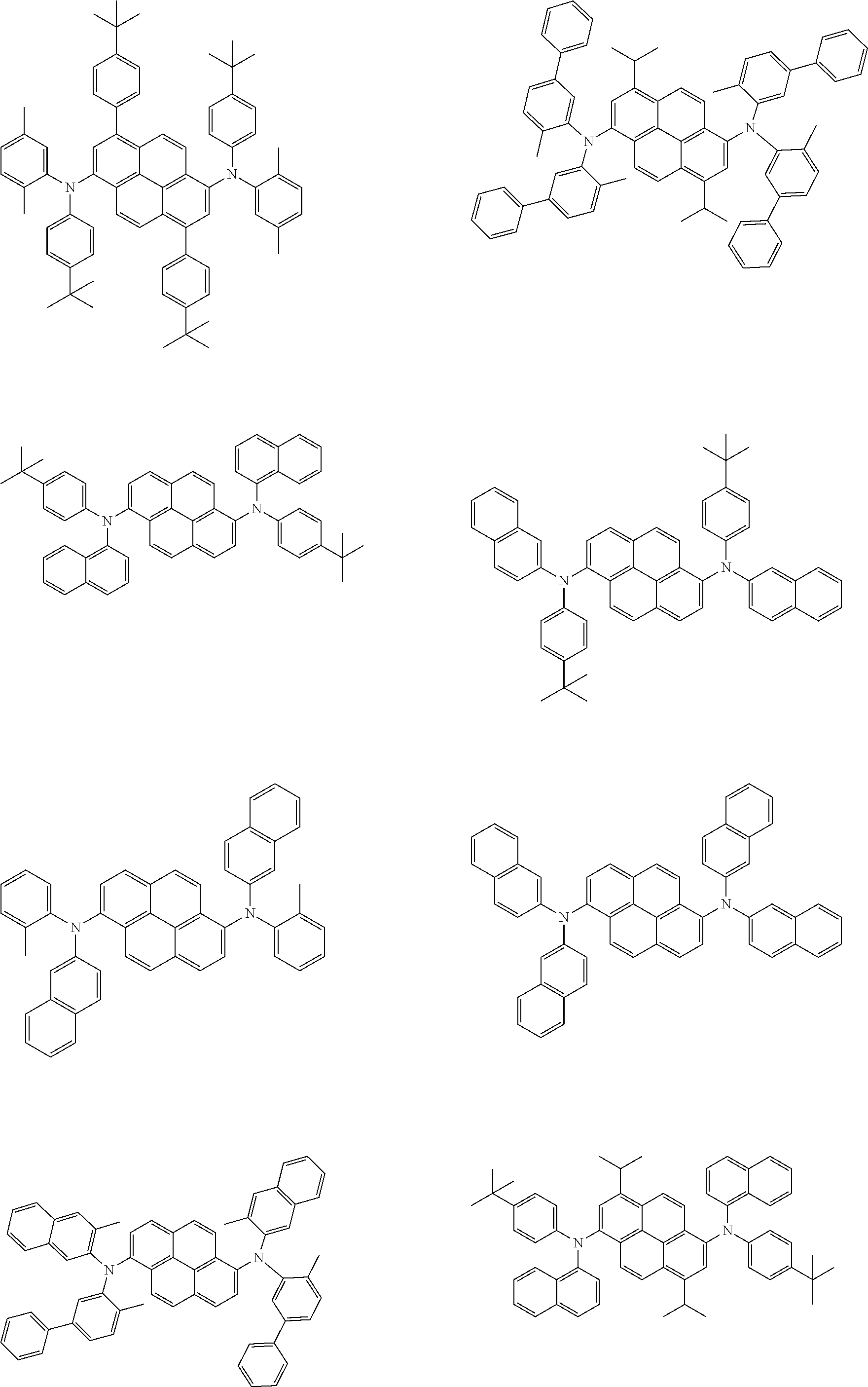

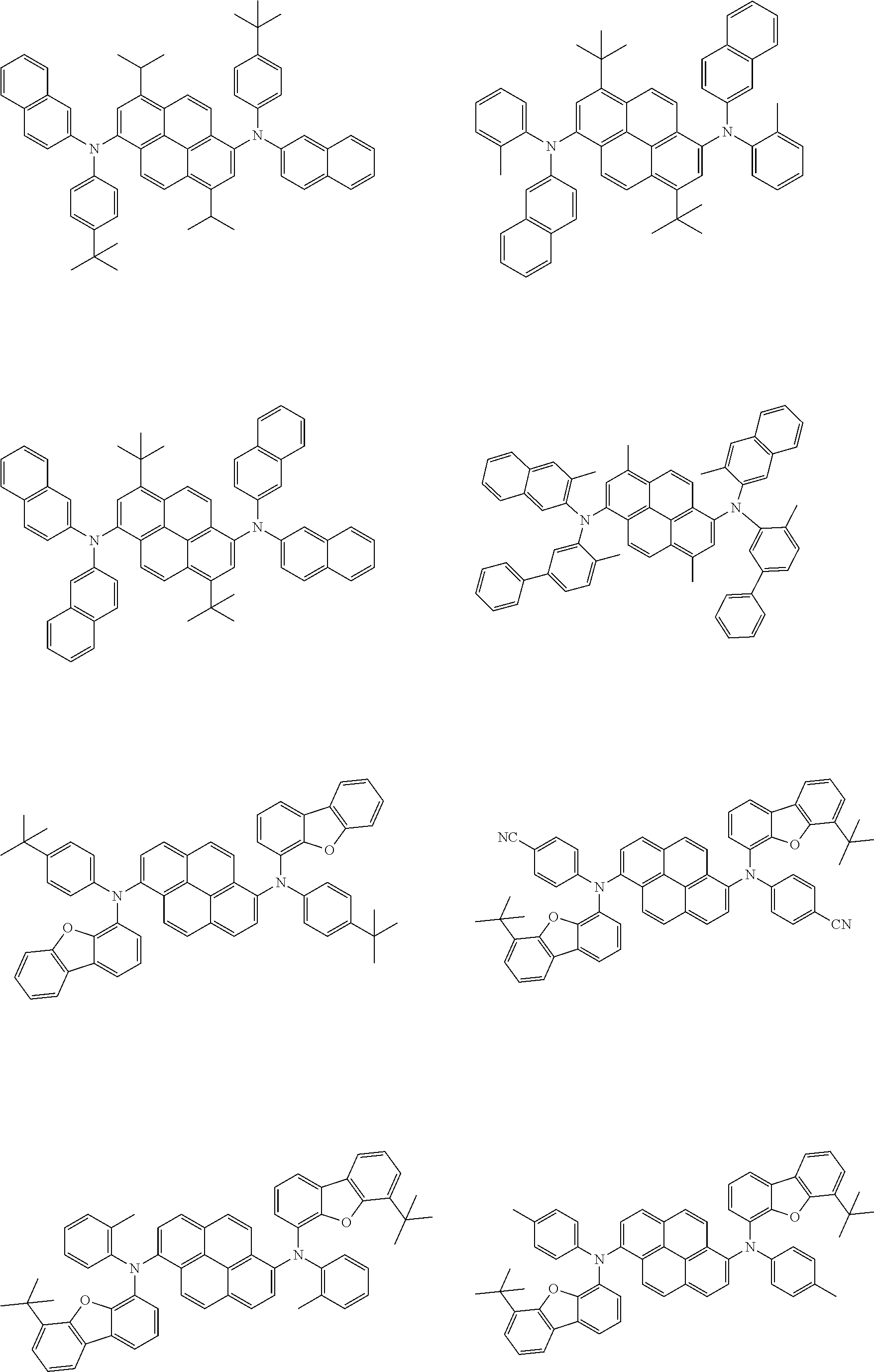

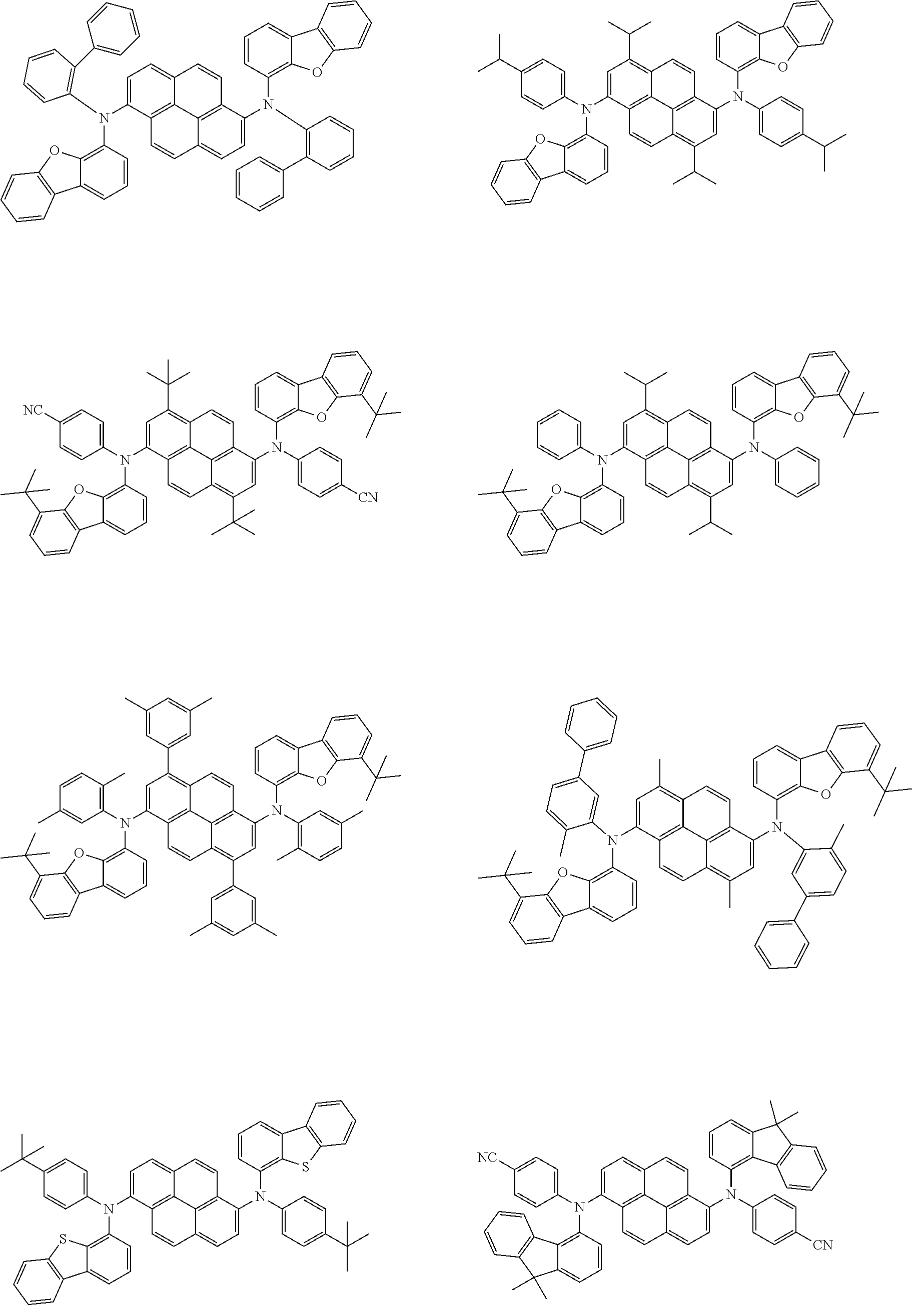

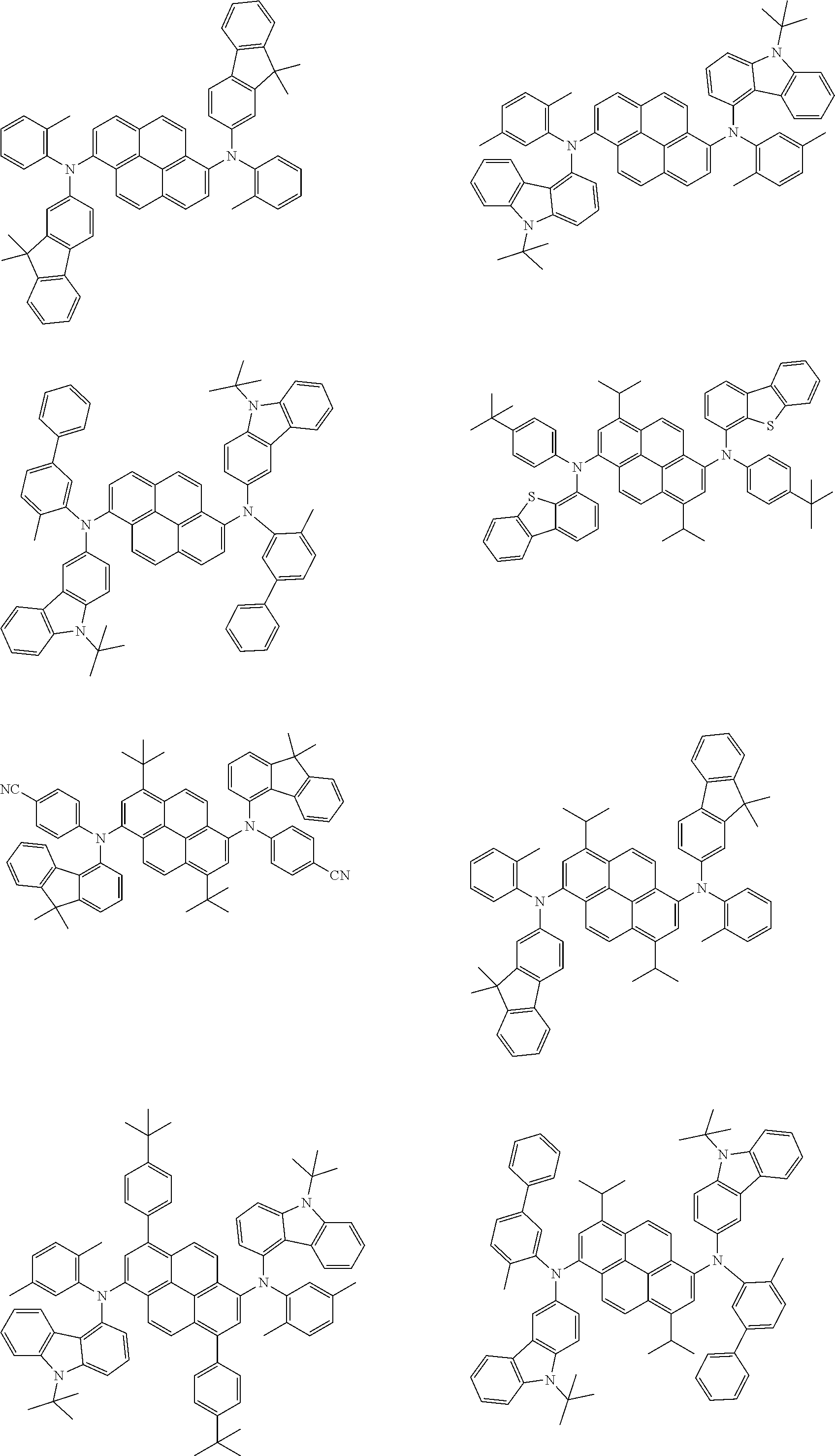

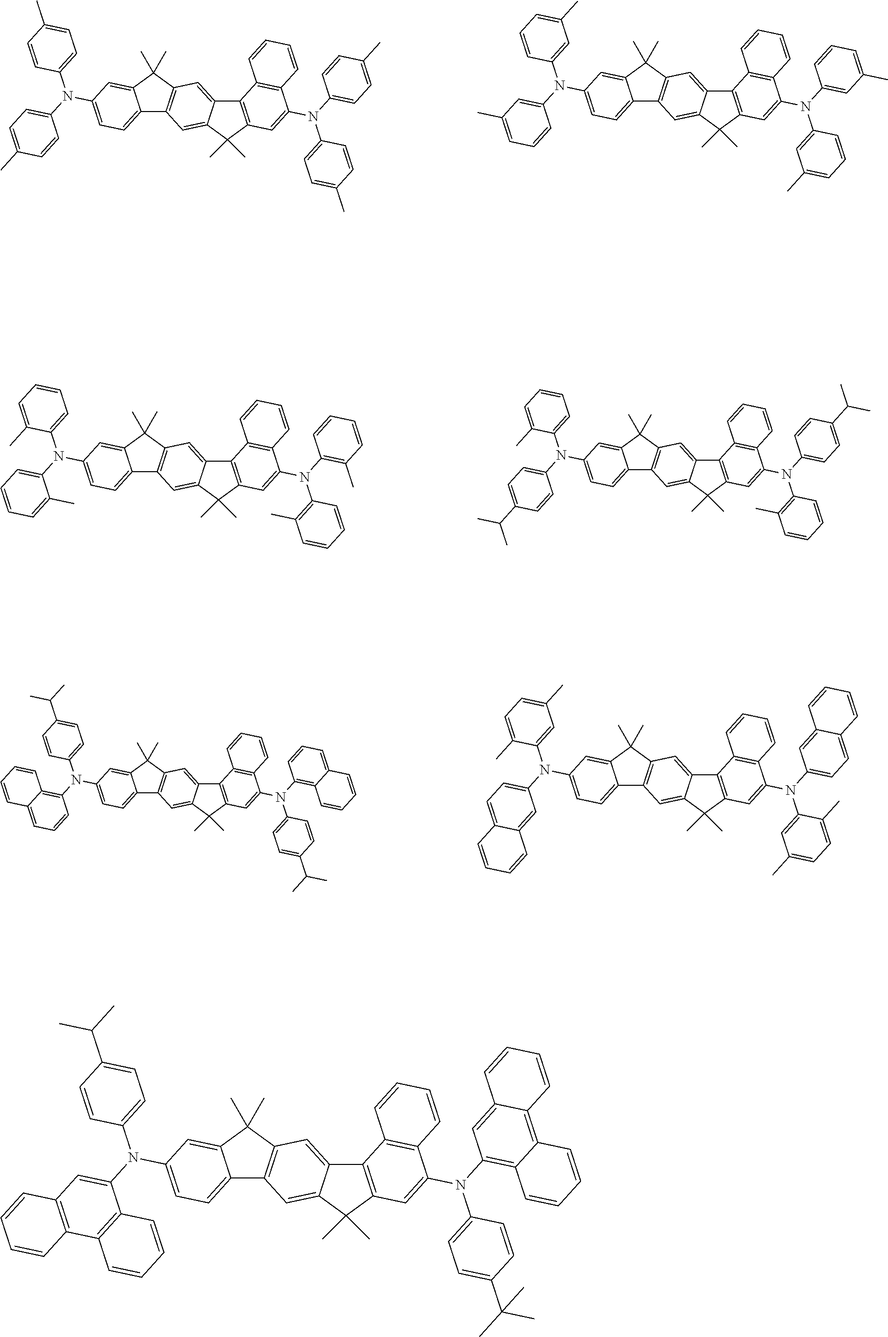

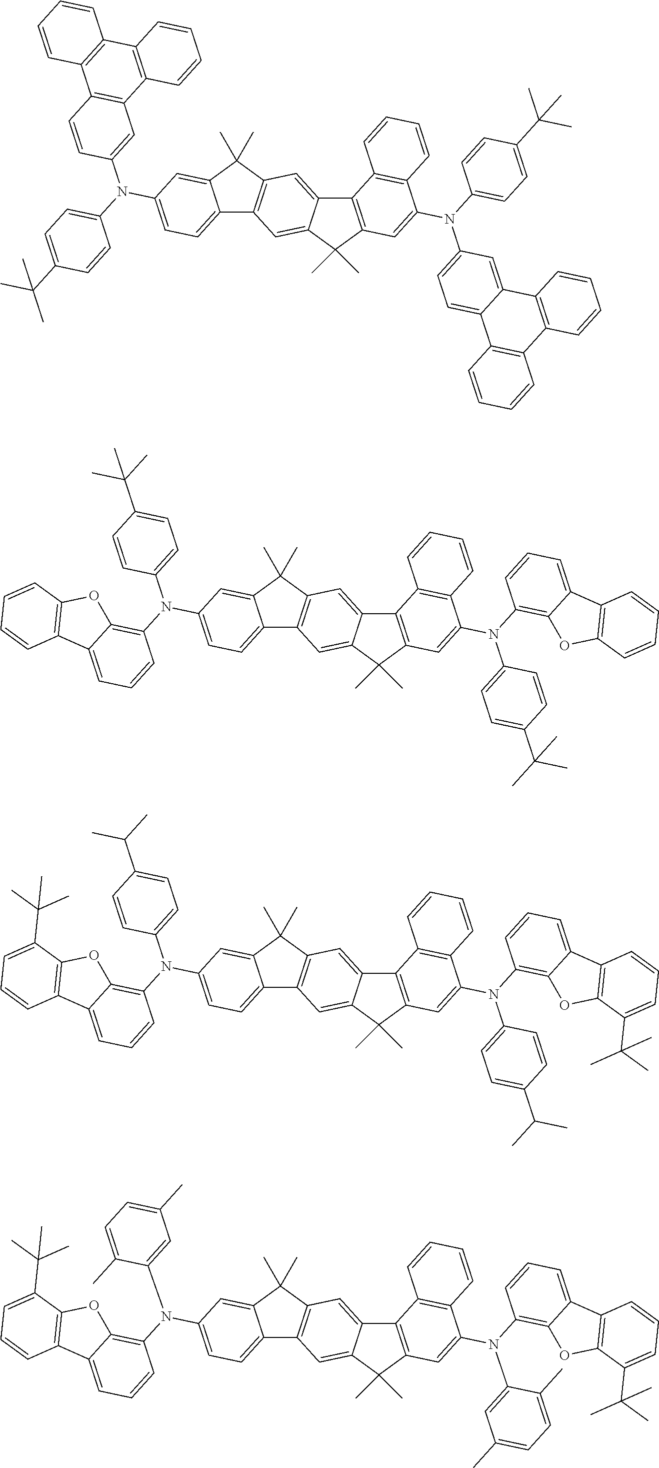

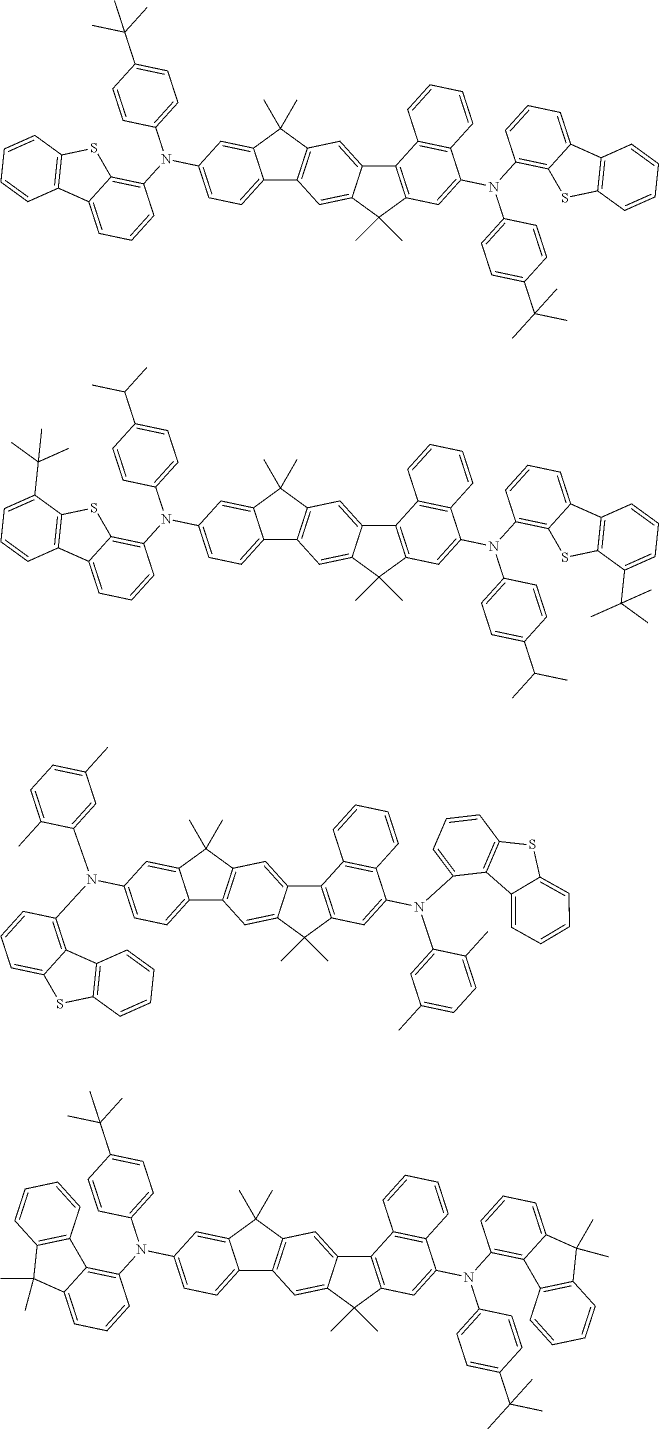

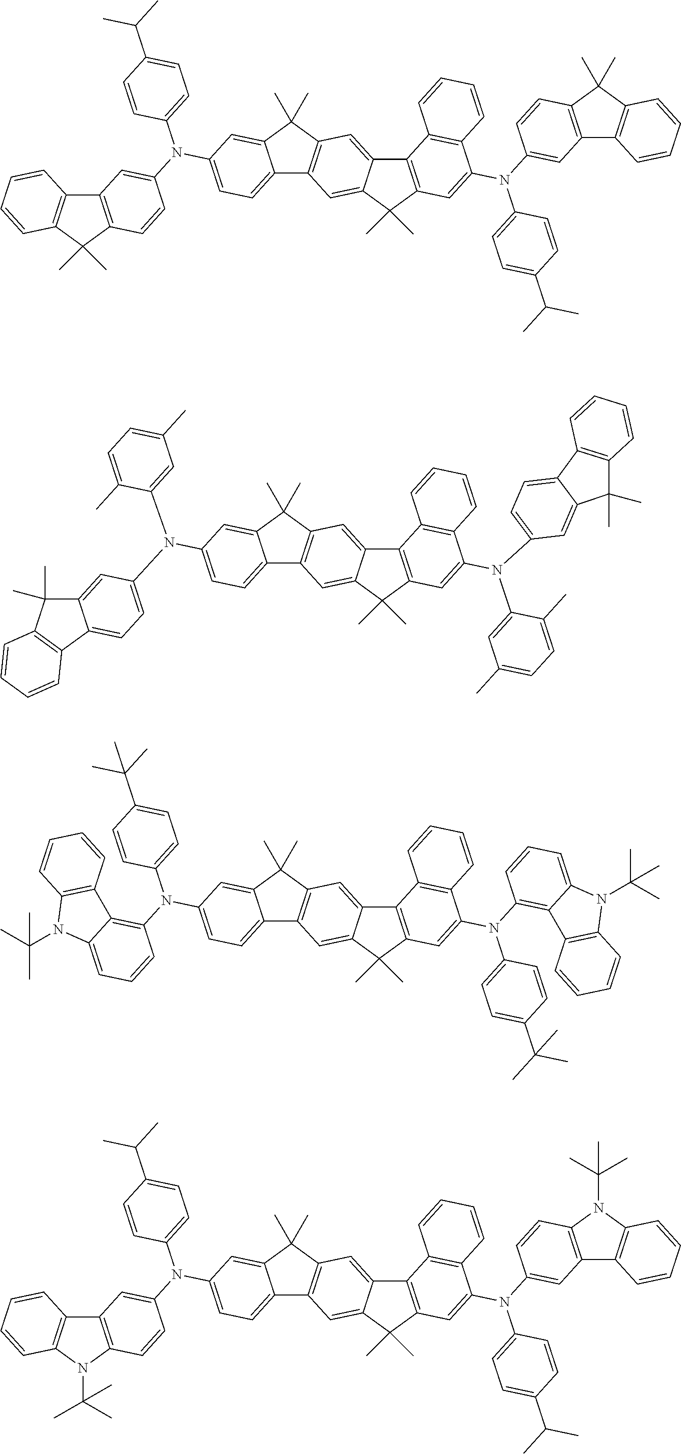

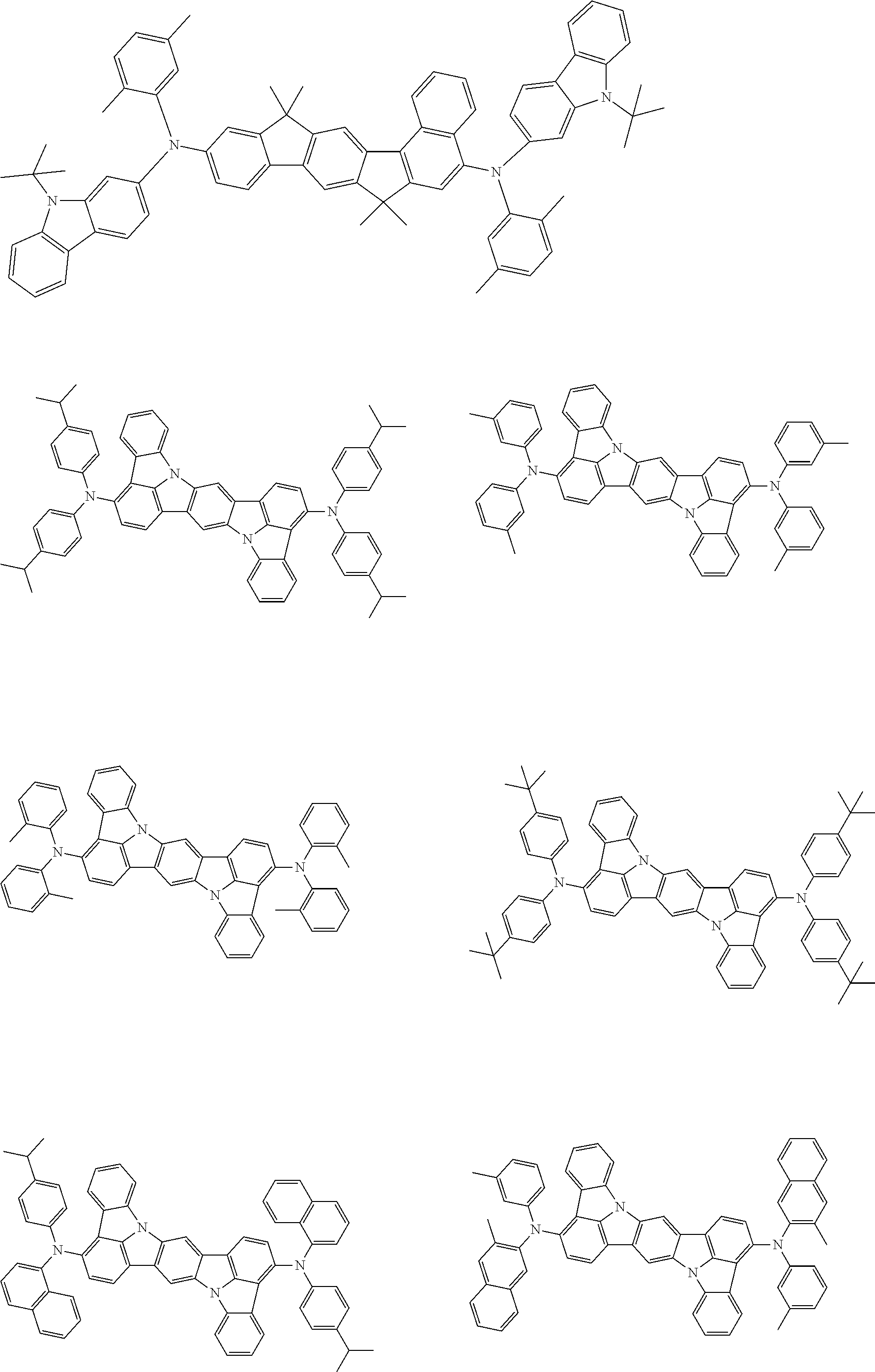

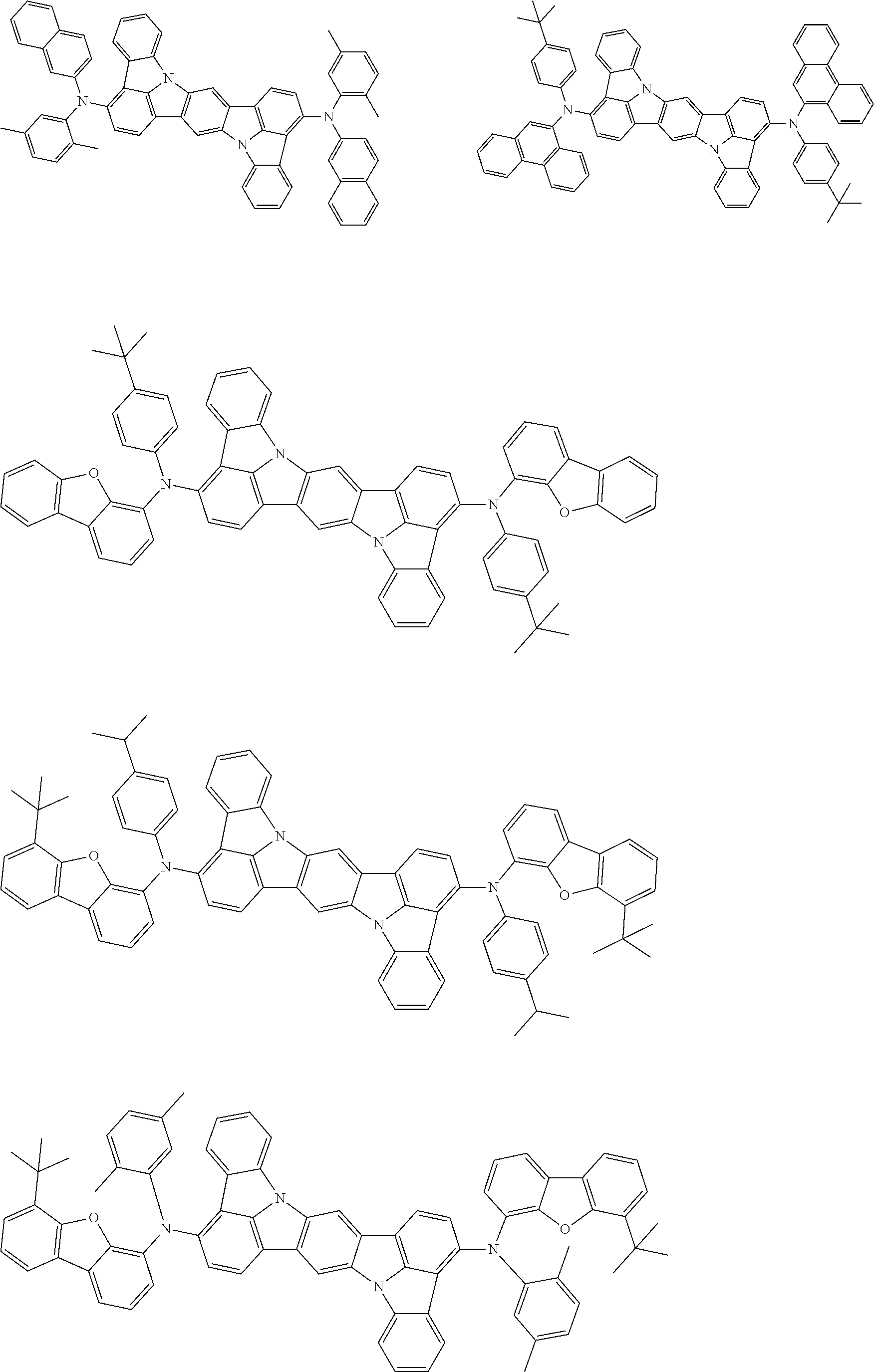

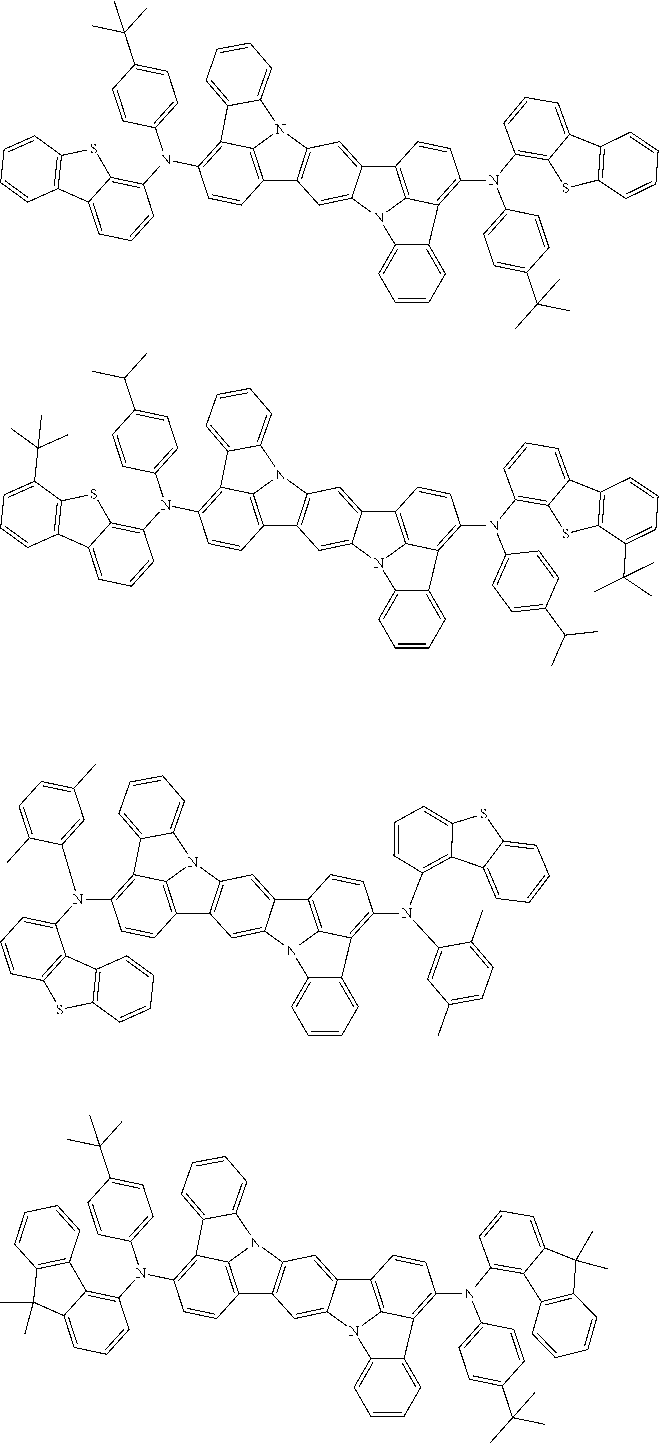

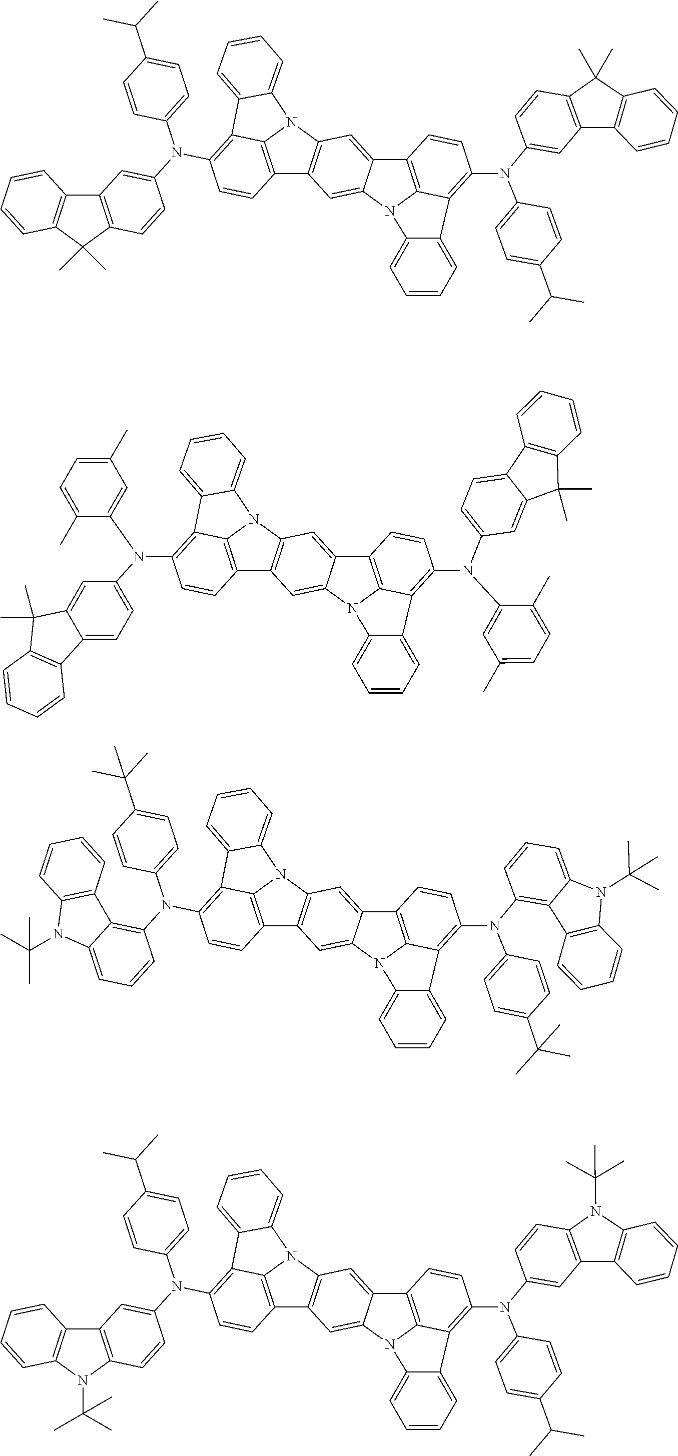

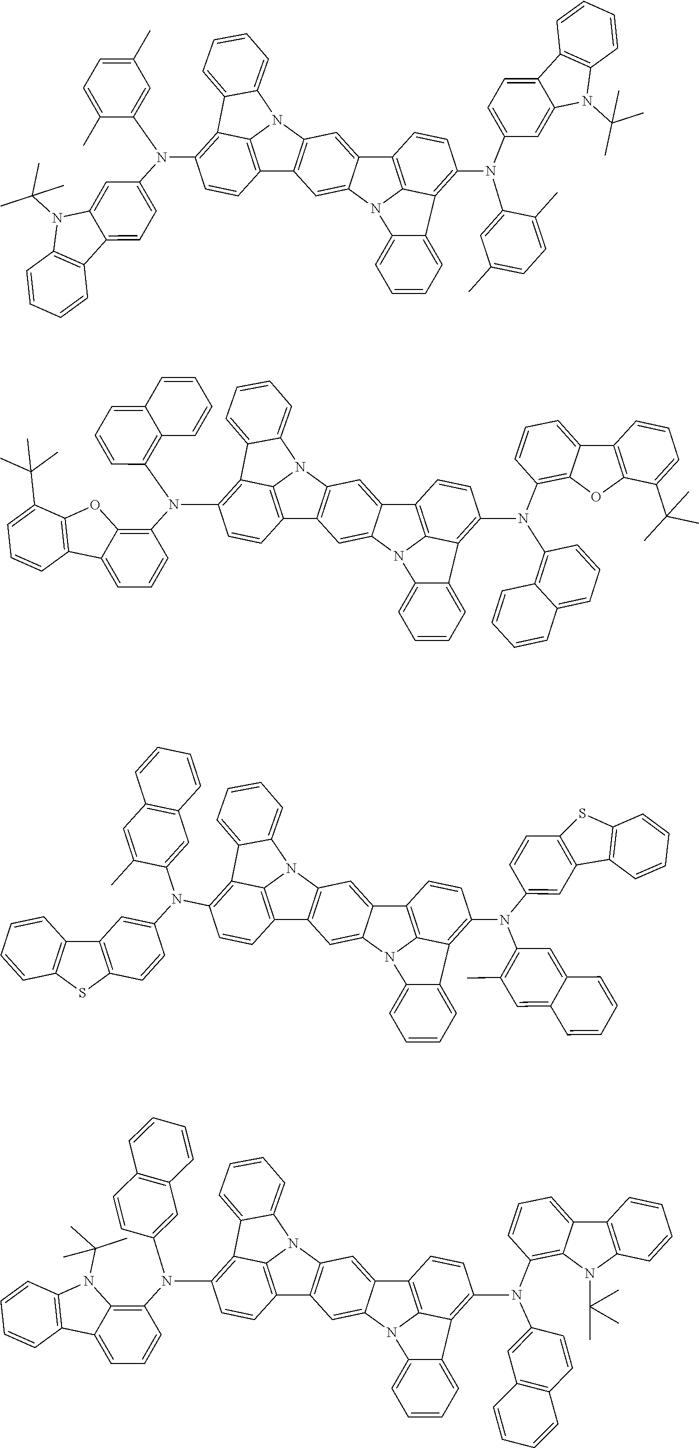

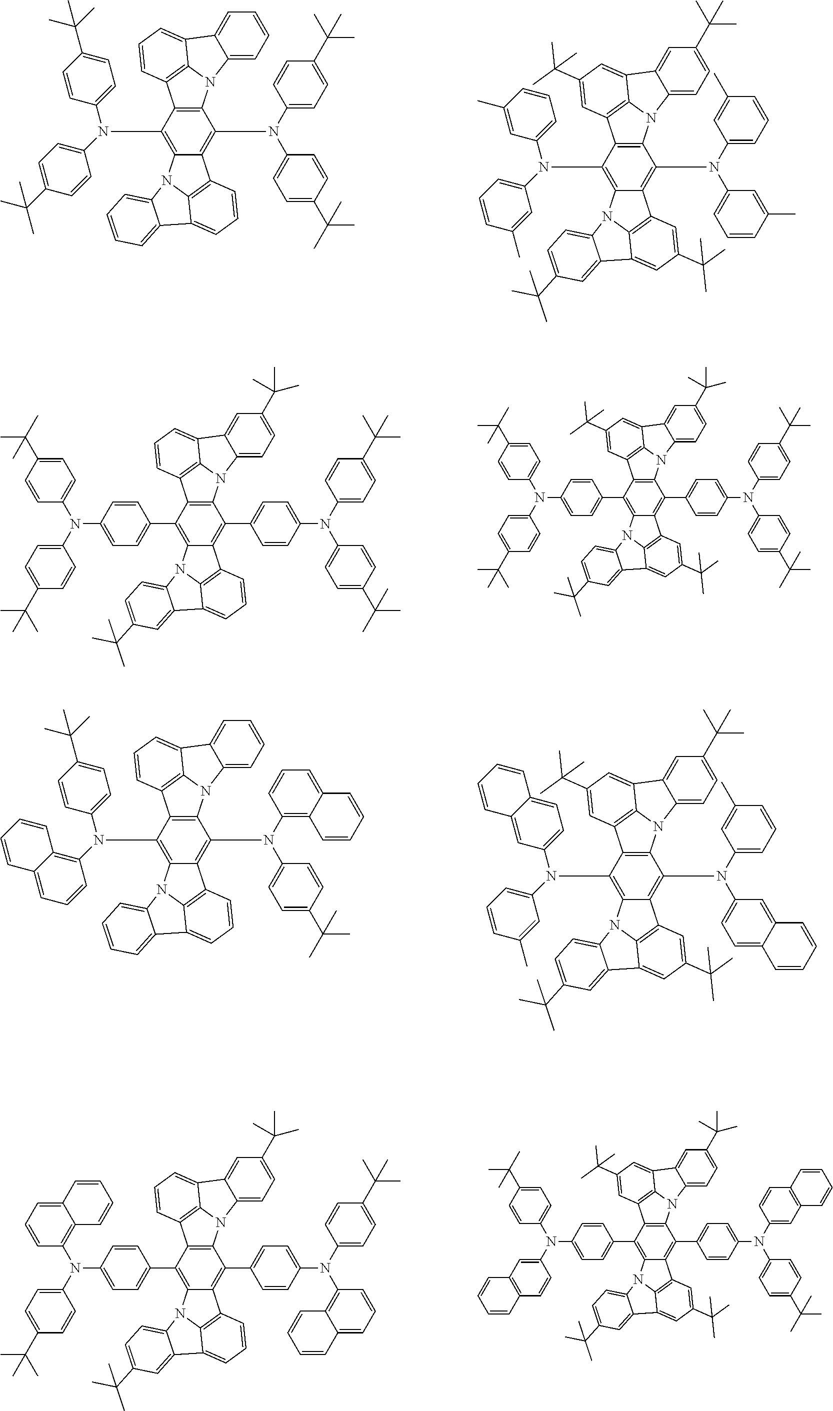

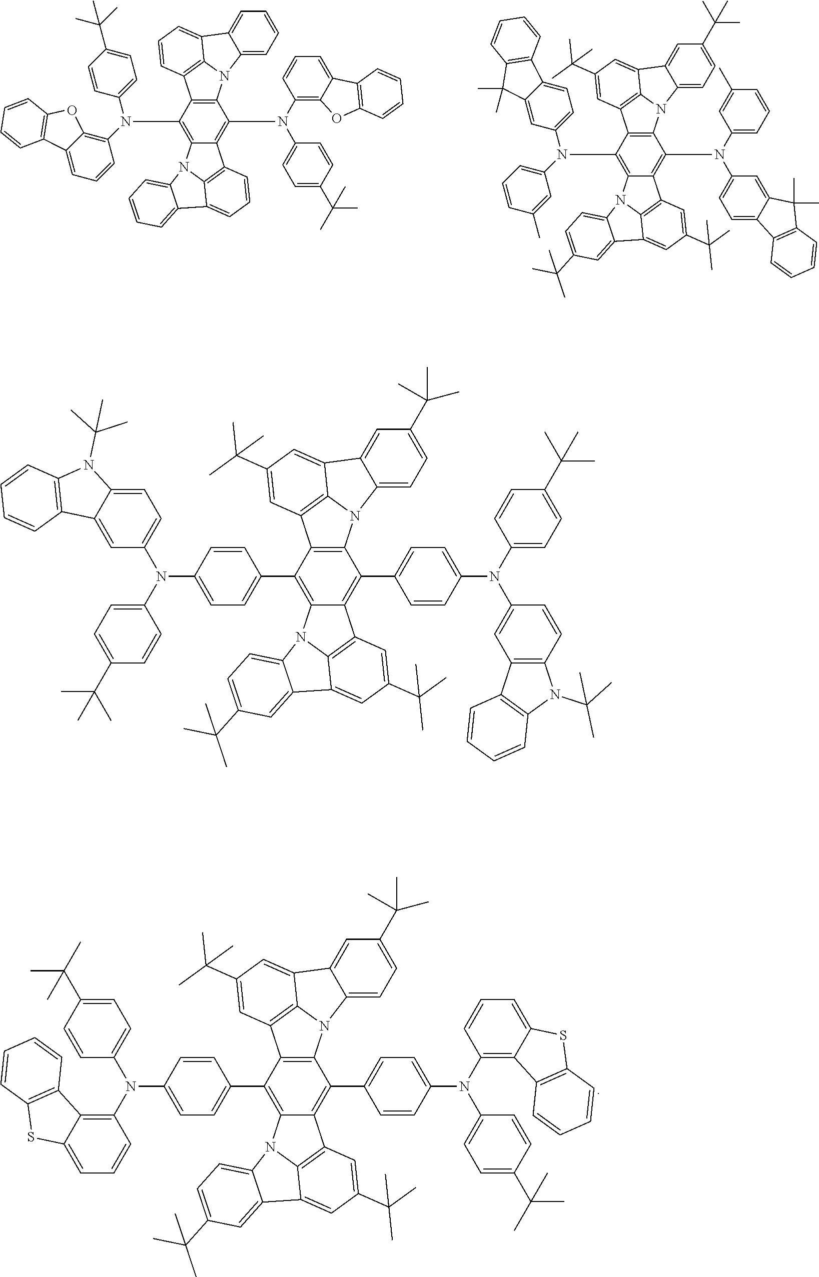

In one embodiment, an organic guest material represented by the formula (III-2) is selected from, but not limited thereto:

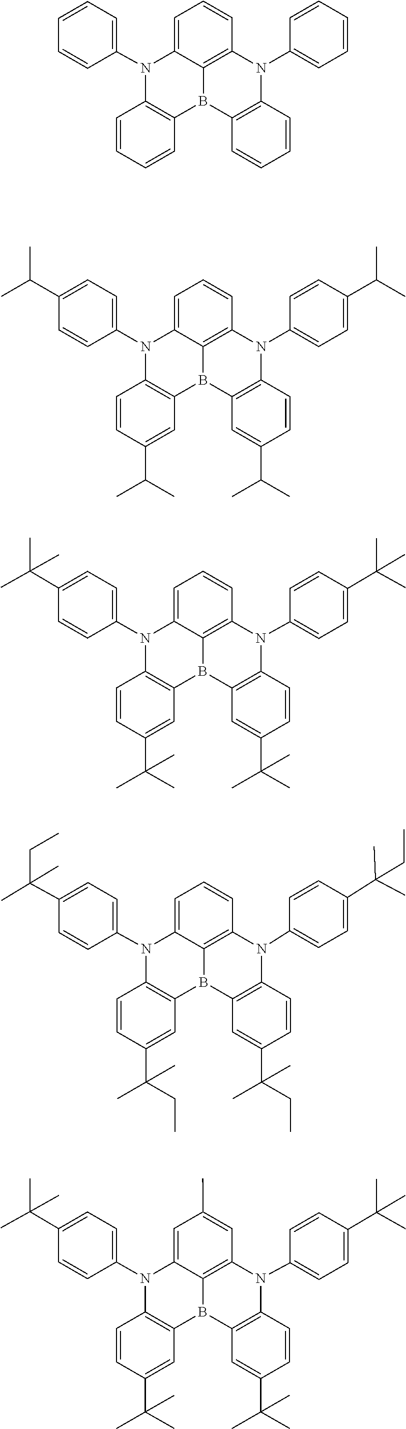

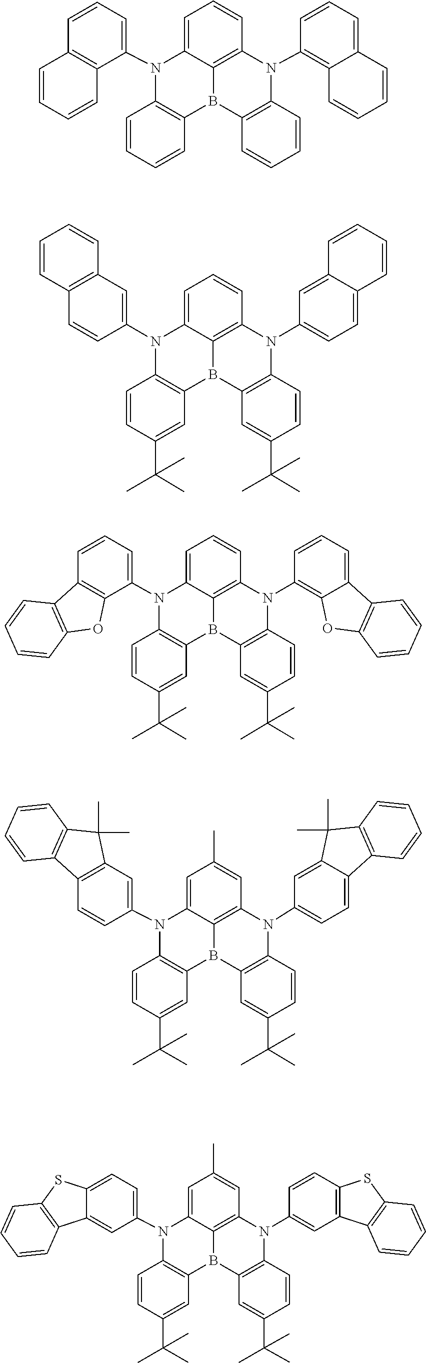 [SMILES: c1ccc2c(c1)B1c3ccccc3N(c3cccc4ccccc34)c3cccc(c31)N2c1cccc2ccccc12 ; CC(C)(C)c1ccc2c(c1)B1c3cc(C(C)(C)C)ccc3N(c3ccc4ccccc4c3)c3cccc(c31)N2c1ccc2ccccc2c1 ; CC(C)(C)c1ccc2c(c1)B1c3cc(C(C)(C)C)ccc3N(c3cccc4c3oc3ccccc34)c3cccc(c31)N2c1cccc2c1oc1ccccc12 ; Cc1cc2c3c(c1)N(c1ccc4c(c1)C(C)(C)c1ccccc1-4)c1ccc(C(C)(C)C)cc1B3c1cc(C(C)(C)C)ccc1N2c1ccc2c(c1)C(C)(C)c1ccccc1-2 ; Cc1cc2c3c(c1)N(c1ccc4sc5ccccc5c4c1)c1ccc(C(C)(C)C)cc1B3c1cc(C(C)(C)C)ccc1N2c1ccc2sc3ccccc3c2c1]

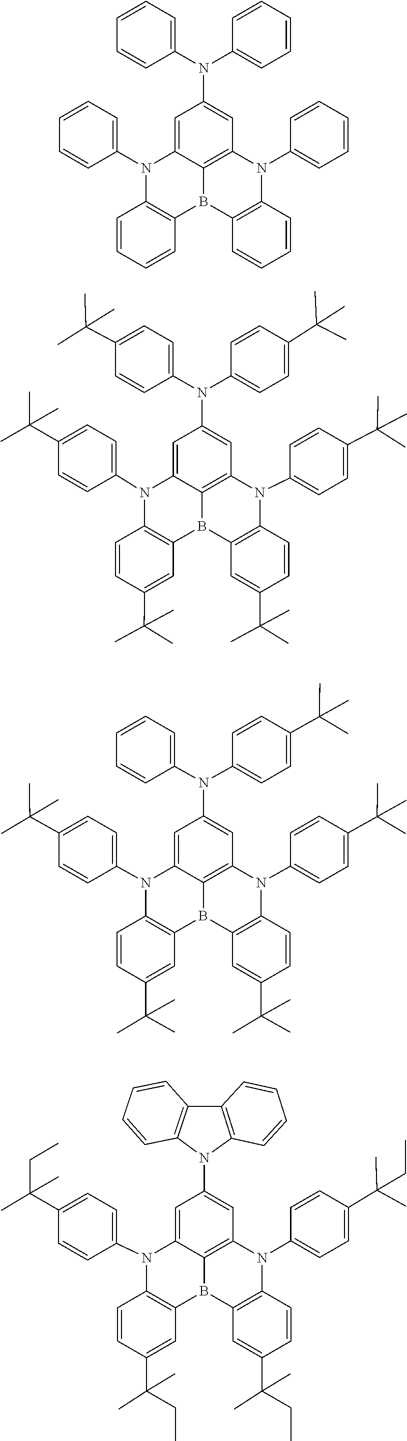

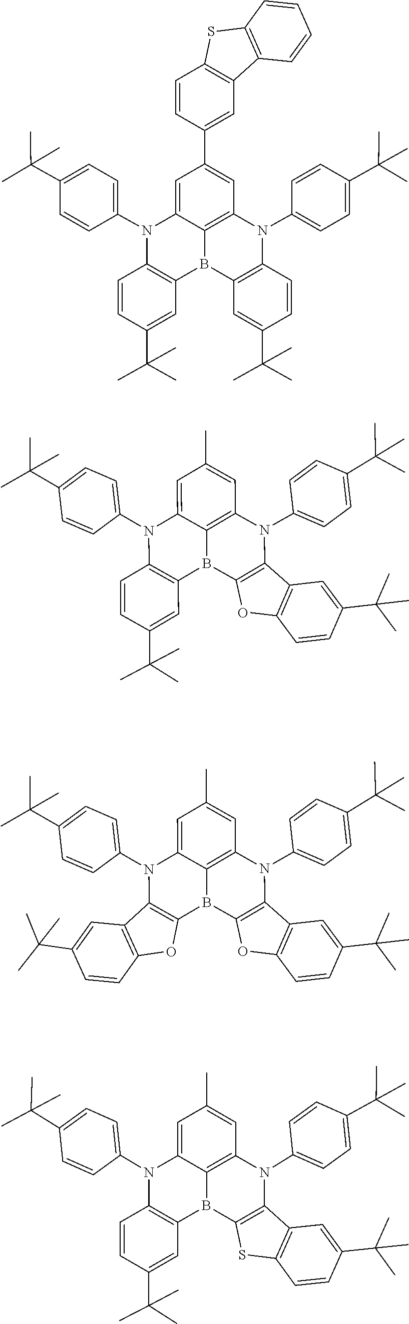

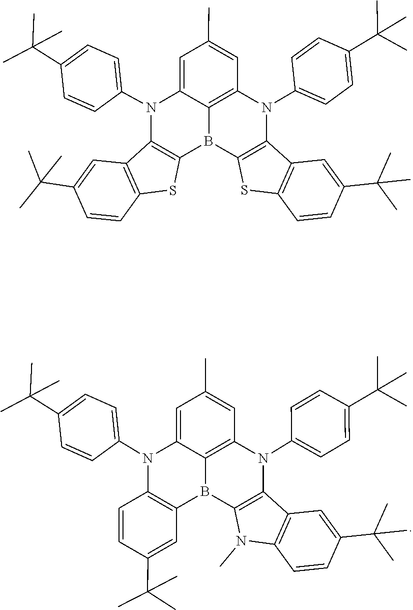

In one embodiment, in the mixture, a mass ratio of the first organic compound H1 to the second organic compound H2 ranges from 99:1 to 70:30. Further, the mass ratio of the first organic compound H1 to the second organic compound H2 ranges from 99:1 to 90:10.

The present application further provides a composition. The composition includes at least one organic compound or mixture described above and at least one first organic solvent. Wherein the at least one first organic solvent is used to dissolve the organic compound or the mixture.

In some embodiments, the at least one first organic solvent is a solvent selected from aromatic, a heteroaromatic, ester, aromatic ketone, aromatic ether, aliphatic ketone, aliphatic ether, alicyclic, olefin, borate ester, and phosphate ester, or mixtures of two or more solvents.

In some embodiments, the at least one first organic solvent is selected from the aromatic and heteroaromatic based solvent.

Examples of the aromatic or heteroaromatic based solvents suitable for the present application include, but are not limited thereto: p-diisopropylbenzene, pentyl benzene, tetrahydronaphthalene, cyclohexylbenzene, chloronaphthalene, 1,4-dimethylnaphthalene, 3-isopropylbiphenyl, p-methylisopropyl benzene, dipentyl benzene, tripentyl benzene, pentyl toluene, o-diethylbenzene, m-diethylbenzene, p-diethylbenzene, 1,2,3,4-tetratoluene, 1,2,3,5-tetratoluene, 1,2,4,5-tetratoluene, butadiene benzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylcumene, 1-methylnaphthalene, 1,2,4-trichlorobenzene, 4,4-difluorodiphenylmethane, 1,2-dimethoxy-4-(1-propenyl) benzene, diphenylmethane, 2-phenylpyridine, 3-phenylpyridine, N-methyldiphenylamine, 4-isopropylbiphenyl, α,α-dichlorodiphenylmethane, 4-(3-phenylpropyl) pyridine, benzyl benzoate, 1,1-bis (3,4-dimethylphenyl) ethane, 2-isopropylnaphthalene, quinoline, isoquinoline, methyl 2-furanoate, ethyl 2-furanoate, etc.

Examples of the aromatic ketone-based solvents suitable for the present application include, but are not limited thereto: 1-tetrahydronaphthalenone, 2-tetrahydronaphthalenone, 2-(phenyl epoxy) tetrahydronaphthalenone, 6-(methoxy) tetrahydronaphthalenone, acetophenone, phenylacetone, benzophenone, and derivatives of these compounds, such as 4-methylacetophenone, 3-methylacetophenone, 2-methylacetophenone, 4-methylphenylacetone, 3-methylphenylacetone, and 2-methylphenylacetone.

Examples of the aromatic ether-based solvents suitable for the present application include, but are not limited thereto: 3-phenoxytoluene, butoxybenzene, p-anisaldehyde dimethyl acetal, tetrahydro-2-phenoxy-2H-pyran, 1,2-dimethoxy-4-(1-propenyl) benzene, 1,4-benzodioxane, 1,3-dipropylbenzene, 2,5-dimethoxytoluene, 4-ethylbasic ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-p-propenylanisole, 1,2-dimethoxybenzene, 1-methoxynaphthalene, diphenyl ether, 2-phenoxymethyl ether, 2-phenoxytetrahydrofuran, and ethyl-2-naphthyl ether.

In some embodiments, the at least one first organic solvent may be selected from aliphatic ketone, such as 2-nonone, 3-nonone, 5-nonone, 2-decanone, 2,5-hexanedione, 2,6,8-trimethyl-4-nonone, fenone, phorone, isophorone, and di-n-pentyl ketone, and aliphatic ether, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, and tetraethylene glycol dimethyl ether.

In other embodiments, the at least one first organic solvent may be selected from ester-based solvents including octanoate, sebacate, stearate, benzoate, phenylacetate, cinnamate, oxalate, maleate, alkyl lactone, oleate, etc. In some embodiments, the ester-based solvents may be selected from octyl octanoate, diethyl sebacate, diallyl phthalate, and isononyl isononanoate.

In some embodiments, the composition further includes a second organic solvent.

Examples of the second organic solvent include, but are not limited thereto: methanol, ethanol, 2-methoxyethanol, dichloromethane, chloroform, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethyl sulfoxide, tetralin, naphthane, indene, and/or mixtures of these solvents.

In some embodiments, the first organic solvent and the second organic solvent of the present application are solvents with Hansen solubility parameters within following ranges:
δd (dispersion force) ranges from 17.0 MPa½ to 23.2 MPa½, and further ranges from 18.5 MPa½ to 21.0 MPa½;
δp (polarity force) ranges from 0.2 MPa½ to 12.5 MPa½, and further ranges from 2.0 MPa½ to 6.0 MPa½; and
δh (hydrogen bonding force) ranges from 0.9 MPa½ to 14.2 MPa½, and further ranges from 2.0 MPa½ to 6.0 MPa½.

Selection of the first organic solvent and the second organic solvent may consider boiling points. The boiling points of the first organic solvent and the second organic solvent are both greater than or equal to 150° C. In some embodiments, the above boiling points are greater than or equal to 180° C. In some embodiments, the above boiling points are greater than or equal to 200° C. In some embodiments, the above boiling points are greater than or equal to 250° C. In some embodiments, the above boiling points are greater than or equal to 275° C., or are greater than or equal to 300° C. The boiling points selected within these ranges are useful to prevent nozzles of inkjet printing heads from clogging. A film containing functional materials may be form by evaporating the first organic solvent and the second organic solvent from a solvent system.

The composition may be a solution or a suspension.

In some embodiments, a mass fraction of the anthracene-based compound in the composition or the mixture in the composition may range from 0.01 wt % to 10 wt %. In some embodiments, the mass fraction ranges from 0.1 wt % to 15 wt %. In some embodiments, the mass fraction ranges from 0.2 wt % to 5 wt %. In some embodiments, the mass fraction ranges from 0.25 wt % to 3 wt %.

When the composition is used as a coating material or printing ink to manufacture an organic electronic device, an organic layer may be formed by a printing or coating process. A suitable printing or coating process includes (but are not limited to) inkjet printing, nozzle printing, letterpress printing, silk-screen printing, dip coating, rotary coating, scraper coating, roller printing, rotary roller printing, lithographic printing, flexographic printing, rotary printing, spraying, brush coating, pad printing, slot extrusion coating, etc. In some embodiments, the suitable printing or coating processes are the letterpress printing, the nozzle printing, and the inkjet printing. The solution or the suspension may further include one or more components such as surfactant, lubricant, wetting agent, dispersant, hydrophobic agent, or adhesive, so as to adjust viscosity and performances of formed films, to improve an adhesion performance, etc.

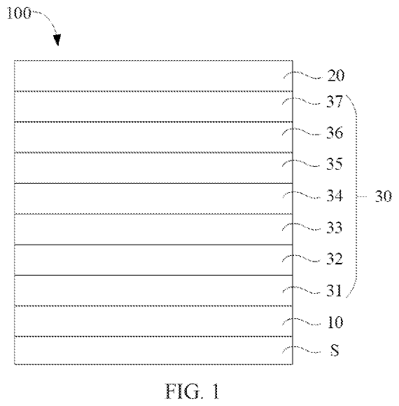

Please refer to FIG. 1, the present application further provides an organic electronic device 100. The organic electronic device 100 includes the anthracene-based compound or the mixture described above, or is prepared from the composition described above.

The organic electronic device may be selected from, but are not limited thereto: an organic light-emitting diode (OLED), an organic photovoltaic cell (OPV), an organic light-emitting cell (OLEEC), an organic field effect tube (OFET), an organic light-emitting field effect tube, an organic laser, an organic spin electronic device, an organic sensor, an organic plasmon emitting diode, etc. In some embodiments, the organic electronic device may be an organic electroluminescent device, such as the OLED, the OLEEC, or the organic light-emitting field effect tube. In detail, the organic electronic device 100 includes a first electrode 10, a second electrode 20, and an organic functional layer 30 disposed between the first electrode 10 and the second electrode 20. The organic functional layer 30 includes the organic compound, the mixture, or the composition described above. In some embodiments, the first electrode is an anode and the second electrode is a cathode.

The organic functional layer 30 may be selected from a hole injection layer (HIL), a hole transport layer (HTL), a light-emitting layer (EML), an electron barrier layer (EBL), an electron injection layer (EIL), an electron transport layer (ETL), and a hole barrier layer (HBL).

In this embodiment, the organic electronic device 100 includes a substrate S and an anode 10, a hole injection layer 31, a hole transport layer 32, an electronic barrier layer 33, a light-emitting layer 34, a hole barrier layer 35, an electronic transport layer 36, an electronic injection layer 37, and a cathode 20 stacked on the substrate S in sequence.

In one embodiment, the organic electronic device 100 may only include the anode 10, the cathode 20, and at least one light-emitting layer 34 disposed between the anode 10 and the cathode 20. The light-emitting layer 34 includes the organic compound or the mixture described above, or is prepared from the composition described above.

It can be understood that a structure of the organic electronic device is not limited thereto.

The substrate S may be transparent or opaque. The transparent substrate S may be used to make a transparent light-emitting element. For example, see a literature of Bulovic et al., Nature 1996, 380, p 29, Gu et al., Appl Phys. Lett. 1996, 68, p 2606. Further, the substrate S may be rigid or elastic. In some embodiments, the substrate S is made of plastic, metal, semiconductor chips, or glass. In some embodiments, the substrate S has a smooth surface. In other embodiments, the substrate S is flexible, and may be a polymer film or be made of plastic, wherein a glass transition temperature Tg of the substrate S is greater than 150° C. In some embodiments, the glass transition temperature Tg is greater than 200° C. In some embodiments, the glass transition temperature Tg is greater than 250° C. In some embodiments, the glass transition temperature Tg is greater than 300° C. Examples of suitable materials of the flexible substrate S include polyethylene terephthalate (PET) and polyethylene glycol (2,6-naphthalene) (PEN).

The anode 10 is an electrode used for injecting holes, and the holes in the anode 10 may be easily injected into the hole injection layer 31, the hole transport layer 32, or the light-emitting layer 34. In some embodiments, materials of the anode 10 may include conductive metal, conductive metal oxide, or conductive polymer. In some embodiments, absolute value of a difference between work function of the anode 10 and highest occupied molecular orbital (HOMO) energy level or valence band energy level of an emitter of the light-emitting layer 34, or a p-type semiconductor material of the HIL, the HTL, or the EBL is less than 0.5 eV. In some embodiments, the above absolute value is less than 0.3 eV. In some embodiments, the above absolute value is less than 0.2 eV. Examples of the materials of the anode 10 include but are not limited thereto: aluminum (Al), copper (Cu), aurum (Au), argentum (Ag), magnesium (Mg), ferrum (Fe), cobalt (Co), niccolum (Ni), manganese (Mn), palladium (Pd), platinum (Pt), indium tin oxide (ITO), aluminum doped zinc oxide (AZO), etc. The materials of the anode 10 may be applied to by any suitable technology, such as a suitable physical vapor deposition method including RF magnetron sputtering, vacuum thermal evaporation, electron beam (e-beam), etc. In some embodiments, the anode 10 is patterned. A patterned ITO conductive substrate is commercially available and may be used to manufacture devices of the present application.

The cathode 20 is an electrode used for injecting electrons, and the electrons in the cathode 20 may be injected easily into the electron injection layer 37, the electron transport layer 36, or the light-emitting layer 34. In some embodiments, materials of the cathode 20 may include conductive metal or conductive metal oxide. In some embodiments, absolute value of a difference between work function of the cathode 20 and LUMO energy level or conduction band energy level of the emitter of the light-emitting layer 34 or a n-type semiconductor material of the EIL, the ETL, or the HBL is less than 0.5 eV. In some embodiments, the above absolute value is less than 0.3 eV. In some embodiments, the above absolute value is less than 0.2 eV. Examples of the materials of the cathode 20 include but are not limited thereto: Al, Au, Ag, calcium (Ca), barium (Ba), Mg, LiF/Al, MgAg alloy, BaF₂/Al, Cu, Fe, Co, Ni, Mn, Pd, Pt, ITO, etc. The materials of the cathode 20 may be applied to by any suitable technology, such as a suitable physical vapor deposition method including RF magnetron sputtering, vacuum thermal evaporation, or electron beam (e-beam).

The hole injection layer 31 is a layer used for promoting an injection of holes from the anode 10 to the light-emitting layer 34. A hole injection material used in the hole injection layer 31 is a material that may receive holes injected from a positive electrode under low voltages. In some embodiments, highest occupied molecular orbital (HOMO) energy of the hole injection material is between work function of a positive electrode material and HOMO energy of surrounding organic material layers. The hole injection material includes, but are not limited thereto: metalloporphyrins, oligothiophenes, organic materials based on arylamines, organic materials based on hexacyano hexaazabenzophenanthrene, organic materials based on quinacridone, organic materials based on perylene, anthraquinone, conductive polymers based on polyaniline and polythiophene, etc.

The hole transport layer 32 may be used to transmit holes smoothly. The hole transport layer 32 is provided with materials having high hole mobility and can receive holes transmitted from the anode 10 or the hole injection layer 31 and transfer the holes to the light-emitting layer 34. The materials of the hole transport layer 32 include, but are not limited thereto: organic materials based on arylamine, conductive polymer, block copolymer containing both conjugated and non-conjugated portions, etc.

Materials of the electronic barrier layer 33 may include any one or more of aromatic amine electronic barrier materials, dimethylfluorene electronic barrier materials, and carbazole electronic barrier materials.

The hole blocking layer 35 is a layer that blocks holes from reaching a negative electrode, and may be formed under the same conditions as those of the hole injection layer 31. Examples of materials of the hole blocking layer 35 include, but are not limited thereto, diazole derivatives, triazole derivatives, phenanthroline derivatives, BCP, aluminum complexes, etc.

The electron transport layer 36 may be used to transmit electrons smoothly. The electron transport layer 36 is provided with materials having high electron mobility and can expertly receive electrons injected from the negative electrode and transfer electrons to the light-emitting layer 34. The materials of the electron transport layer 36 may include, but are not limited thereto: Al complexes of 8-hydroxyquinoline, complexes containing Alq₃, organic radical compounds, hydroxyflavone metal complexes, 8-hydroxyquinoline lithium (LiQ), or compounds based on benzimidazole.

The electron injection layer 37 may be used to inject electrons smoothly. The electron injection layer 37 is provided with materials having ability to transmit electrons, may inject electrons from the negative electrode, is excellent on injecting electrons into light-emitting materials of the light-emitting layer 34, and prevents excitons generated by the light-emitting layer 34 from moving to the hole injection layer 31. The materials of the electron injection layer 37 further have excellent ability to form thin films. The materials of the electron injection layer 37 include, but are not limited thereto: 8-hydroxyquinoline lithium (LiQ), fluorenone, anthraquinone dimethyl, biphenylquinone, thian dioxide, azole, diazole, triazole, imidazole, perylene tetracarboxylic acid, fluorene methane, anthrone, derivatives of these compounds, metal complexes, nitrogen containing 5-membered ring derivatives, etc.

In some embodiments, a wavelength of light emitting from the organic electronic device ranges from 300 nm to 1000 nm. In some embodiments, the wavelength ranges from 350 nm to 900 nm. In some embodiments, the wavelength ranges from 400 nm to 800 nm.

The organic electronic device of the present application may be applied to a display device, an illumination device, a light source, a sensor, etc.

The present application is described below with reference to the embodiments, but the present application is not limited to the following embodiments. It should be understood that the appended claims summarize a scope of the present application. Under a guidance of a concept of the present application, those skilled in the art should realize that certain changes to the embodiments of the present application will be covered by a spirit and the scope of the claims of the present application.

### EXAMPLE 1: SYNTHESIS OF COMPOUND (M1)

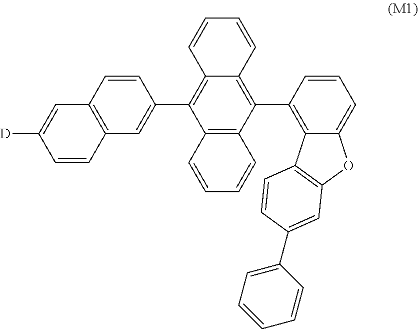 [SMILES: [2H]c1ccc2cc(-c3c4ccccc4c(-c4cccc5oc6cc(-c7ccccc7)ccc6c45)c4ccccc34)ccc2c1]

### Synthetic Route

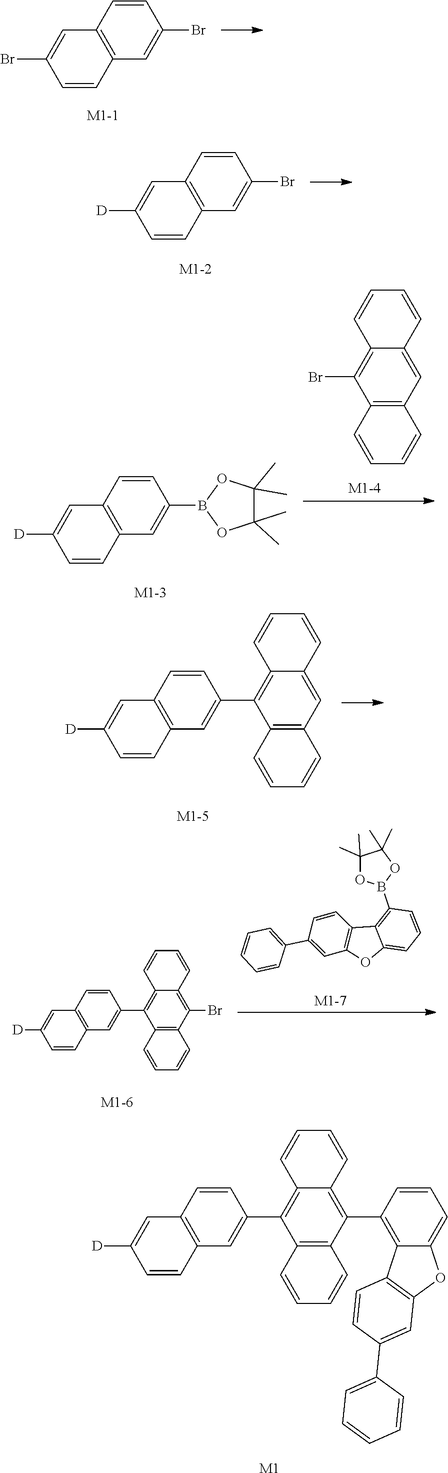

1) Synthesis of intermediate M1-2: in a nitrogen atmosphere, add 28.6 g (100 mmol) of compound M1-1 and 100 mL of anhydrous tetrahydrofuran into a three neck-flask (300 mL). Stir the compound M1-1 to dissolve in the anhydrous tetrahydrofuran. Cool the solution to a temperature of −78° C. Then slowly add 100 mmol of n-butyllithium dropwise to the solution. After 2 hours, add all 150 mmol of deuterated water to the solution at once. Then slowly raise the temperature of the reaction solution to room temperature. Continue stirring the reaction solution for 4 hours. After the reaction is completed, rotary evaporate the reaction solution to remove most of the solvent, and dissolve with dichloromethane. Wash with water for 3 times. Collect the organic solution and stir it with silica gel. Purify the product in column chromatography. A yield is 74%.

2) Synthesis of intermediate M1-3: in a nitrogen atmosphere, add 12.5 g (60 mmol) of compound M1-2 and 100 mL of anhydrous tetrahydrofuran into a three neck-flask (300 mL) and cool to a temperature of −78° C. Slowly add 60 mmol of n-butyllithium dropwise to the solution. After 2 hours, add all 65 mmol of isopropanol pinacol borate at once. Let the temperature of the reaction solution raising to room temperature, and let the reaction continue for 12 hours. Add purified water to quench the reaction. Rotary evaporate the reaction solution to remove most of the solvent. Dissolve with dichloromethane. Wash with water for 3 times, and then collect the organic phase. Dry the organic phase by rotary evaporation. Recrystallize the product for purification. A yield is 80%.

3) Synthesis of intermediate M1-5: in a nitrogen atmosphere, add 9 g (40 mmol) of compound M1-3, 10.3 g (40 mmol) of compound M1-4, 2.2 g (2 mmol) of tetrakis (triphenylphosphine) palladium, 20 mL of aqueous solution of 11 g (80 mmol) of potassium carbonate, and 100 mL of toluene into a three neck-flask (500 mL). Heat and stir the solution to a temperature of 110° C. for 12 hours. After the reaction is completed, cool the solution to room temperature. Filter the filtrate by suction, and collect the filtrate. Rotary evaporated the reaction solution to remove most of the solvent. Dissolve with dichloromethane. Wash with water for 3 times. Collect the organic solution, and stir it with silica gel. Purify the product in column chromatography. A yield is 72%.

4) Synthesis of intermediate M1-6: add 6.1 g (20 mmol) of compound M1-5 and 80 mL of trichloromethane into a two neck-flask (250 mL). Slowly add trichloromethane solution of 20 mmol of N-bromosuccinimide dropwise to the two neck-flask at a room temperature. Then continue stirring for 6 hours. Add water to quench the reaction. Wash the reaction solution with water for 3 times. Collect the organic solution and stir it with silica gel. Purify the product in column chromatography. A yield is 85%.

5) Synthesis of compound M1: in a nitrogen atmosphere, add 3.84 g (10 mmol) of compound M1-6, 3.7 g (10 mmol) of compound M1-7, 0.55 g (0.5 mmol) of tetrakis (triphenylphosphine) palladium, 10 mL of aqueous solution of 2.8 g (20 mmol) of potassium carbonate, and 50 mL of toluene into a three neck-flask (250 mL). Heat and stir the solution to a temperature of 110° C. for 12 hours. After the reaction was completed, cool the solution to room temperature. Filter the filtrate by suction, and collect the filtrate after filtering. Rotary evaporate the reaction solution to remove most of the solvent, dissolve it with dichloromethane, and wash it with water for 3 times. Collect the organic solution and stir it with silica gel. Purify the product in column chromatography purification. A yield is 70%. MS(ASAP)=548.

### EXAMPLE 2: SYNTHESIS OF COMPOUND (M2)

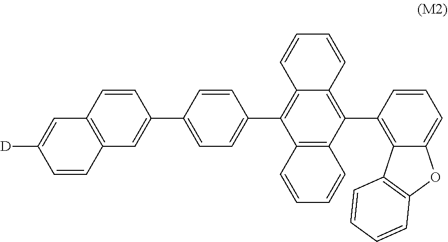 [SMILES: [2H]c1ccc2cc(-c3ccc(-c4c5ccccc5c(-c5cccc6oc7ccccc7c56)c5ccccc45)cc3)ccc2c1]

### Synthetic Route

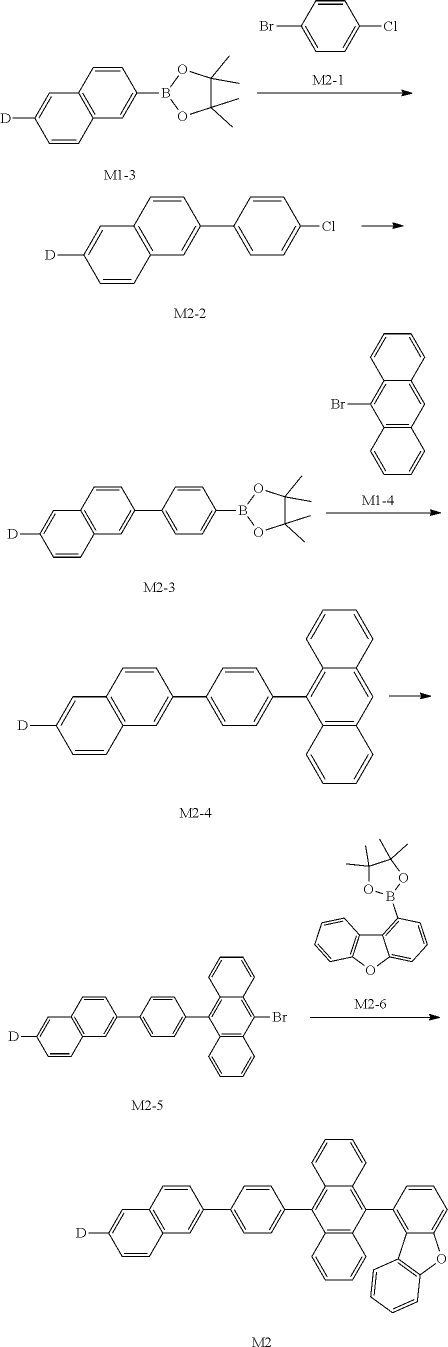

1) Synthesis of intermediate M2-2: substitute compound M2-1 for compound M1-4 according to a synthetic method of compound M1-5. A yield is 83%.

2) Synthesis of intermediate M2-3: in a nitrogen atmosphere, add 7.2 g (30 mmol) of compound M2-2, 7.6 g (30 mmol) of pinacol diboronate, 4.9 g (50 mmol) of potassium acetate, 1.32 g (1.8 mmol) of Pd (ppf)Cl₂, and 60 mL of 1,4-dioxane into a three neck-flask (250 mL). Heat the solution to a temperature of 110° C. for 12 hours. After the reaction is completed, cool the solution to room temperature. Filter the filtrate by suction, and collect the filtrate. Rotary evaporated the reaction solution to remove most of the solvent. Dissolve with dichloromethane. Wash with water for 3 times. Collect the organic solution, and stir it with silica gel. Purify the product in column chromatography. A yield is 77%.

3) Synthesis of intermediate M2-4: substitute compound M2-3 for compound M1-3 according to the synthetic method of compound M1-5. A yield is 82%.

4) Synthesis of intermediate M2-5: substitute compound M2-4 for compound M1-5 according to a synthetic method of compound M1-6. A yield is 84%.

5) Synthesis of compound M2: substitute compound M2-5 and compound M2-6 for compound M1-6 and compound M1-7 respectively according to a synthetic method of compound M1. A yield is 72%. MS(ASAP)=548.

### EXAMPLE 3: SYNTHESIS OF COMPOUND (M3)

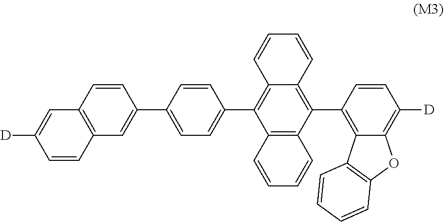 [SMILES: [2H]c1ccc2cc(-c3ccc(-c4c5ccccc5c(-c5ccc([2H])c6oc7ccccc7c56)c5ccccc45)cc3)ccc2c1]

### Synthetic Route

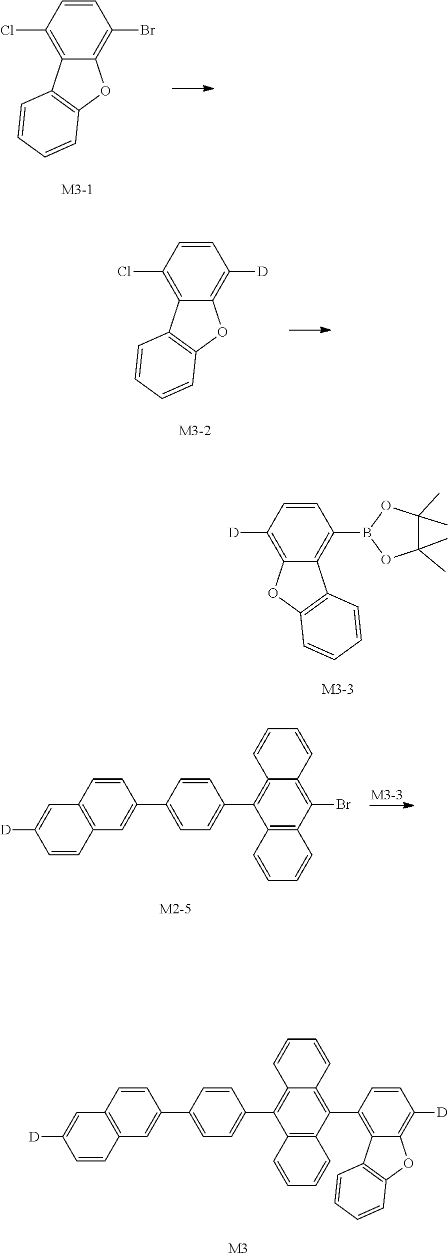 [SMILES: Clc1ccc(Br)c2oc3ccccc3c12 ; [2H]c1ccc(Cl)c2c1oc1ccccc12 ; [2H]c1ccc(B2OC(C)(C)C(C)(C)O2)c2c1oc1ccccc12 ; [2H]c1ccc2cc(-c3ccc(-c4c5ccccc5c(Br)c5ccccc45)cc3)ccc2c1 ; [2H]c1ccc2cc(-c3ccc(-c4c5ccccc5c(-c5ccc([2H])c6oc7ccccc7c56)c5ccccc45)cc3)ccc2c1 ; "M3"]

1) Synthesis of intermediate M3-2: substitute compound M3-1 for compound M1-1 according to a synthetic method of compound M1-2. A yield is 83%.

2) Synthesis of intermediate M3-3: substitute compound M3-2 for compound M2-2 according to a synthetic method of compound M2-3. A yield is 75%.

3) Synthesis of compound M3: substitute compound M2-5 and compound M3-3 for compound M1-6 and compound M1-7 respectively according to the synthetic method of compound M1. A yield is 70%. MS(ASAP)=549.

### EXAMPLE 4: SYNTHESIS OF COMPOUND (M4)

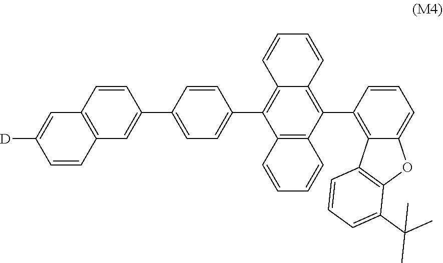 [SMILES: [2H]c1ccc2cc(-c3ccc(-c4c5ccccc5c(-c5cccc6oc7c(C(C)(C)C)cccc7c56)c5ccccc45)cc3)ccc2c1]

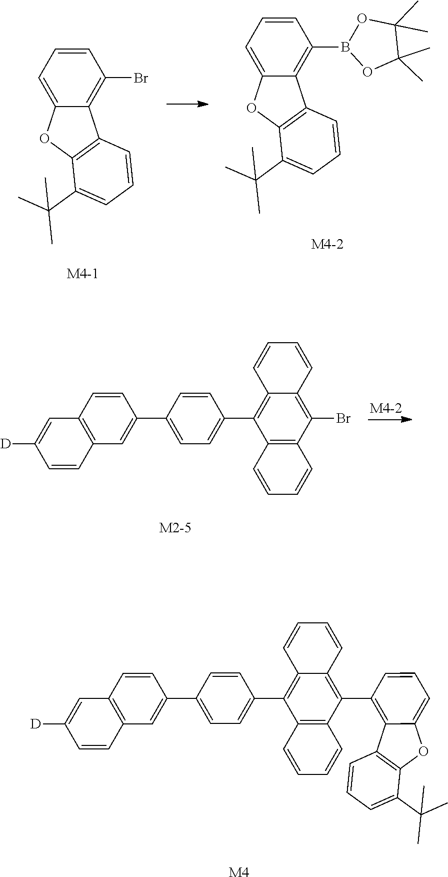 [SMILES: CC(C)(C)c1cccc2c1oc1cccc(Br)c12 ; CC(C)(C)c1cccc2c1oc1cccc(B3OC(C)(C)C(C)(C)O3)c12 ; [2H]c1ccc2cc(-c3ccc(-c4c5ccccc5c(Br)c5ccccc45)cc3)ccc2c1 ; [2H]c1ccc2cc(-c3ccc(-c4c5ccccc5c(-c5cccc6oc7c(C(C)(C)C)cccc7c56)c5ccccc45)cc3)ccc2c1 ; "M4"]

1) Synthesis of intermediates M4-2: substitute compound M4-1 for compound M2-2 according to the synthetic method of compound M2-3. A yield is 78%.

2) Synthesis of compound M4: substitute compound M2-5 and compound M4-2 for compound M1-6 and compound M1-7 respectively according to the synthetic method of compound M1. A yield is 72%. MS(ASAP)=604.

### EXAMPLE 5: SYNTHESIS OF COMPOUND (M5)

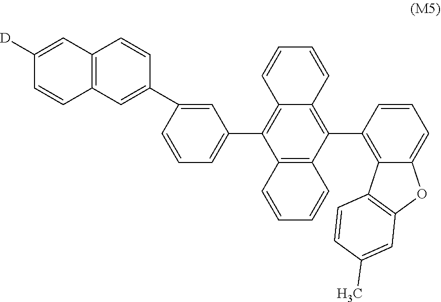 [SMILES: [2H]c1ccc2cc(-c3cccc(-c4c5ccccc5c(-c5cccc6oc7cc(C)ccc7c56)c5ccccc45)c3)ccc2c1]

### Synthetic Route

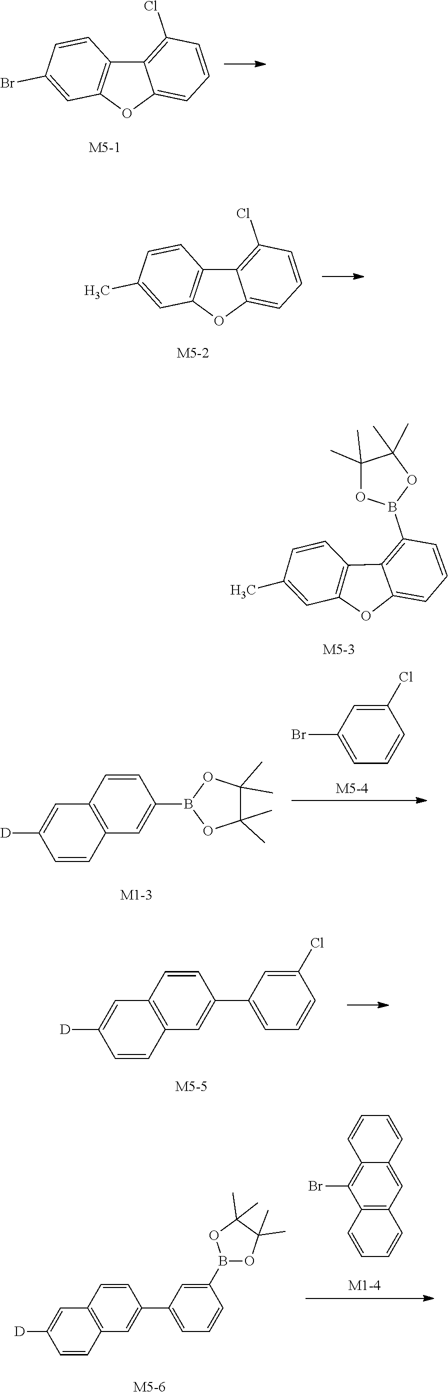

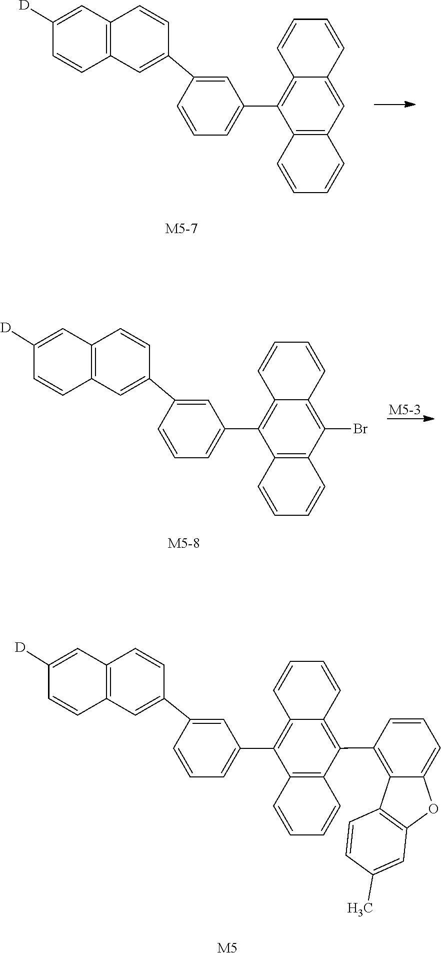 [SMILES: [2H]c1ccc2cc(-c3cccc(-c4c5ccccc5cc5ccccc45)c3)ccc2c1 ; [2H]c1ccc2cc(-c3cccc(-c4c5ccccc5c(Br)c5ccccc45)c3)ccc2c1 ; [2H]c1ccc2cc(-c3cccc(-c4c5ccccc5c(-c5cccc6oc7cc(C)ccc7c56)c5ccccc45)c3)ccc2c1 ; "M5"]

1) Synthesis of intermediates M5-2: in a nitrogen atmosphere, add 25.2 g (100 mmol) of compound M5-1, 4.6 g (5 mmol) of tris (dibenzylideneacetone) dipalladium, 4.8 g (10 mmol) of 2-dicyclohexylphosphorous-2′,4′,6′-triisopropylbiphenyl, and 150 mL of anhydrous tetrahydrofuran into a three neck-flask (500 mL). Then slowly add 100 mmol of methyl magnesium bromide dropwise to the solution under an ice bath. Continue stirring the reaction for 12 hours. After the reaction is completed, add water to quench the reaction. Rotary evaporate the reaction solution to remove most of the solvent, and dissolve with dichloromethane. Wash with water for 3 times. Collect the organic solution and stir it with silica gel. Purify the product in column chromatography. A yield is 85%.

2) Synthesis of intermediate M5-3: substitute compound M5-2 for compound M2-2 according to the synthetic method of compound M2-3. A yield is 76%.

3) Synthesis of intermediate M5-5: substitute compound M5-4 for compound M1-4 according to the synthetic method of compound M1-5. A yield is 81%.

4) Synthesis of intermediate M5-6: substitute compound M5-5 for compound M2-2 according to the synthetic method of compound M2-3. A yield is 74%.

5) Synthesis of intermediate M5-7: substitute compound M5-6 for compound M1-3 according to the synthetic method of compound M1-5. A yield is 74%.

6) Synthesis of intermediate M5-8: substitute compound M5-7 for compound M1-5 according to the synthetic method of compound M1-6. A yield is 82%.

7) Synthesis of compound MS: substitute compound M5-8 and compound M5-3 for compound M1-6 and compound M1-7 respectively according to the synthetic method of compound M1. A yield is 71%. MS(ASAP)=562.

### EXAMPLE 6: SYNTHESIS OF COMPOUND (M6)

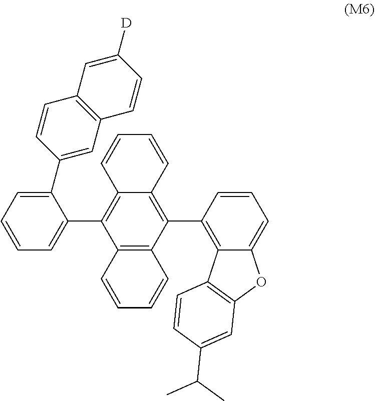 [SMILES: [2H]c1ccc2cc(-c3ccccc3-c3c4ccccc4c(-c4cccc5oc6cc(C(C)C)ccc6c45)c4ccccc34)ccc2c1]

### Synthetic Route

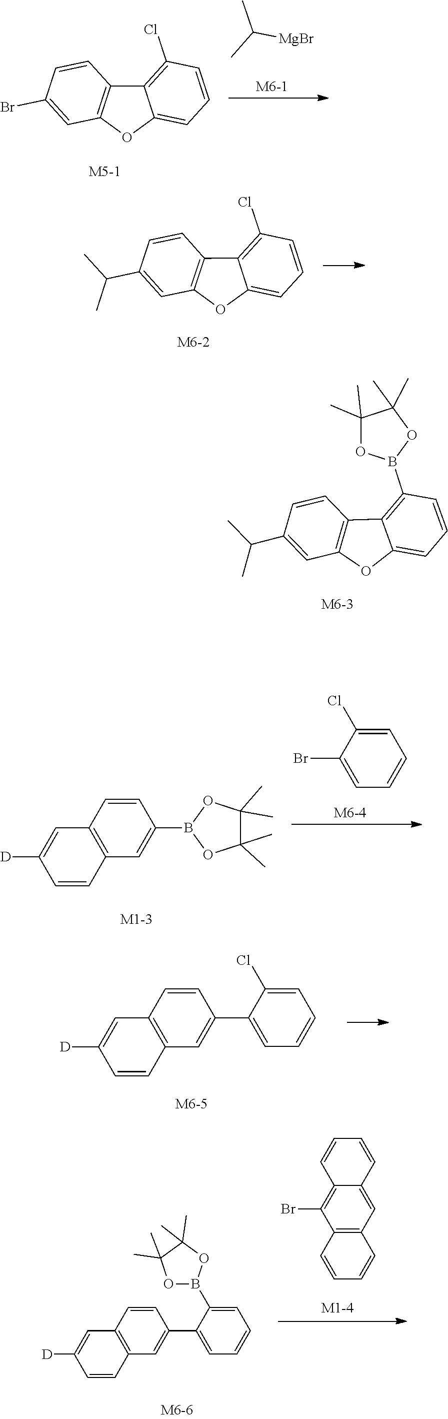

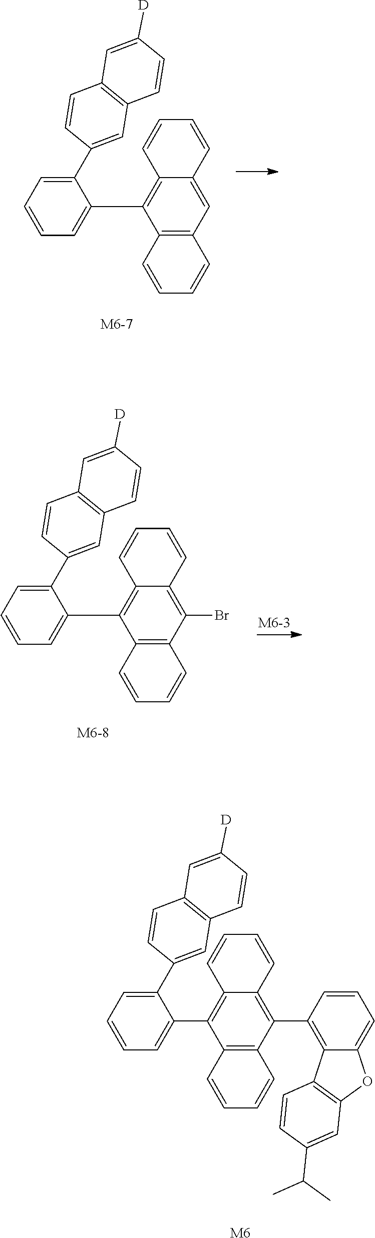 [SMILES: [2H]c1ccc2cc(-c3ccccc3-c3c4ccccc4cc4ccccc34)ccc2c1 ; [2H]c1ccc2cc(-c3ccccc3-c3c4ccccc4c(Br)c4ccccc34)ccc2c1 ; [2H]c1ccc2cc(-c3ccccc3-c3c4ccccc4c(-c4cccc5oc6cc(C(C)C)ccc6c45)c4ccccc34)ccc2c1 ; "M6"]

1) Synthesis of intermediate M6-2: substitute compound M6-1 for methyl magnesium bromide according to a synthetic method of compound M5-2. A yield is 72%.

2) Synthesis of intermediate M6-3: substitute compound M6-2 for compound M2-2 according to the synthetic method of compound M2-3. A yield is 74%.

Synthesis of intermediate M6-5: substitute compound M6-4 for compound M1-4 according to the synthetic method of compound M1-5. A yield is 78%.

4) Synthesis of intermediate M6-6: substitute compound M6-5 for compound M2-2 according to the synthetic method of compound M2-3. A yield is 72%.

5) Synthesis of intermediate M6-7: substitute compound M6-6 for compound M1-3 according to the synthetic method of compound M1-5. A yield is 70%.

6) Synthesis of intermediate M6-8: substitute compound M6-7 for compound M1-5 according to the synthetic method of compound M1-6. A yield is 80%.

7) Synthesis of compound M6: substitute compound M6-8 and compound M6-3 for compound M1-6 and compound M1-7 respectively according to the synthetic method of compound M1. A yield is 70%. MS(ASAP)=590.

### EXAMPLE 7: SYNTHESIS OF COMPOUND (M7)

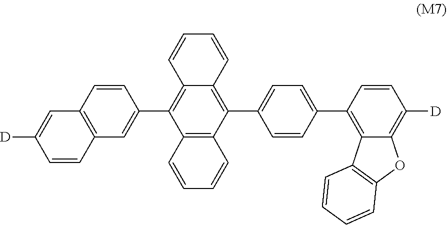 [SMILES: [2H]c1ccc2cc(-c3c4ccccc4c(-c4ccc(-c5ccc([2H])c6oc7ccccc7c56)cc4)c4ccccc34)ccc2c1]

### Synthetic Route

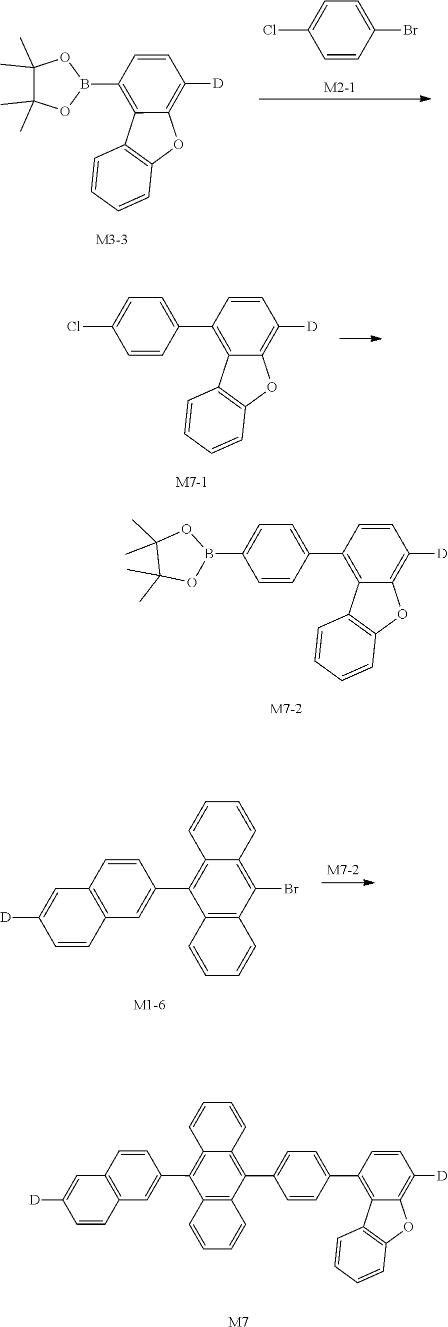

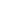

1) Synthesis of intermediate M7-1: substitute compound M3-3 and compound M2-1 for compound M1-3 and M1-4 respectively according to the synthetic method of compound M1-5. A yield is 80%.

2) Synthesis of intermediate M7-2: substitute compound M7-1 for compound M2-2 according to the synthetic method of compound M2-3. A yield is 74%.

3) Synthesis of compound M7: substitute compound M7-2 for compound M1-7 according to the synthetic method of compound M1. A yield is 75%. MS(ASAP)=549.

### EXAMPLE 8: SYNTHESIS OF COMPOUND (M8)

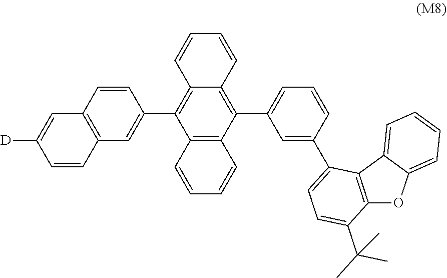 [SMILES: [2H]c1ccc2cc(-c3c4ccccc4c(-c4cccc(-c5ccc(C(C)(C)C)c6oc7ccccc7c56)c4)c4ccccc34)ccc2c1]

### Synthetic Route

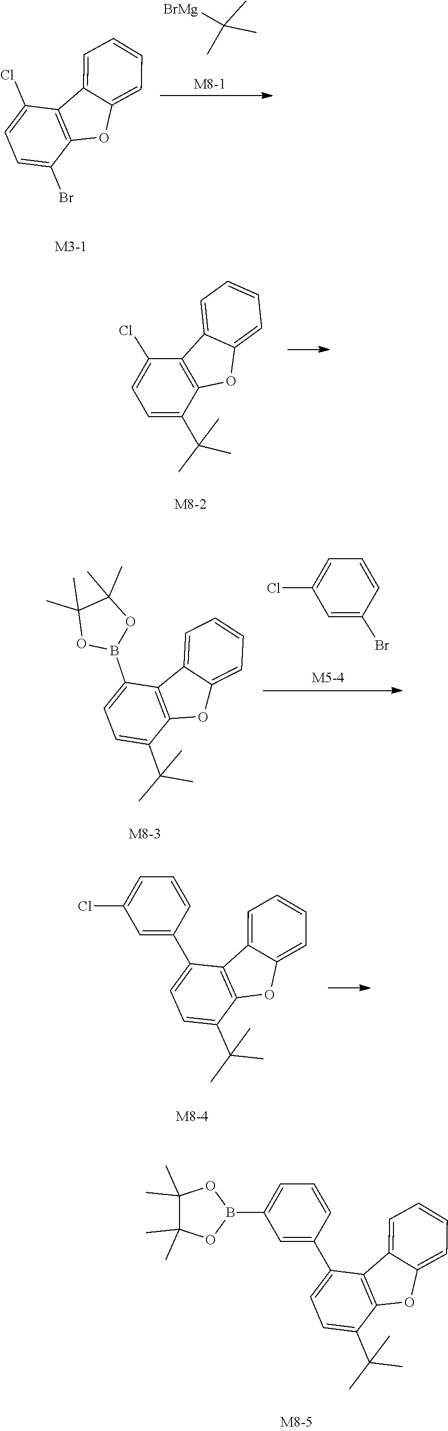

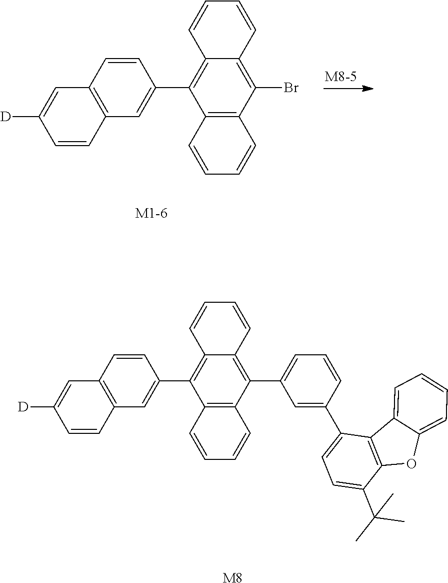 [SMILES: [2H]c1ccc2cc(-c3c4ccccc4c(Br)c4ccccc34)ccc2c1 ; [2H]c1ccc2cc(-c3c4ccccc4c(-c4cccc(-c5ccc(C(C)(C)C)c6oc7ccccc7c56)c4)c4ccccc34)ccc2c1 ; "M8"]

1) Synthesis of intermediate M8-2: substitute compound M3-1 for compound M5-1 and substitute compound M8-1 for methyl magnesium bromide according to a synthetic method of compound M5-2. A yield is 71%.

2) Synthesis of intermediate M8-3: substitute compound M8-2 for compound M2-2 according to the synthetic method of compound M2-3. A yield is 72%.

3) Synthesis of intermediate M8-4: substitute compound M8-3 and compound M5-4 for compound M1-3 and compound M1-4 respectively according to the synthetic method of compound M1-5. A yield is 82%.

4) Synthesis of intermediate M8-5: substitute compound M8-4 for compound M2-2 according to the synthetic method of compound M2-3. A yield is 75%.

5) Synthesis of compound M8: substitute compound M8-5 for compound M1-7 according to the synthetic method of compound M1. A yield is 77%. MS(ASAP)=604.

### EXAMPLE 9: Synthesis of Compound (M9)

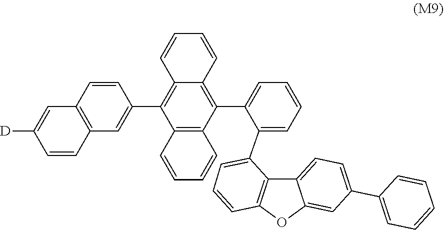 [SMILES: [2H]c1ccc2cc(-c3c4ccccc4c(-c4ccccc4-c4cccc5oc6cc(-c7ccccc7)ccc6c45)c4ccccc34)ccc2c1]

### Synthetic Route

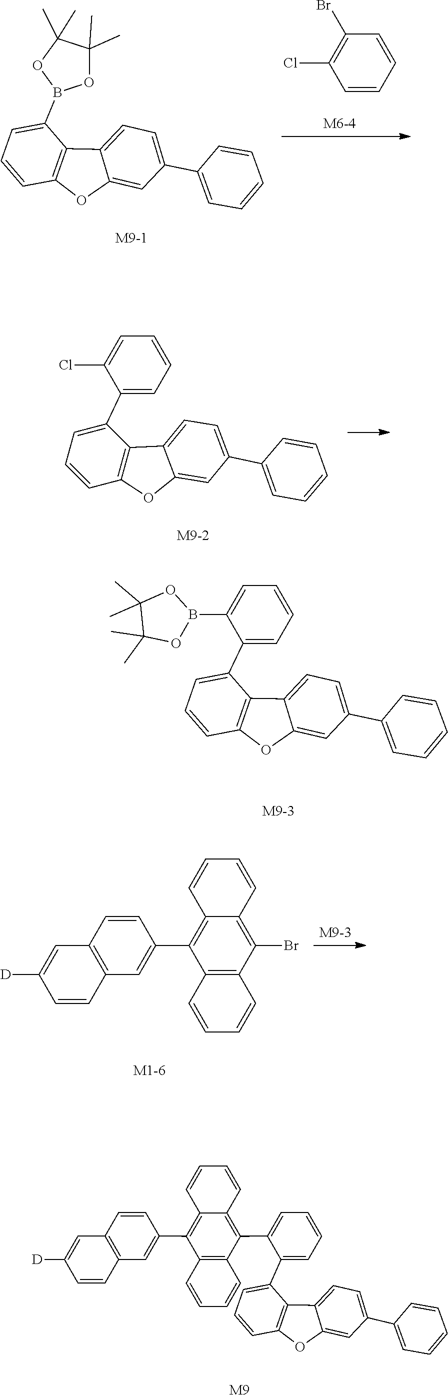

1) Synthesis of intermediate M9-2: substitute compound M9-1 and compound M6-4 for compound M1-3 and compound M1-4 respectively according to the synthetic method of compound M1-5. A yield is 80%.

2) Synthesis of intermediate M9-3: substitute compound M9-2 for compound M2-2 according to the synthetic method of compound M2-3. A yield is 73%.

3) Synthesis of compound M9: substitute compound M9-3 for compound M1-7 according to the synthetic method of compound M1. A yield is 76%. MS(ASAP)=624.

### EXAMPLE 10: SYNTHESIS OF COMPOUND (M10)

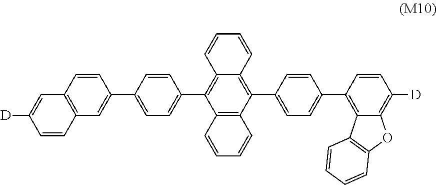 [SMILES: [2H]c1ccc2cc(-c3ccc(-c4c5ccccc5c(-c5ccc(-c6ccc([2H])c7oc8ccccc8c67)cc5)c5ccccc45)cc3)ccc2c1]

### Synthetic Route

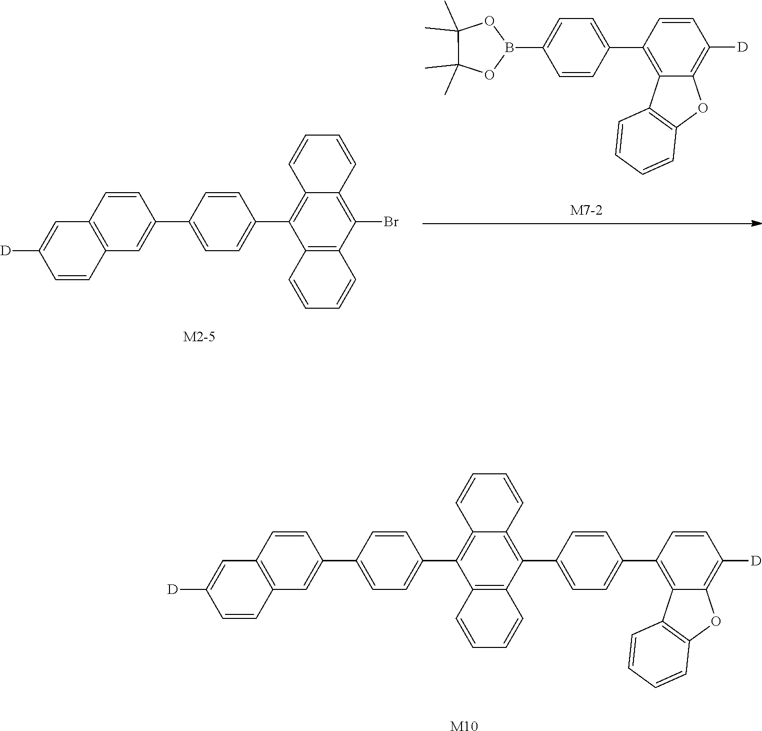 [SMILES: [2H]c1ccc2cc(-c3ccc(-c4c5ccccc5c(Br)c5ccccc45)cc3)ccc2c1 ; [2H]c1ccc(-c2ccc(B3OC(C)(C)C(C)(C)O3)cc2)c2c1oc1ccccc12 ; [2H]c1ccc2cc(-c3ccc(-c4c5ccccc5c(-c5ccc(-c6ccc([2H])c7oc8ccccc8c67)cc5)c5ccccc45)cc3)ccc2c1 ; "M10"]

1) Synthesis of compound M10: substitute compound M2-5 and compound M7-2 for compound M1-6 and compound M1-7 respectively according to the synthetic method of compound M1. A yield is 74%. MS(ASAP)=625.

### EXAMPLE 11: SYNTHESIS OF COMPOUND (M11)

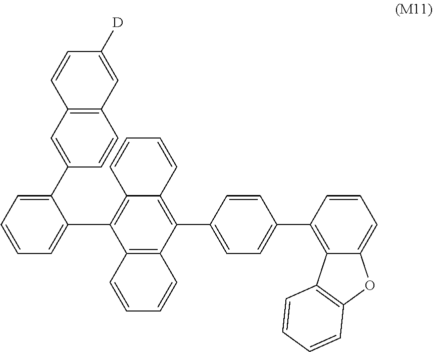 [SMILES: [2H]c1ccc2cc(-c3ccccc3-c3c4ccccc4c(-c4ccc(-c5cccc6oc7ccccc7c56)cc4)c4ccccc34)ccc2c1]

### Synthetic Route

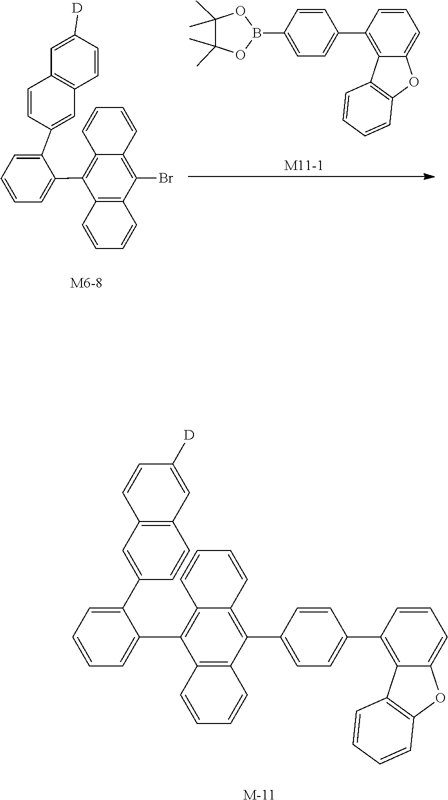 [SMILES: [2H]c1ccc2cc(-c3ccccc3-c3c4ccccc4c(Br)c4ccccc34)ccc2c1 ; CC1(C)OB(c2ccc(-c3cccc4oc5ccccc5c34)cc2)OC1(C)C ; [2H]c1ccc2cc(-c3ccccc3-c3c4ccccc4c(-c4ccc(-c5cccc6oc7ccccc7c56)cc4)c4ccccc34)ccc2c1 ; "M-11"]

1) Synthesis of compound M11: substitute compound M6-8 and compound M11-1 for compound M1-6 and compound M1-7 according to the synthetic method of compound M1. A yield is 72%. MS(ASAP)=624.

### EXAMPLE 12: SYNTHESIS OF COMPOUND (M12)

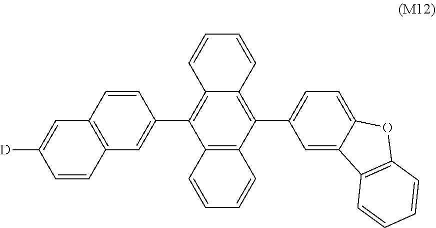 [SMILES: [2H]c1ccc2cc(-c3c4ccccc4c(-c4ccc5oc6ccccc6c5c4)c4ccccc34)ccc2c1]

### Synthetic Route

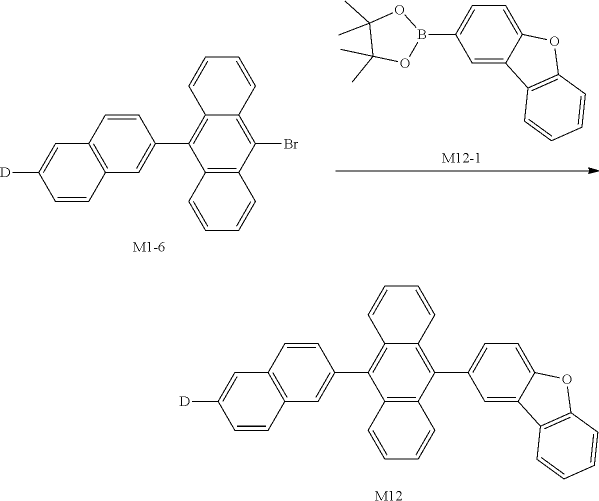

1) Synthesis of compound M12: substitute compound M12-1 for compound M1-7 according to the synthetic method of compound M1. A yield is 75%. MS(ASAP)=472.

### EXAMPLE 13: SYNTHESIS OF COMPOUND (M13)

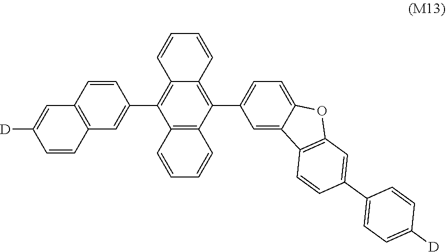 [SMILES: [2H]c1ccc(-c2ccc3c(c2)oc2ccc(-c4c5ccccc5c(-c5ccc6cc([2H])ccc6c5)c5ccccc45)cc23)cc1]

### Synthetic Route

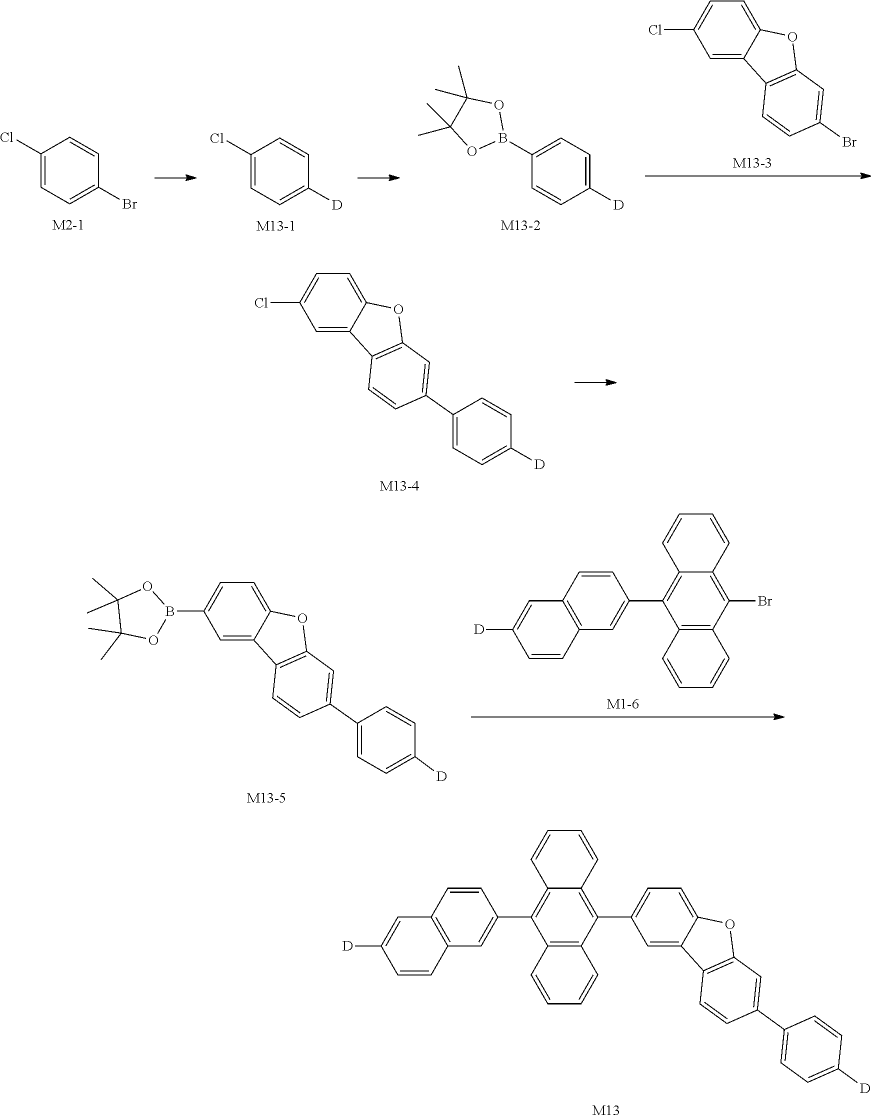

1) Synthesis of intermediate M13-1: substitute compound M2-1 for compound M1-1 according to the synthetic method of compound M1-2. A yield is 85%.

2) Synthesis of intermediate M13-2: substitute compound M13-1 for compound M2-2 according to the synthetic method of compound M2-3. A yield is 78%.

3) Synthesis of intermediate M13-4: substitute compound M13-2 and compound M13-3 for compound M1-3 and compound M1-4 respectively according to the synthetic method of compound M1-5. A yield is 83%.

4) Synthesis of intermediate M13-5: substitute compound M13-4 for compound M2-2 according to the synthetic method of compound M2-3. A yield is 76%.

5) Synthesis of compound M13: substitute compound M13-5 for compound M1-7 according to the synthetic method of compound M1. A yield is 72%. MS(ASAP)=549.

### EXAMPLE 14: SYNTHESIS OF COMPOUND (M14)

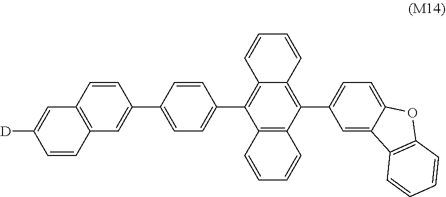 [SMILES: [2H]c1ccc2cc(-c3ccc(-c4c5ccccc5c(-c5ccc6oc7ccccc7c6c5)c5ccccc45)cc3)ccc2c1]

### Synthetic Route

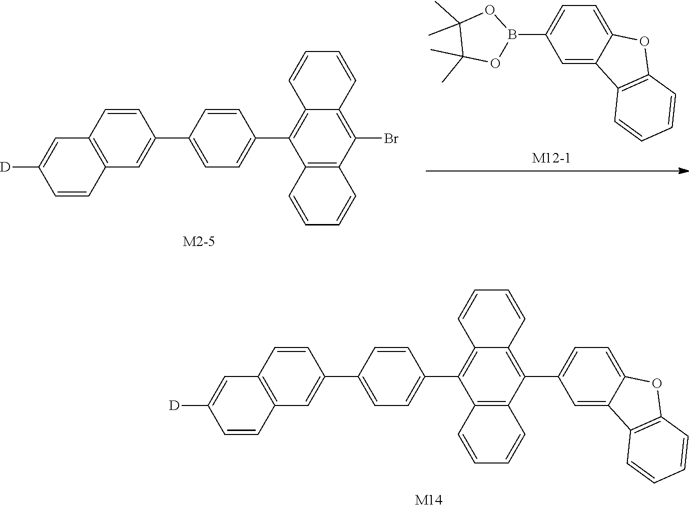 [SMILES: [2H]c1ccc2cc(-c3ccc(-c4c5ccccc5c(Br)c5ccccc45)cc3)ccc2c1 ; CC1(C)OB(c2ccc3oc4ccccc4c3c2)OC1(C)C ; [2H]c1ccc2cc(-c3ccc(-c4c5ccccc5c(-c5ccc6oc7ccccc7c6c5)c5ccccc45)cc3)ccc2c1 ; "M14"]

1) Synthesis of compound M14: substitute compound M2-5 and compound M12-1 for compound M1-6 and compound M1-7 respectively according to the synthetic method of compound M1. A yield is 81%. MS(ASAP)=548.

### EXAMPLE 15: SYNTHESIS OF COMPOUND (M15)

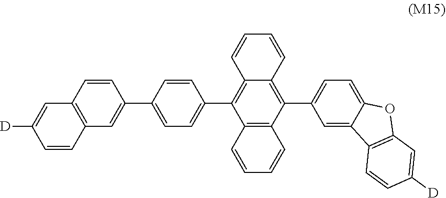 [SMILES: [2H]c1ccc2cc(-c3ccc(-c4c5ccccc5c(-c5ccc6oc7cc([2H])ccc7c6c5)c5ccccc45)cc3)ccc2c1]

### Synthetic Route

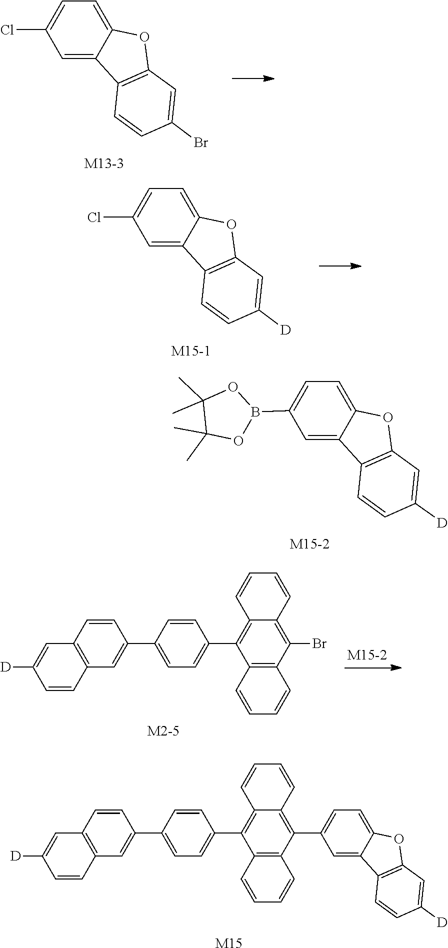 [SMILES: Clc1ccc2oc3cc(Br)ccc3c2c1 ; [2H]c1ccc2c(c1)oc1ccc(Cl)cc12 ; [2H]c1ccc2c(c1)oc1ccc(B3OC(C)(C)C(C)(C)O3)cc12 ; [2H]c1ccc2cc(-c3ccc(-c4c5ccccc5c(Br)c5ccccc45)cc3)ccc2c1 ; [2H]c1ccc2cc(-c3ccc(-c4c5ccccc5c(-c5ccc6oc7cc([2H])ccc7c6c5)c5ccccc45)cc3)ccc2c1 ; "M15"]

1) Synthesis of intermediate M15-1: substitute compound M13-3 for compound M1-1 according to the synthetic method of compound M1-2. A yield is 82%.

2) Synthesis of intermediate M15-2: substitute compound M15-1 for compound M2-2 according to the synthetic method of compound M2-3. A yield is 76%.

3) Synthesis of compound M15: substitute compound M2-5 and compound M15-2 for compound M1-6 and compound M1-7 respectively according to the synthetic method of compound M1. A yield is 82%. MS(ASAP)=549.

### EXAMPLE 16: SYNTHESIS OF COMPOUND (M16)

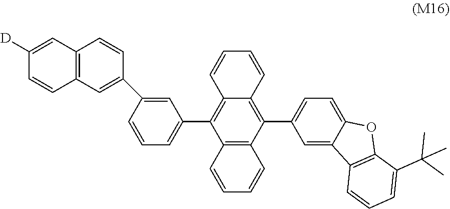 [SMILES: [2H]c1ccc2cc(-c3cccc(-c4c5ccccc5c(-c5ccc6oc7c(C(C)(C)C)cccc7c6c5)c5ccccc45)c3)ccc2c1]

### Synthetic Route

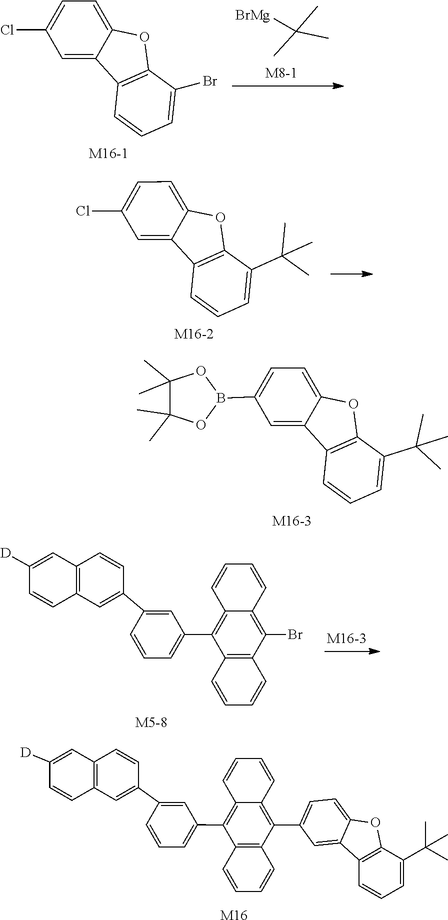

1) Synthesis of intermediate M16-2: substitute compound M16-1 for compound M5-1 and substitute compound M8-1 for methyl magnesium bromide according to the synthetic method of compound M5-2. A yield is 70%.

2) Synthesis of intermediate M16-3: substitute compound M16-2 for compound M2-2 according to the synthetic method of compound M2-3. A yield is 75%.

3) Synthesis of compound M16: substitute compound M5-8 and compound M16-3 for compound M1-6 and compound M1-7 respectively according to the synthetic method of compound M1. A yield is 84%. MS(ASAP)=604.

### EXAMPLE 17: SYNTHESIS OF COMPOUND (M17)

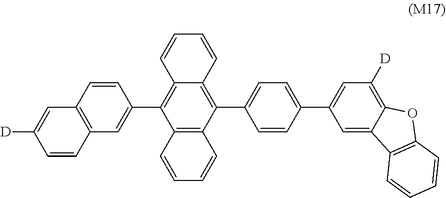 [SMILES: [2H]c1ccc2cc(-c3c4ccccc4c(-c4ccc(-c5cc([2H])c6oc7ccccc7c6c5)cc4)c4ccccc34)ccc2c1]

### Synthetic Route

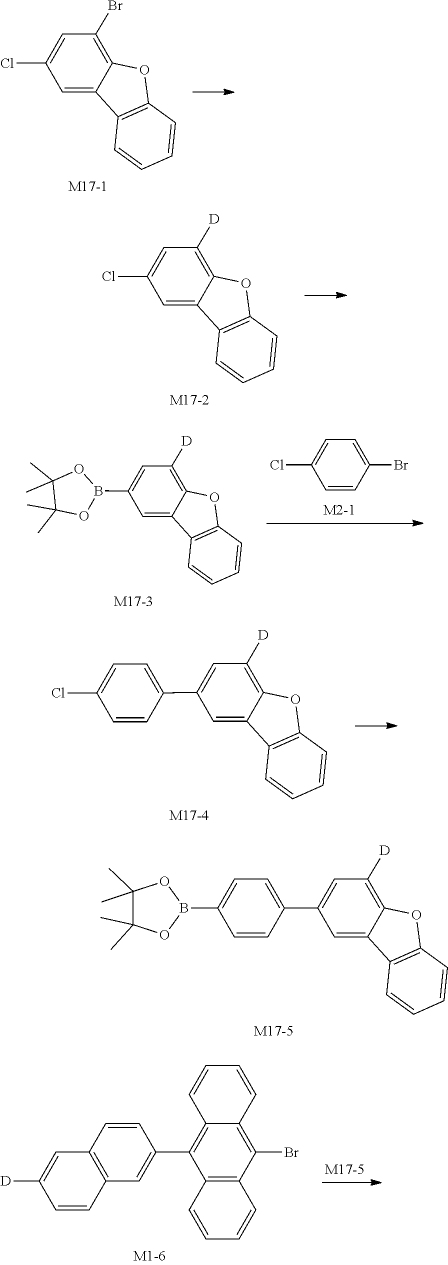

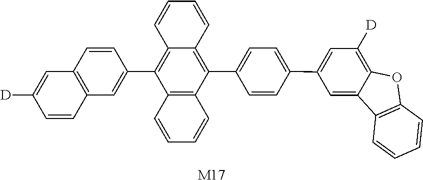

1) Synthesis of intermediate M17-2: substitute compound M17-1 for compound M1-1 according to the synthetic method of compound M1-2. A yield is 81%.

2) Synthesis of intermediate M17-3: substitute compound M17-2 for compound M2-2 according to the synthetic method of compound M2-3. A yield is 73%.

3) Synthesis of intermediate M17-4: substitute compound M17-3 and compound M2-1 for compound M1-3 and compound M1-4 respectively according to the synthetic method of compound M1-5. A yield is 85%.

4) Synthesis of intermediate M17-5: substitute compound M17-4 for compound M2-2 according to the synthetic method of compound M2-3. A yield is 75%.

5) Synthesis of compound M17: substitute compound M17-5 for compound M1-7 according to the synthetic method of compound M1. A yield is 82%. MS(ASAP)=549.

### EXAMPLE 18: SYNTHESIS OF COMPOUND (M18)

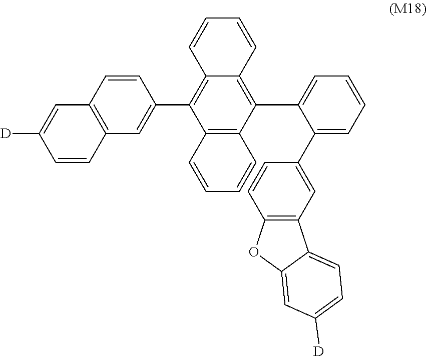 [SMILES: [2H]c1ccc2cc(-c3c4ccccc4c(-c4ccccc4-c4ccc5oc6cc([2H])ccc6c5c4)c4ccccc34)ccc2c1]

### Synthetic Route

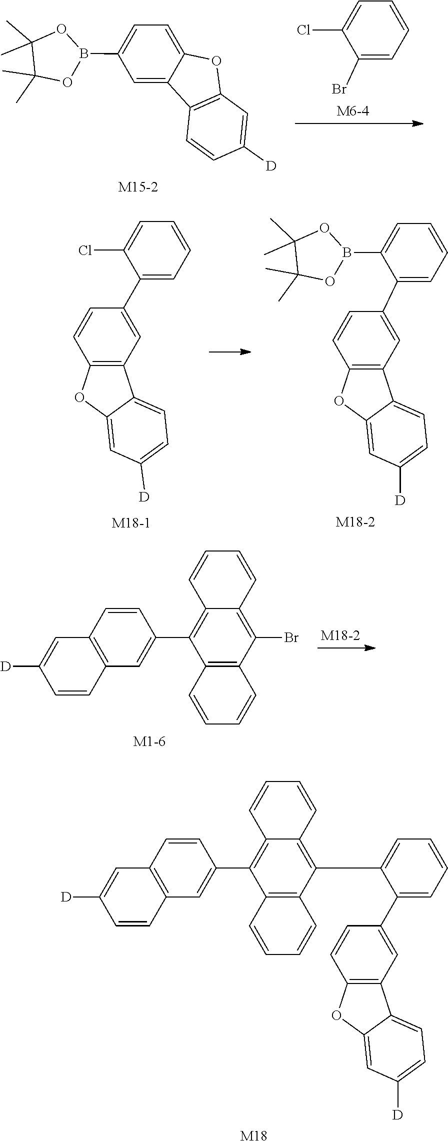

1) Synthesis of intermediate M18-1: substitute compound M15-2 and compound M6-4 for compound M1-3 and compound M1-4 respectively according to the synthetic method of compound M1-5. A yield is 78%.

2) Synthesis of intermediate M18-2: substitute compound M18-1 for compound M2-2 according to the synthetic method of compound M2-3. A yield is 70%.

3) Synthesis of compound M18: substitute compound M18-2 for compound M1-7 according to the synthetic method of compound M1. A yield is 76%. MS(ASAP)=549.

### EXAMPLE 19: SYNTHESIS OF COMPOUND (M19)

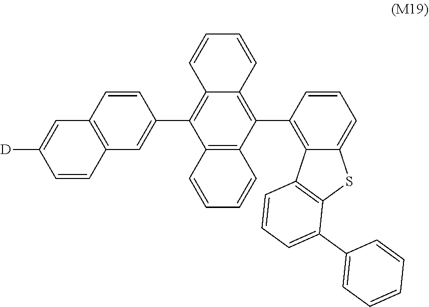 [SMILES: [2H]c1ccc2cc(-c3c4ccccc4c(-c4cccc5sc6c(-c7ccccc7)cccc6c45)c4ccccc34)ccc2c1]

### Synthetic Route

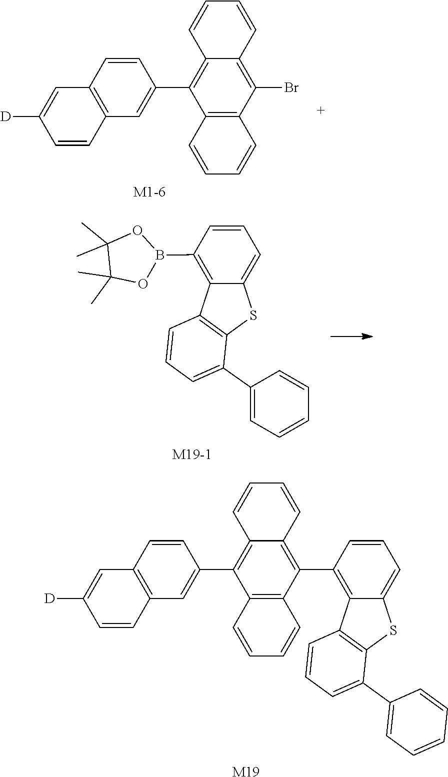

1) Synthesis of compound M19: substitute compound M19-1 for compound M1-7 according to the synthetic method of compound M1. A yield is 82%. MS(ASAP)=564.

### EXAMPLE 20: SYNTHESIS OF COMPOUND (M20)

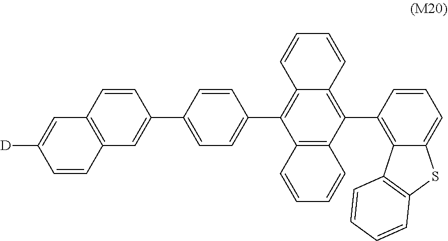 [SMILES: [2H]c1ccc2cc(-c3ccc(-c4c5ccccc5c(-c5cccc6sc7ccccc7c56)c5ccccc45)cc3)ccc2c1]

### Synthetic Route

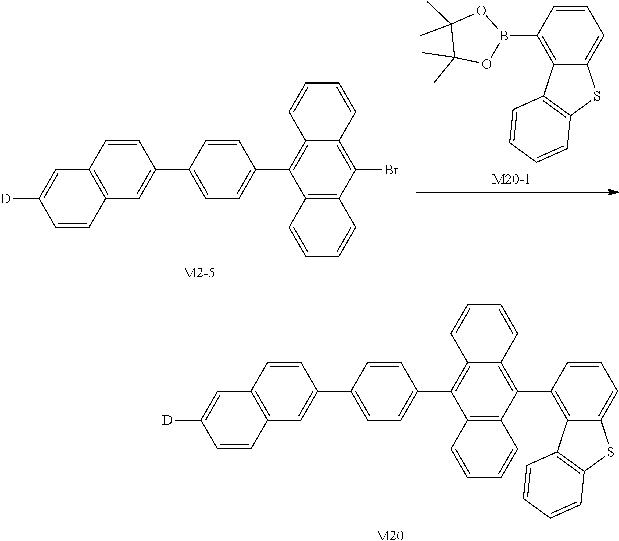 [SMILES: [2H]c1ccc2cc(-c3ccc(-c4c5ccccc5c(Br)c5ccccc45)cc3)ccc2c1 ; CC1(C)OB(c2cccc3sc4ccccc4c23)OC1(C)C ; [2H]c1ccc2cc(-c3ccc(-c4c5ccccc5c(-c5cccc6sc7ccccc7c56)c5ccccc45)cc3)ccc2c1 ; "M20"]

1) Synthesis of compound M20: substitute compound M2-5 and compound M20-1 for compound M1-6 and compound M1-7 respectively according to the synthetic method of compound M1. A yield is 80%. MS(ASAP)=564.

### EXAMPLE 21: SYNTHESIS OF COMPOUND (M21)

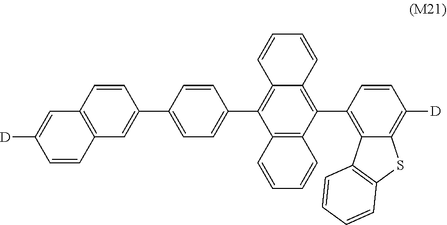 [SMILES: [2H]c1ccc2cc(-c3ccc(-c4c5ccccc5c(-c5ccc([2H])c6sc7ccccc7c56)c5ccccc45)cc3)ccc2c1]

### Synthetic Route

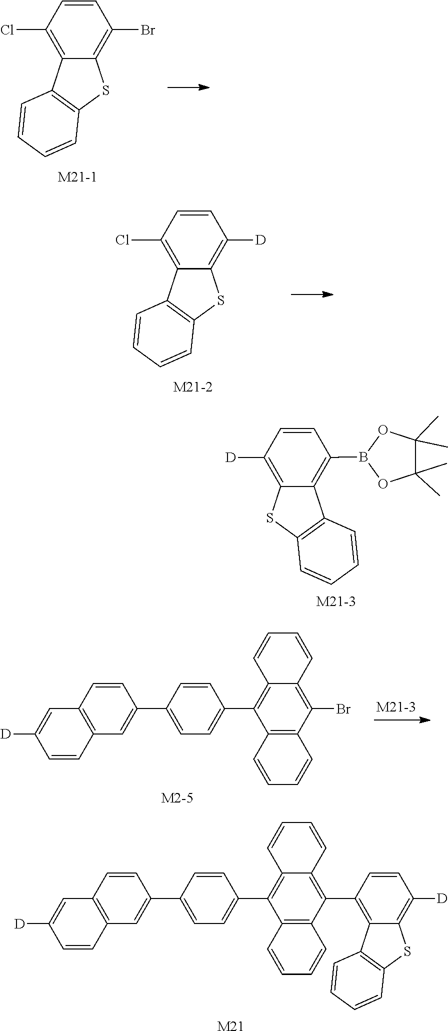

1) Synthesis of intermediate M21-2: substitute compound M21-1 for compound M1-1 according to the synthetic method of compound M1-2. A yield is 82%.

2) Synthesis of intermediate M21-3: substitute compound M21-2 for M2-2 according to the synthetic method of compound M2-3. A yield is 74%.

3) Synthesis of compound M21: substitute compound M2-5 and compound M21-3 for compound M1-6 and compound M1-7 respectively according to the synthetic method of compound M1. A yield is 75%. MS(ASAP)=565.

### EXAMPLE 22: SYNTHESIS OF COMPOUND (M22)

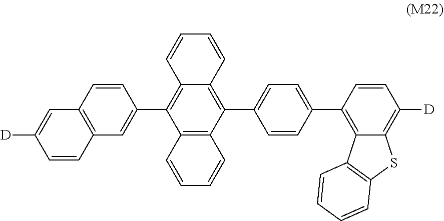 [SMILES: [2H]c1ccc2cc(-c3c4ccccc4c(-c4ccc(-c5ccc([2H])c6sc7ccccc7c56)cc4)c4ccccc34)ccc2c1]

### Synthetic Route

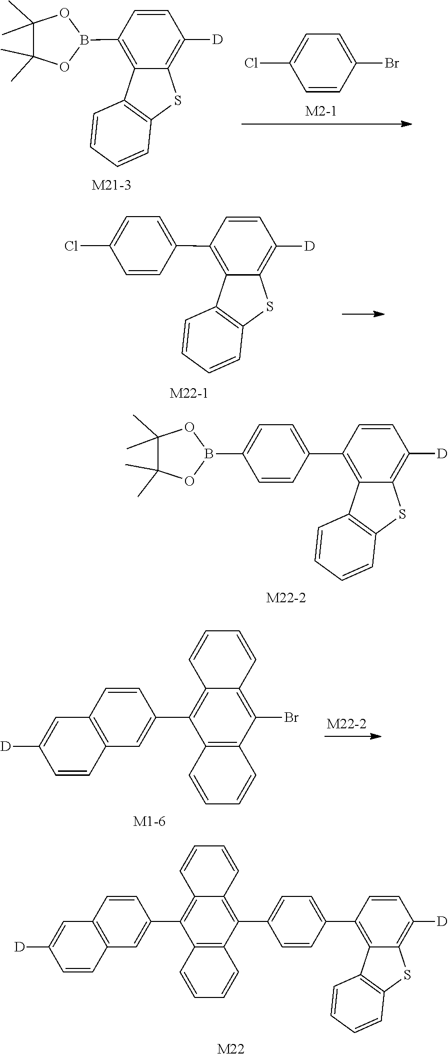

1) Synthesis of intermediate M22-1: substitute compound M21-3 and compound M2-1 for compound M1-3 and compound M1-4 respectively according to the synthetic method of compound M1-5. A yield is 83%.

2) Synthesis of intermediate M22-2: substitute compound M22-1 for compound M2-2 according to the synthetic method of compound M2-3. A yield is 76%.

3) Synthesis of compound M22: substitute compound M22-2 for compound M1-7 according to the synthetic method of compound M1. A yield is 77%. MS(ASAP)=565.

### EXAMPLE 23: SYNTHESIS OF COMPOUND (M23)

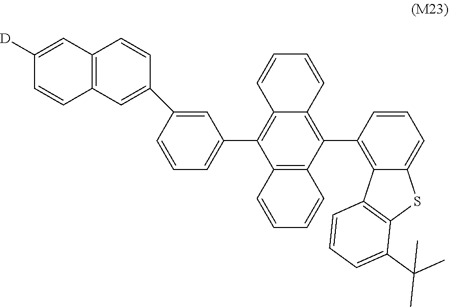 [SMILES: [2H]c1ccc2cc(-c3cccc(-c4c5ccccc5c(-c5cccc6sc7c(C(C)(C)C)cccc7c56)c5ccccc45)c3)ccc2c1]

### Synthetic Route

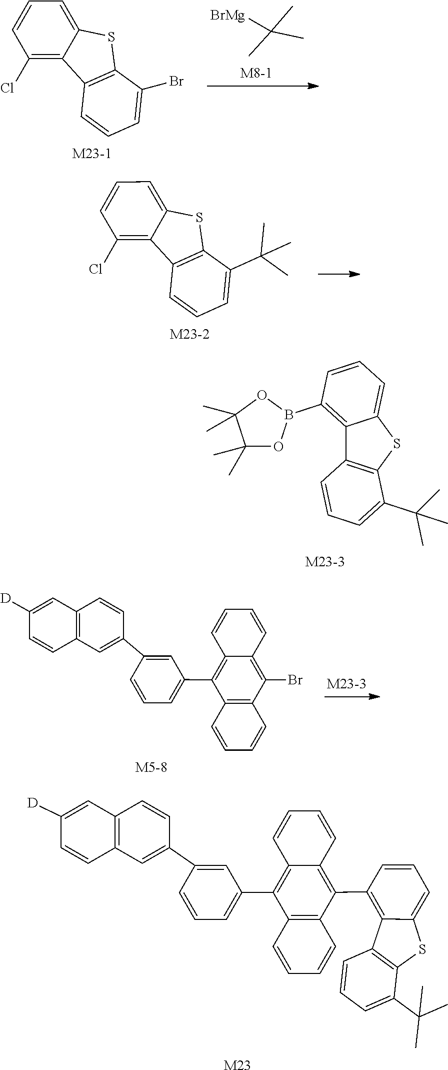

1) Synthesis of intermediate M23-2: substitute compound M23-1 for compound M5-1 and substitute compound M8-1 for methyl magnesium bromide according to the synthetic method of compound M5-2. A yield is 72%.

2) Synthesis of intermediate M23-3: substitute compound M23-2 for M2-2 according to the synthetic method of compound M2-3. A yield is 76%.

3) Synthesis of compound M23: substitute compound M5-8 and compound M23-3 for compound M1-6 and compound M1-7 respectively according to the synthetic method of compound M1. A yield is 82%. MS(ASAP)=620.

### EXAMPLE 24: SYNTHESIS OF COMPOUND (M24)

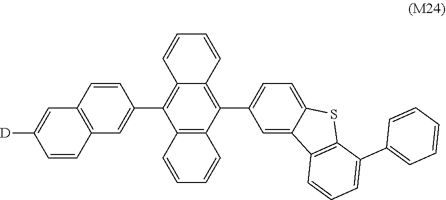 [SMILES: [2H]c1ccc2cc(-c3c4ccccc4c(-c4ccc5sc6c(-c7ccccc7)cccc6c5c4)c4ccccc34)ccc2c1]

### Synthetic Route

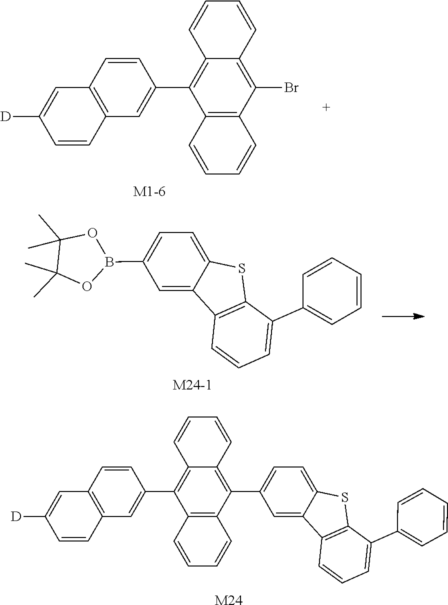

1) Synthesis of compound M24: substitute compound M24-1 for compound M1-7 according to the synthetic method of compound M1. A yield is 80%. MS(ASAP)=564.

### EXAMPLE 25: SYNTHESIS OF COMPOUND (M25)

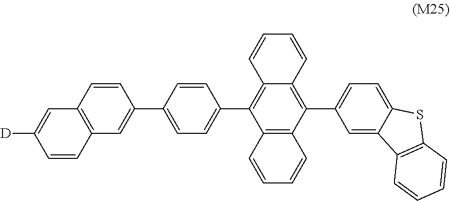 [SMILES: [2H]c1ccc2cc(-c3ccc(-c4c5ccccc5c(-c5ccc6sc7ccccc7c6c5)c5ccccc45)cc3)ccc2c1]

### Synthetic Route

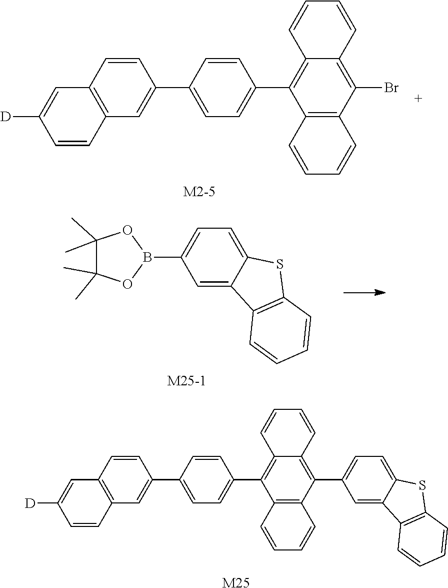

1) Synthesis of compound M25: substitute compound M2-5 and compound M25-1 for compound M1-6 and compound M1-7 respectively according to the synthetic method of compound M1. A yield is 81%. MS(ASAP)=564.

### EXAMPLE 26: SYNTHESIS OF COMPOUND (M26)

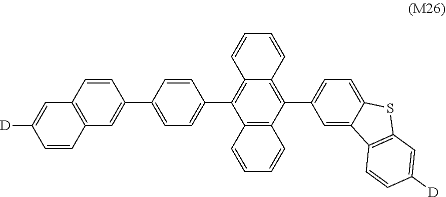 [SMILES: [2H]c1ccc2cc(-c3ccc(-c4c5ccccc5c(-c5ccc6sc7cc([2H])ccc7c6c5)c5ccccc45)cc3)ccc2c1]

### Synthetic Route

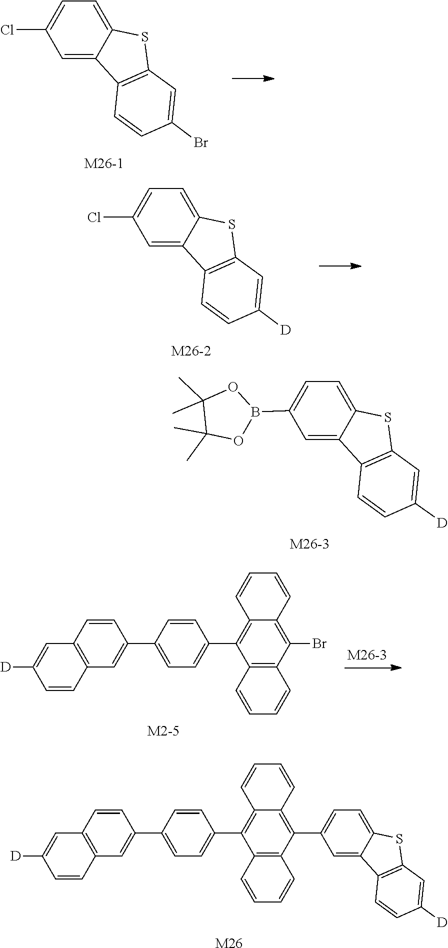 [SMILES: Clc1ccc2sc3cc(Br)ccc3c2c1 ; [2H]c1ccc2c(c1)sc1ccc(Cl)cc12 ; [2H]c1ccc2c(c1)sc1ccc(B3OC(C)(C)C(C)(C)O3)cc12 ; [2H]c1ccc2cc(-c3ccc(-c4c5ccccc5c(Br)c5ccccc45)cc3)ccc2c1 ; [2H]c1ccc2cc(-c3ccc(-c4c5ccccc5c(-c5ccc6sc7cc([2H])ccc7c6c5)c5ccccc45)cc3)ccc2c1 ; "M26"]

1) Synthesis of intermediate M26-2: substitute compound M26-1 for compound M1-1 according to the synthetic method of compound M1-2. A yield is 80%.

2) Synthesis of intermediate M26-3: substitute compound M26-2 for compound M2-2 according to the synthetic method of compound M2-3. A yield is 75%.

3) Synthesis of compound M26: substitute compound M2-5 and compound M26-3 for compound M1-6 and compound M1-7 respectively according to the synthetic method of compound M1. A yield is 78%. MS(ASAP)=565.

### EXAMPLE 27: SYNTHESIS OF COMPOUND (M27)

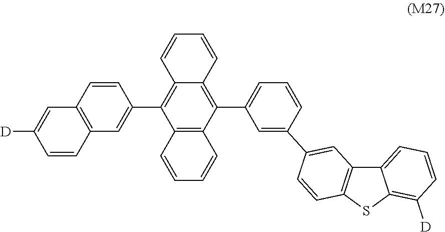 [SMILES: [2H]c1ccc2cc(-c3c4ccccc4c(-c4cccc(-c5ccc6sc7c([2H])cccc7c6c5)c4)c4ccccc34)ccc2c1]

### Synthetic Route

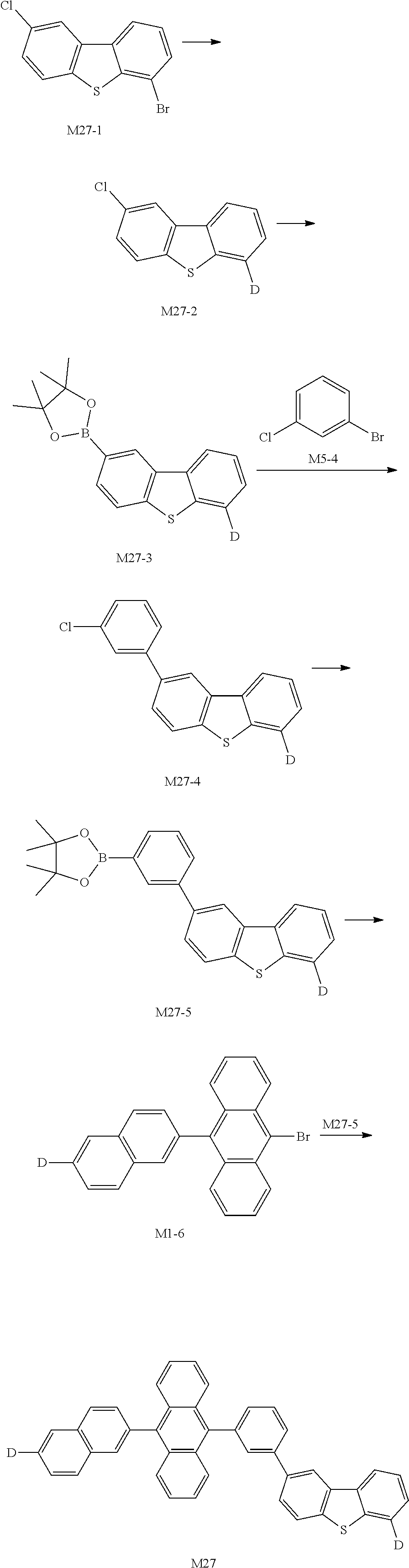

1) Synthesis of intermediate M27-2: substitute compound M27-1 for compound M1-1 according to the synthetic method of compound M1-2. A yield is 78%.

2) Synthesis of intermediate M27-3: substitute compound M27-2 for compound M2-2 according to the synthetic method of compound M2-3. A yield is 74%.

3) Synthesis of intermediate M27-4: substitute compound M27-3 and compound M5-4 for compound M1-3 and compound M1-4 respectively according to the synthetic method of compound M1-5. A yield is 80%.

4) Synthesis of intermediate M27-5: substitute compound M27-4 for compound M2-2 according to the synthetic method of compound M2-3. A yield is 76%.

5) Synthesis of compound M27: substitute compound M27-5 for compound M1-7 according to the synthetic method of compound M1. A yield is 76%. MS(ASAP)=565.

### EXAMPLE 28: SYNTHESIS OF COMPOUND (M28)

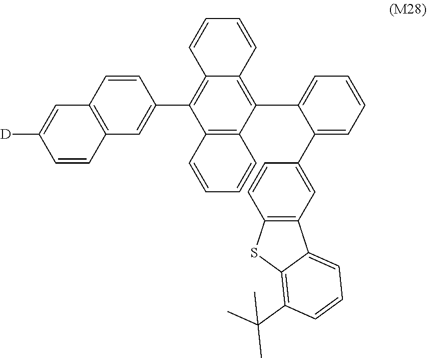 [SMILES: [2H]c1ccc2cc(-c3c4ccccc4c(-c4ccccc4-c4ccc5sc6c(C(C)(C)C)cccc6c5c4)c4ccccc34)ccc2c1]

### Synthetic Route

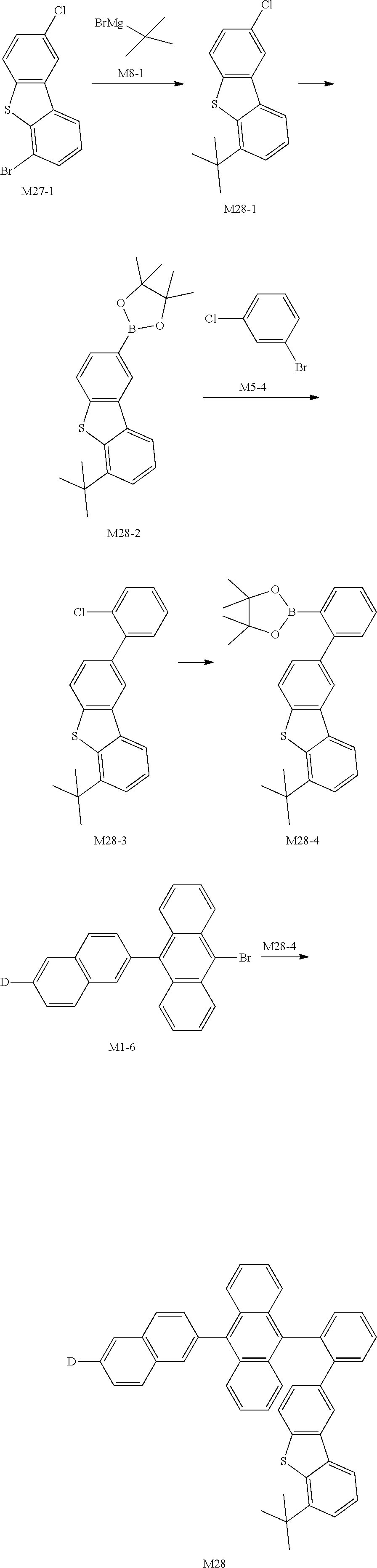

1) Synthesis of intermediate M28-1: substitute compound M27-1 for compound M5-1 and substitute compound M8-1 for methyl magnesium bromide according to the synthetic method of compound M5-2. A yield is 72%.

2) Synthesis of intermediate M28-2: substitute compound M28-1 for compound M2-2 according to the synthetic method of compound M2-3. A yield is 73%.

3) Synthesis of intermediate M28-3: substitute compound M28-2 and compound M5-4 for compound M1-3 and compound M1-4 respectively according to the synthetic method of compound M1-5. A yield is 80%.

4) Synthesis of intermediate M28-4: substitute compound M28-3 for compound M2-2 according to the synthetic method of compound M2-3. A yield is 72%.

5) Synthesis of compound M28: substitute compound M28-4 for compound M1-7 according the synthetic method of compound M1. A yield is 73%. MS(ASAP)=620.

### EXAMPLE 29: SYNTHESIS OF COMPOUND (M29)

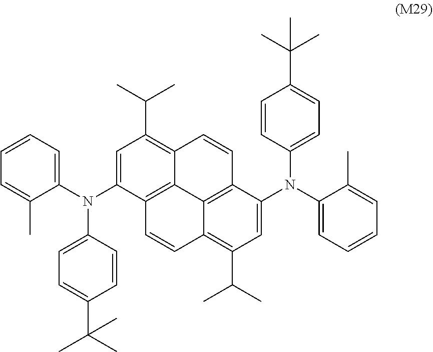 [SMILES: Cc1ccccc1N(c1ccc(C(C)(C)C)cc1)c1cc(C(C)C)c2ccc3c(N(c4ccc(C(C)(C)C)cc4)c4ccccc4C)cc(C(C)C)c4ccc1c2c43]

### Synthetic Route

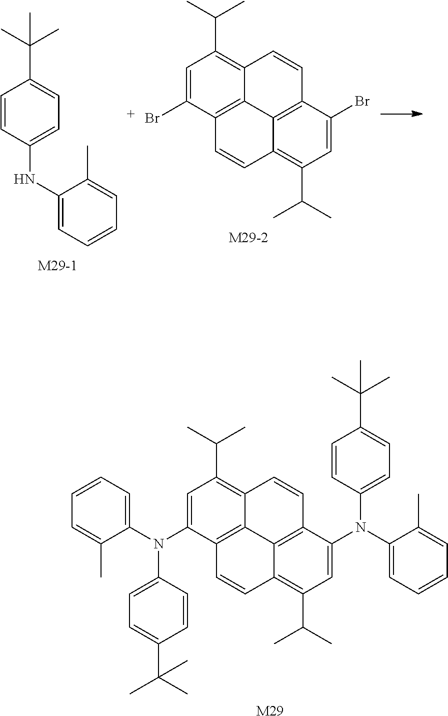 [SMILES: Cc1ccccc1Nc1ccc(C(C)(C)C)cc1 ; CC(C)c1cc(Br)c2ccc3c(C(C)C)cc(Br)c4ccc1c2c43 ; Cc1ccccc1N(c1ccc(C(C)(C)C)cc1)c1cc(C(C)C)c2ccc3c(N(c4ccc(C(C)(C)C)cc4)c4ccccc4C)cc(C(C)C)c4ccc1c2c43 ; "M29"]

1) Synthesis of compound M29: in a nitrogen atmosphere, add 14.3 g (60 mmol) of compound M29-1, 13.3 g (30 mmol) of compound M29-2, 5.73 g (30 mmol) of copper iodide, 3.42 g (30 mmol) of trans cyclohexanediamine, 19.1 g (60 mmol) of potassium phosphate, and 150 mL of toluene into a three neck-flask (300 mL). Heat the solution to a temperature of 110° C., and stir it for 12 hours. After the reaction is completed, cool the solution to room temperature. Filter the filtrate by suction, and collect the filtrate after filtering. Rotary evaporate the reaction solution to remove most of the solvent, dissolve it with dichloromethane, and wash it with water for 3 times. Collect the organic solution and stir it with silica gel. Purify the product in column chromatography purification. A yield is 65%. MS(ASAP)=761.

### EXAMPLE 30: SYNTHESIS OF COMPOUND (M30)

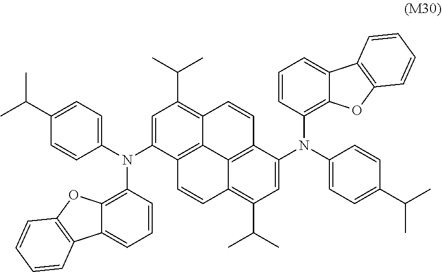 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1]

### Synthetic Route

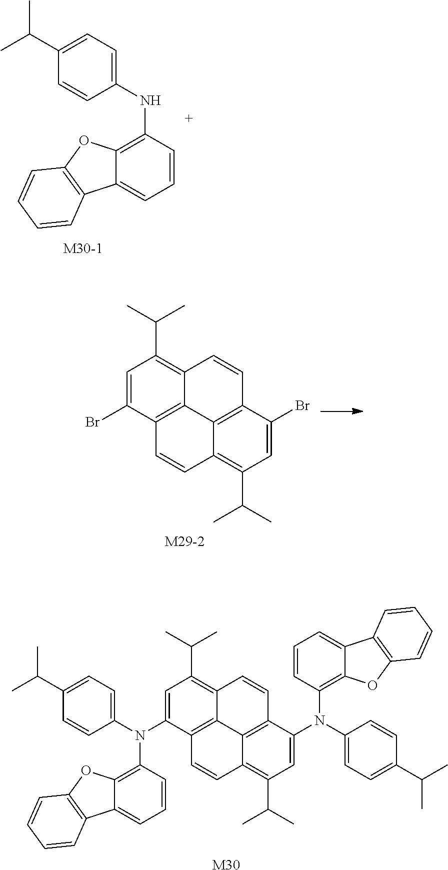 [SMILES: CC(C)c1ccc(Nc2cccc3c2oc2ccccc23)cc1 ; CC(C)c1cc(Br)c2ccc3c(C(C)C)cc(Br)c4ccc1c2c43 ; CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; "M30"]

1) Synthesis of compound M30: substitute compound M30-1 for compound M29-1 according to a synthetic method of compound M29. A yield is 63%. MS(ASAP)=885.

### EXAMPLE 31: SYNTHESIS OF COMPOUND (M31)

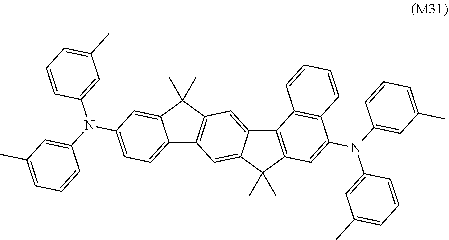

### Synthetic Route

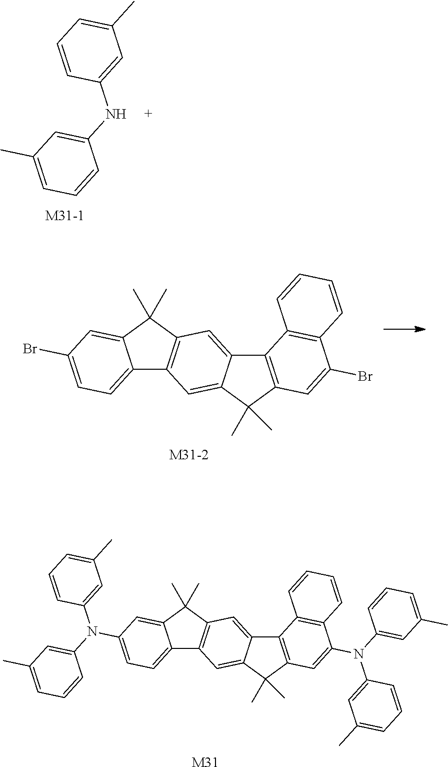

1) Synthesis of compound M31: substitute compound M31-1 and compound M31-2 for compound M29-1 and compound M29-2 respectively according to the synthetic method of compound M29. A yield is 68%. MS(ASAP)=751.

### EXAMPLE 32: SYNTHESIS OF COMPOUND (M32)

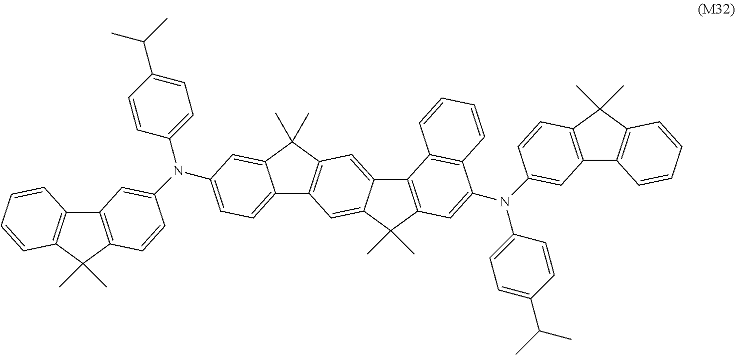 [SMILES: CC(C)c1ccc(N(c2ccc3c(c2)-c2ccccc2C3(C)C)c2ccc3c(c2)C(C)(C)c2cc4c(cc2-3)C(C)(C)c2cc(N(c3ccc(C(C)C)cc3)c3ccc5c(c3)-c3ccccc3C5(C)C)c3ccccc3c2-4)cc1]

### Synthetic Route

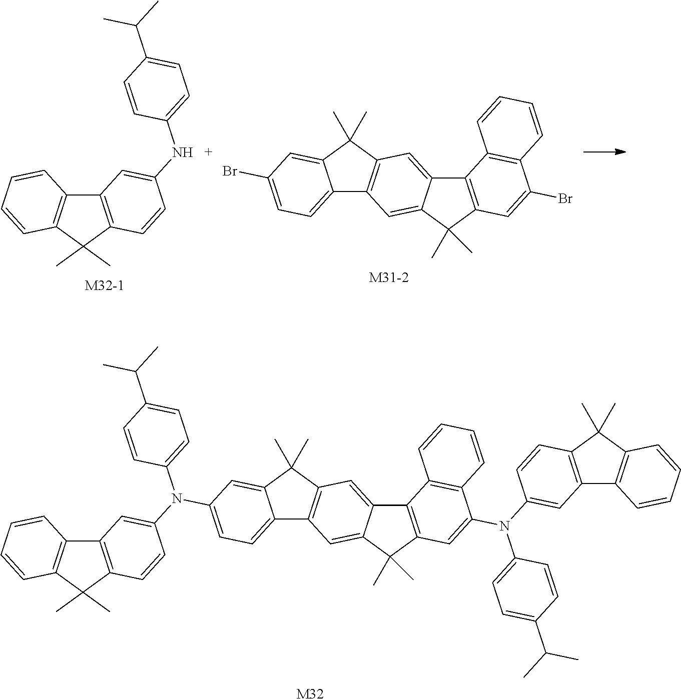 [SMILES: CC(C)c1ccc(Nc2ccc3c(c2)-c2ccccc2C3(C)C)cc1 ; CC1(C)c2cc(Br)ccc2-c2cc3c(cc21)-c1c(cc(Br)c2ccccc12)C3(C)C ; CC(C)c1ccc(N(c2ccc3c(c2)-c2ccccc2C3(C)C)c2ccc3c(c2)C(C)(C)c2cc4c(cc2-3)C(C)(C)c2cc(N(c3ccc(C(C)C)cc3)c3ccc5c(c3)-c3ccccc3C5(C)C)c3ccccc3c2-4)cc1 ; "M32"]

1) Synthesis of compound M32: substitute compound M31-1 and compound M31-2 for compound M29-1 and compound M29-2 respectively according to the synthetic method of compound M29. A yield is 68%. MS(ASAP)=751.

### EXAMPLE 33: SYNTHESIS OF COMPOUND (M33)

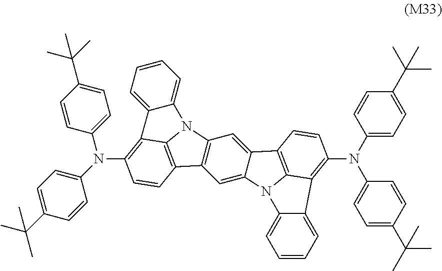 [SMILES: CC(C)(C)c1ccc(N(c2ccc(C(C)(C)C)cc2)c2ccc3c4cc5c(cc4n4c6ccccc6c2c34)c2ccc(N(c3ccc(C(C)(C)C)cc3)c3ccc(C(C)(C)C)cc3)c3c4ccccc4n5c23)cc1]

### Synthetic Route

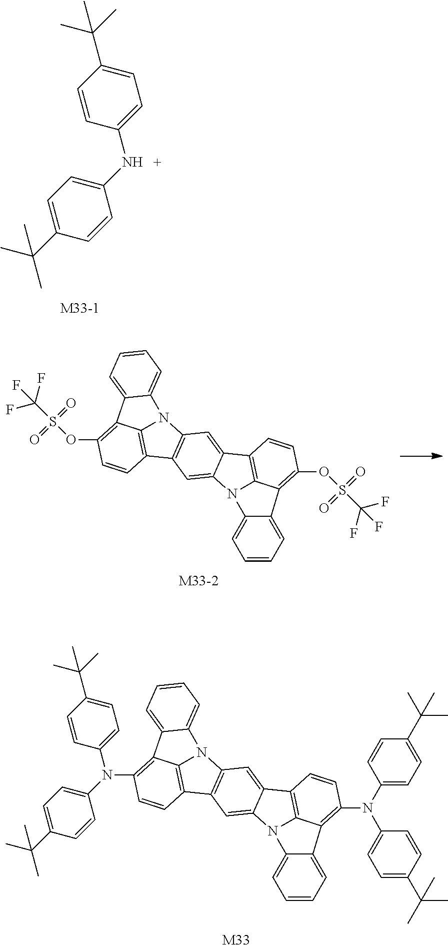 [SMILES: CC(C)(C)c1ccc(Nc2ccc(C(C)(C)C)cc2)cc1 ; O=S(=O)(Oc1ccc2c3cc4c(cc3n3c5ccccc5c1c23)c1ccc(OS(=O)(=O)C(F)(F)F)c2c3ccccc3n4c12)C(F)(F)F ; CC(C)(C)c1ccc(N(c2ccc(C(C)(C)C)cc2)c2ccc3c4cc5c(cc4n4c6ccccc6c2c34)c2ccc(N(c3ccc(C(C)(C)C)cc3)c3ccc(C(C)(C)C)cc3)c3c4ccccc4n5c23)cc1 ; "M33"]

Synthesis of compound M33: in a nitrogen atmosphere, add 16.9 g (60 mmol) of compound M33-1, 21 g (30 mmol) of compound M33-2, 2.75 g (3 mmol) of tris (dibenzylideneacetone) dipalladium, 2.43 g (6 mmol) of dicyclohexyl (2,2-diphenyl-1-methylcyclopropyl) phosphine, 60 mmol of bis (trimethylsilyl) amide lithium, and 150 mL of xylene into a three neck-flask (300 mL). Heat the solution to a temperature of 150° C., and stir it for 12 hours. After the reaction is completed, cool the solution to room temperature. Filter the filtrate by suction, and collect the filtrate after filtering. Rotary evaporate the reaction solution to remove most of the solvent. Dissolve it with dichloromethane. Wash with water for 3 times, and then collect the organic phase. Dry the organic phase by rotary evaporation. Recrystallize the product for purification. A yield is 77%. MS(ASAP)=964.

### EXAMPLE 34: SYNTHESIS OF COMPOUND (M34)

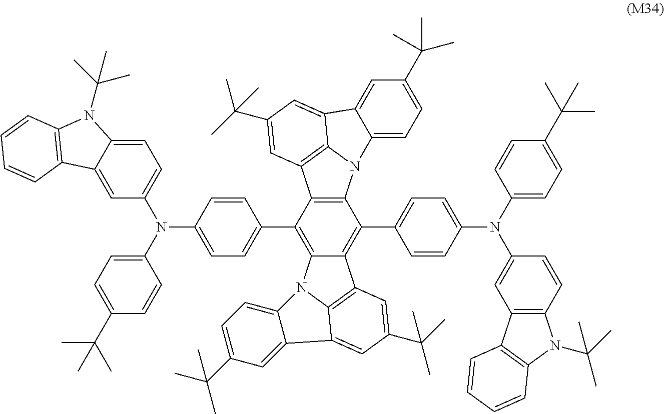 [SMILES: CC(C)(C)c1ccc(N(c2ccc(-c3c4c5cc(C(C)(C)C)cc6c7cc(C(C)(C)C)ccc7n(c4c(-c4ccc(N(c7ccc(C(C)(C)C)cc7)c7ccc8c(c7)c7ccccc7n8C(C)(C)C)cc4)c4c7cc(C(C)(C)C)cc8c9cc(C(C)(C)C)ccc9n(c34)c87)c65)cc2)c2ccc3c(c2)c2ccccc2n3C(C)(C)C)cc1]

### Synthetic Route

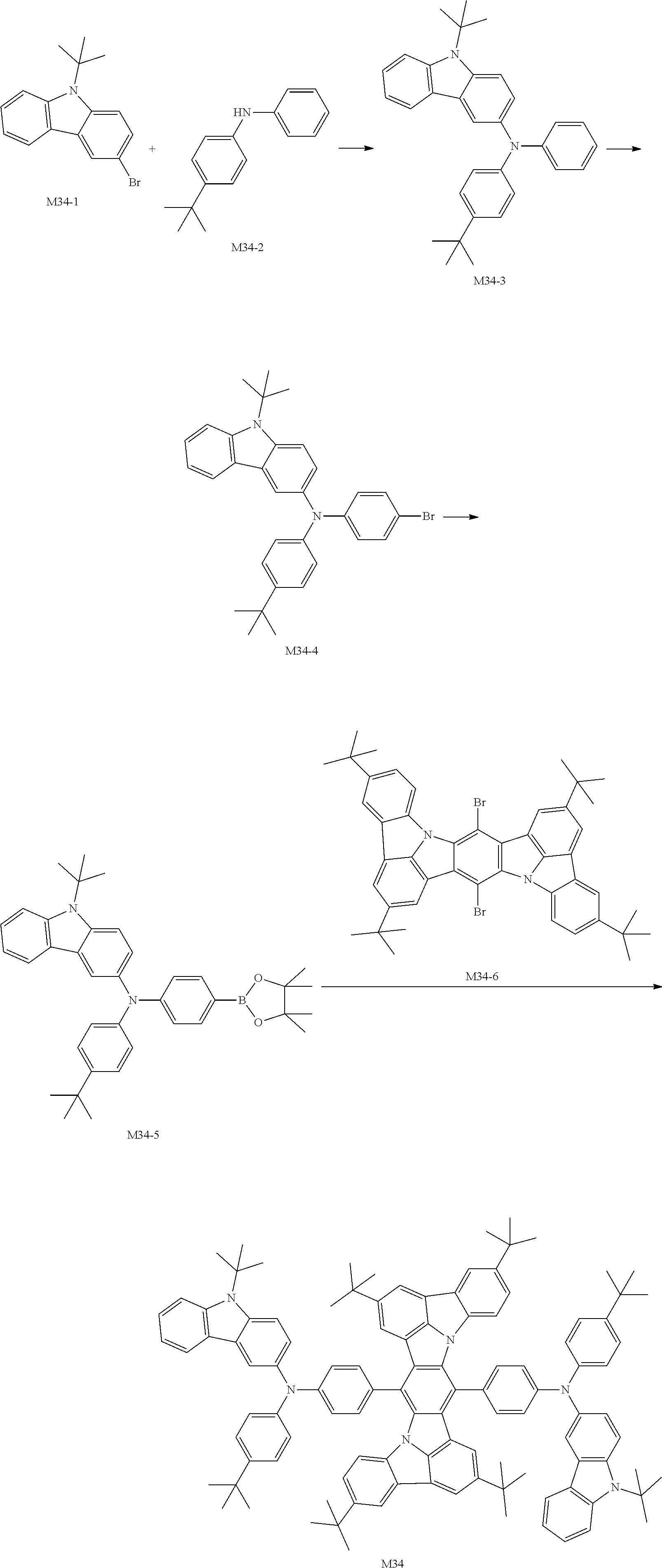

1) Synthesis of intermediate M34-3: in a nitrogen atmosphere, add 30.2 g (100 mmol) of compound M34-1, 22.5 g (100 mmol) of compound M34-2, 19.1 g (100 mmol) of copper iodide, 11.4 g (100 mmol) of trans cyclohexanediamine, 15.2 g (200 mmol) of potassium phosphate, and 250 mL of methylbenzene into a three neck-flask (500 mL). Heat the solution to a temperature of 110° C., and stir it for 12 hours. After the reaction is completed, cool the solution to room temperature. Filter the filtrate by suction, and collect the filtrate after filtering. Rotary evaporate the reaction solution to remove most of the solvent, dissolve it with dichloromethane, and wash it with water for 3 times. Collect the organic solution and stir it with silica gel. Purify the product in column chromatography purification. A yield is 83%.

2) Synthesis of intermediate M34-4: add 26.8 g (60 mmol) of compound M34-3 and 100 mL of trichloromethane into a three neck-flask (500 mL). Then slowly add trichloromethane solution of 60 mmol of N-bromosuccinimide dropwise to the solution, and stir it under an ice bath. After the dropwise addition, let the temperature of the reaction raising to room temperature, and let the reaction continue for 6 hours. After the reaction is completed, add water to quench the reaction. Wash with water for 3 times, and then collect the organic phase. Dry the organic phase by rotary evaporation. Recrystallize the product for purification. A yield is 80%.

3) Synthesis of intermediate M34-5: substitute compound M34-4 for compound M2-2 according to the synthetic method of compound M2-3. A yield is 82%.

4) Synthesis of compound M34: substitute compound M34-6 and twofold molar amounts of compound M34-5 for compound M1-6 and compound M1-7 respectively according to the synthetic method of compound M1. A yield is 70%. MS(ASAP)=1518.

### EXAMPLE 35: SYNTHESIS OF COMPOUND (M35)

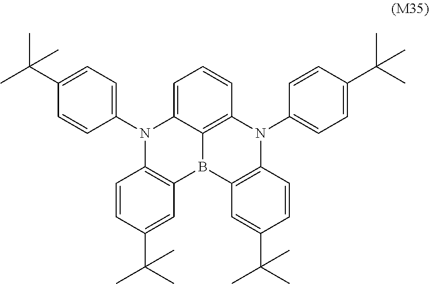 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1]

### Synthetic Route

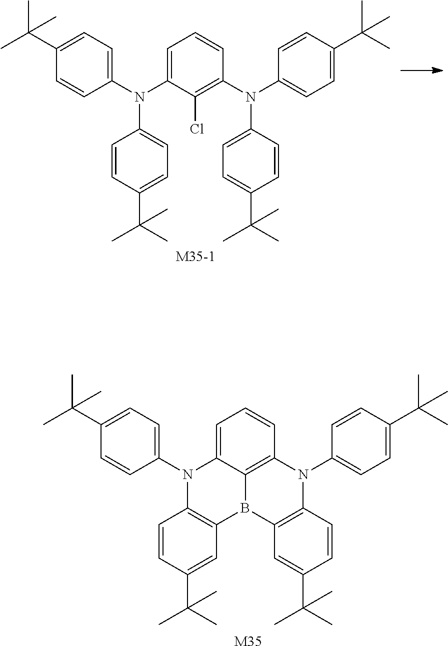

Synthesis of compound M35: in a nitrogen atmosphere, add 33.6 g (50 mmol) of compound M35-1 and 150 mL of anhydrous tetrahydrofuran into a three neck-flask (500 mL). Cool the solution to a temperature of −30° C. Then slowly add 55 mmol of Tert-butyllithium solution dropwise to the solution. After the dropwise addition, let the temperature of the reaction solution raising to a temperature of 60° C., and stir it for 2 hours. Then cool the solution to a temperature of −30° C. Add all 60 mmol of boron tribromide at once. Let the temperature of the reaction raising to room temperature, and let the reaction continue for 1 hours. Then add 100 mmol of N,N-diisopropylethylamine, and slowly let the temperature of the reaction raising to a temperature of 100° C. for 3 hours. After the reaction is completed, cool the solution to room temperature. Then add aqueous sodium acetate solution to quench the reaction. Rotary evaporated the reaction solution to remove most of the solvent. Dissolve with dichloromethane. Wash with water for 3 times. Collect the organic solution, and stir it with silica gel. Purify the product in column chromatography. A yield is 24%. MS(ASAP)=645.

### EXAMPLE 36: SYNTHESIS OF COMPOUND (M36)

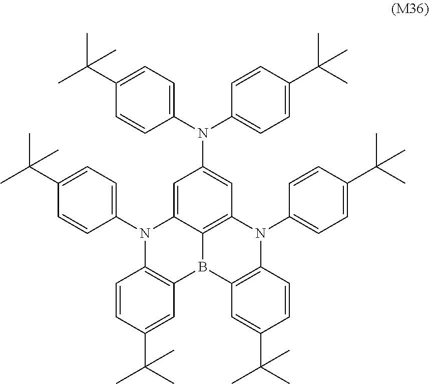 [SMILES: CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1]

### Synthetic Route

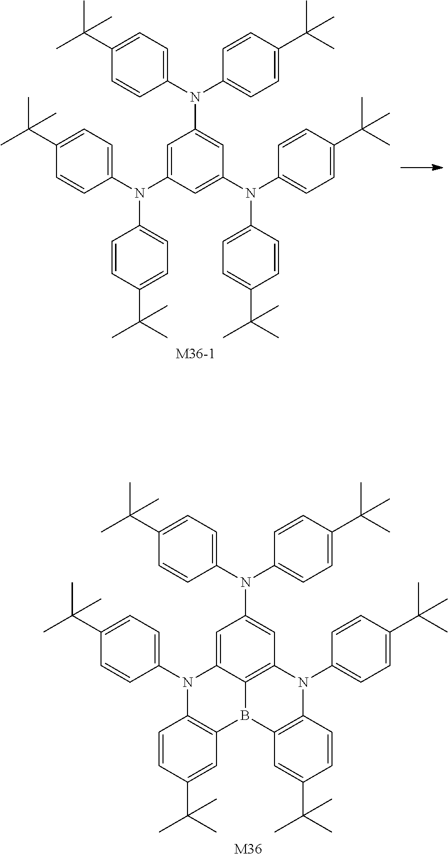

Synthesis of compound M36: in a nitrogen atmosphere, add 45.8 g (50 mmol) of compound M36-1, 50 mmol of boron tribromide, and 150 mL of o-dichlorobenzene into a three neck-flask (500 mL). Heat the solution to a temperature of 170° C., and stir it for 24 hours. After the reaction is completed, cool the solution to room temperature. Then add aqueous sodium acetate solution to quench the reaction. Rotary evaporated the reaction solution to remove most of the solvent. Wash with water for 3 times. Collect the organic solution, and stir it with silica gel. Purify the product in column chromatography. A yield is 53%. MS(ASAP)=924.

### EXAMPLE 37: SYNTHESIS OF COMPOUND (M37)

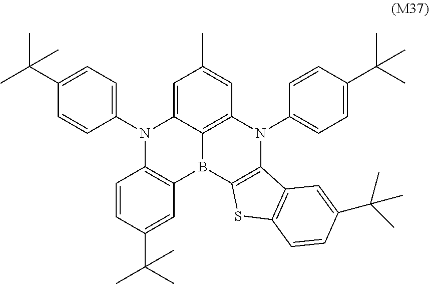 [SMILES: Cc1cc2c3c(c1)N(c1ccc(C(C)(C)C)cc1)c1c(sc4ccc(C(C)(C)C)cc14)B3c1cc(C(C)(C)C)ccc1N2c1ccc(C(C)(C)C)cc1]

### Synthetic Route

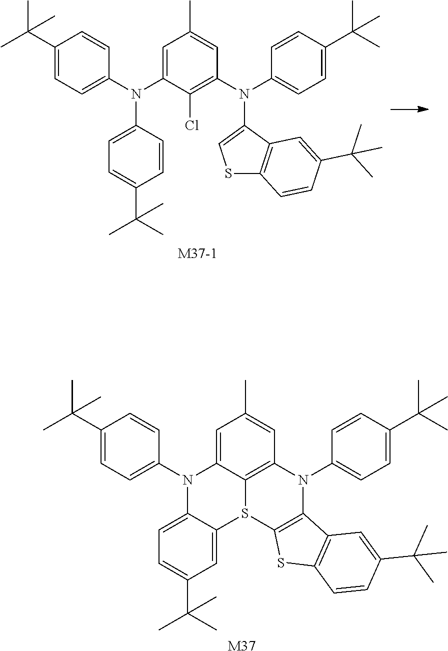

1) Synthesis of compound M37: substitute compound M37-1 for compound M35-1 according to a synthetic method of compound M35. A yield is 25%. MS(ASAP)=715.

### Manufacturing and Characterization of OLED Devices

A structure of the OLED device is as follows: ITO/HIL (a thickness of 80 nm)/HTL (a thickness of 100 nm)/host: 5% of dopant (a thickness of 60 nm)/ETL (a thickness of 25 nm)/LiQ (a thickness of 1 nm)/Al (a thickness of 150 nm)/cathode. Steps of manufacturing the OLED devices include:

1) Cleaning glass substrates with ITO transparent electrodes (anodes): ultrasonically cleaning the glass substrates with aqueous solution of 5% decon90 cleaning solution for 30 mins, ultrasonically cleaning with deionized water for several times, ultrasonically cleaning with isopropanol, and then blowing dry with nitrogen. Exposing the ITO transparent electrodes in oxygen plasma for 5 mins to clean surfaces of the ITO transparent electrodes and to elevate work function of the ITO transparent electrodes.

2) Manufacturing HIL layers and HTL layers: spinning coating PEDOT: PSS (Clevios™ PEDOT: PSS Al4083) on oxygen plasma treated glass substrates to form thin films with a thickness of 80 nm. After the spinning coating is complete, annealing the glass substrates at a temperature of 150° C. in atmosphere for 20 mins Spinning coating poly TBB (CAS: 223569-31-1, purchased from Lumtec. Corp; 5 mg/mL of toluene) with a thickness of 20 nm on PEDOT: PSS layers, and then heating the glass substrates on a hotplate with a temperature of 180° C. for 60 mins.

3) Manufacturing light-emitting layers: first, dissolving a host material (selected from compound M1 to compound M28 and compound Ref-1 to compound Ref-4) and a guest material (selected from compound M29 to compound M37) in xylene at a weight ratio of 95:5 to form a solution with a concentration of 20 mg/mL. Spinning coating the solution on the glass substrates in a nitrogen glovebox to obtain a thin film with a thickness of 60 nm. Annealing the glass substrates at a temperature of 120° C. for 10 mins.

4) Manufacturing electron transport layers, electron injection layers, and cathodes: putting the spin coated devices into a vacuum evaporation chamber to evaporate a ETL material with a thickness of 25 nm, LiQ with a thickness of 1 nm, and Al with a thickness of 150 nm in sequence to complete the manufacturing of the light-emitting devices.

5) Encapsulation: encapsulating all devices in a nitrogen glovebox with UV cured resin and glass covers.

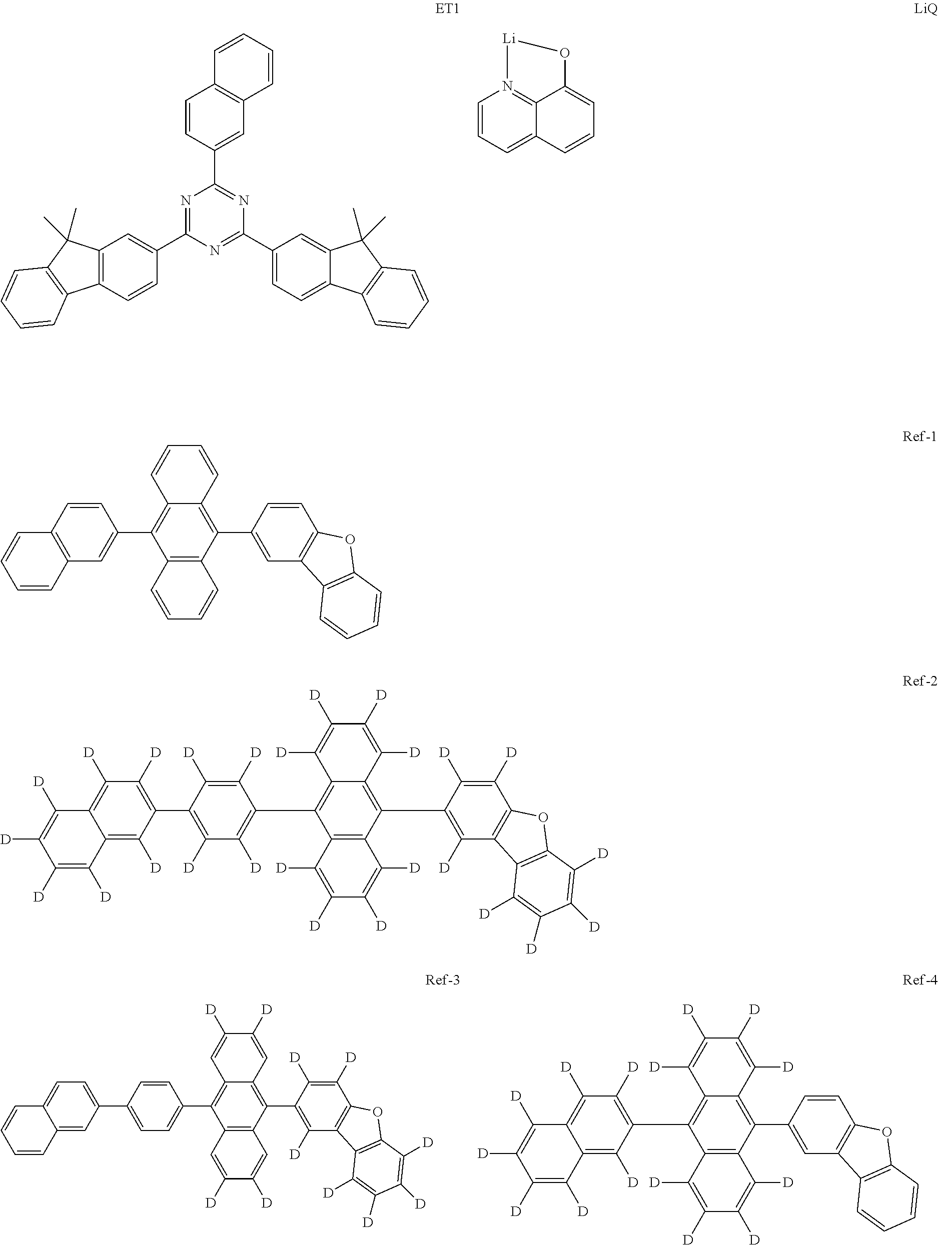

Wherein Refers to patent WO2018164510 for compound Ref-1.

Current-voltage (J-V) characteristics of blue light devices of embodiments A1 to A28 and organic light-emitting diodes of comparative examples 1 to 4 were tested by characterized equipment, and at the same time, external quantum efficiency (EQE) and T90@1000 nits were recorded. The external quantum efficiency and the T90@1000 nits of all examples were divided by the external quantum efficiency and the T90@1000 nits of the comparative example 1 respectively to obtain relative values. The T90@1000 nits indicates a time it takes when a luminance of a display panel from a manual maximum luminance decaying to 95% of the manual maximum luminance, wherein the manual maximum luminance is 1000 nit.

TABLE 1

| | Host | Gust | Relative value | Relative value of |
|---|---|---|---|---|
| OLED device | material | material | of EQE | T90@1000 nits |
| Embodiment A1 | M1 | M29 | 1.60 | 1.68 |
| Embodiment A2 | M2 | M35 | 1.80 | 1.90 |
| Embodiment A3 | M3 | M37 | 1.92 | 2.05 |
| Embodiment A4 | M4 | M31 | 1.56 | 1.64 |
| Embodiment A5 | M5 | M32 | 1.55 | 1.62 |
| Embodiment A6 | M6 | M34 | 1.67 | 1.74 |
| Embodiment A7 | M7 | M36 | 1.89 | 2.00 |
| Embodiment A8 | M8 | M30 | 1.66 | 1.73 |
| Embodiment A9 | M9 | M29 | 1.59 | 1.66 |
| Embodiment A10 | M10 | M37 | 1.81 | 1.88 |
| Embodiment A11 | M11 | M35 | 1.74 | 1.82 |
| Embodiment A12 | M12 | M29 | 1.58 | 1.65 |
| Embodiment A13 | M13 | M29 | 1.65 | 1.71 |
| Embodiment A14 | M14 | M35 | 1.78 | 1.86 |
| Embodiment A15 | M15 | M37 | 1.85 | 1.94 |
| Embodiment A16 | M16 | M36 | 1.73 | 1.80 |
| Embodiment A17 | M17 | M35 | 1.83 | 1.93 |
| Embodiment A18 | M18 | M33 | 1.79 | 1.88 |
| Embodiment A19 | M19 | M29 | 1.63 | 1.70 |
| Embodiment A20 | M20 | M35 | 1.76 | 1.85 |
| Embodiment A21 | M21 | M37 | 1.90 | 2.02 |
| Embodiment A22 | M22 | M36 | 1.88 | 1.98 |
| Embodiment A23 | M23 | M33 | 1.70 | 1.76 |
| Embodiment A24 | M24 | M29 | 1.62 | 1.69 |
| Embodiment A25 | M25 | M35 | 1.75 | 1.84 |
| Embodiment A26 | M26 | M37 | 1.86 | 1.96 |
| Embodiment A27 | M27 | M36 | 1.82 | 1.91 |
| Embodiment A28 | M28 | M33 | 1.68 | 1.75 |
| Comparative | Ref-1 | M29 | 1 | 1 |
| example 1 | | | | |
| Comparative | Ref-2 | M35 | 1.21 | 1.28 |
| example 2 | | | | |
| Comparative | Ref-3 | M37 | 1.17 | 1.24 |
| example 3 | | | | |
| Comparative | Ref-4 | M29 | 1.19 | 1.26 |
| example 4 | | | | |

From table 1, the EQEs of the OLED devices of the embodiments A1 to A28 were divided by the EQEs of the OLED of the comparative example 1 to obtain relative values of the EQEs, wherein a minimum EQE relative value is 1.62, and a maximum EQE relative value is 1.92. While compared with the comparative examples 2 to 4, the maximum EQE relative value is only 1.21. It can be concluded that light-emitting efficiency of the OLED devices can be greatly improved by using the anthracene-based compound and the composition in the present application. And, the naphthalene group with only one hydrogen be deuterated is more effective than the naphthalene group with all hydrogen be deuterated. The T90@1000 nits of the OLED devices of the embodiments A1 to A28 were divided by the T90@1000 nits of the OLED devices of the comparative example 1 to obtain relative values of the T90@1000 nits, wherein a minimum T90@1000 nits relative value is 1.62, and a maximum T90@1000 nits relative value is 2.05. While compared with the comparative examples 2 to 4, whose maximum T90@1000 nits relative values are only 1.28, it can be concluded that lifespan of the OLED devices can be greatly prolonged by using the anthracene-based compound and the composition in the present application.

When Q is represented by formula (A-1), R₁ is -D, R₂ and R₃ are both —H, and one of L₁ and L₂ is a single bond and another one is a phenyl group. Light-emitting efficiency of host materials is greater, and lifespan is longer. When guest materials are represented by formula (III-2), the light-emitting efficiency of the host materials is much greater and the lifespan is much longer. The above conclusions are verified from examples A7, A21 and A3, wherein compound M7 is used as a host material and compound M36 is used as a guest material in example A7, compound M21 is used as the host material and compound M37 is used as the guest material in example A21, and compound M3 is used as the host material and compound M37 is used as the guest material in example A3. The EQE values and the T90@1000 nits values obtained in these three examples are the greatest.

Individual technical features of the above described embodiments may be arbitrarily combined. To keep the description concise, not all possible combinations of the individual technical features in the above examples are described. However, so long as the combinations of these technical features are not contradictory, they should be considered to within a scope of the specification of the present application.

The above embodiments include a few embodiments of the present application. The descriptions of the embodiments are more specific and detailed, but are not to be construed as limitations to the scope of the present application. It should be noted that, without departing from a conception of the present application, several modifications and improvements may be made by those skilled in the art and still fall within the protection scope of the present application. Therefore, the protection scope of the patent application shall be subject to the appended claims.

The above provides a detailed description of the embodiments of the present application. In the present application, specific examples are applied to illustrate a principle and embodiments of the present application. The above description of the embodiments is just used to help understand the present application. At the same time, those skilled in the art, can change the specific implementation and application scope of embodiments according to an idea of the present application. In conclusion, contents of the specification should not be construed as limitations to the present application.

What is claimed is:

1. An anthracene-based compound represented by formula (I): 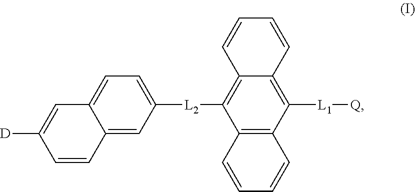
wherein Q is selected from groups represented by formula (A-1) and formula (A-2): 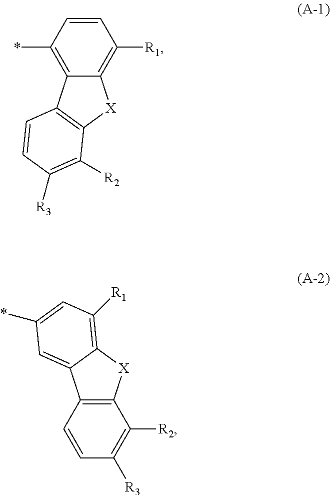
X is O or S;
each of R₁, R₂, and R₃ is independently selected from —H, -D, a C₁-C₆ alkyl group, a phenyl group, a biphenyl group, a phenyl group substituted by a C₁-C₆ alkyl group, and a biphenyl group substituted by a C₁-C₆ alkyl group;
each of L₁ and L₂ is independently selected from a single bond, a phenyl group, a biphenyl group, a phenyl group substituted by a C₁-C₆ alkyl group, and a biphenyl group substituted by a C₁-C₆ alkyl group; and
* indicates a binding site.

2. The anthracene-based compound according to claim 1, wherein R₁, R₂, and R₃ are all H.

3. The anthracene-based compound according to claim 1, wherein one of R₁, R₂, and R₃ is selected from -D, a methyl group, an ethyl group, a n-propyl group, an isopropyl group, a n-butyl group, a tert-butyl group, and a phenyl group, and the other two are both H.

4. The anthracene-based compound according to claim 1, wherein the anthracene-based compound is selected from compounds represented by formula (II-1) and formula (II-2): 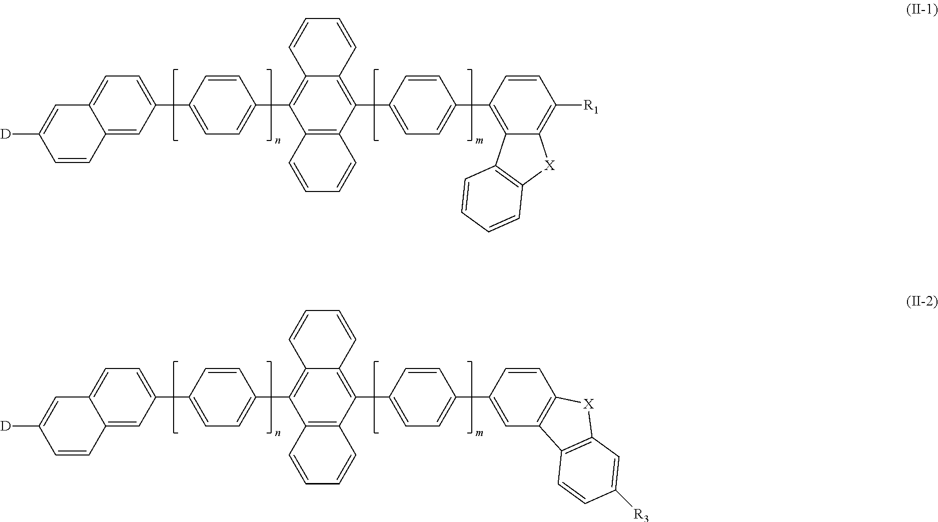
wherein n is selected from 0, 1, and 2, and m is selected from 0, 1, and 2.

5. The anthracene-based compound according to claim 4, wherein R₁ of the formula (II-1) is selected from —H and -D, R₃ of the formula (II-2) is selected from —H and -D, and m+n=1.

6. The anthracene-based compound according to claim 1, wherein Q is selected from groups represented by formula (B-1) to (B-4):

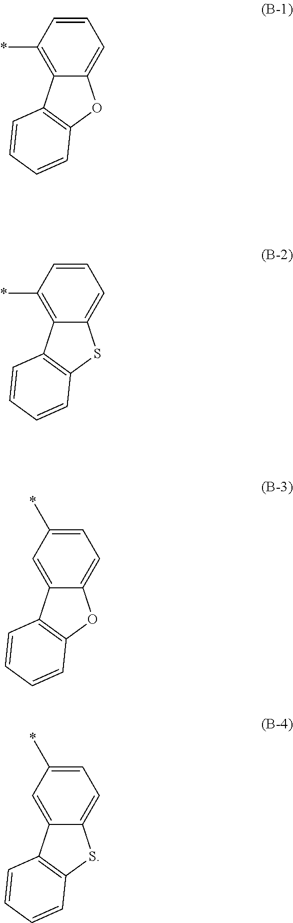 [SMILES: *c1cccc2oc3ccccc3c12 ; *c1cccc2sc3ccccc3c12 ; *c1ccc2oc3ccccc3c2c1 ; *c1ccc2sc3ccccc3c2c1]

7. The anthracene-based compound according to claim 1, wherein Q is selected from groups represented by formula (B-5) to (B-23):

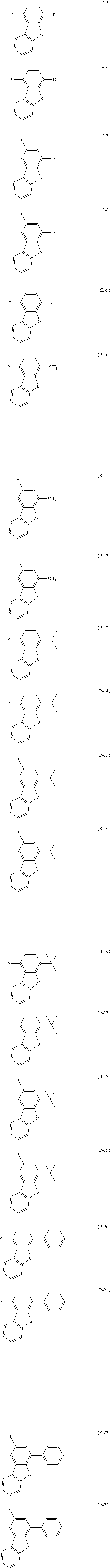

8. The anthracene-based compound according to claim 1, wherein Q is selected from groups represented by formula (B-24) to (B-43):

[chemical structure]

9. The anthracene-based compound according to claim 1, wherein Q is selected from groups represented by formula (B-44) to (B-63):

[chemical structure]

10. The anthracene-based compound as claimed in claim 1, wherein each of L₁ and L₂ is independently selected from groups represented by formula (C-1) to (C-3): 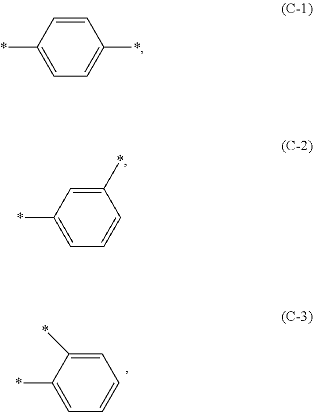
* indicates a binding site.

11. The anthracene-based compound according to claim 1, wherein the anthracene-based compound is selected from following compounds:

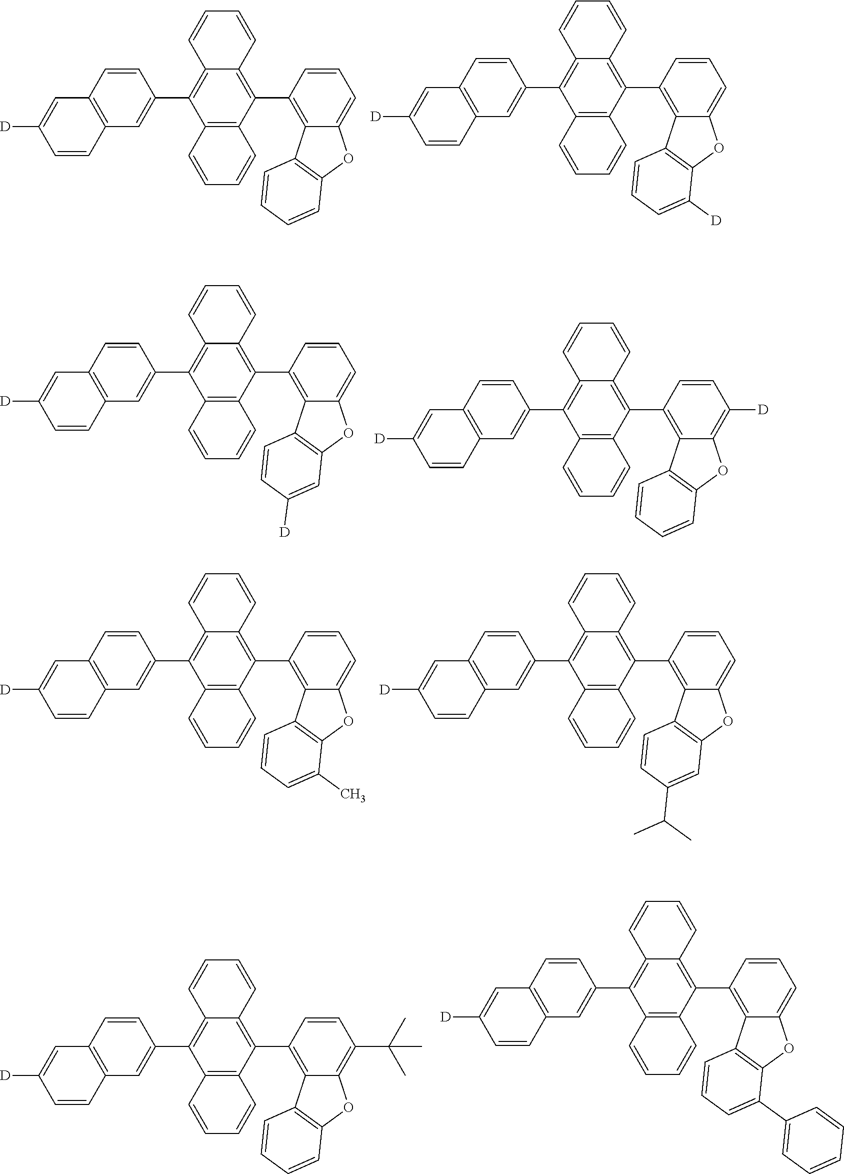

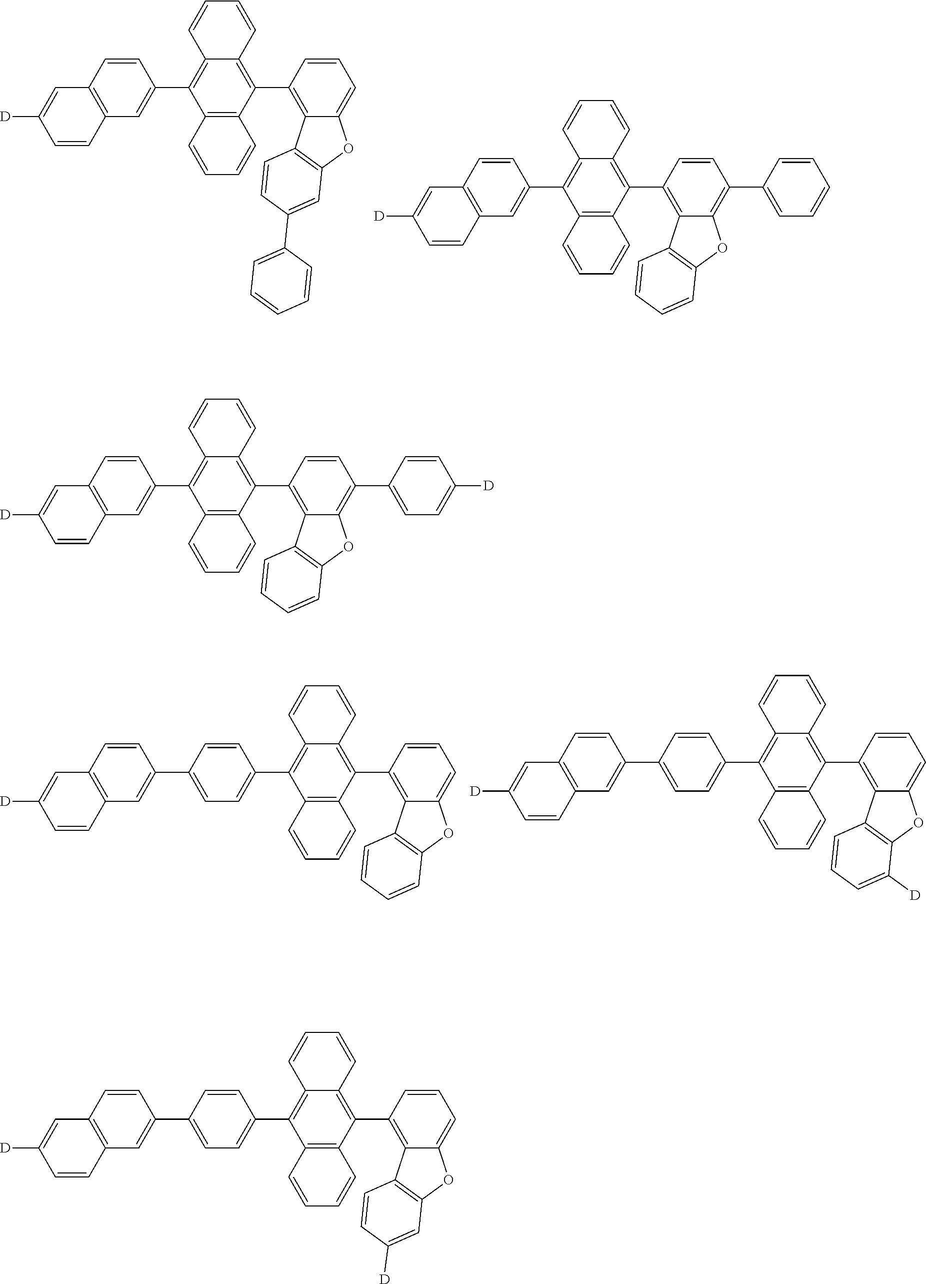

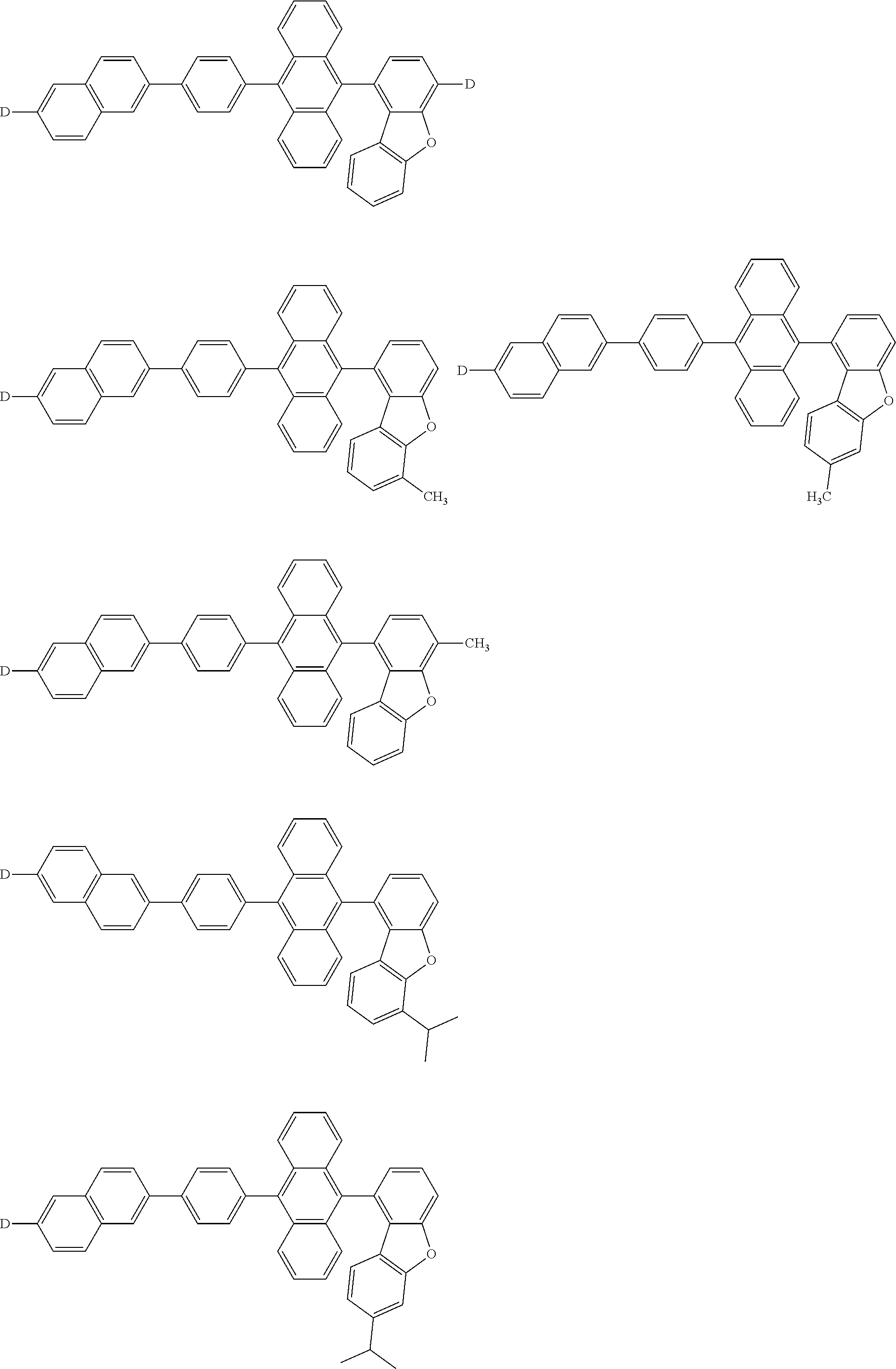

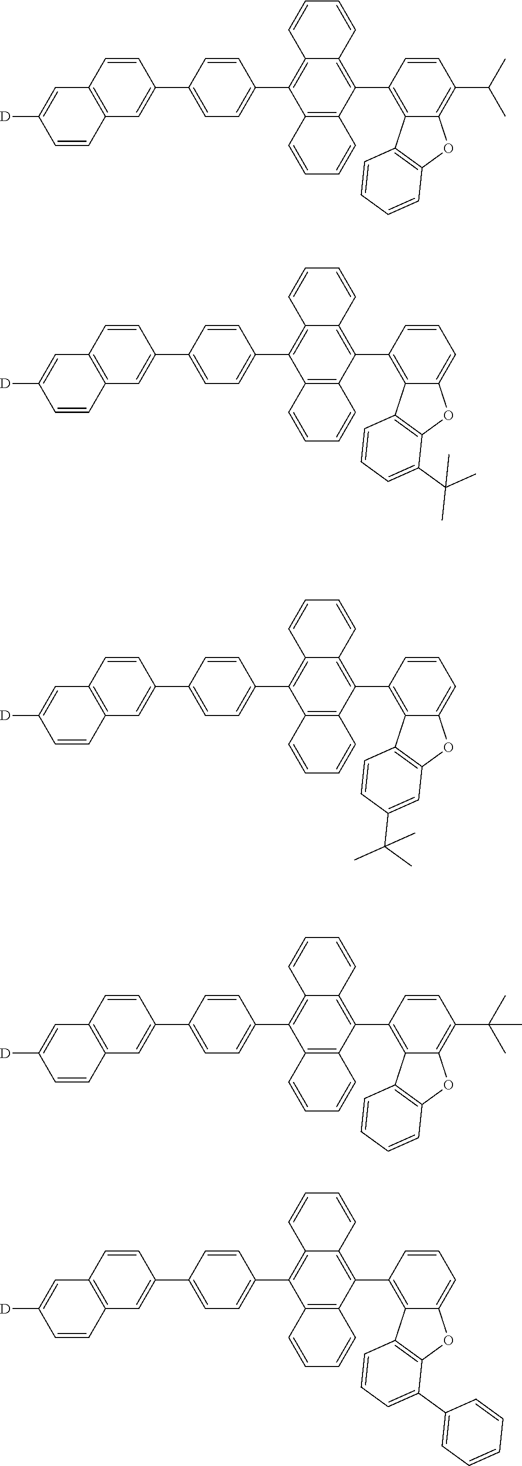 [SMILES: [2H]c1ccc2cc(-c3ccc(-c4c5ccccc5c(-c5ccc(C(C)C)c6oc7ccccc7c56)c5ccccc45)cc3)ccc2c1 ; [2H]c1ccc2cc(-c3ccc(-c4c5ccccc5c(-c5cccc6oc7c(C(C)(C)C)cccc7c56)c5ccccc45)cc3)ccc2c1 ; [2H]c1ccc2cc(-c3ccc(-c4c5ccccc5c(-c5cccc6oc7cc(C(C)(C)C)ccc7c56)c5ccccc45)cc3)ccc2c1 ; [2H]c1ccc2cc(-c3ccc(-c4c5ccccc5c(-c5ccc(C(C)(C)C)c6oc7ccccc7c56)c5ccccc45)cc3)ccc2c1 ; [2H]c1ccc2cc(-c3ccc(-c4c5ccccc5c(-c5cccc6oc7c(-c8ccccc8)cccc7c56)c5ccccc45)cc3)ccc2c1]

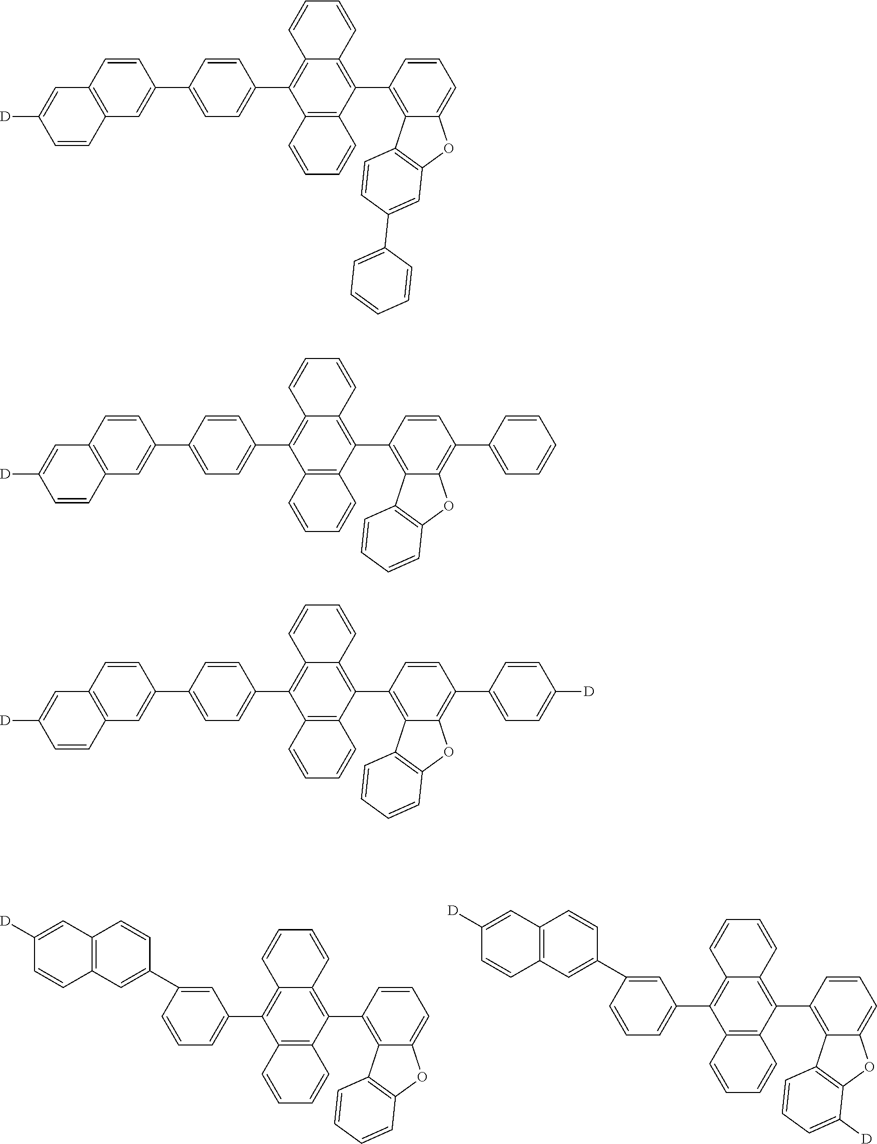 [SMILES: [2H]c1ccc2cc(-c3ccc(-c4c5ccccc5c(-c5cccc6oc7cc(-c8ccccc8)ccc7c56)c5ccccc45)cc3)ccc2c1 ; [2H]c1ccc2cc(-c3ccc(-c4c5ccccc5c(-c5ccc(-c6ccccc6)c6oc7ccccc7c56)c5ccccc45)cc3)ccc2c1 ; [2H]c1ccc(-c2ccc(-c3c4ccccc4c(-c4ccc(-c5ccc6cc([2H])ccc6c5)cc4)c4ccccc34)c3c2oc2ccccc23)cc1 ; [2H]c1ccc2cc(-c3cccc(-c4c5ccccc5c(-c5cccc6oc7ccccc7c56)c5ccccc45)c3)ccc2c1 ; [2H]c1ccc2cc(-c3cccc(-c4c5ccccc5c(-c5cccc6oc7c([2H])cccc7c56)c5ccccc45)c3)ccc2c1]

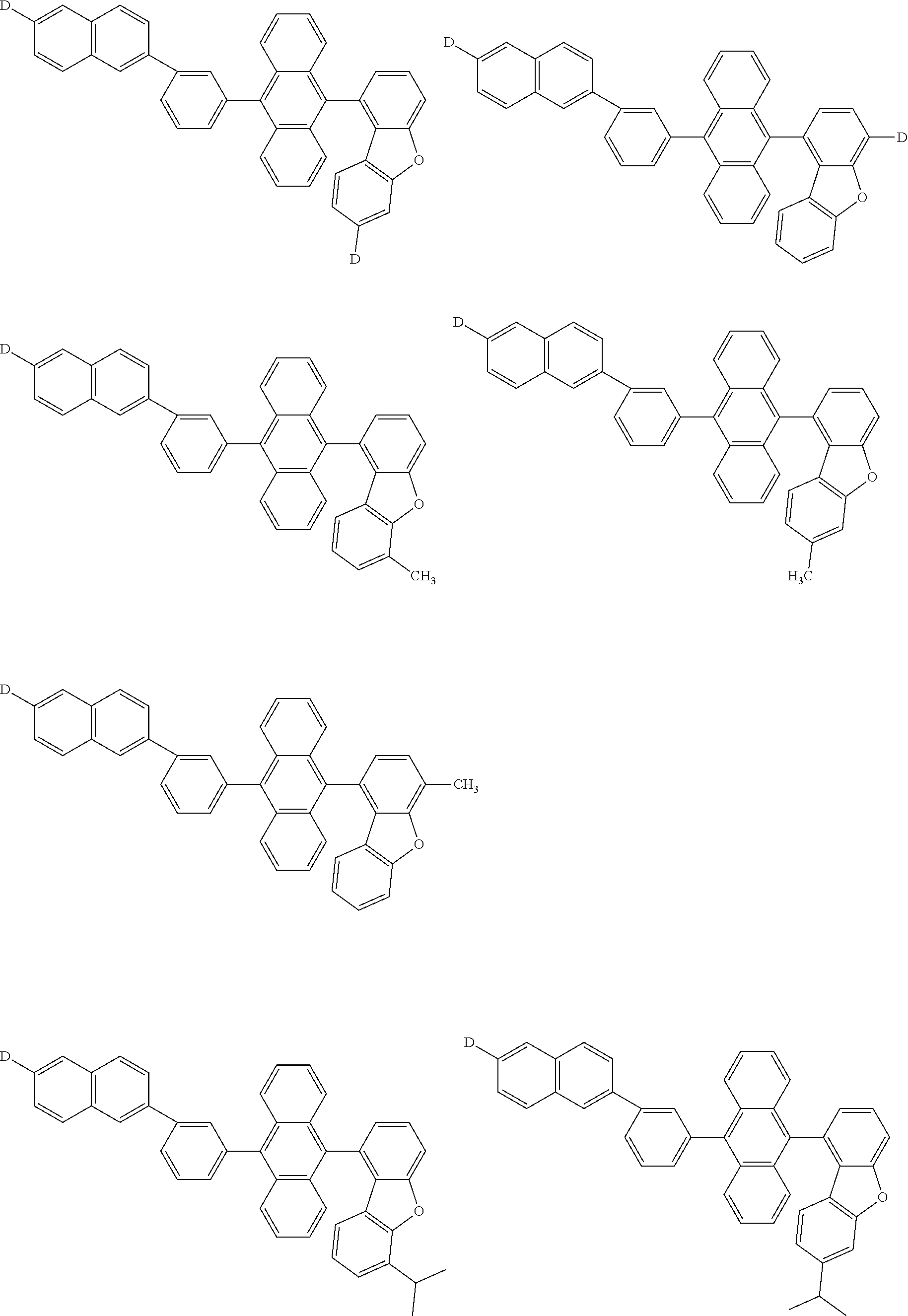

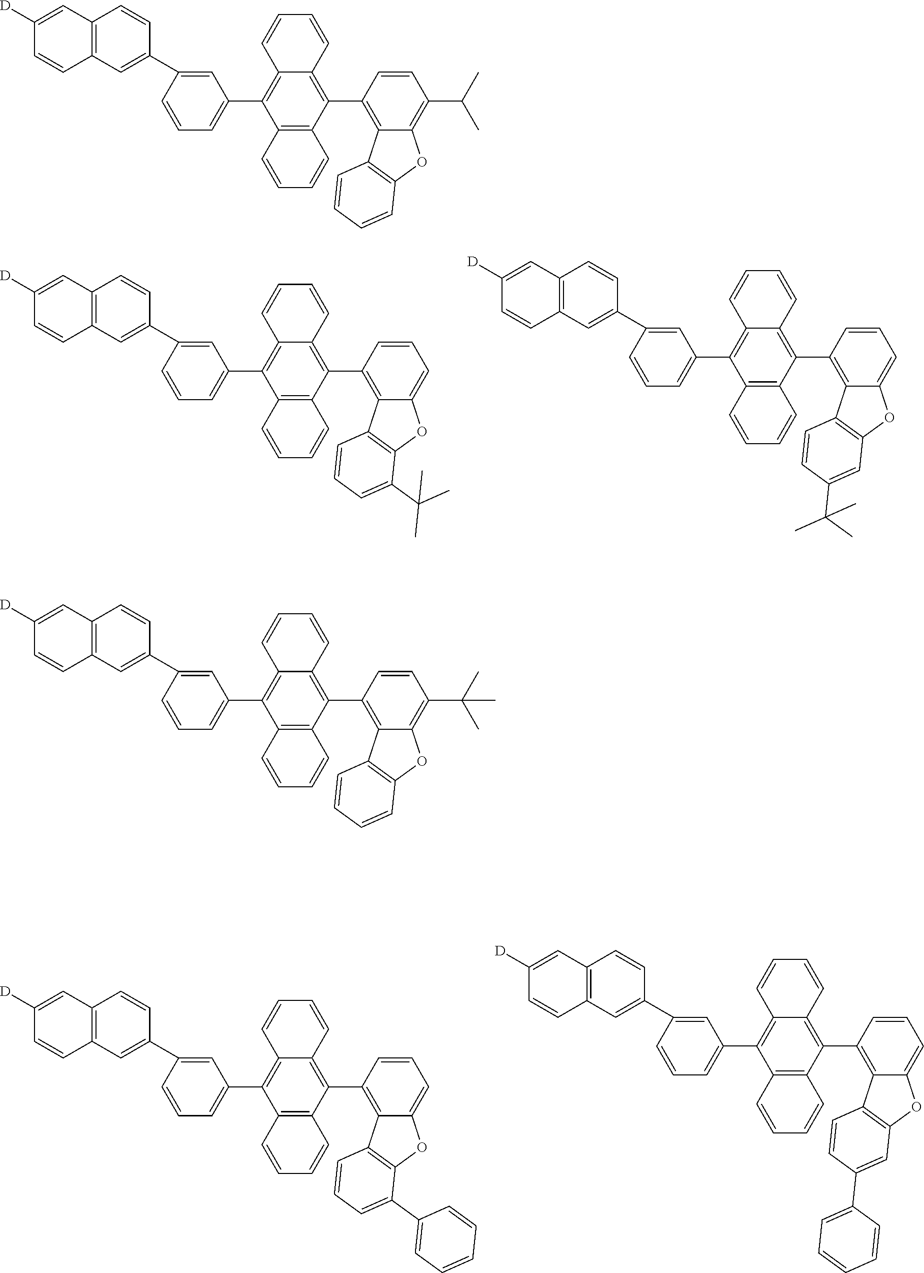

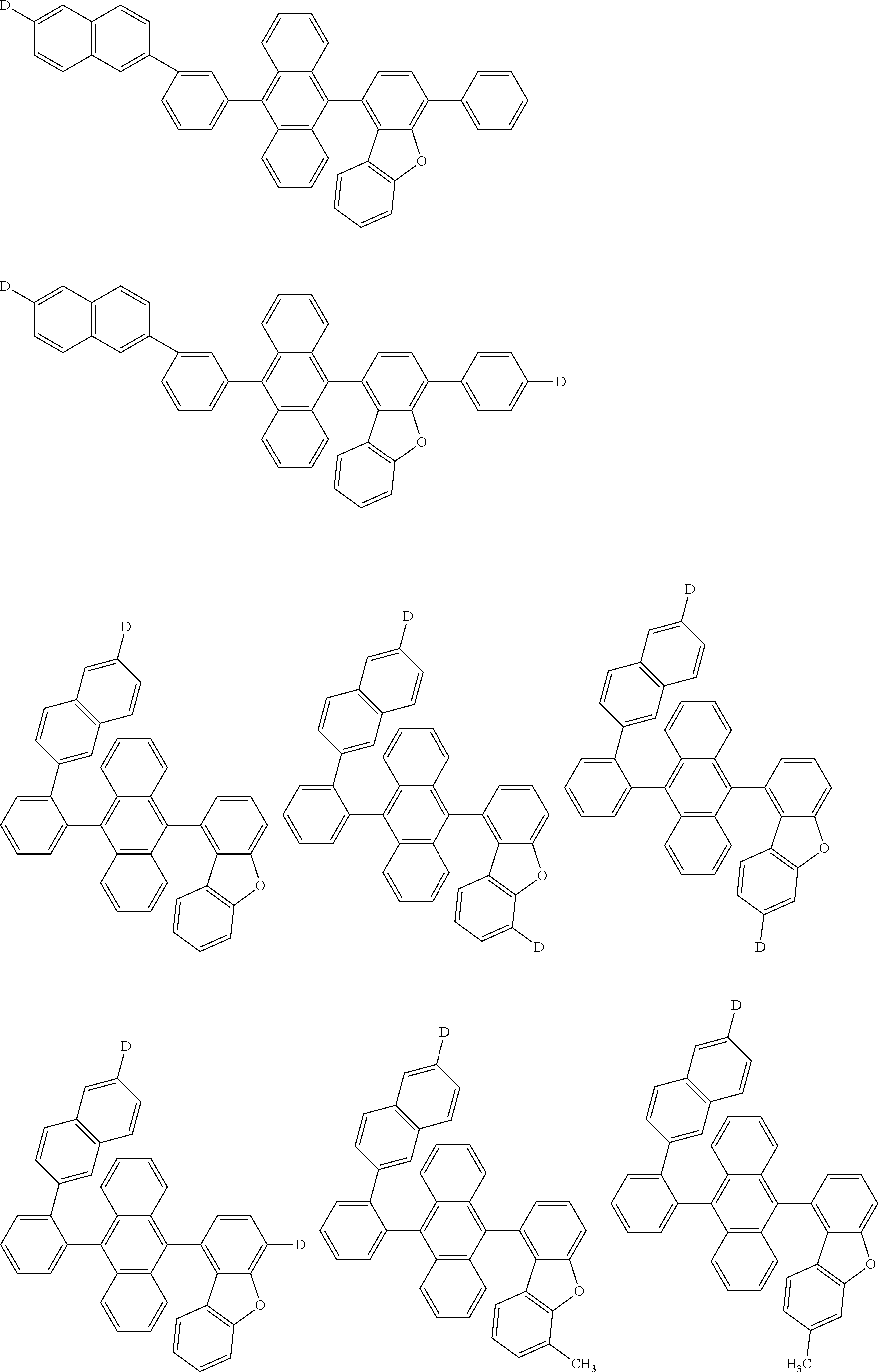

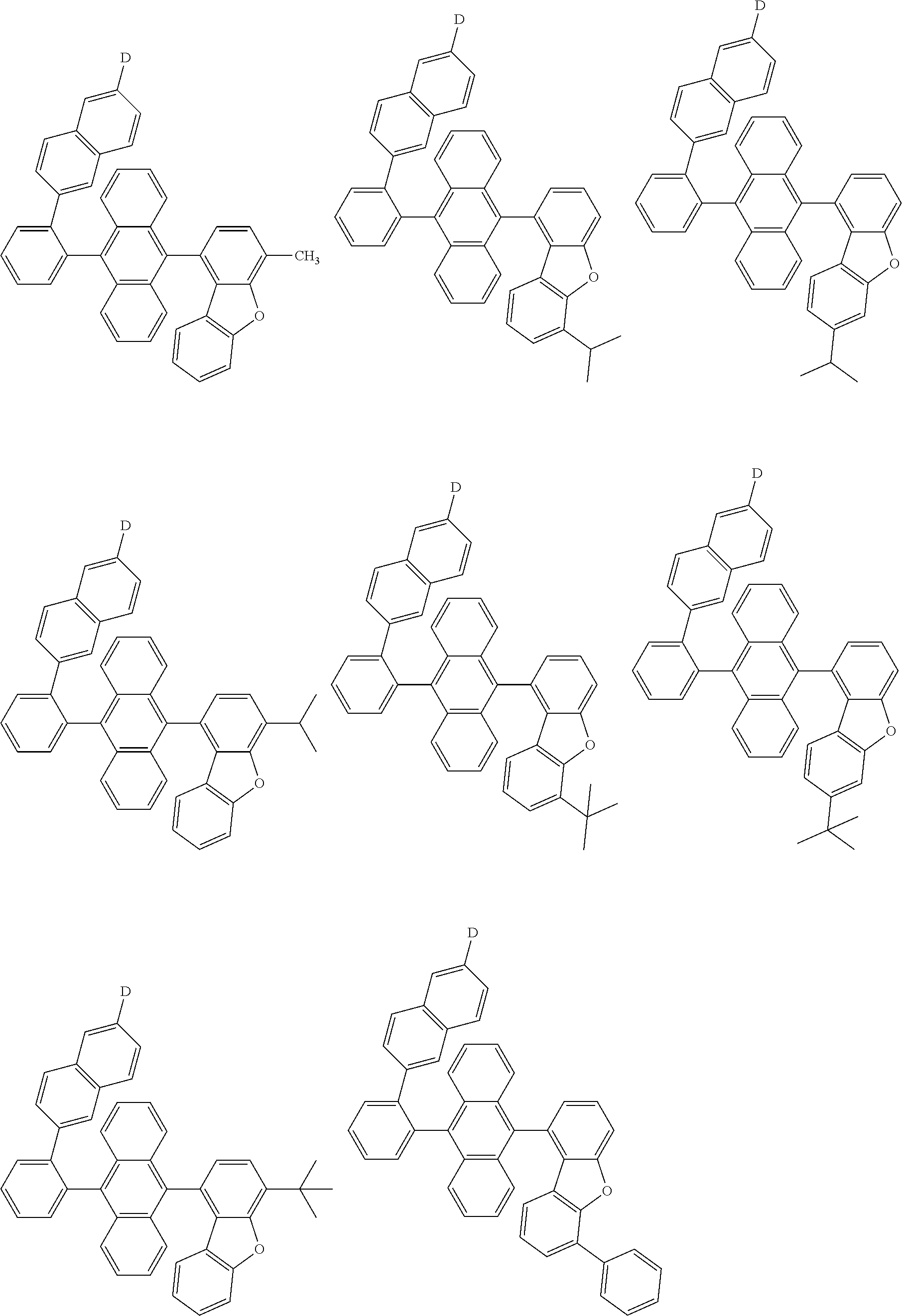

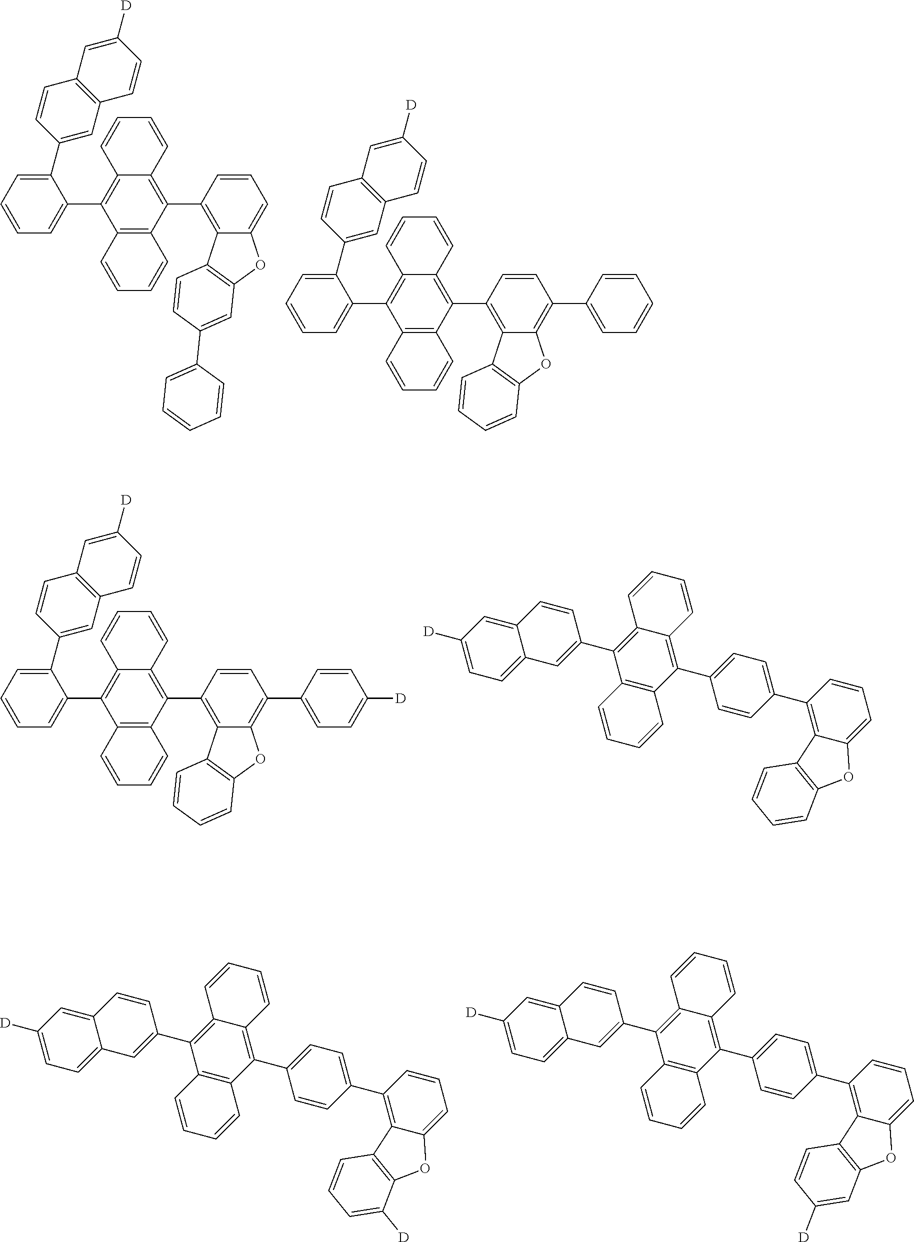

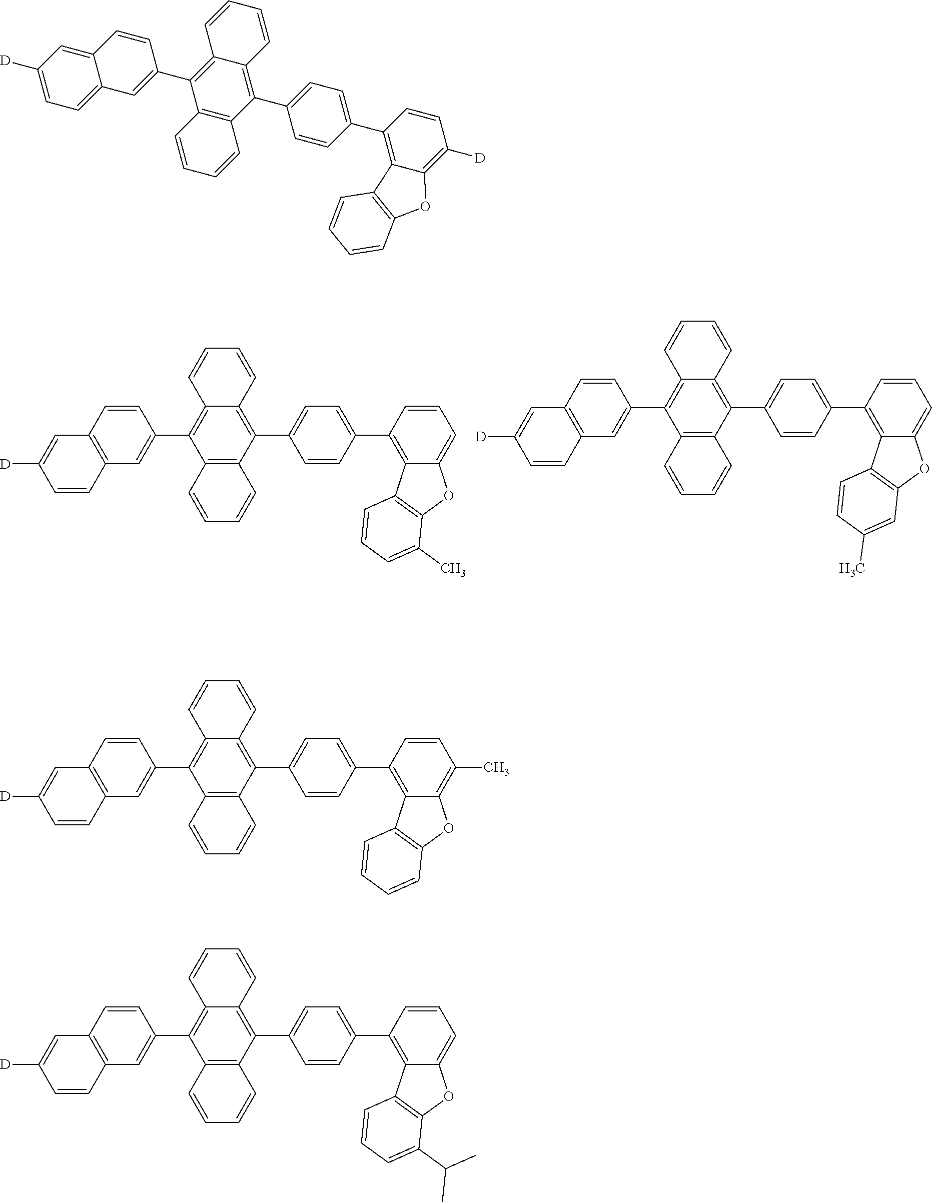

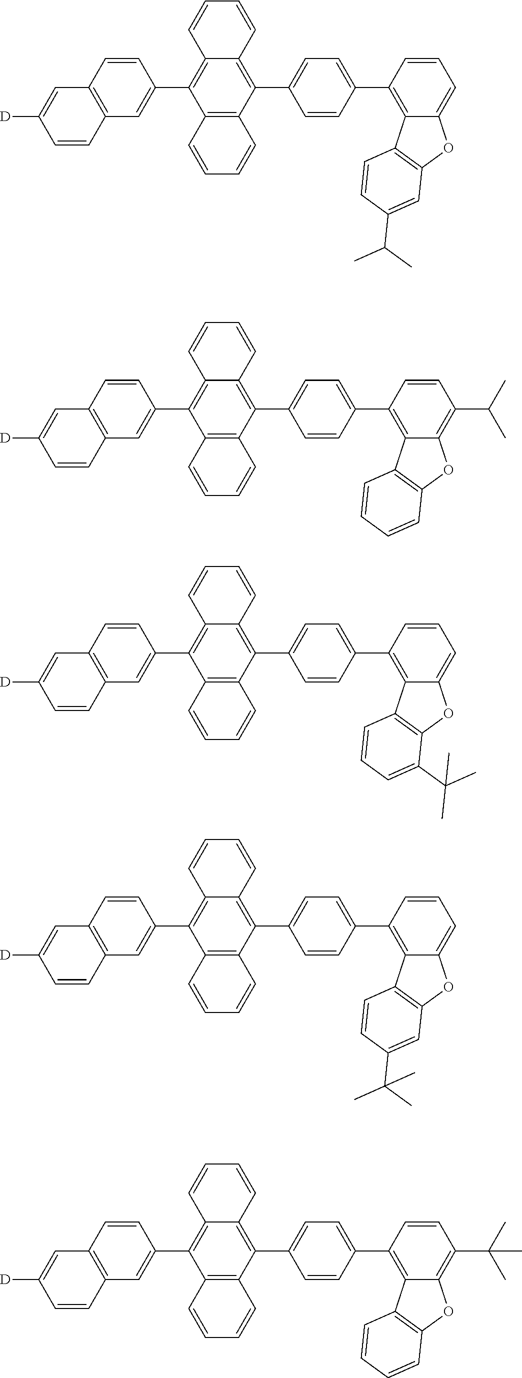 [SMILES: [2H]c1ccc2cc(-c3c4ccccc4c(-c4ccc(-c5cccc6oc7cc(C(C)C)ccc7c56)cc4)c4ccccc34)ccc2c1 ; [2H]c1ccc2cc(-c3c4ccccc4c(-c4ccc(-c5ccc(C(C)C)c6oc7ccccc7c56)cc4)c4ccccc34)ccc2c1 ; [2H]c1ccc2cc(-c3c4ccccc4c(-c4ccc(-c5cccc6oc7c(C(C)(C)C)cccc7c56)cc4)c4ccccc34)ccc2c1 ; [2H]c1ccc2cc(-c3c4ccccc4c(-c4ccc(-c5cccc6oc7cc(C(C)(C)C)ccc7c56)cc4)c4ccccc34)ccc2c1 ; [2H]c1ccc2cc(-c3c4ccccc4c(-c4ccc(-c5ccc(C(C)(C)C)c6oc7ccccc7c56)cc4)c4ccccc34)ccc2c1]

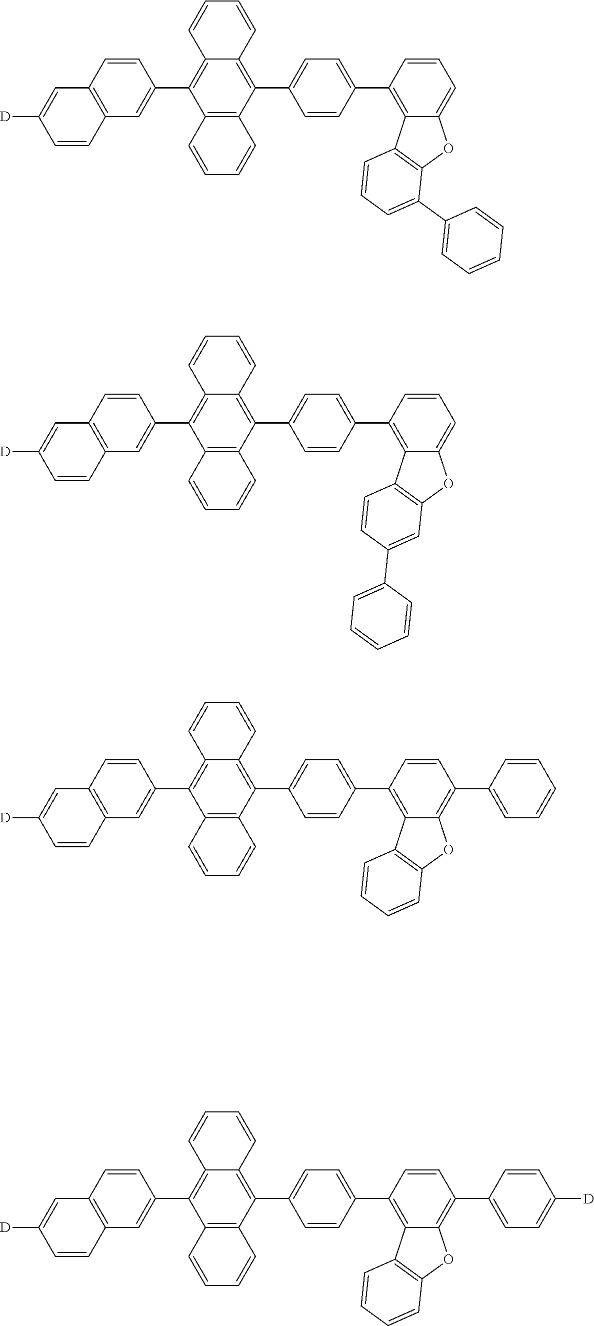 [SMILES: [2H]c1ccc2cc(-c3c4ccccc4c(-c4ccc(-c5cccc6oc7c(-c8ccccc8)cccc7c56)cc4)c4ccccc34)ccc2c1 ; [2H]c1ccc2cc(-c3c4ccccc4c(-c4ccc(-c5cccc6oc7cc(-c8ccccc8)ccc7c56)cc4)c4ccccc34)ccc2c1 ; [2H]c1ccc2cc(-c3c4ccccc4c(-c4ccc(-c5ccc(-c6ccccc6)c6oc7ccccc7c56)cc4)c4ccccc34)ccc2c1 ; [2H]c1ccc(-c2ccc(-c3ccc(-c4c5ccccc5c(-c5ccc6cc([2H])ccc6c5)c5ccccc45)cc3)c3c2oc2ccccc23)cc1]

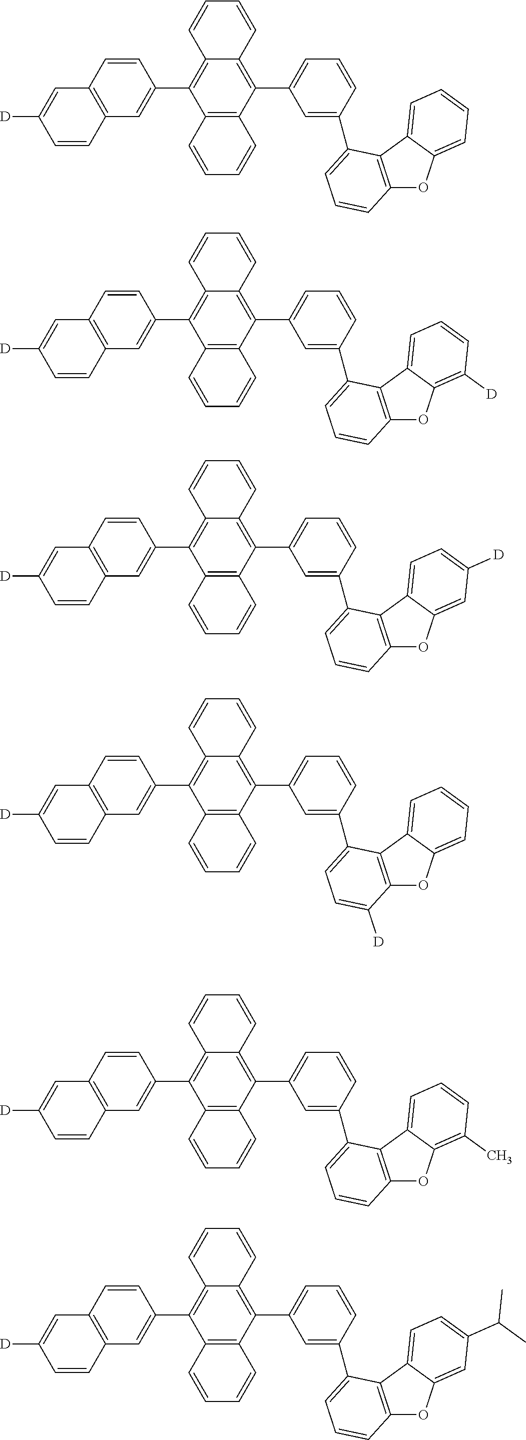 [SMILES: [2H]c1ccc2cc(-c3c4ccccc4c(-c4cccc(-c5cccc6oc7ccccc7c56)c4)c4ccccc34)ccc2c1 ; [2H]c1ccc2cc(-c3c4ccccc4c(-c4cccc(-c5cccc6oc7c([2H])cccc7c56)c4)c4ccccc34)ccc2c1 ; [2H]c1ccc2cc(-c3c4ccccc4c(-c4cccc(-c5cccc6oc7cc([2H])ccc7c56)c4)c4ccccc34)ccc2c1 ; [2H]c1ccc2cc(-c3c4ccccc4c(-c4cccc(-c5ccc([2H])c6oc7ccccc7c56)c4)c4ccccc34)ccc2c1 ; [2H]c1ccc2cc(-c3c4ccccc4c(-c4cccc(-c5cccc6oc7c(C)cccc7c56)c4)c4ccccc34)ccc2c1 ; [2H]c1ccc2cc(-c3c4ccccc4c(-c4cccc(-c5cccc6oc7cc(C(C)C)ccc7c56)c4)c4ccccc34)ccc2c1]

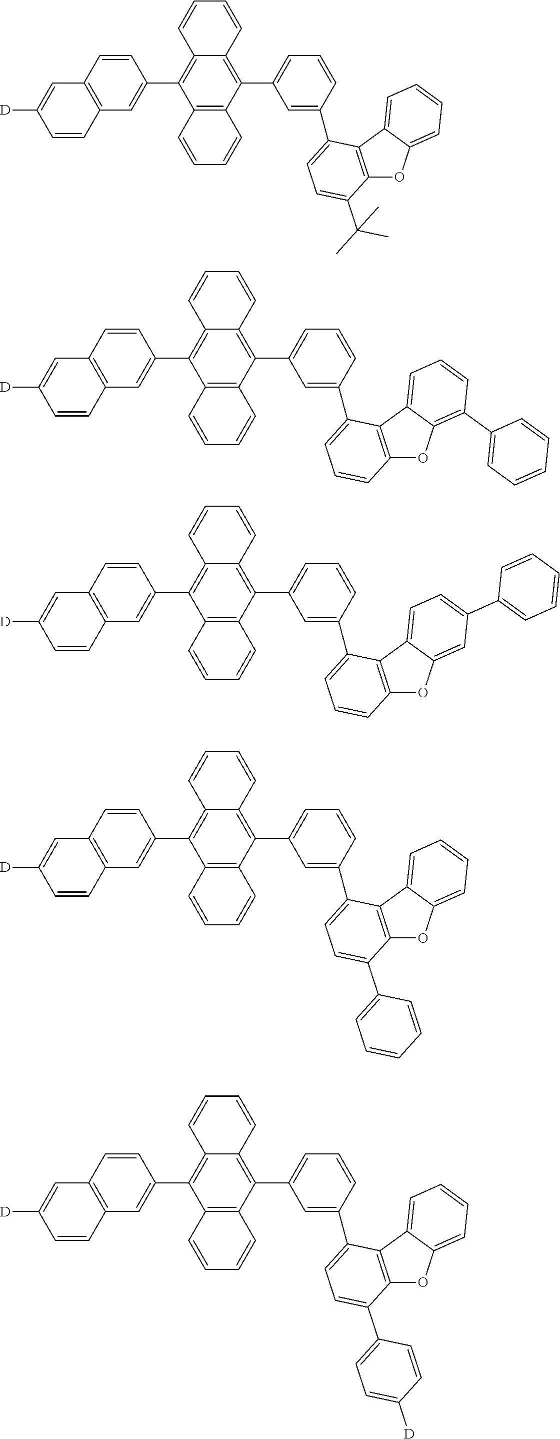 [SMILES: [2H]c1ccc2cc(-c3c4ccccc4c(-c4cccc(-c5ccc(C(C)(C)C)c6oc7ccccc7c56)c4)c4ccccc34)ccc2c1 ; [2H]c1ccc2cc(-c3c4ccccc4c(-c4cccc(-c5cccc6oc7c(-c8ccccc8)cccc7c56)c4)c4ccccc34)ccc2c1 ; [2H]c1ccc2cc(-c3c4ccccc4c(-c4cccc(-c5cccc6oc7cc(-c8ccccc8)ccc7c56)c4)c4ccccc34)ccc2c1 ; [2H]c1ccc2cc(-c3c4ccccc4c(-c4cccc(-c5ccc(-c6ccccc6)c6oc7ccccc7c56)c4)c4ccccc34)ccc2c1 ; [2H]c1ccc(-c2ccc(-c3cccc(-c4c5ccccc5c(-c5ccc6cc([2H])ccc6c5)c5ccccc45)c3)c3c2oc2ccccc23)cc1]

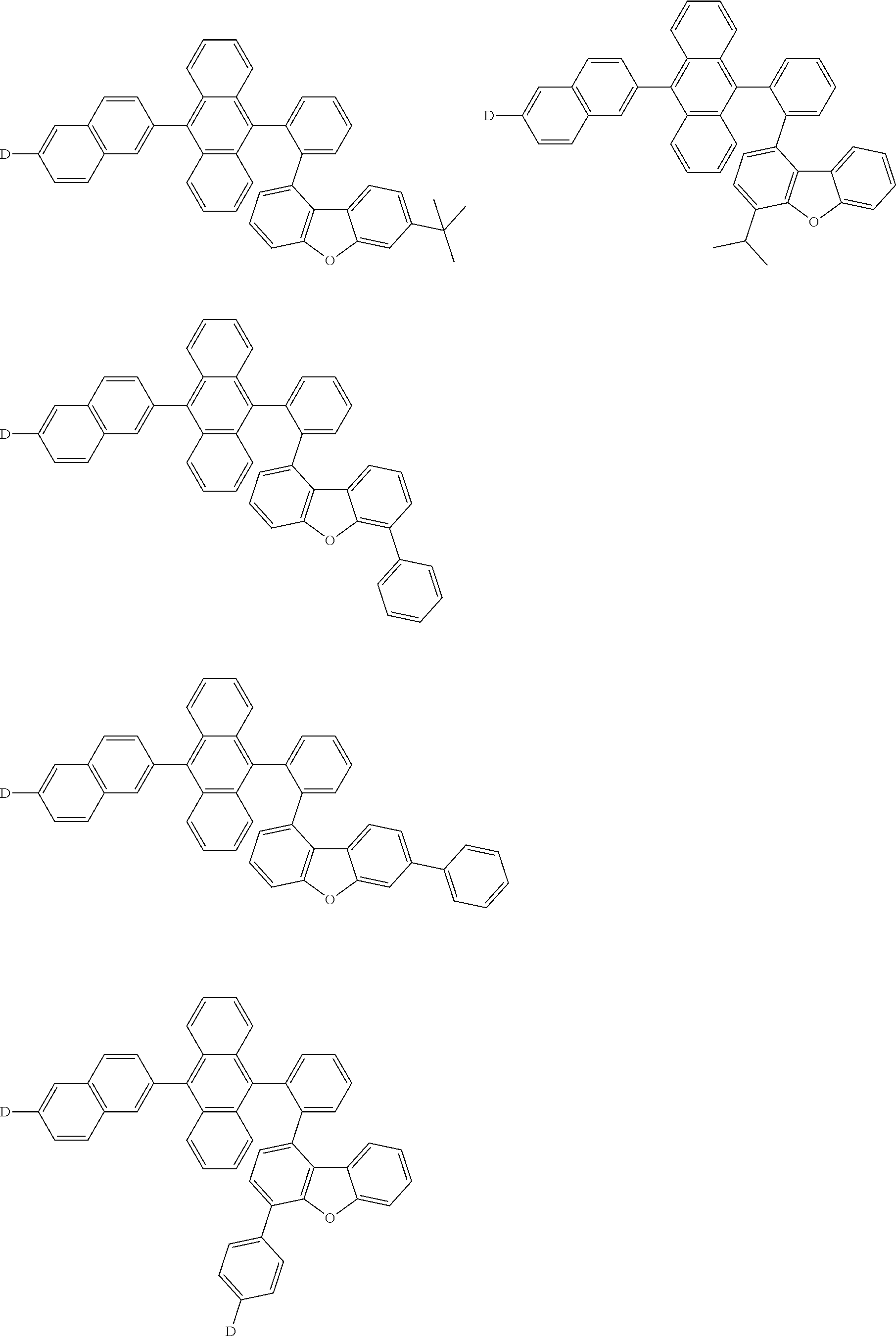 [SMILES: [2H]c1ccc2cc(-c3c4ccccc4c(-c4ccccc4-c4cccc5oc6cc(C(C)(C)C)ccc6c45)c4ccccc34)ccc2c1 ; [2H]c1ccc2cc(-c3c4ccccc4c(-c4ccccc4-c4ccc(C(C)C)c5oc6ccccc6c45)c4ccccc34)ccc2c1 ; [2H]c1ccc2cc(-c3c4ccccc4c(-c4ccccc4-c4cccc5oc6c(-c7ccccc7)cccc6c45)c4ccccc34)ccc2c1 ; [2H]c1ccc2cc(-c3c4ccccc4c(-c4ccccc4-c4cccc5oc6cc(-c7ccccc7)ccc6c45)c4ccccc34)ccc2c1 ; [2H]c1ccc(-c2ccc(-c3ccccc3-c3c4ccccc4c(-c4ccc5cc([2H])ccc5c4)c4ccccc34)c3c2oc2ccccc23)cc1]

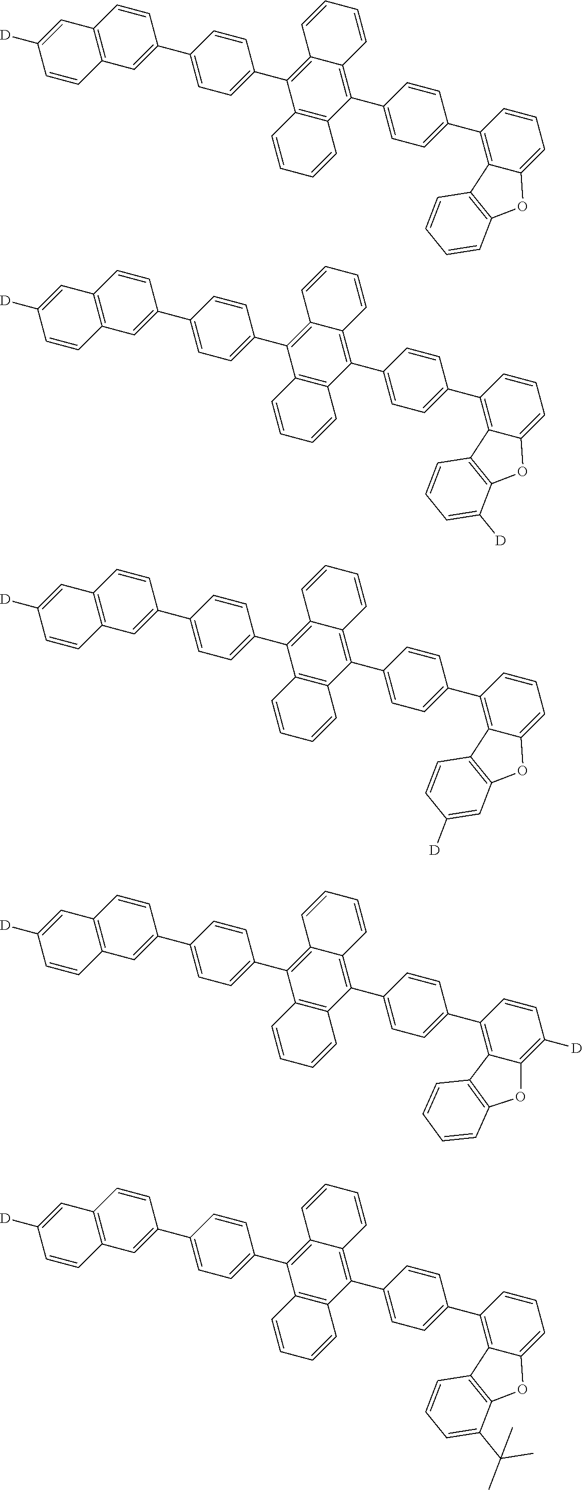 [SMILES: [2H]c1ccc2cc(-c3ccc(-c4c5ccccc5c(-c5ccc(-c6cccc7oc8ccccc8c67)cc5)c5ccccc45)cc3)ccc2c1 ; [2H]c1ccc2cc(-c3ccc(-c4c5ccccc5c(-c5ccc(-c6cccc7oc8c([2H])cccc8c67)cc5)c5ccccc45)cc3)ccc2c1 ; [2H]c1ccc2cc(-c3ccc(-c4c5ccccc5c(-c5ccc(-c6cccc7oc8cc([2H])ccc8c67)cc5)c5ccccc45)cc3)ccc2c1 ; [2H]c1ccc2cc(-c3ccc(-c4c5ccccc5c(-c5ccc(-c6ccc([2H])c7oc8ccccc8c67)cc5)c5ccccc45)cc3)ccc2c1 ; [2H]c1ccc2cc(-c3ccc(-c4c5ccccc5c(-c5ccc(-c6cccc7oc8c(C(C)(C)C)cccc8c67)cc5)c5ccccc45)cc3)ccc2c1]

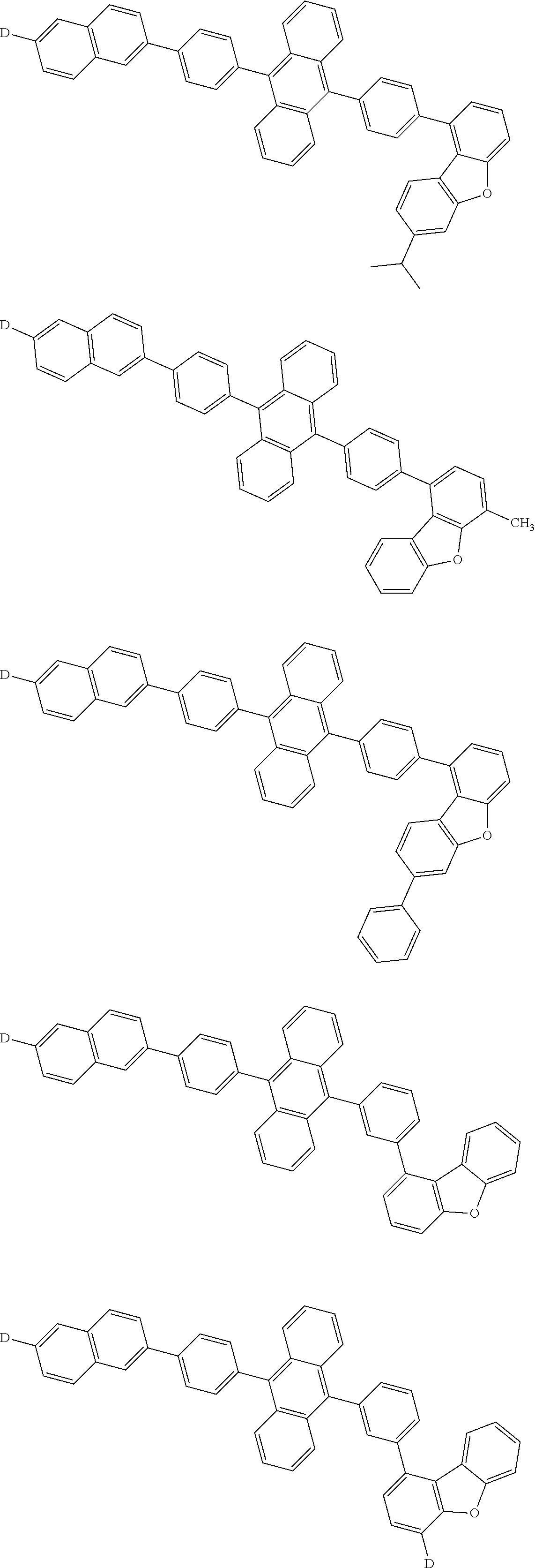 [SMILES: [2H]c1ccc2cc(-c3ccc(-c4c5ccccc5c(-c5ccc(-c6cccc7oc8cc(C(C)C)ccc8c67)cc5)c5ccccc45)cc3)ccc2c1 ; [2H]c1ccc2cc(-c3ccc(-c4c5ccccc5c(-c5ccc(-c6ccc(C)c7oc8ccccc8c67)cc5)c5ccccc45)cc3)ccc2c1 ; [2H]c1ccc2cc(-c3ccc(-c4c5ccccc5c(-c5ccc(-c6cccc7oc8cc(-c9ccccc9)ccc8c67)cc5)c5ccccc45)cc3)ccc2c1 ; [2H]c1ccc2cc(-c3ccc(-c4c5ccccc5c(-c5cccc(-c6cccc7oc8ccccc8c67)c5)c5ccccc45)cc3)ccc2c1 ; [2H]c1ccc2cc(-c3ccc(-c4c5ccccc5c(-c5cccc(-c6ccc([2H])c7oc8ccccc8c67)c5)c5ccccc45)cc3)ccc2c1]

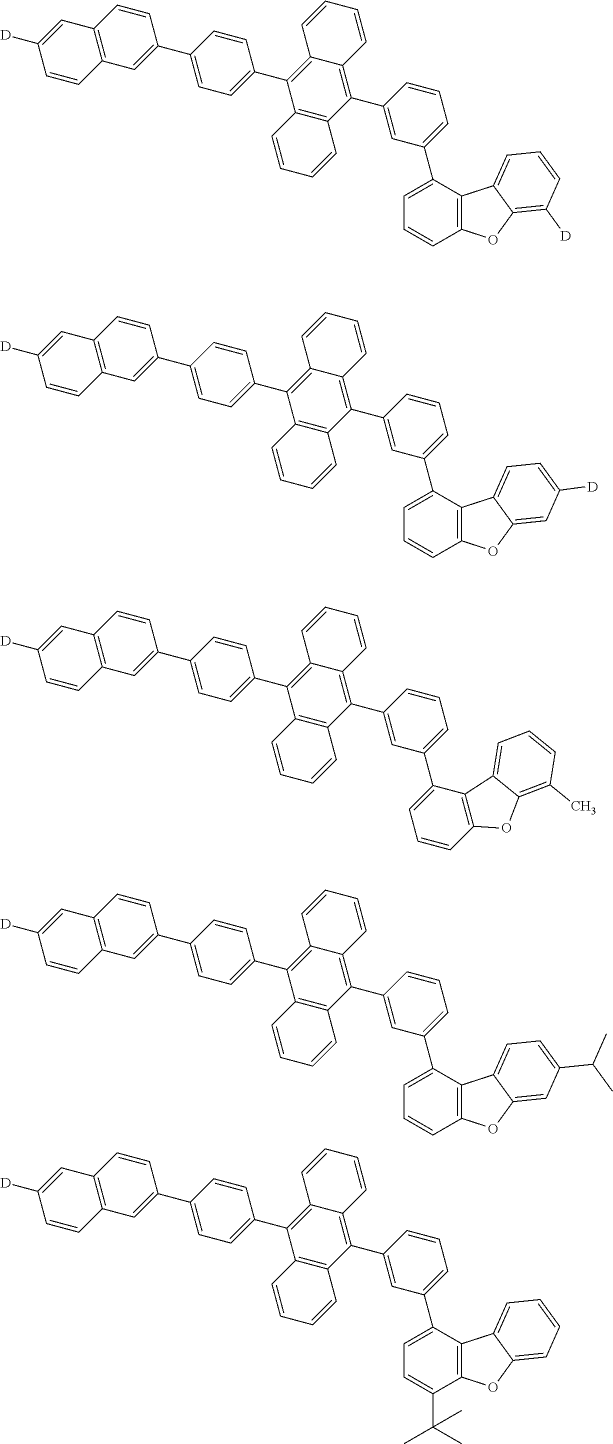 [SMILES: [2H]c1ccc2cc(-c3ccc(-c4c5ccccc5c(-c5cccc(-c6cccc7oc8c([2H])cccc8c67)c5)c5ccccc45)cc3)ccc2c1 ; [2H]c1ccc2cc(-c3ccc(-c4c5ccccc5c(-c5cccc(-c6cccc7oc8cc([2H])ccc8c67)c5)c5ccccc45)cc3)ccc2c1 ; [2H]c1ccc2cc(-c3ccc(-c4c5ccccc5c(-c5cccc(-c6cccc7oc8c(C)cccc8c67)c5)c5ccccc45)cc3)ccc2c1 ; [2H]c1ccc2cc(-c3ccc(-c4c5ccccc5c(-c5cccc(-c6cccc7oc8cc(C(C)C)ccc8c67)c5)c5ccccc45)cc3)ccc2c1 ; [2H]c1ccc2cc(-c3ccc(-c4c5ccccc5c(-c5cccc(-c6ccc(C(C)(C)C)c7oc8ccccc8c67)c5)c5ccccc45)cc3)ccc2c1]

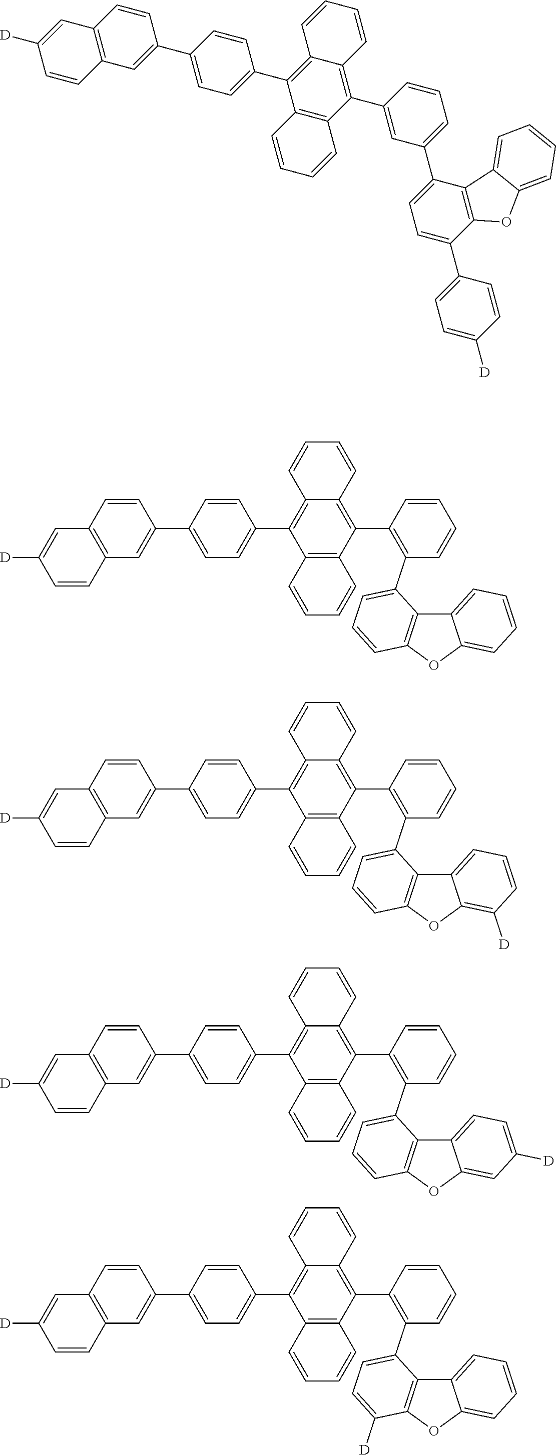

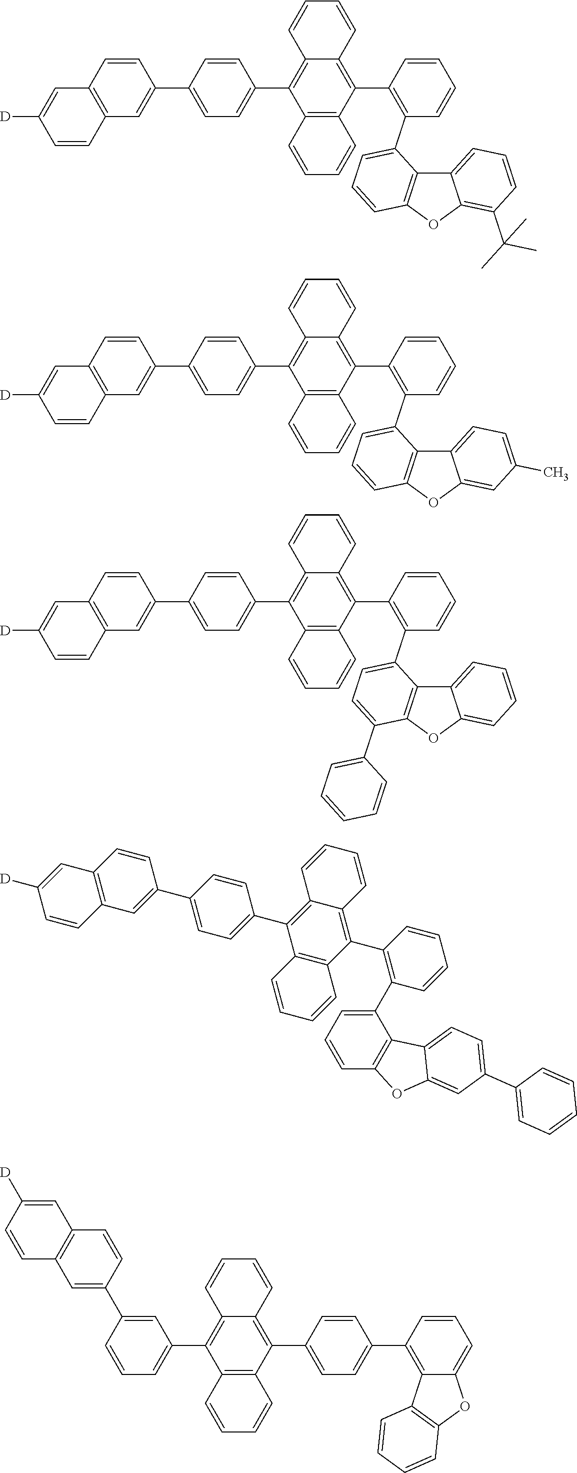

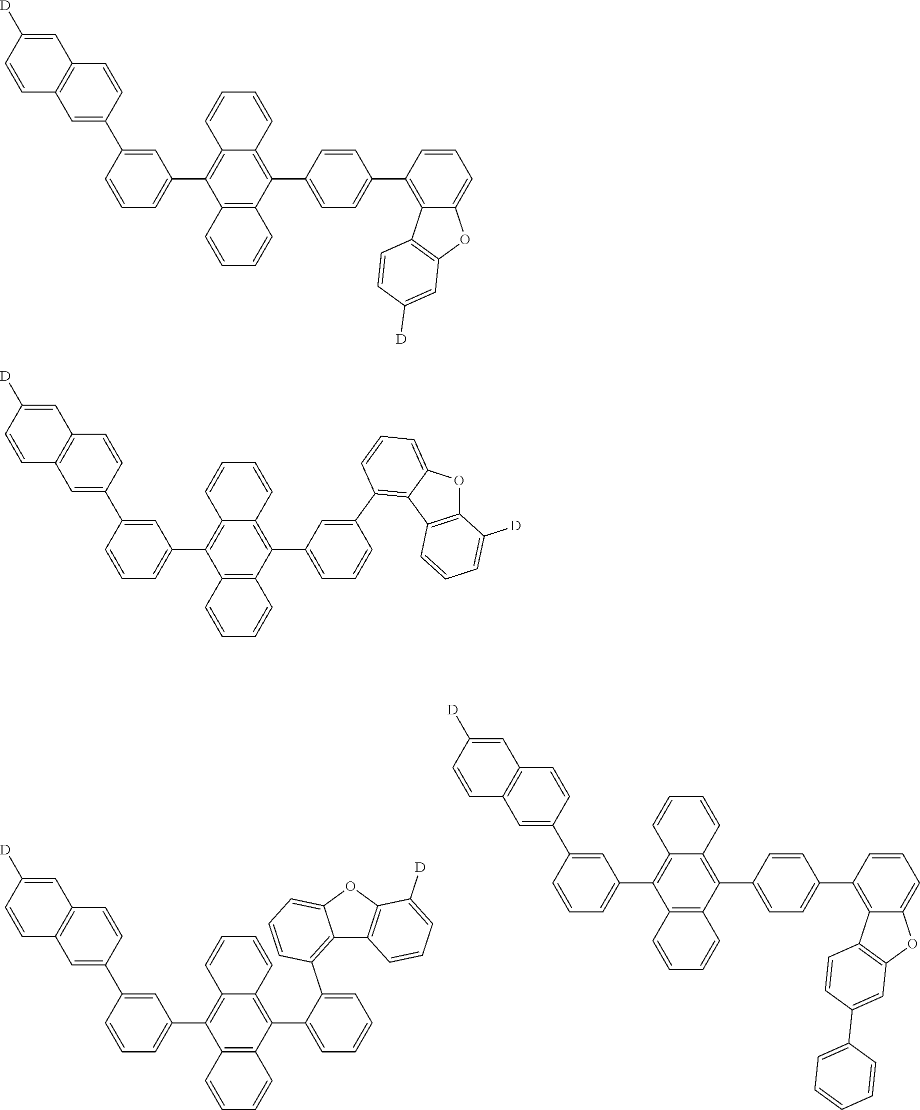 [SMILES: [2H]c1ccc2cc(-c3cccc(-c4c5ccccc5c(-c5ccc(-c6cccc7oc8cc([2H])ccc8c67)cc5)c5ccccc45)c3)ccc2c1 ; [2H]c1ccc2cc(-c3cccc(-c4c5ccccc5c(-c5cccc(-c6cccc7oc8c([2H])cccc8c67)c5)c5ccccc45)c3)ccc2c1 ; [2H]c1ccc2cc(-c3cccc(-c4c5ccccc5c(-c5ccccc5-c5cccc6oc7c([2H])cccc7c56)c5ccccc45)c3)ccc2c1 ; [2H]c1ccc2cc(-c3cccc(-c4c5ccccc5c(-c5ccc(-c6cccc7oc8cc(-c9ccccc9)ccc8c67)cc5)c5ccccc45)c3)ccc2c1]

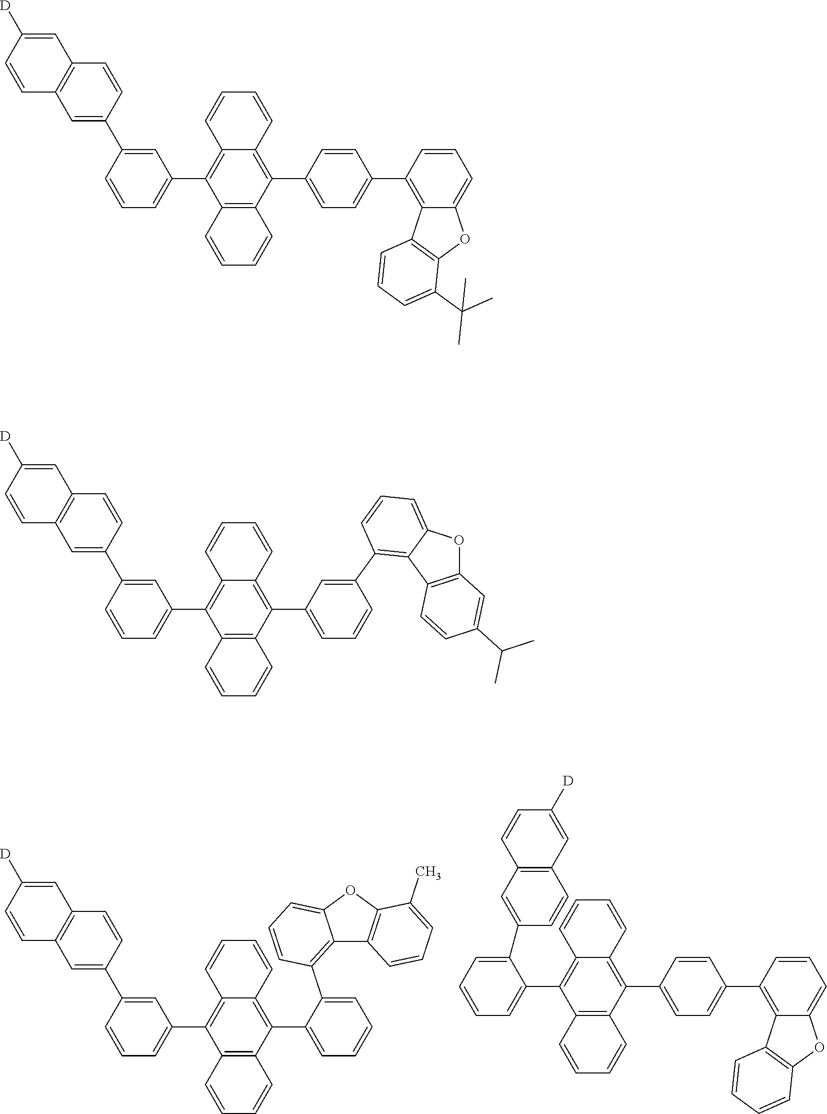

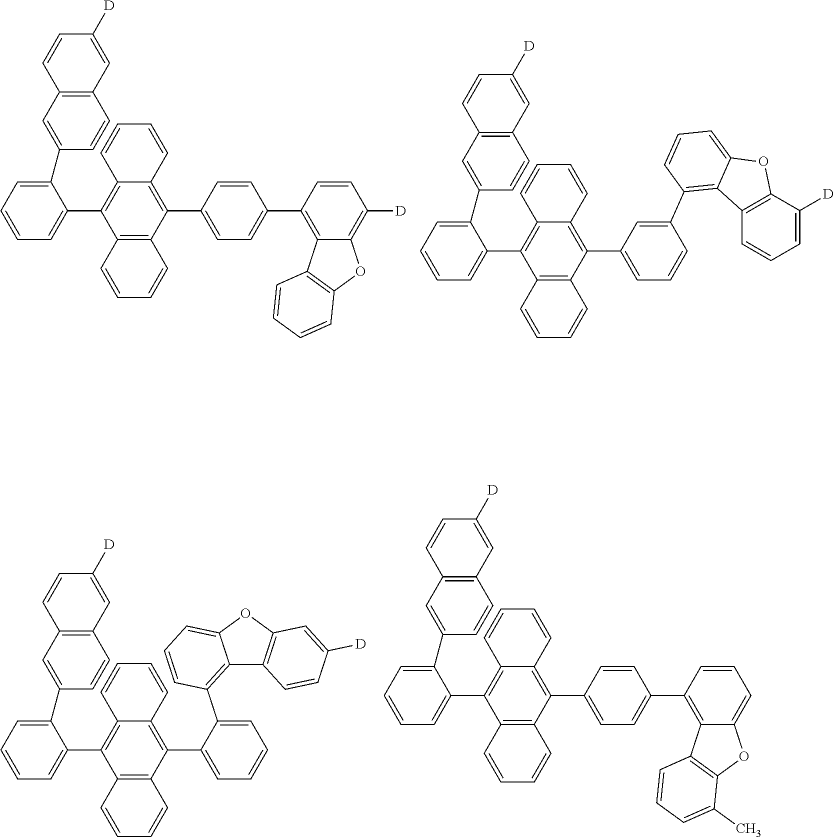

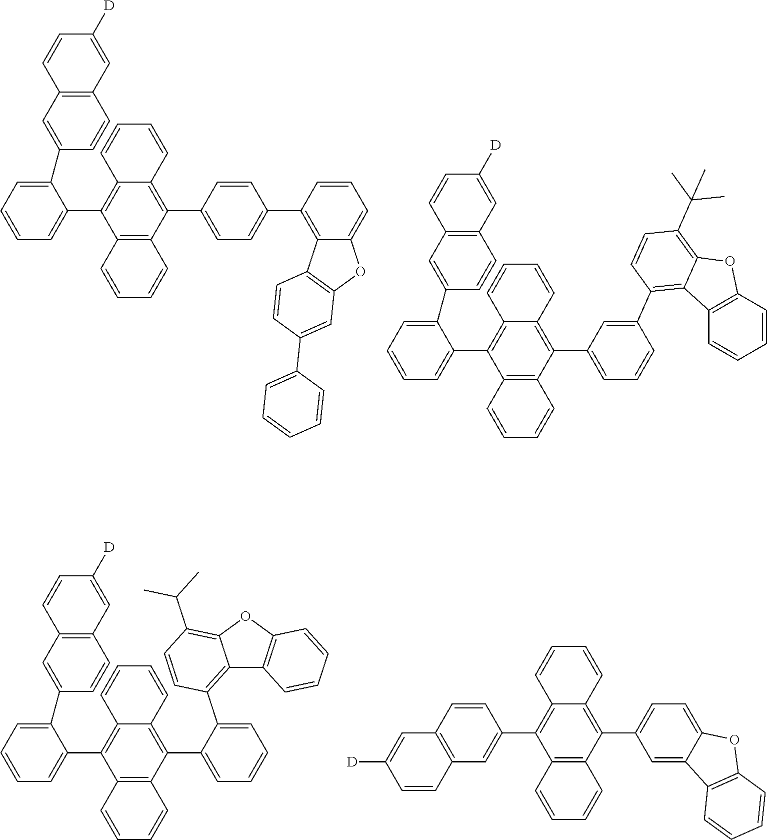 [SMILES: [2H]c1ccc2cc(-c3ccccc3-c3c4ccccc4c(-c4ccc(-c5cccc6oc7cc(-c8ccccc8)ccc7c56)cc4)c4ccccc34)ccc2c1 ; [2H]c1ccc2cc(-c3ccccc3-c3c4ccccc4c(-c4cccc(-c5ccc(C(C)(C)C)c6oc7ccccc7c56)c4)c4ccccc34)ccc2c1 ; [2H]c1ccc2cc(-c3ccccc3-c3c4ccccc4c(-c4ccccc4-c4ccc(C(C)C)c5oc6ccccc6c45)c4ccccc34)ccc2c1 ; [2H]c1ccc2cc(-c3c4ccccc4c(-c4ccc5oc6ccccc6c5c4)c4ccccc34)ccc2c1]

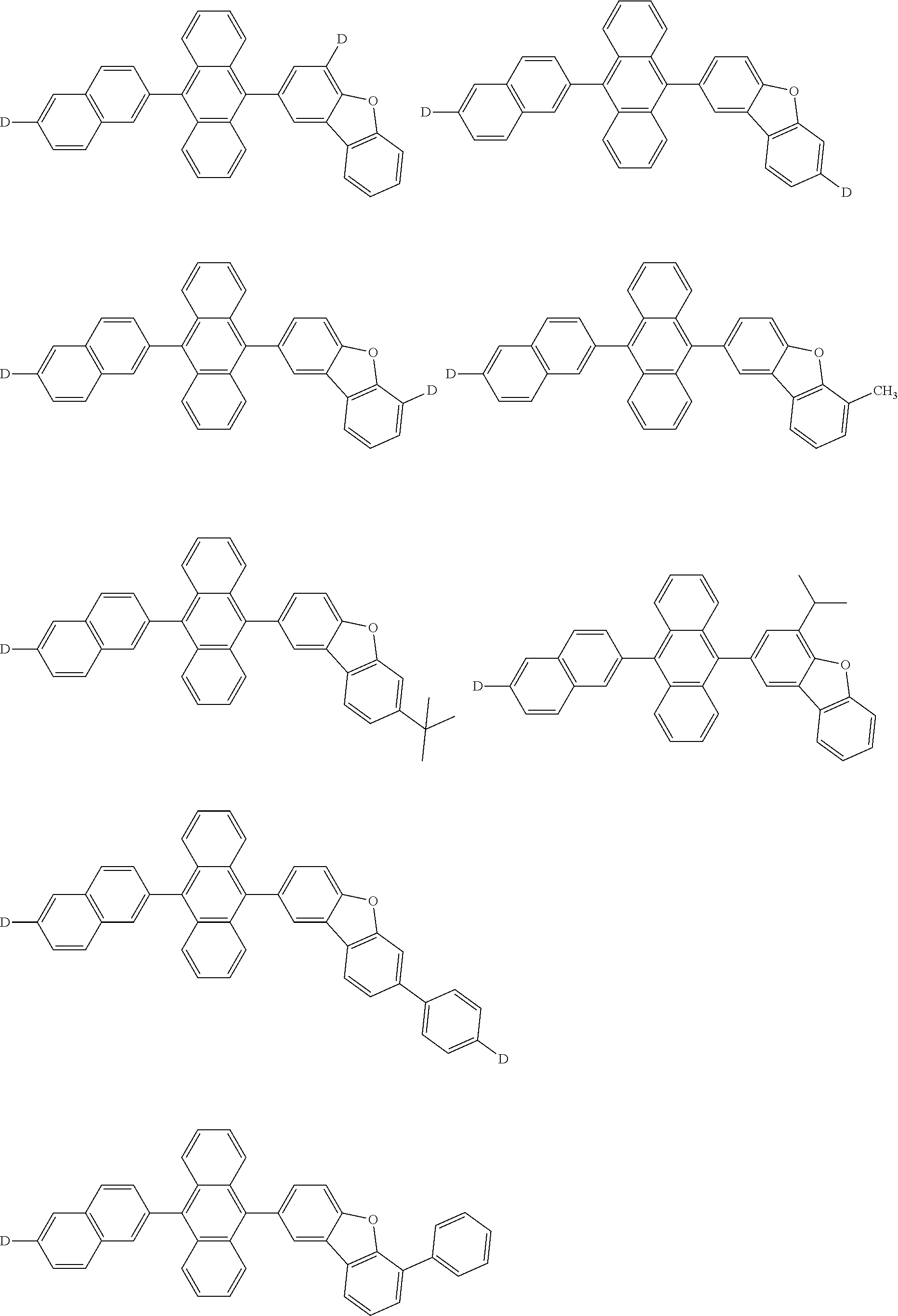

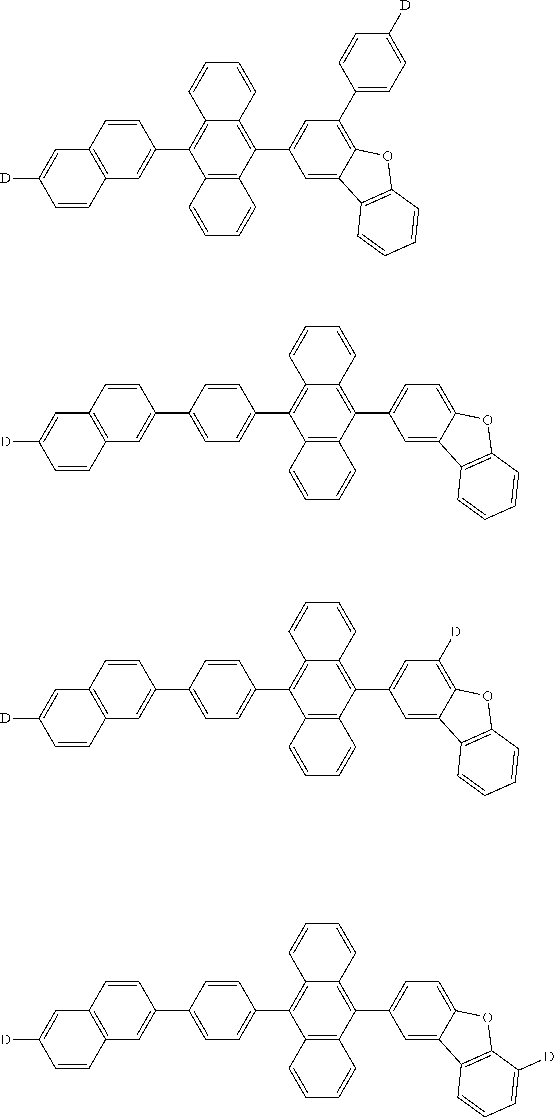 [SMILES: [2H]c1ccc(-c2cc(-c3c4ccccc4c(-c4ccc5cc([2H])ccc5c4)c4ccccc34)cc3c2oc2ccccc23)cc1 ; [2H]c1ccc2cc(-c3ccc(-c4c5ccccc5c(-c5ccc6oc7ccccc7c6c5)c5ccccc45)cc3)ccc2c1 ; [2H]c1ccc2cc(-c3ccc(-c4c5ccccc5c(-c5cc([2H])c6oc7ccccc7c6c5)c5ccccc45)cc3)ccc2c1 ; [2H]c1ccc2cc(-c3ccc(-c4c5ccccc5c(-c5ccc6oc7c([2H])cccc7c6c5)c5ccccc45)cc3)ccc2c1]

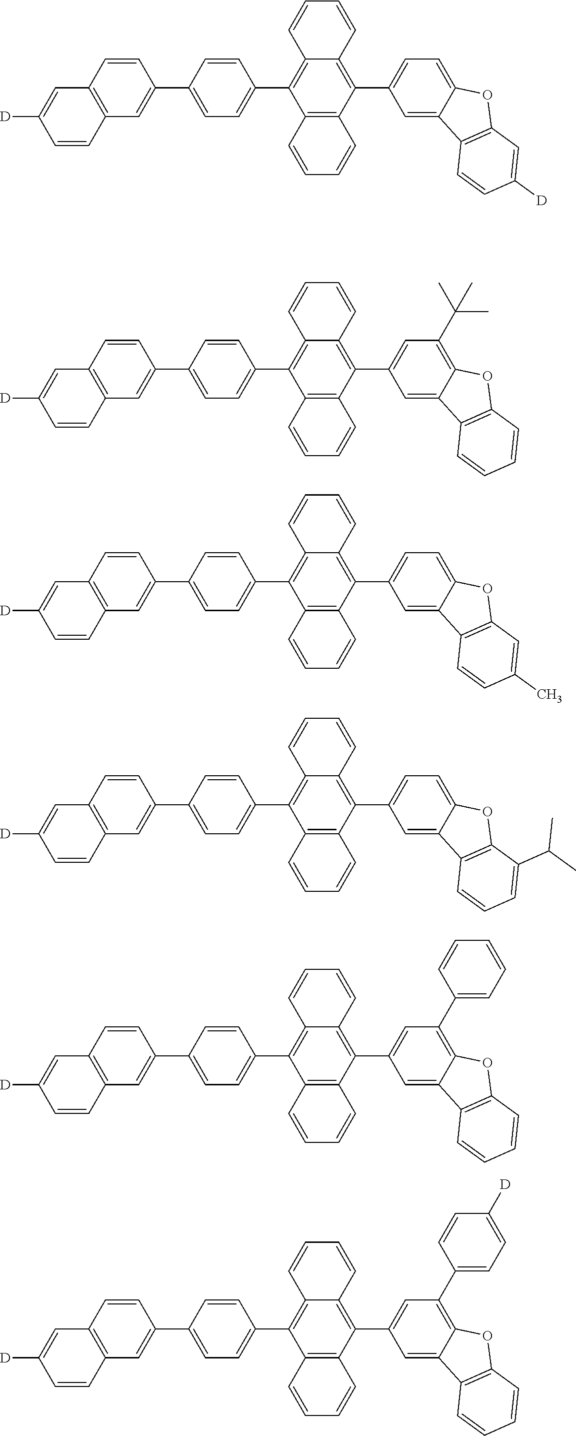

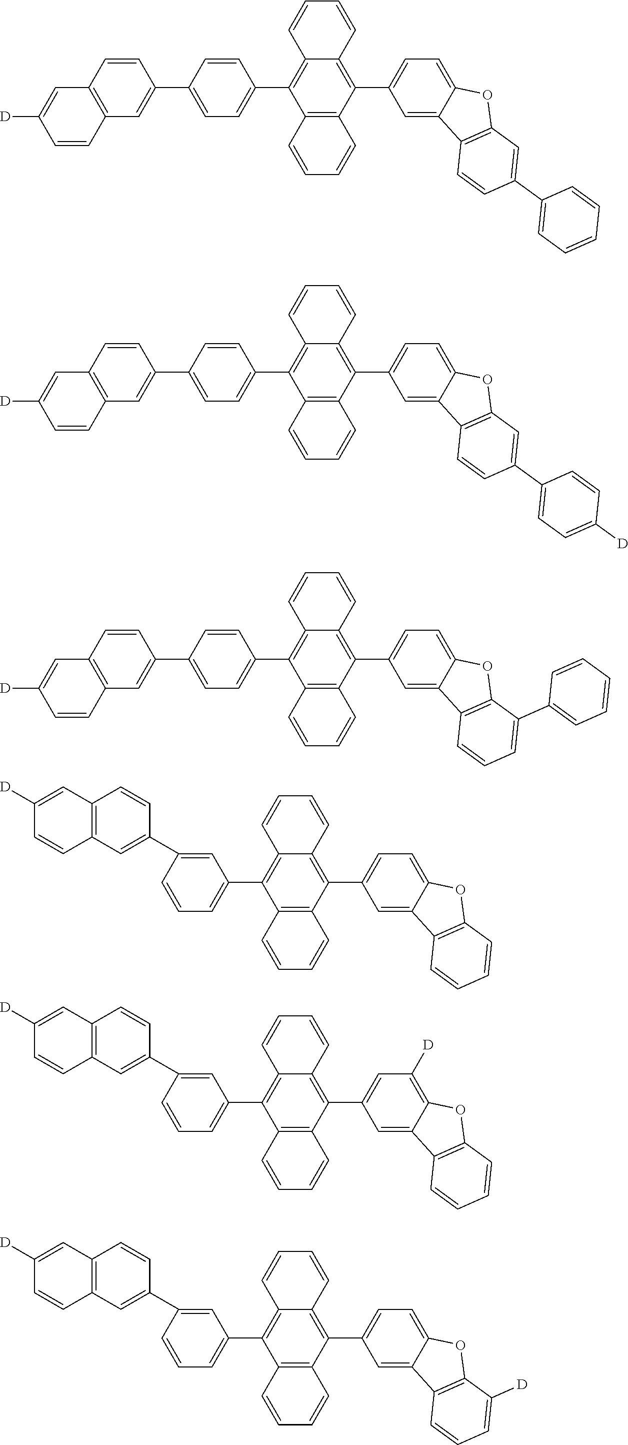

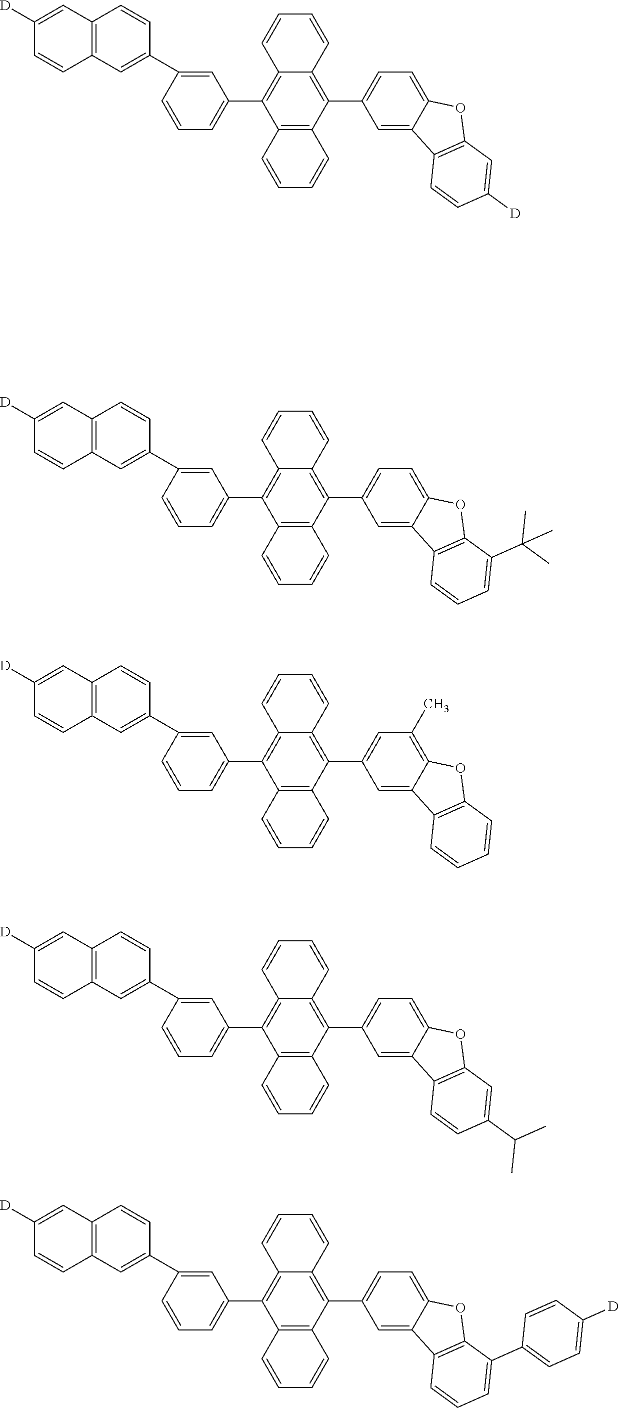 [SMILES: [2H]c1ccc2cc(-c3cccc(-c4c5ccccc5c(-c5ccc6oc7cc([2H])ccc7c6c5)c5ccccc45)c3)ccc2c1 ; [2H]c1ccc2cc(-c3cccc(-c4c5ccccc5c(-c5ccc6oc7c(C(C)(C)C)cccc7c6c5)c5ccccc45)c3)ccc2c1 ; [2H]c1ccc2cc(-c3cccc(-c4c5ccccc5c(-c5cc(C)c6oc7ccccc7c6c5)c5ccccc45)c3)ccc2c1 ; [2H]c1ccc2cc(-c3cccc(-c4c5ccccc5c(-c5ccc6oc7cc(C(C)C)ccc7c6c5)c5ccccc45)c3)ccc2c1 ; [2H]c1ccc(-c2cccc3c2oc2ccc(-c4c5ccccc5c(-c5cccc(-c6ccc7cc([2H])ccc7c6)c5)c5ccccc45)cc23)cc1]

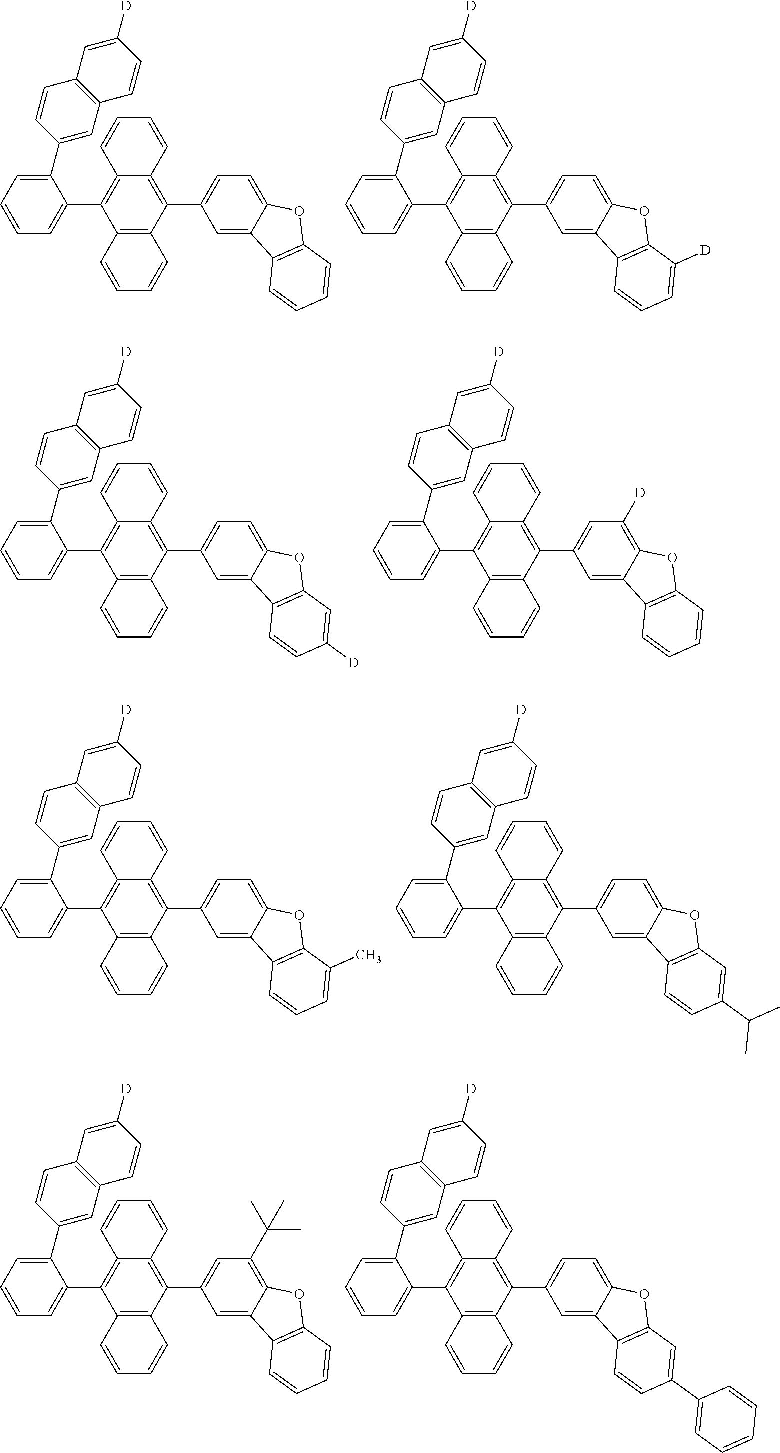

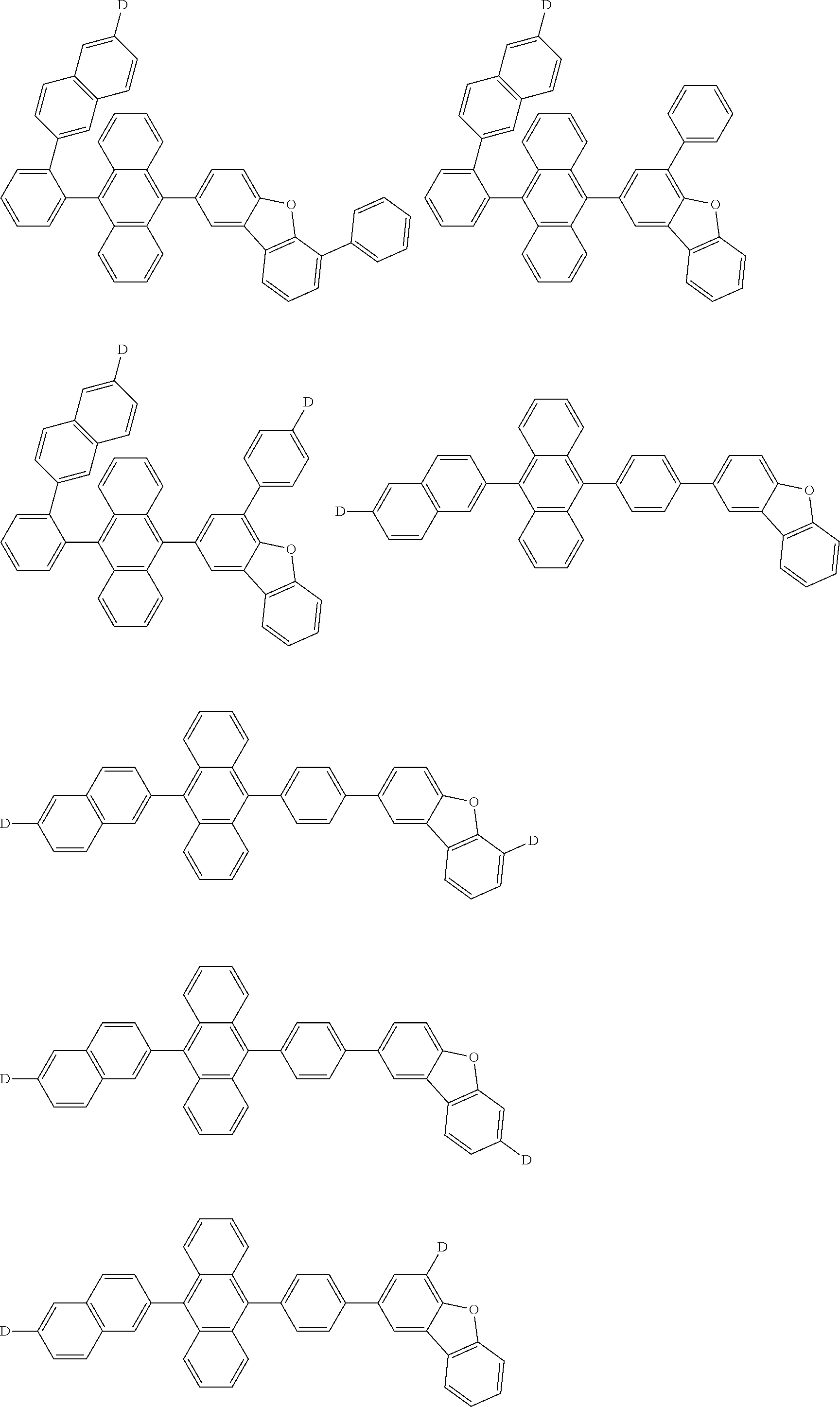

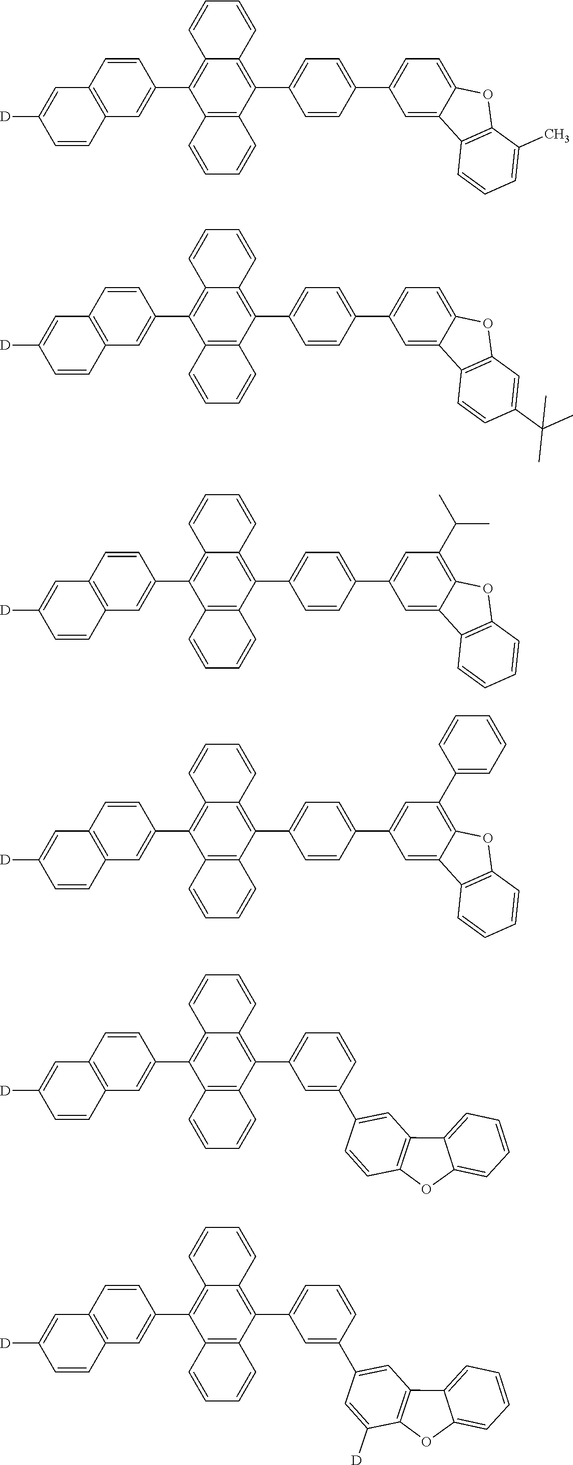

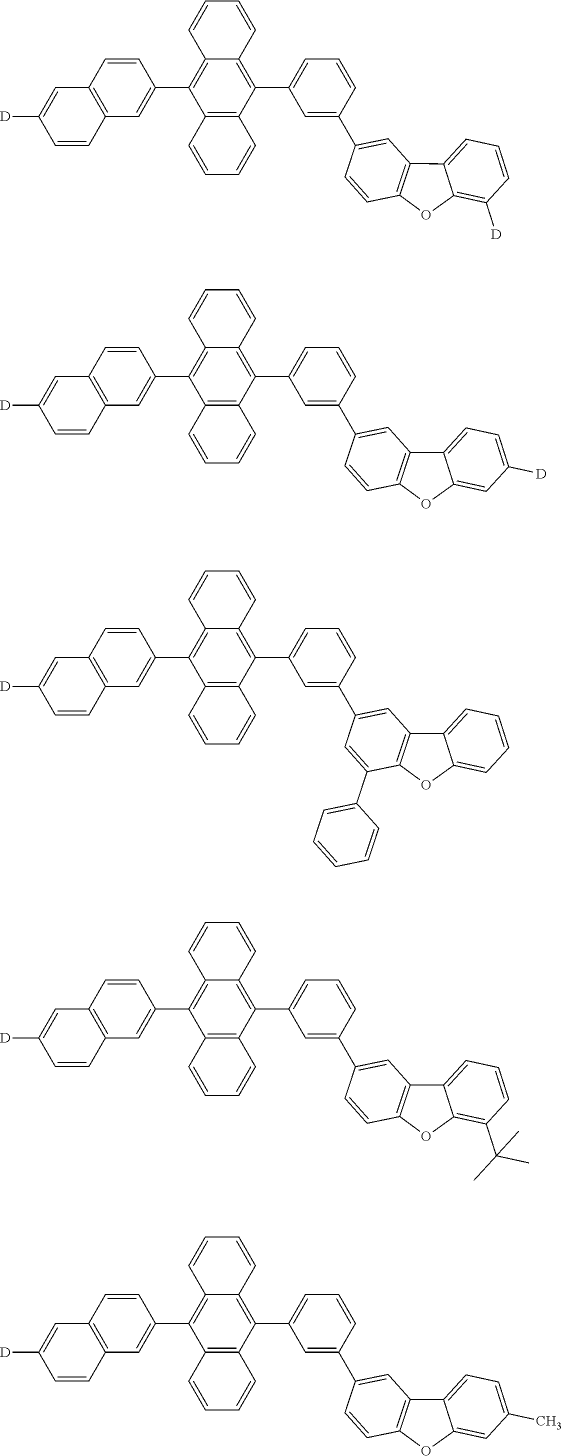 [SMILES: [2H]c1ccc2cc(-c3c4ccccc4c(-c4cccc(-c5ccc6oc7c([2H])cccc7c6c5)c4)c4ccccc34)ccc2c1 ; [2H]c1ccc2cc(-c3c4ccccc4c(-c4cccc(-c5ccc6oc7cc([2H])ccc7c6c5)c4)c4ccccc34)ccc2c1 ; [2H]c1ccc2cc(-c3c4ccccc4c(-c4cccc(-c5cc(-c6ccccc6)c6oc7ccccc7c6c5)c4)c4ccccc34)ccc2c1 ; [2H]c1ccc2cc(-c3c4ccccc4c(-c4cccc(-c5ccc6oc7c(C(C)(C)C)cccc7c6c5)c4)c4ccccc34)ccc2c1 ; [2H]c1ccc2cc(-c3c4ccccc4c(-c4cccc(-c5ccc6oc7cc(C)ccc7c6c5)c4)c4ccccc34)ccc2c1]

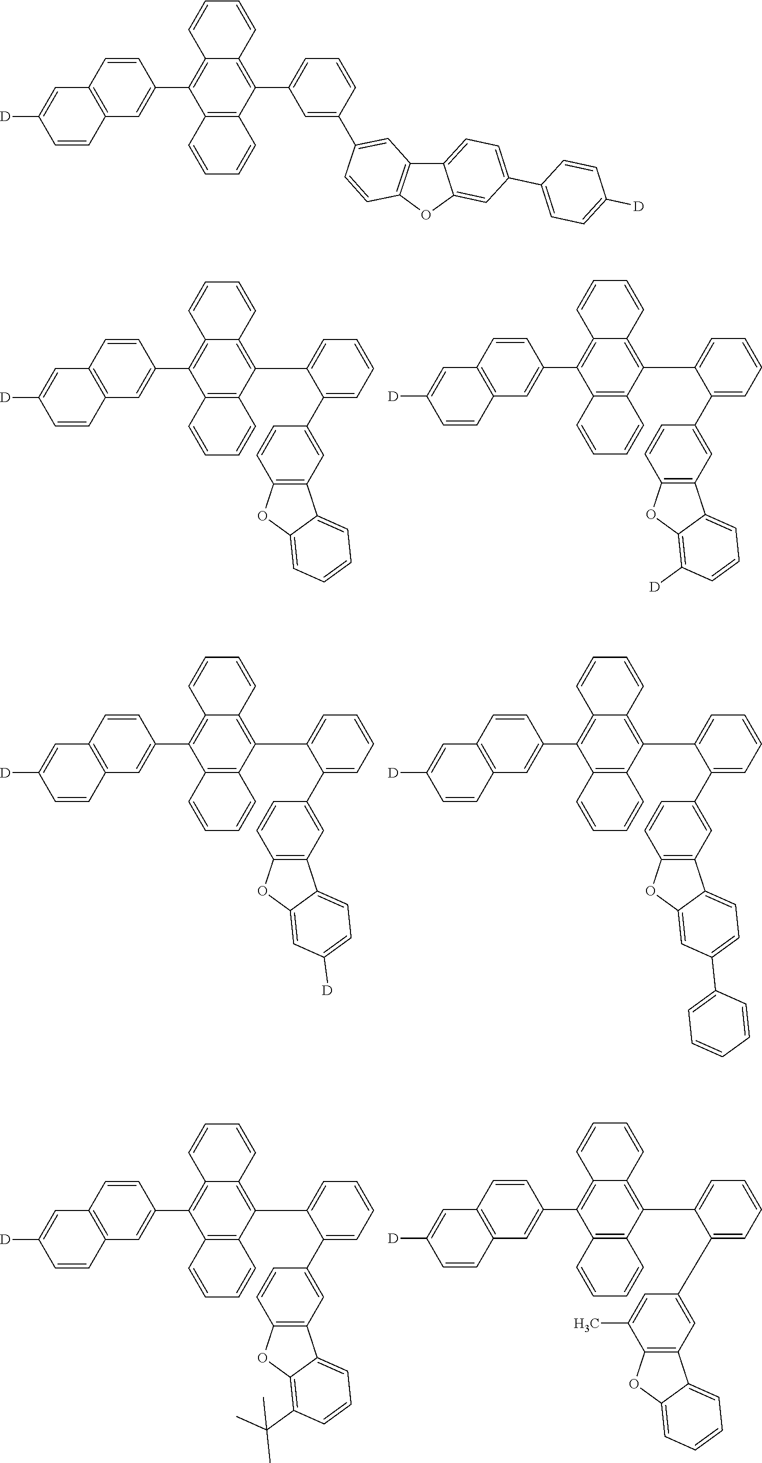

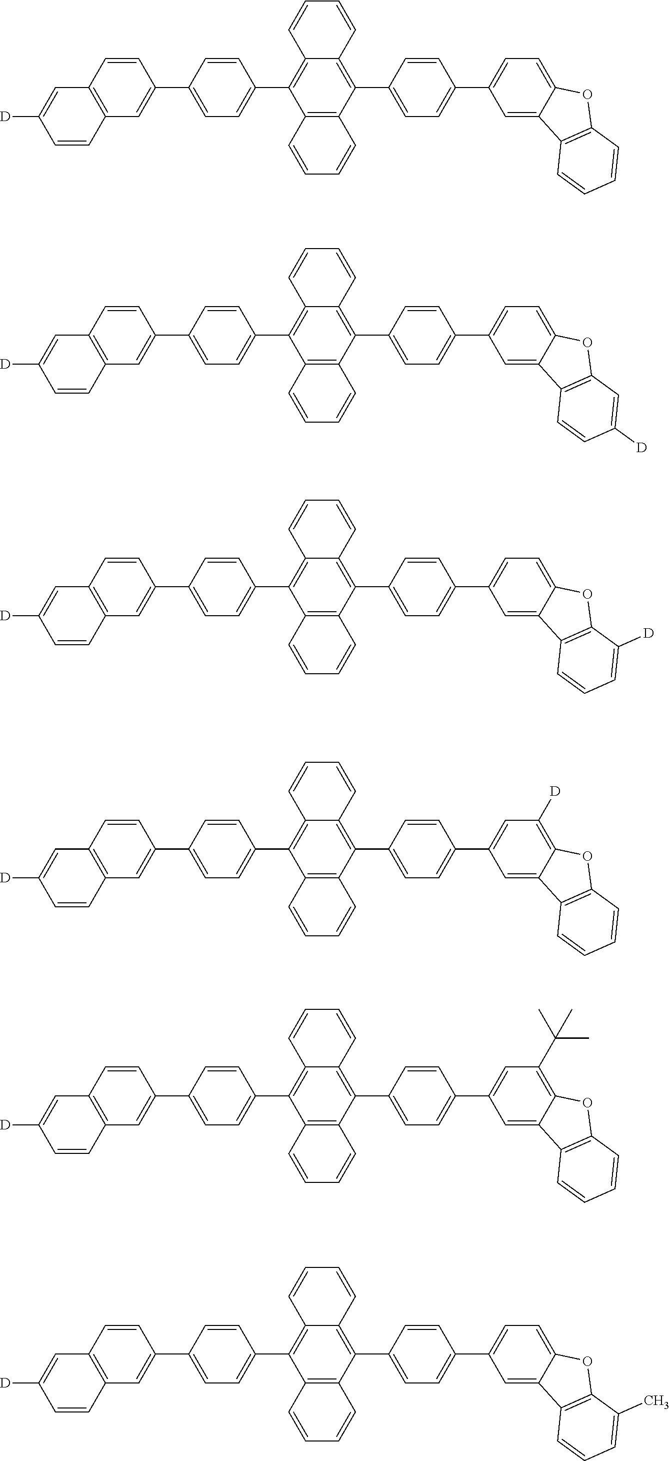

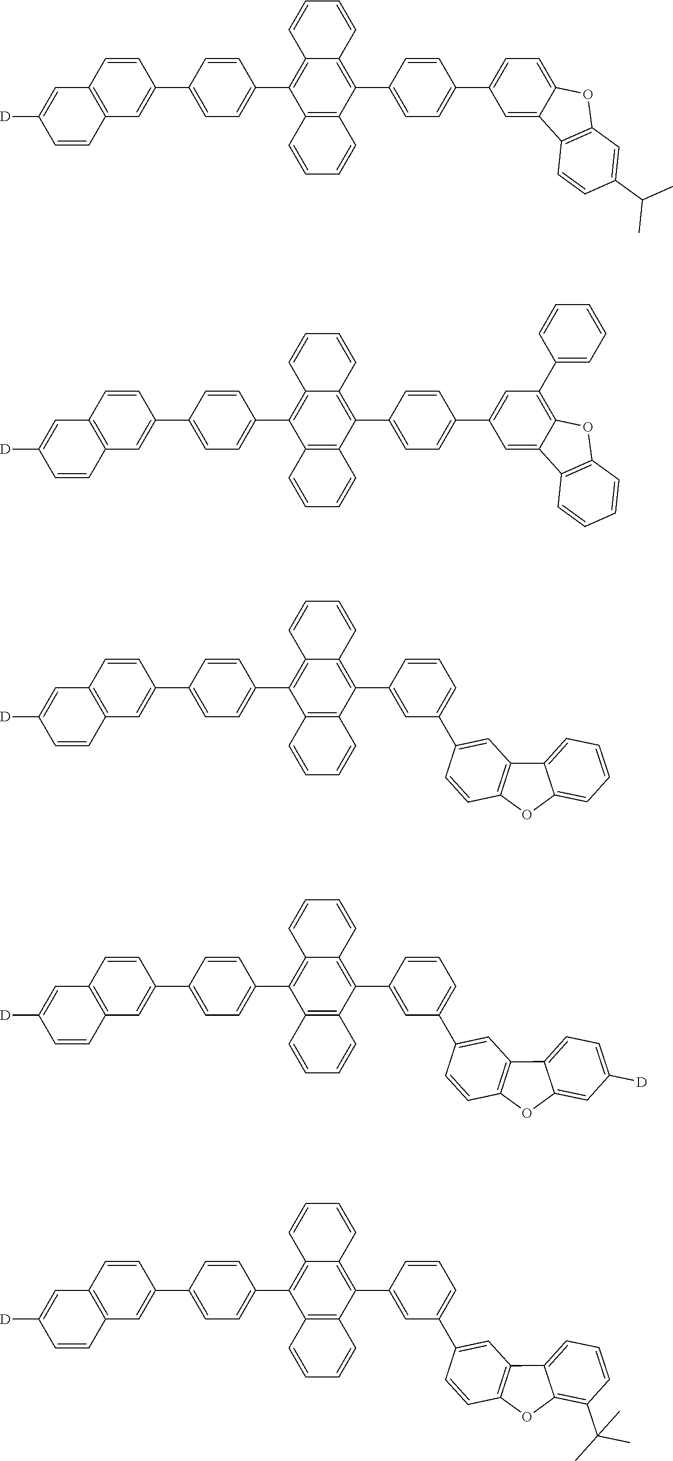 [SMILES: [2H]c1ccc2cc(-c3ccc(-c4c5ccccc5c(-c5ccc(-c6ccc7oc8cc(C(C)C)ccc8c7c6)cc5)c5ccccc45)cc3)ccc2c1 ; [2H]c1ccc2cc(-c3ccc(-c4c5ccccc5c(-c5ccc(-c6cc(-c7ccccc7)c7oc8ccccc8c7c6)cc5)c5ccccc45)cc3)ccc2c1 ; [2H]c1ccc2cc(-c3ccc(-c4c5ccccc5c(-c5cccc(-c6ccc7oc8ccccc8c7c6)c5)c5ccccc45)cc3)ccc2c1 ; [2H]c1ccc2cc(-c3ccc(-c4c5ccccc5c(-c5cccc(-c6ccc7oc8cc([2H])ccc8c7c6)c5)c5ccccc45)cc3)ccc2c1 ; [2H]c1ccc2cc(-c3ccc(-c4c5ccccc5c(-c5cccc(-c6ccc7oc8c(C(C)(C)C)cccc8c7c6)c5)c5ccccc45)cc3)ccc2c1]

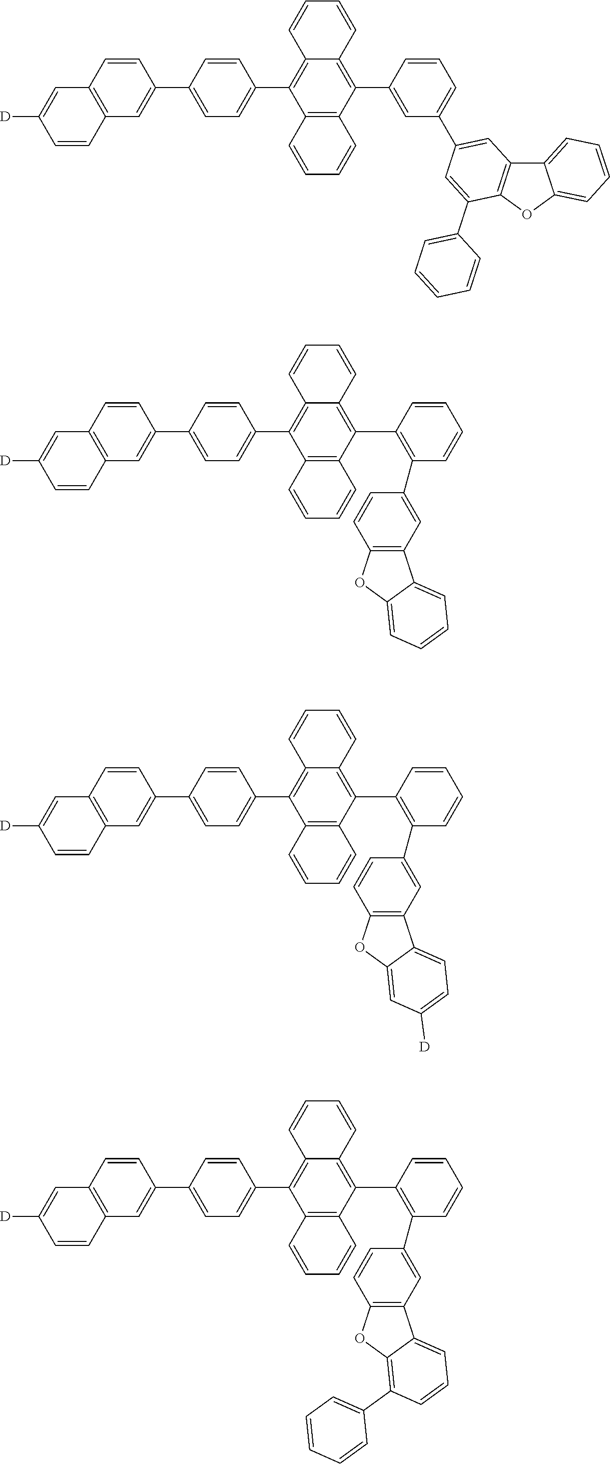 [SMILES: [2H]c1ccc2cc(-c3ccc(-c4c5ccccc5c(-c5cccc(-c6cc(-c7ccccc7)c7oc8ccccc8c7c6)c5)c5ccccc45)cc3)ccc2c1 ; [2H]c1ccc2cc(-c3ccc(-c4c5ccccc5c(-c5ccccc5-c5ccc6oc7ccccc7c6c5)c5ccccc45)cc3)ccc2c1 ; [2H]c1ccc2cc(-c3ccc(-c4c5ccccc5c(-c5ccccc5-c5ccc6oc7cc([2H])ccc7c6c5)c5ccccc45)cc3)ccc2c1 ; [2H]c1ccc2cc(-c3ccc(-c4c5ccccc5c(-c5ccccc5-c5ccc6oc7c(-c8ccccc8)cccc7c6c5)c5ccccc45)cc3)ccc2c1]

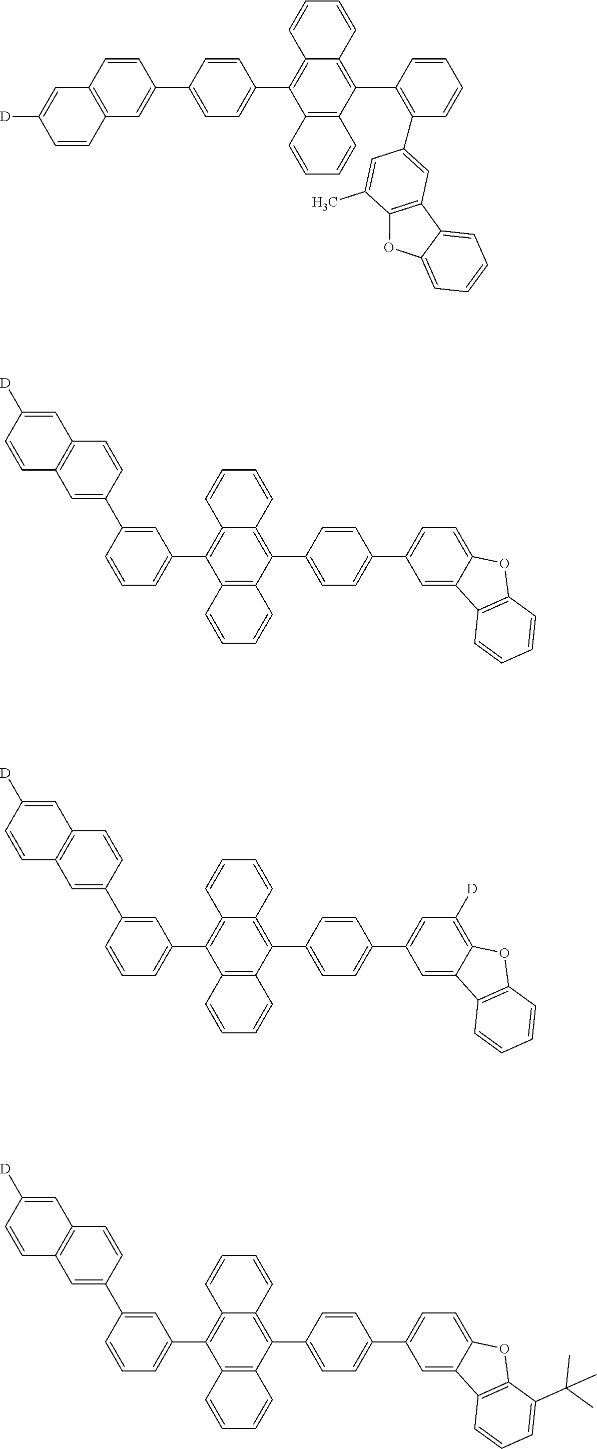 [SMILES: [2H]c1ccc2cc(-c3ccc(-c4c5ccccc5c(-c5ccccc5-c5cc(C)c6oc7ccccc7c6c5)c5ccccc45)cc3)ccc2c1 ; [2H]c1ccc2cc(-c3cccc(-c4c5ccccc5c(-c5ccc(-c6ccc7oc8ccccc8c7c6)cc5)c5ccccc45)c3)ccc2c1 ; [2H]c1ccc2cc(-c3cccc(-c4c5ccccc5c(-c5ccc(-c6cc([2H])c7oc8ccccc8c7c6)cc5)c5ccccc45)c3)ccc2c1 ; [2H]c1ccc2cc(-c3cccc(-c4c5ccccc5c(-c5ccc(-c6ccc7oc8c(C(C)(C)C)cccc8c7c6)cc5)c5ccccc45)c3)ccc2c1]

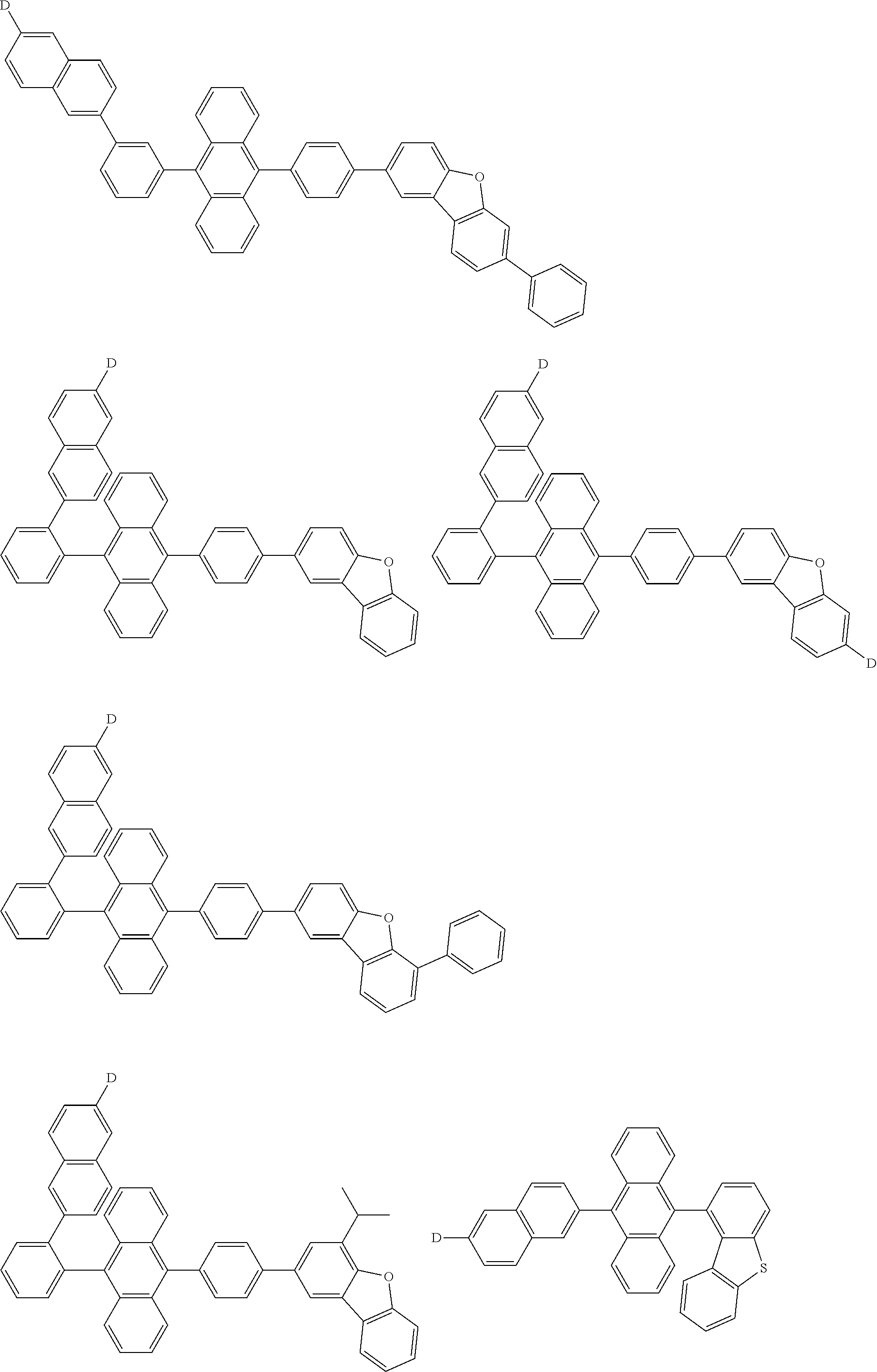

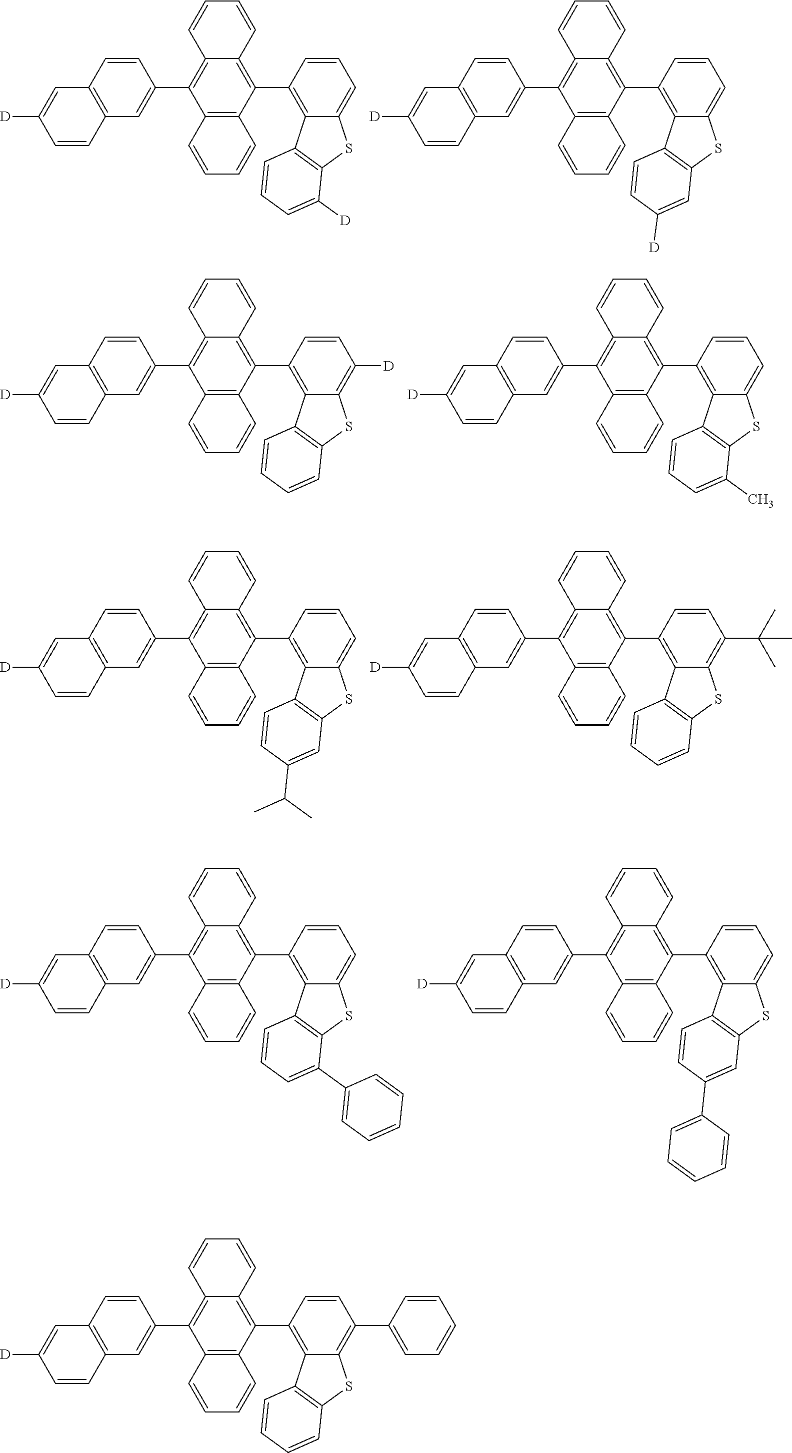

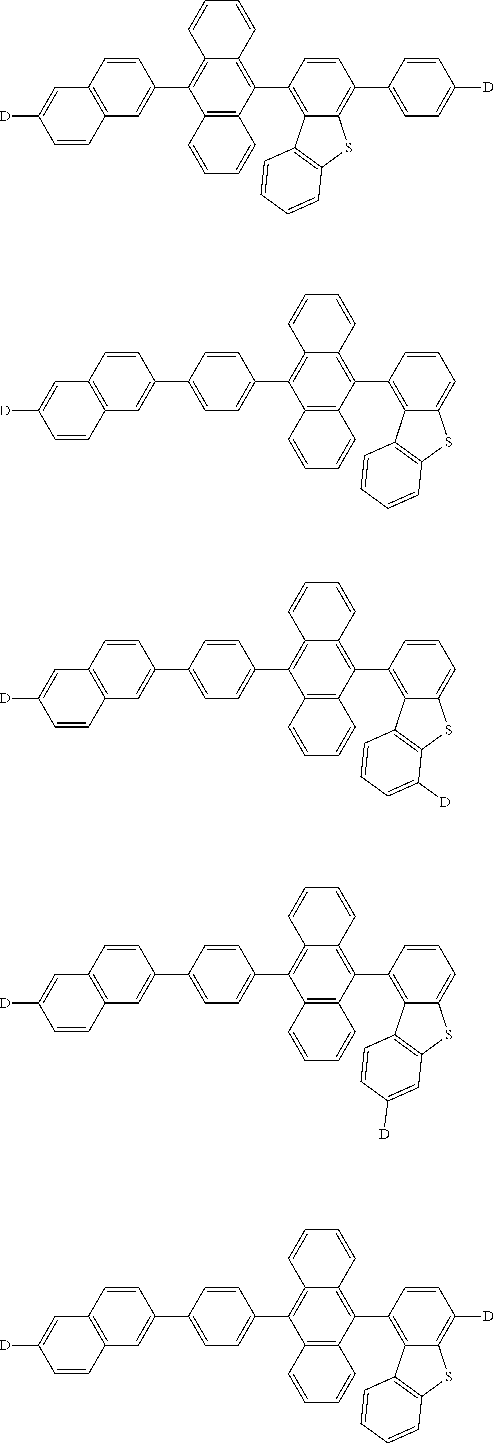 [SMILES: [2H]c1ccc(-c2ccc(-c3c4ccccc4c(-c4ccc5cc([2H])ccc5c4)c4ccccc34)c3c2sc2ccccc23)cc1 ; [2H]c1ccc2cc(-c3ccc(-c4c5ccccc5c(-c5cccc6sc7ccccc7c56)c5ccccc45)cc3)ccc2c1 ; [2H]c1ccc2cc(-c3ccc(-c4c5ccccc5c(-c5cccc6sc7c([2H])cccc7c56)c5ccccc45)cc3)ccc2c1 ; [2H]c1ccc2cc(-c3ccc(-c4c5ccccc5c(-c5cccc6sc7cc([2H])ccc7c56)c5ccccc45)cc3)ccc2c1 ; [2H]c1ccc2cc(-c3ccc(-c4c5ccccc5c(-c5ccc([2H])c6sc7ccccc7c56)c5ccccc45)cc3)ccc2c1]

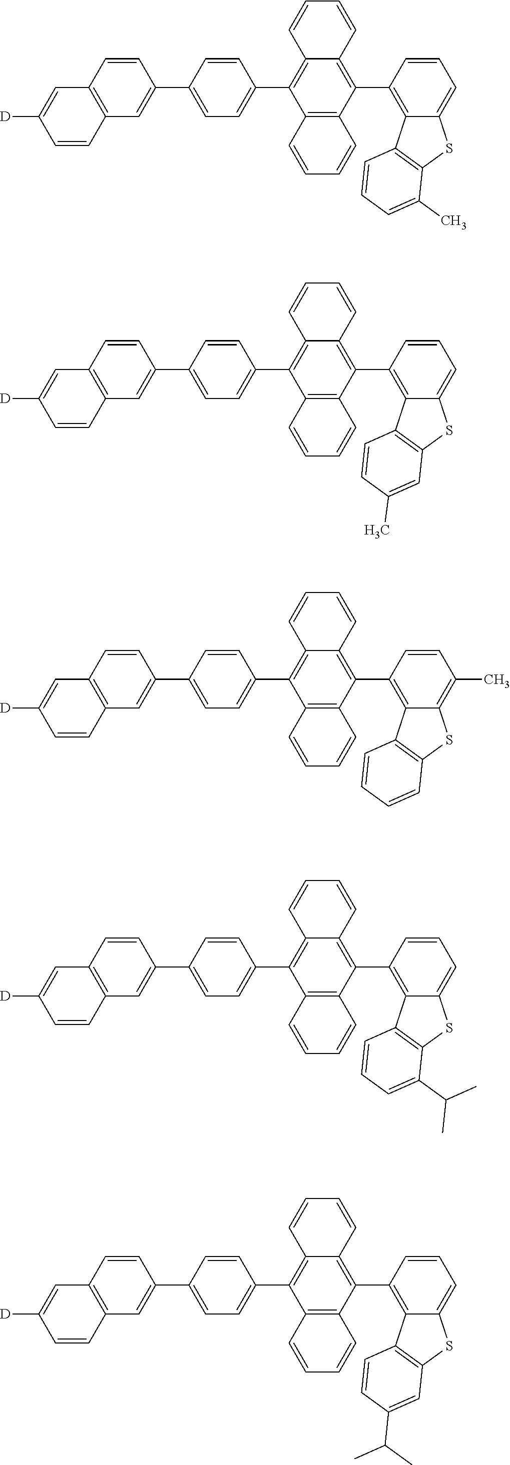 [SMILES: [2H]c1ccc2cc(-c3ccc(-c4c5ccccc5c(-c5cccc6sc7c(C)cccc7c56)c5ccccc45)cc3)ccc2c1 ; [2H]c1ccc2cc(-c3ccc(-c4c5ccccc5c(-c5cccc6sc7cc(C)ccc7c56)c5ccccc45)cc3)ccc2c1 ; [2H]c1ccc2cc(-c3ccc(-c4c5ccccc5c(-c5ccc(C)c6sc7ccccc7c56)c5ccccc45)cc3)ccc2c1 ; [2H]c1ccc2cc(-c3ccc(-c4c5ccccc5c(-c5cccc6sc7c(C(C)C)cccc7c56)c5ccccc45)cc3)ccc2c1 ; [2H]c1ccc2cc(-c3ccc(-c4c5ccccc5c(-c5cccc6sc7cc(C(C)C)ccc7c56)c5ccccc45)cc3)ccc2c1]

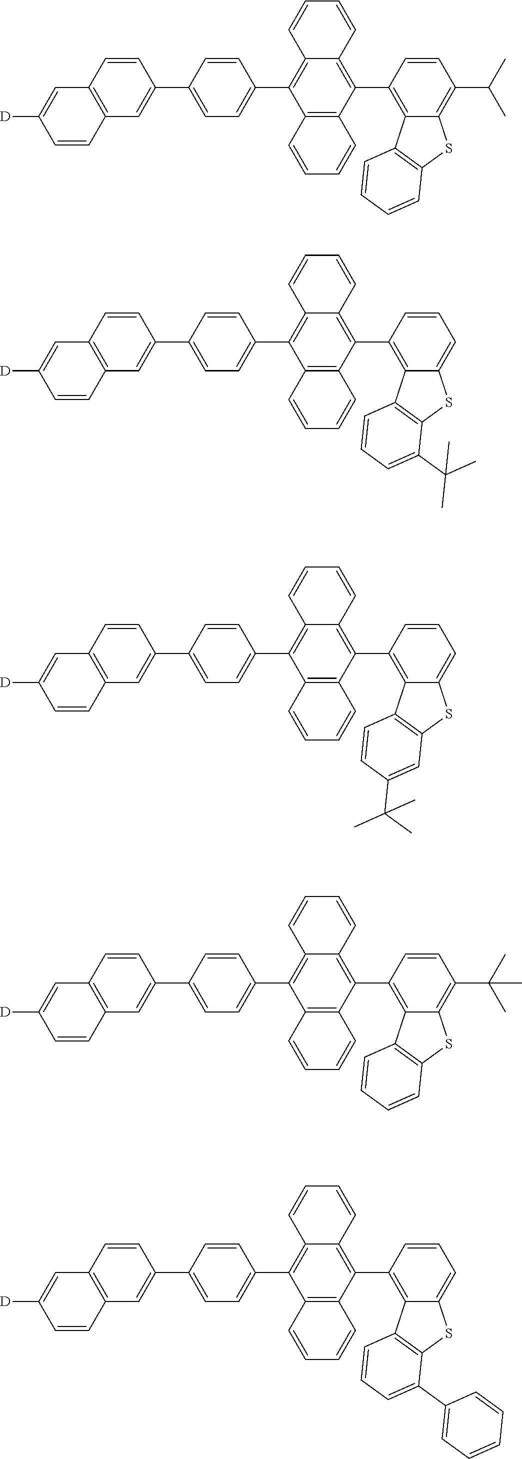 [SMILES: [2H]c1ccc2cc(-c3ccc(-c4c5ccccc5c(-c5ccc(C(C)C)c6sc7ccccc7c56)c5ccccc45)cc3)ccc2c1 ; [2H]c1ccc2cc(-c3ccc(-c4c5ccccc5c(-c5cccc6sc7c(C(C)(C)C)cccc7c56)c5ccccc45)cc3)ccc2c1 ; [2H]c1ccc2cc(-c3ccc(-c4c5ccccc5c(-c5cccc6sc7cc(C(C)(C)C)ccc7c56)c5ccccc45)cc3)ccc2c1 ; [2H]c1ccc2cc(-c3ccc(-c4c5ccccc5c(-c5ccc(C(C)(C)C)c6sc7ccccc7c56)c5ccccc45)cc3)ccc2c1 ; [2H]c1ccc2cc(-c3ccc(-c4c5ccccc5c(-c5cccc6sc7c(-c8ccccc8)cccc7c56)c5ccccc45)cc3)ccc2c1]

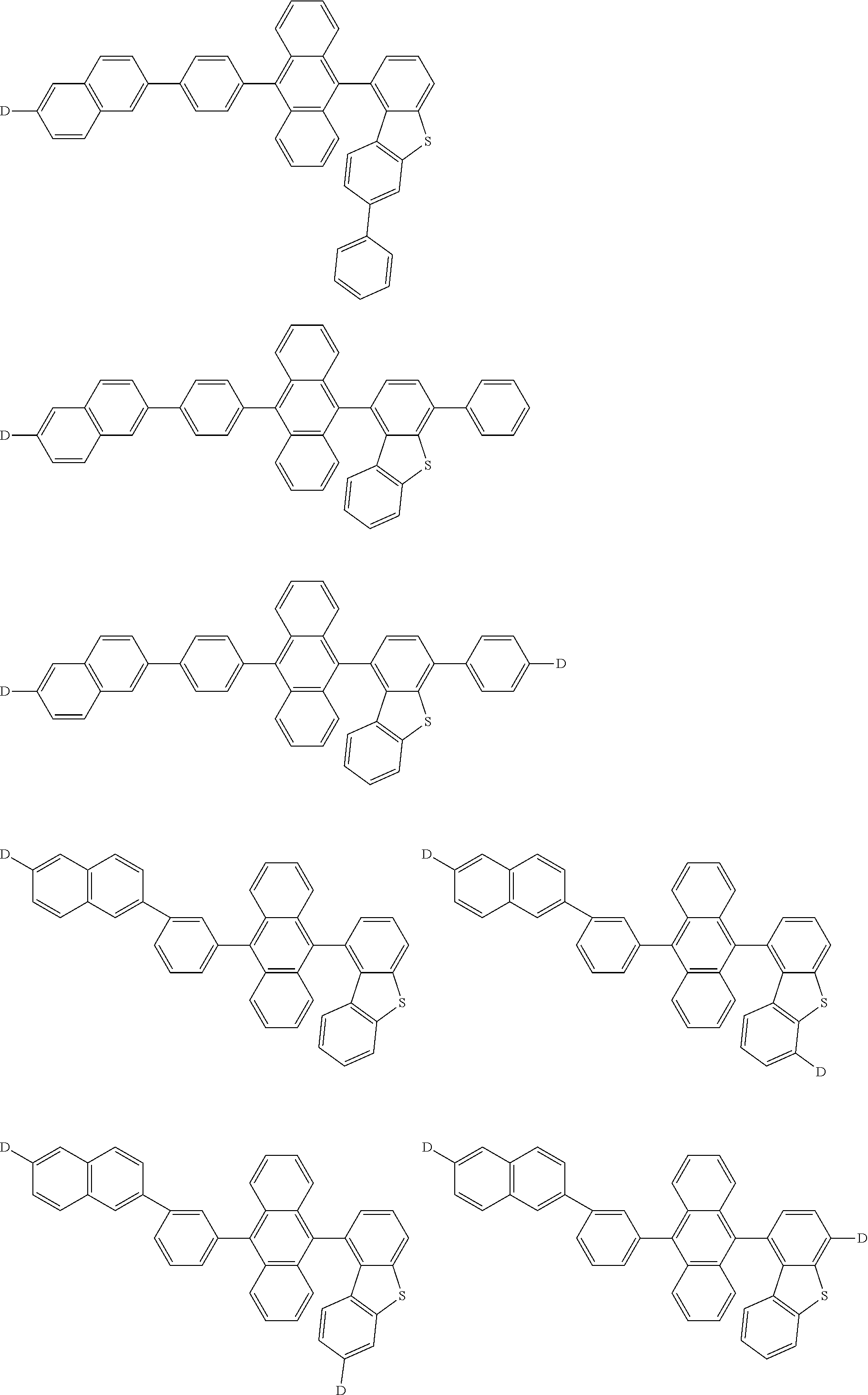

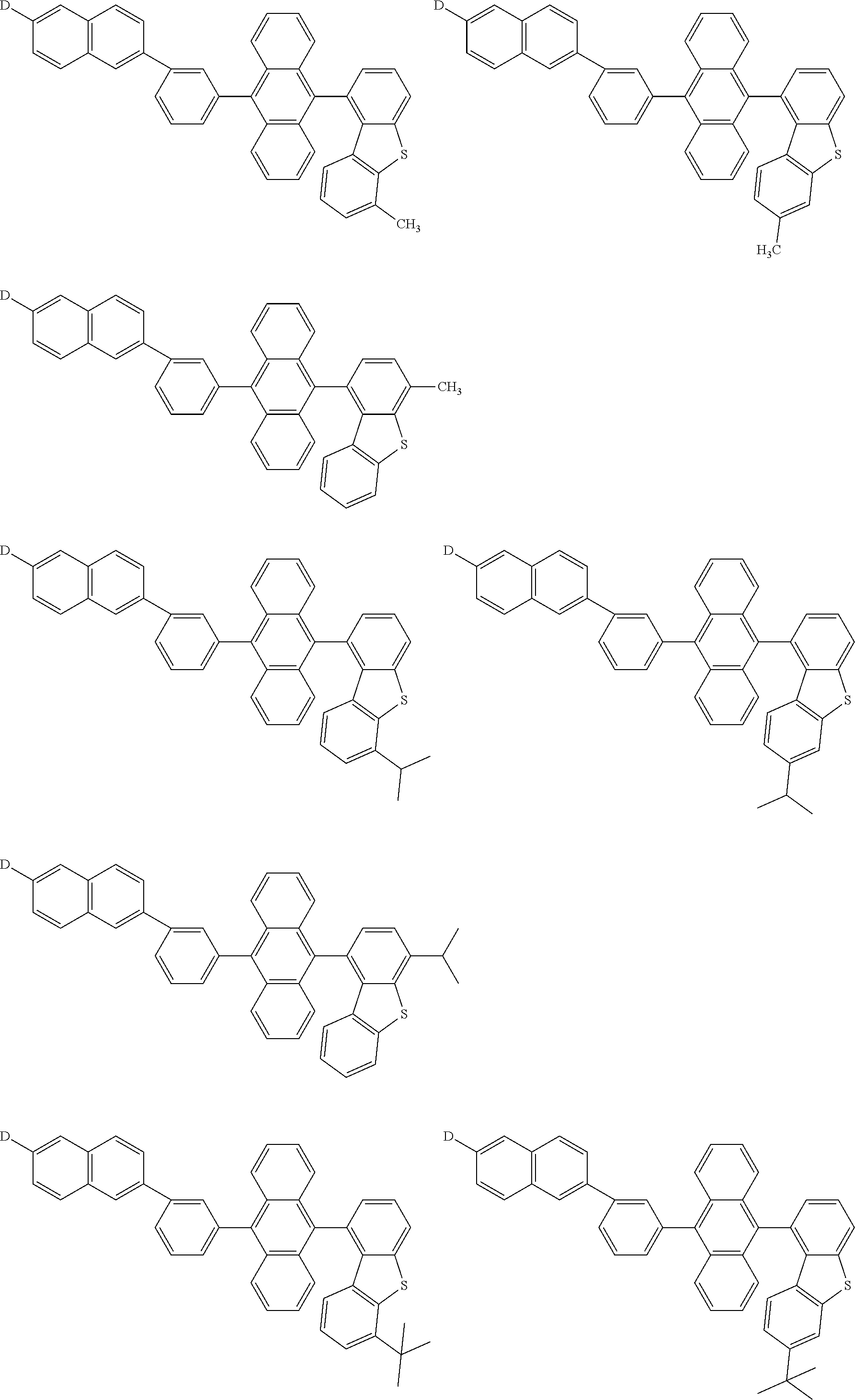

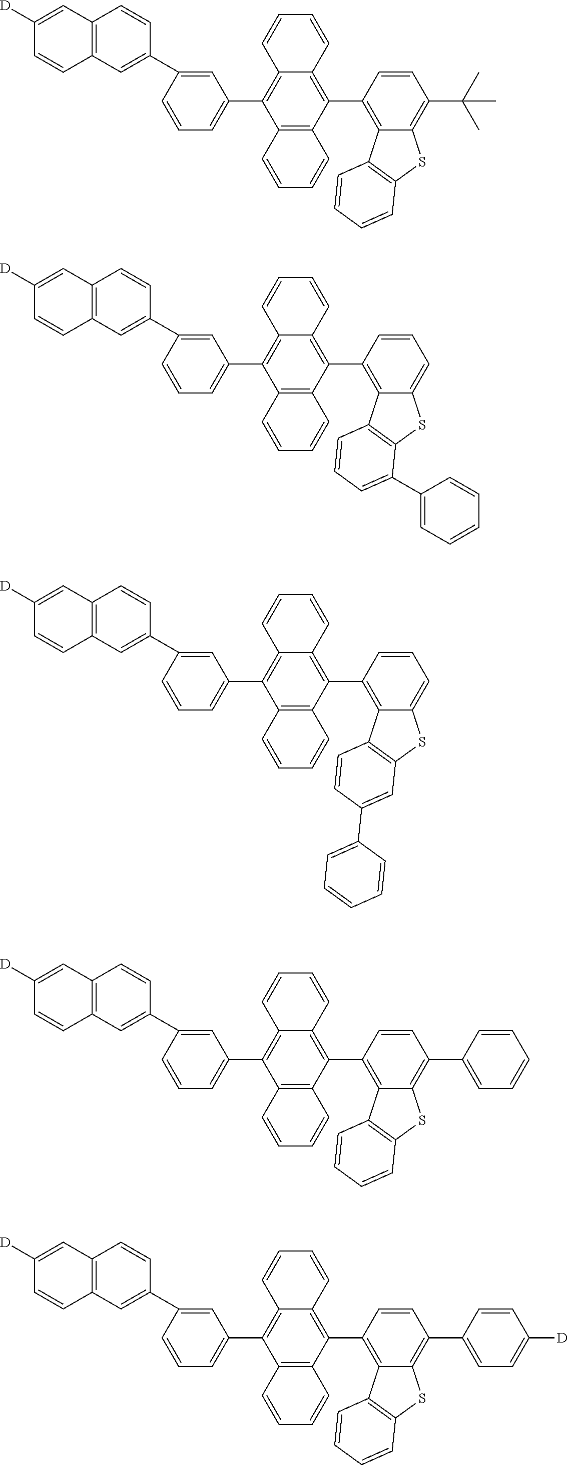 [SMILES: [2H]c1ccc2cc(-c3cccc(-c4c5ccccc5c(-c5ccc(C(C)(C)C)c6sc7ccccc7c56)c5ccccc45)c3)ccc2c1 ; [2H]c1ccc2cc(-c3cccc(-c4c5ccccc5c(-c5cccc6sc7c(-c8ccccc8)cccc7c56)c5ccccc45)c3)ccc2c1 ; [2H]c1ccc2cc(-c3cccc(-c4c5ccccc5c(-c5cccc6sc7cc(-c8ccccc8)ccc7c56)c5ccccc45)c3)ccc2c1 ; [2H]c1ccc2cc(-c3cccc(-c4c5ccccc5c(-c5ccc(-c6ccccc6)c6sc7ccccc7c56)c5ccccc45)c3)ccc2c1 ; [2H]c1ccc(-c2ccc(-c3c4ccccc4c(-c4cccc(-c5ccc6cc([2H])ccc6c5)c4)c4ccccc34)c3c2sc2ccccc23)cc1]

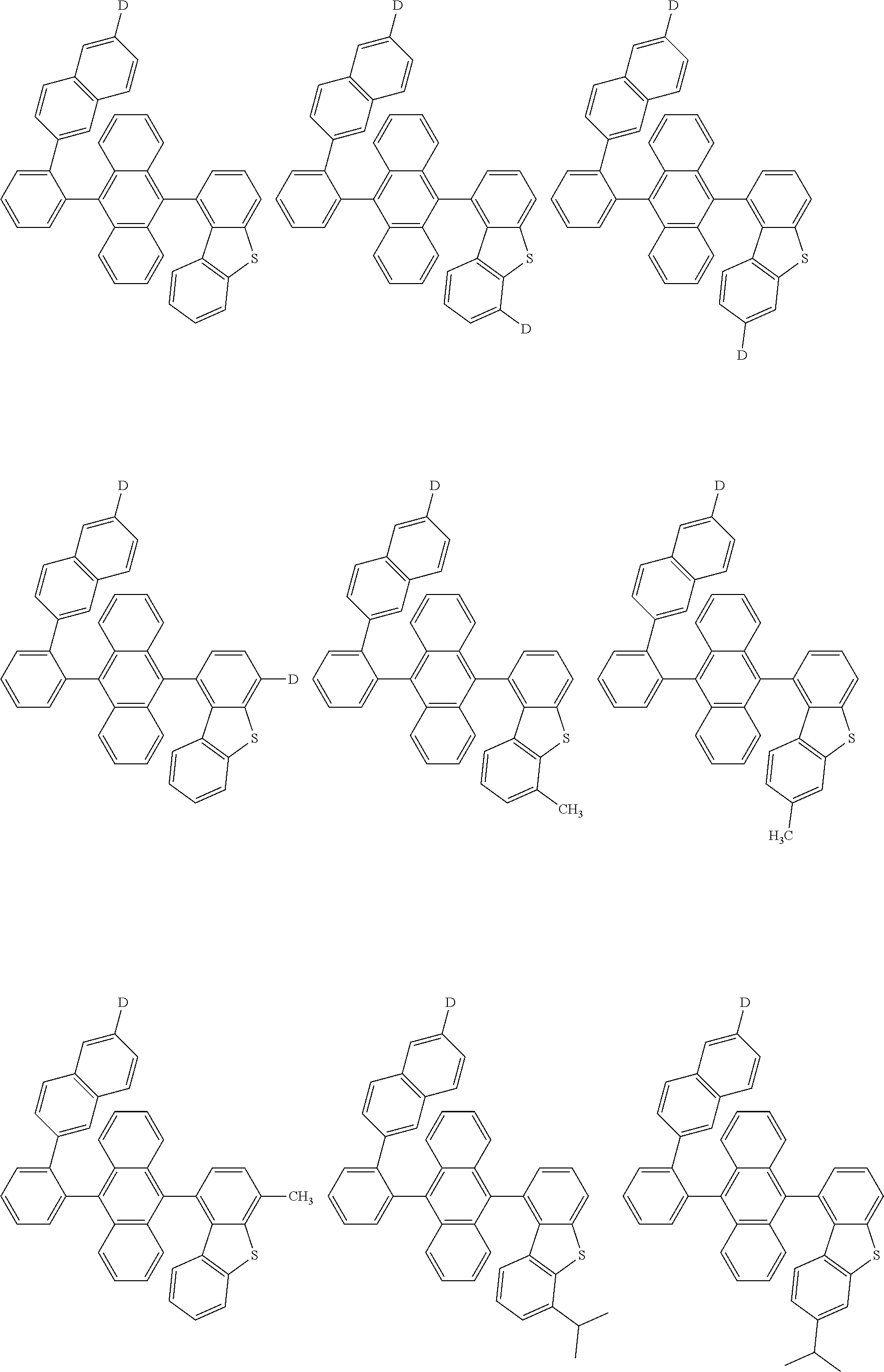

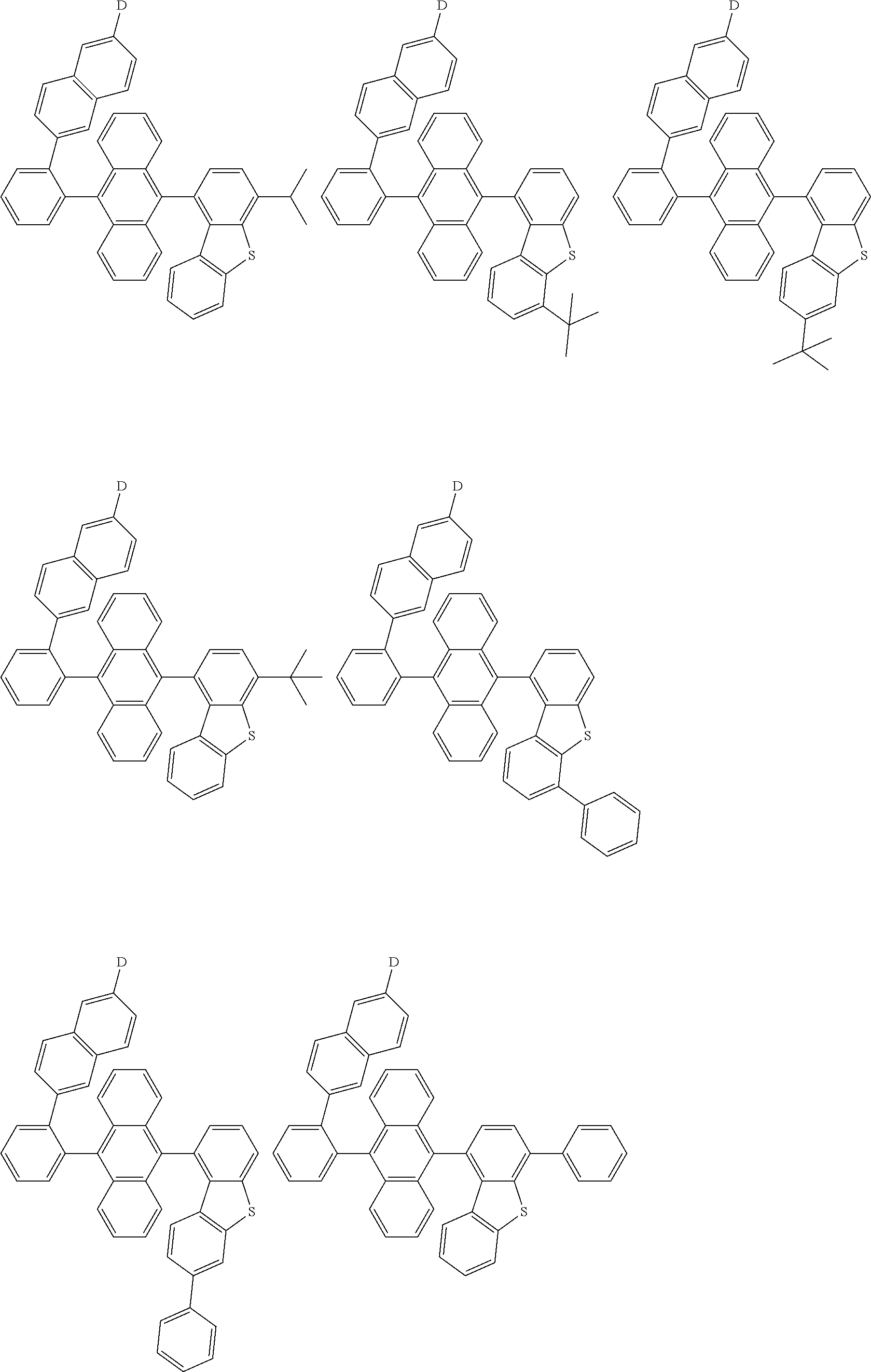

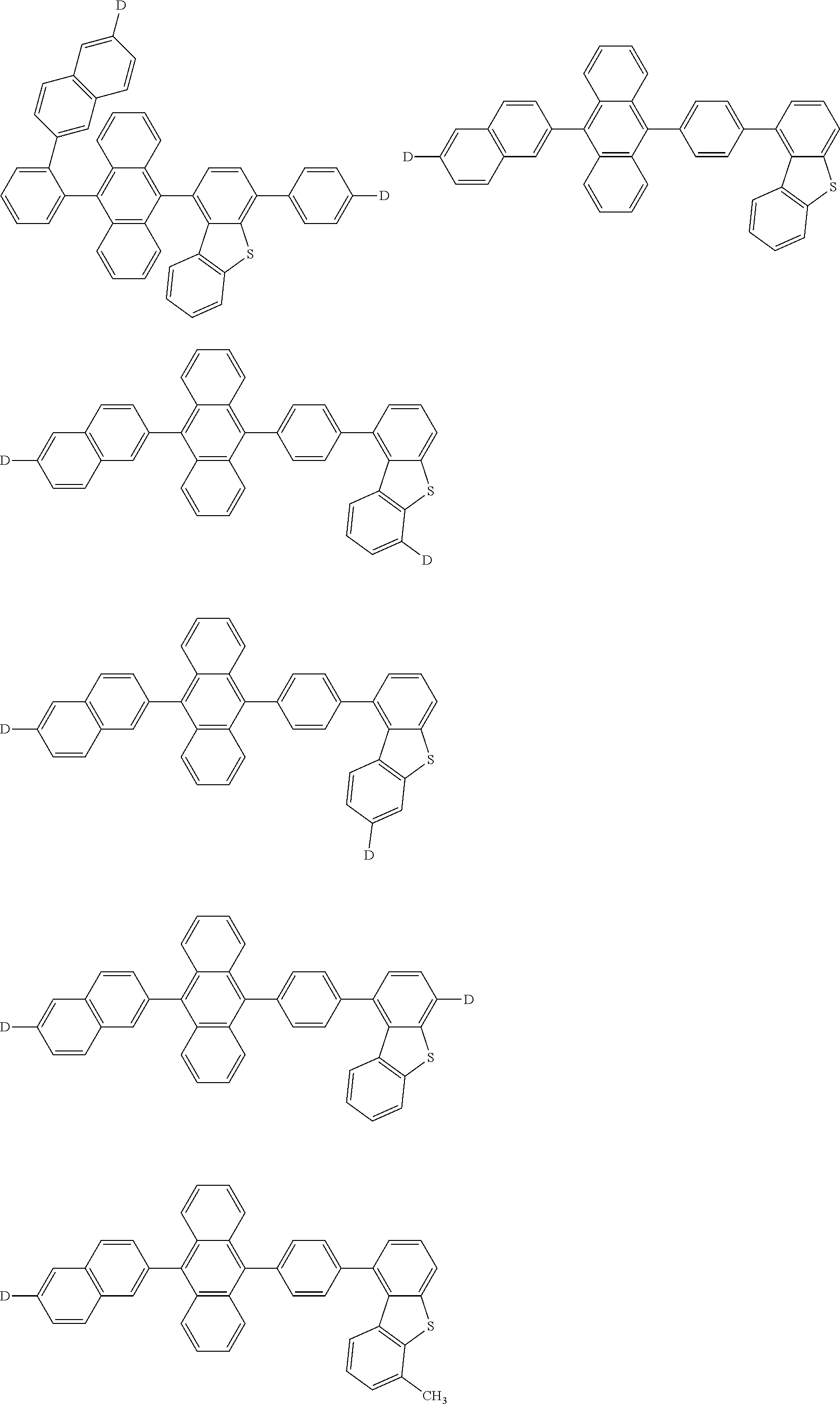

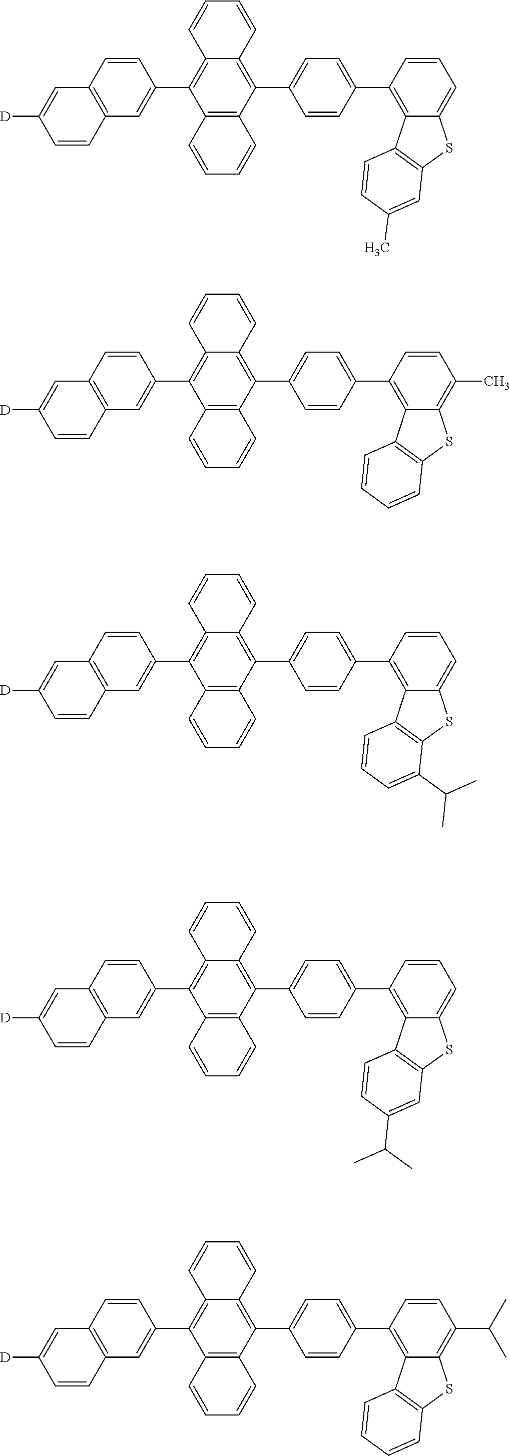 [SMILES: [2H]c1ccc2cc(-c3c4ccccc4c(-c4ccc(-c5cccc6sc7cc(C)ccc7c56)cc4)c4ccccc34)ccc2c1 ; [2H]c1ccc2cc(-c3c4ccccc4c(-c4ccc(-c5ccc(C)c6sc7ccccc7c56)cc4)c4ccccc34)ccc2c1 ; [2H]c1ccc2cc(-c3c4ccccc4c(-c4ccc(-c5cccc6sc7c(C(C)C)cccc7c56)cc4)c4ccccc34)ccc2c1 ; [2H]c1ccc2cc(-c3c4ccccc4c(-c4ccc(-c5cccc6sc7cc(C(C)C)ccc7c56)cc4)c4ccccc34)ccc2c1 ; [2H]c1ccc2cc(-c3c4ccccc4c(-c4ccc(-c5ccc(C(C)C)c6sc7ccccc7c56)cc4)c4ccccc34)ccc2c1]

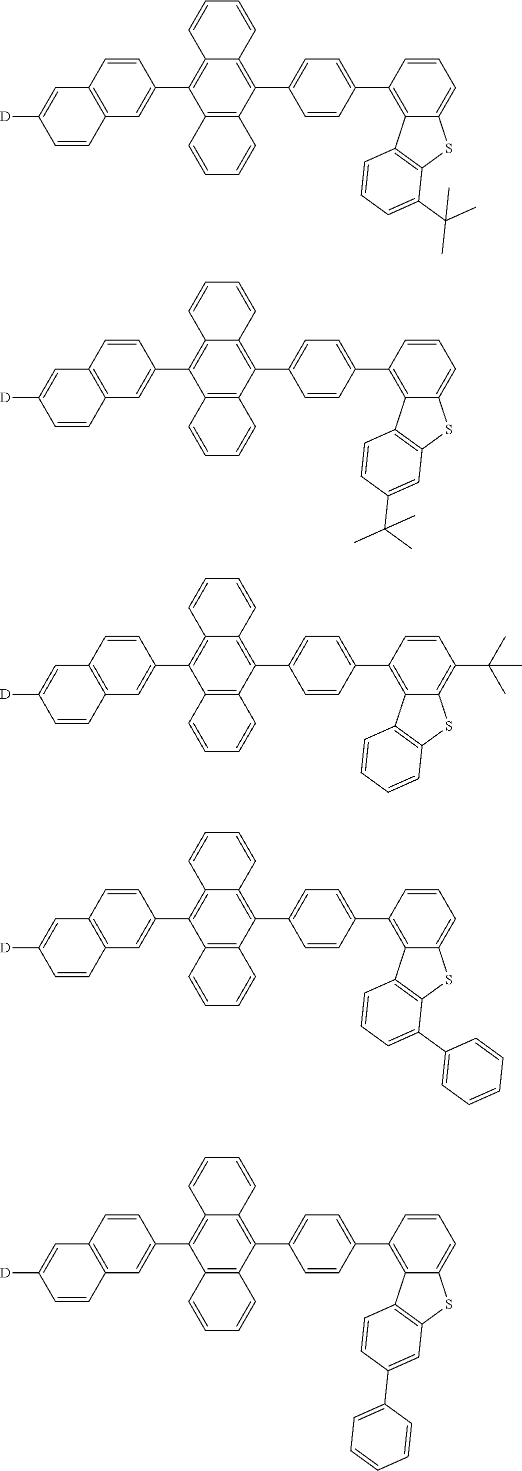 [SMILES: [2H]c1ccc2cc(-c3c4ccccc4c(-c4ccc(-c5cccc6sc7c(C(C)(C)C)cccc7c56)cc4)c4ccccc34)ccc2c1 ; [2H]c1ccc2cc(-c3c4ccccc4c(-c4ccc(-c5cccc6sc7cc(C(C)(C)C)ccc7c56)cc4)c4ccccc34)ccc2c1 ; [2H]c1ccc2cc(-c3c4ccccc4c(-c4ccc(-c5ccc(C(C)(C)C)c6sc7ccccc7c56)cc4)c4ccccc34)ccc2c1 ; [2H]c1ccc2cc(-c3c4ccccc4c(-c4ccc(-c5cccc6sc7c(-c8ccccc8)cccc7c56)cc4)c4ccccc34)ccc2c1 ; [2H]c1ccc2cc(-c3c4ccccc4c(-c4ccc(-c5cccc6sc7cc(-c8ccccc8)ccc7c56)cc4)c4ccccc34)ccc2c1]

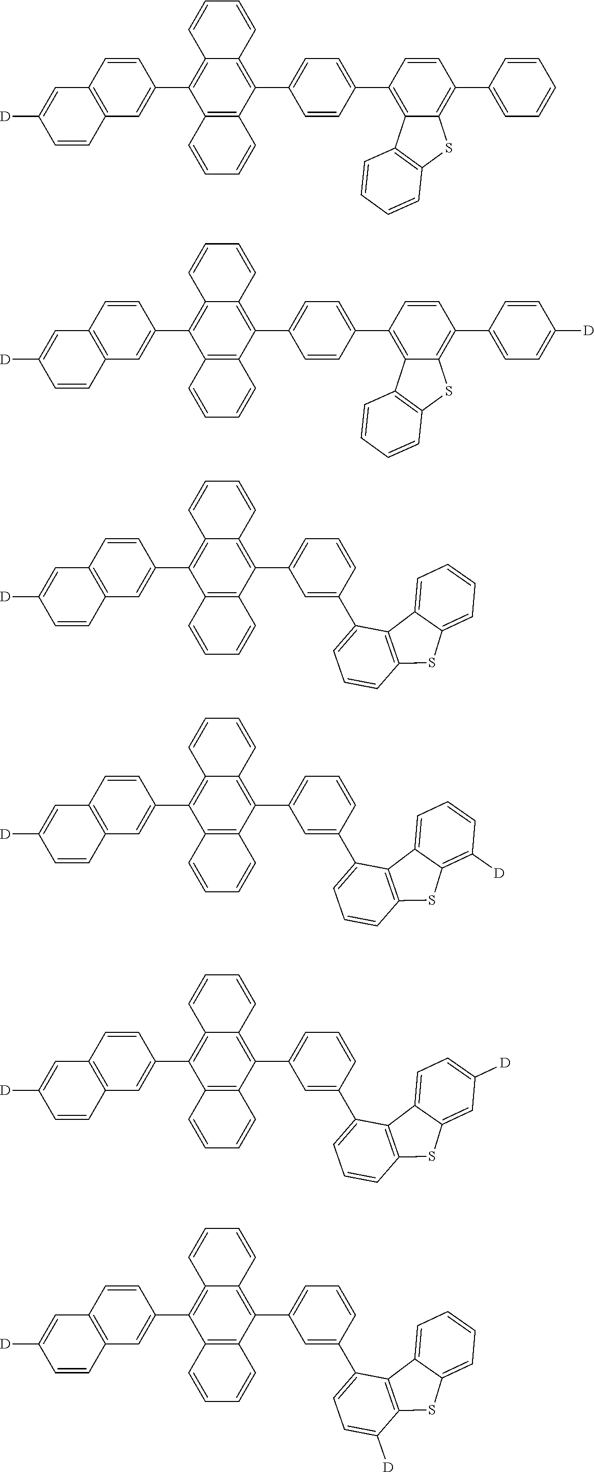

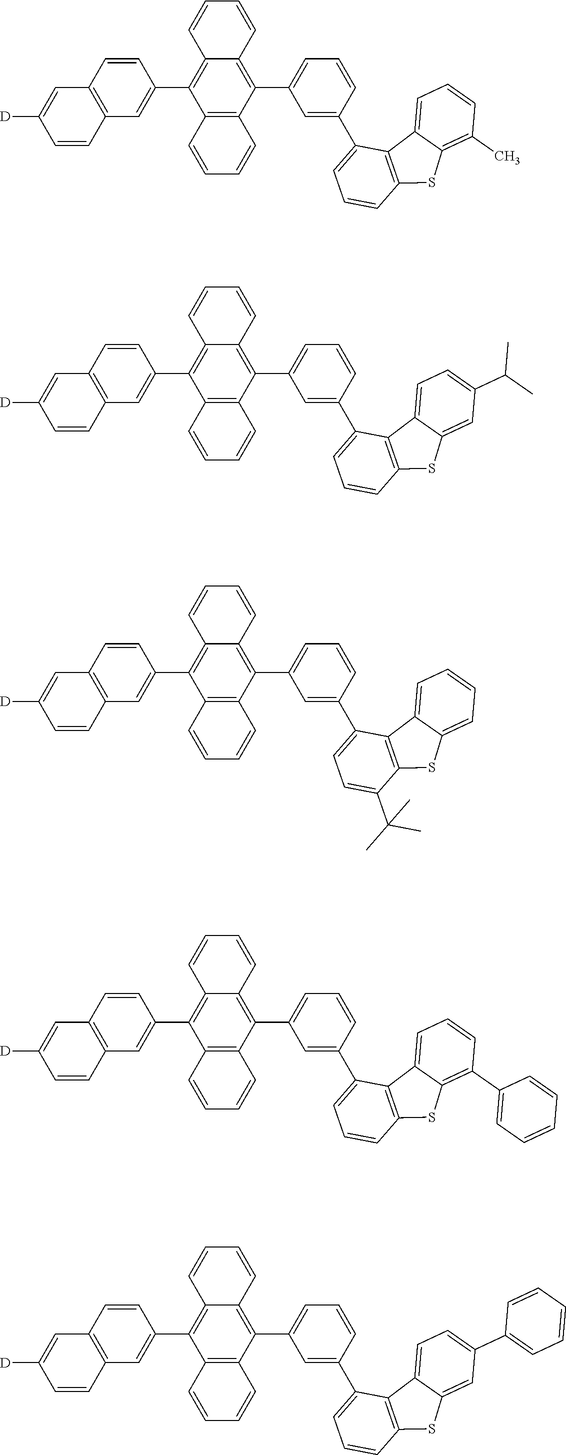 [SMILES: [2H]c1ccc2cc(-c3c4ccccc4c(-c4cccc(-c5cccc6sc7c(C)cccc7c56)c4)c4ccccc34)ccc2c1 ; [2H]c1ccc2cc(-c3c4ccccc4c(-c4cccc(-c5cccc6sc7cc(C(C)C)ccc7c56)c4)c4ccccc34)ccc2c1 ; [2H]c1ccc2cc(-c3c4ccccc4c(-c4cccc(-c5ccc(C(C)(C)C)c6sc7ccccc7c56)c4)c4ccccc34)ccc2c1 ; [2H]c1ccc2cc(-c3c4ccccc4c(-c4cccc(-c5cccc6sc7c(-c8ccccc8)cccc7c56)c4)c4ccccc34)ccc2c1 ; [2H]c1ccc2cc(-c3c4ccccc4c(-c4cccc(-c5cccc6sc7cc(-c8ccccc8)ccc7c56)c4)c4ccccc34)ccc2c1]

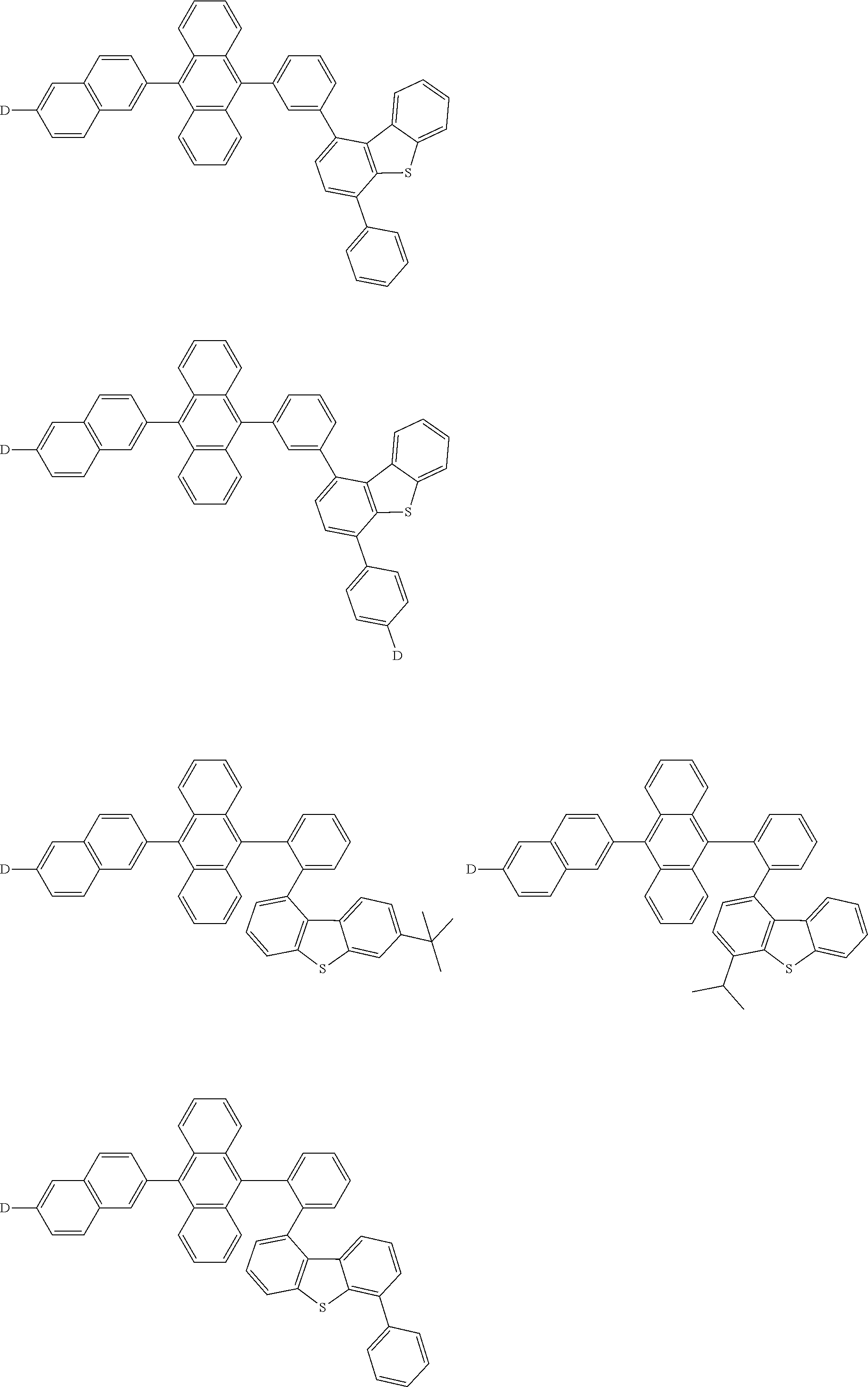 [SMILES: [2H]c1ccc2cc(-c3c4ccccc4c(-c4cccc(-c5ccc(-c6ccccc6)c6sc7ccccc7c56)c4)c4ccccc34)ccc2c1 ; [2H]c1ccc(-c2ccc(-c3cccc(-c4c5ccccc5c(-c5ccc6cc([2H])ccc6c5)c5ccccc45)c3)c3c2sc2ccccc23)cc1 ; [2H]c1ccc2cc(-c3c4ccccc4c(-c4ccccc4-c4cccc5sc6cc(C(C)(C)C)ccc6c45)c4ccccc34)ccc2c1 ; [2H]c1ccc2cc(-c3c4ccccc4c(-c4ccccc4-c4ccc(C(C)C)c5sc6ccccc6c45)c4ccccc34)ccc2c1 ; [2H]c1ccc2cc(-c3c4ccccc4c(-c4ccccc4-c4cccc5sc6c(-c7ccccc7)cccc6c45)c4ccccc34)ccc2c1]

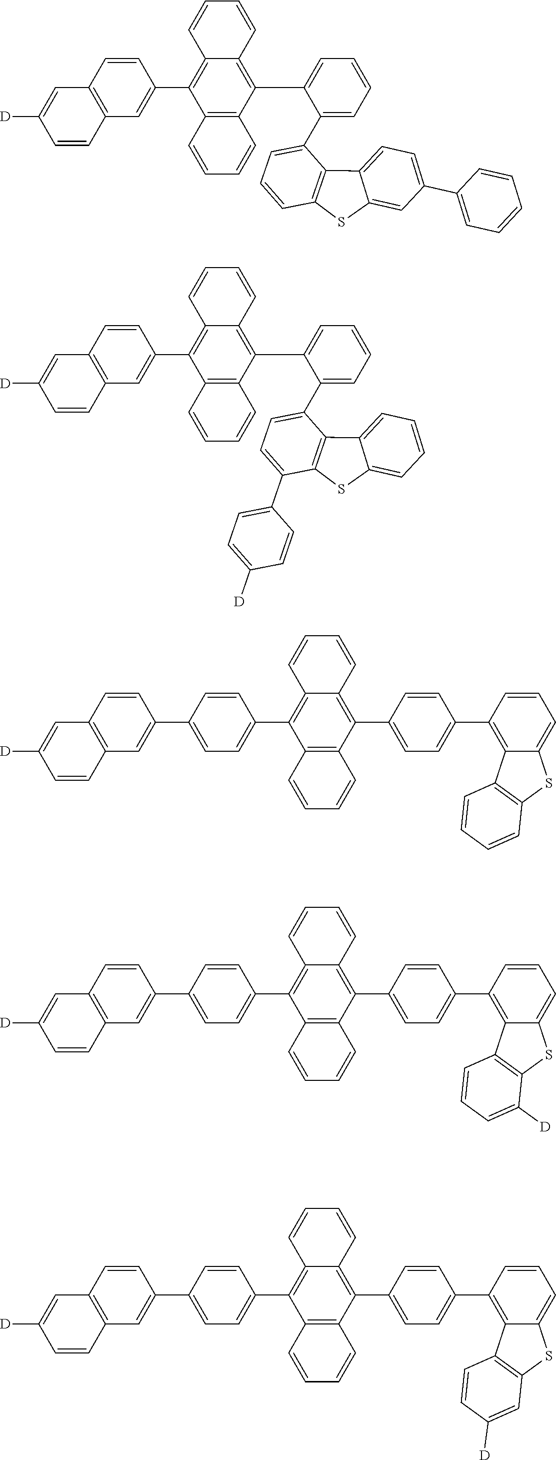 [SMILES: [2H]c1ccc2cc(-c3c4ccccc4c(-c4ccccc4-c4cccc5sc6cc(-c7ccccc7)ccc6c45)c4ccccc34)ccc2c1 ; [2H]c1ccc(-c2ccc(-c3ccccc3-c3c4ccccc4c(-c4ccc5cc([2H])ccc5c4)c4ccccc34)c3c2sc2ccccc23)cc1 ; [2H]c1ccc2cc(-c3ccc(-c4c5ccccc5c(-c5ccc(-c6cccc7sc8ccccc8c67)cc5)c5ccccc45)cc3)ccc2c1 ; [2H]c1ccc2cc(-c3ccc(-c4c5ccccc5c(-c5ccc(-c6cccc7sc8c([2H])cccc8c67)cc5)c5ccccc45)cc3)ccc2c1 ; [2H]c1ccc2cc(-c3ccc(-c4c5ccccc5c(-c5ccc(-c6cccc7sc8cc([2H])ccc8c67)cc5)c5ccccc45)cc3)ccc2c1]

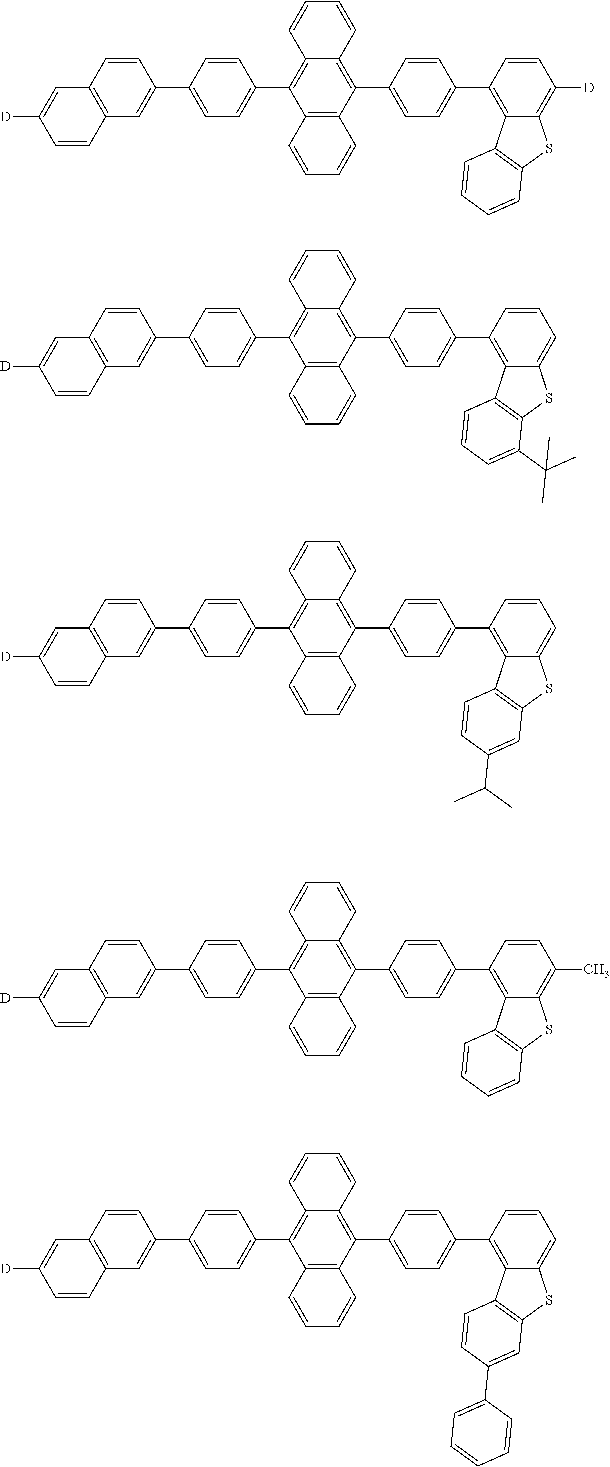 [SMILES: [2H]c1ccc2cc(-c3ccc(-c4c5ccccc5c(-c5ccc(-c6ccc([2H])c7sc8ccccc8c67)cc5)c5ccccc45)cc3)ccc2c1 ; [2H]c1ccc2cc(-c3ccc(-c4c5ccccc5c(-c5ccc(-c6cccc7sc8c(C(C)(C)C)cccc8c67)cc5)c5ccccc45)cc3)ccc2c1 ; [2H]c1ccc2cc(-c3ccc(-c4c5ccccc5c(-c5ccc(-c6cccc7sc8cc(C(C)C)ccc8c67)cc5)c5ccccc45)cc3)ccc2c1 ; [2H]c1ccc2cc(-c3ccc(-c4c5ccccc5c(-c5ccc(-c6ccc(C)c7sc8ccccc8c67)cc5)c5ccccc45)cc3)ccc2c1 ; [2H]c1ccc2cc(-c3ccc(-c4c5ccccc5c(-c5ccc(-c6cccc7sc8cc(-c9ccccc9)ccc8c67)cc5)c5ccccc45)cc3)ccc2c1]

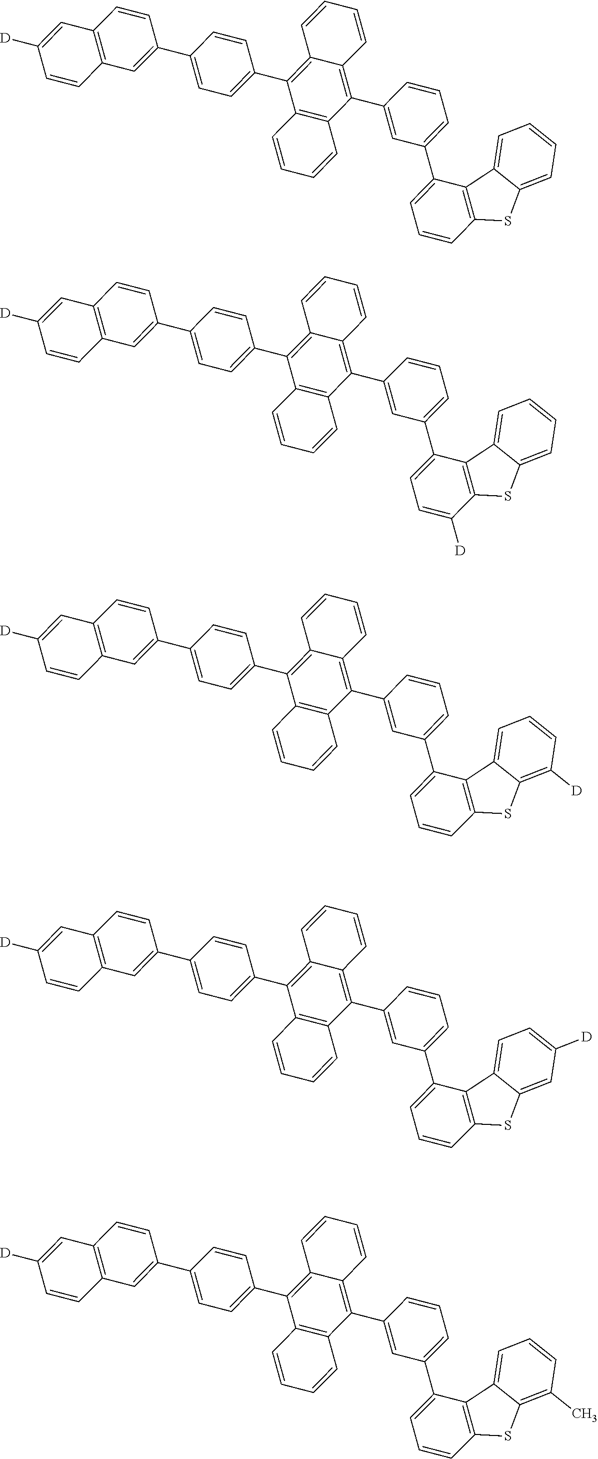 [SMILES: [2H]c1ccc2cc(-c3ccc(-c4c5ccccc5c(-c5cccc(-c6cccc7sc8ccccc8c67)c5)c5ccccc45)cc3)ccc2c1 ; [2H]c1ccc2cc(-c3ccc(-c4c5ccccc5c(-c5cccc(-c6ccc([2H])c7sc8ccccc8c67)c5)c5ccccc45)cc3)ccc2c1 ; [2H]c1ccc2cc(-c3ccc(-c4c5ccccc5c(-c5cccc(-c6cccc7sc8c([2H])cccc8c67)c5)c5ccccc45)cc3)ccc2c1 ; [2H]c1ccc2cc(-c3ccc(-c4c5ccccc5c(-c5cccc(-c6cccc7sc8cc([2H])ccc8c67)c5)c5ccccc45)cc3)ccc2c1 ; [2H]c1ccc2cc(-c3ccc(-c4c5ccccc5c(-c5cccc(-c6cccc7sc8c(C)cccc8c67)c5)c5ccccc45)cc3)ccc2c1]

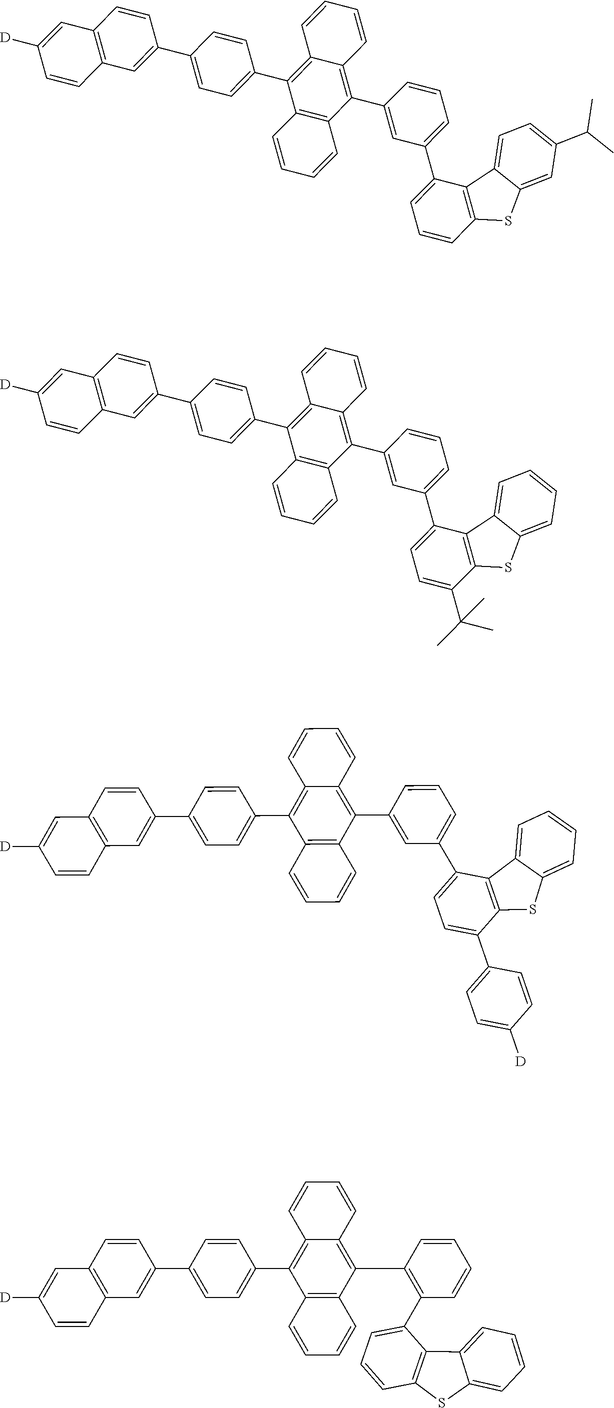 [SMILES: [2H]c1ccc2cc(-c3ccc(-c4c5ccccc5c(-c5cccc(-c6cccc7sc8cc(C(C)C)ccc8c67)c5)c5ccccc45)cc3)ccc2c1 ; [2H]c1ccc2cc(-c3ccc(-c4c5ccccc5c(-c5cccc(-c6ccc(C(C)(C)C)c7sc8ccccc8c67)c5)c5ccccc45)cc3)ccc2c1 ; [2H]c1ccc(-c2ccc(-c3cccc(-c4c5ccccc5c(-c5ccc(-c6ccc7cc([2H])ccc7c6)cc5)c5ccccc45)c3)c3c2sc2ccccc23)cc1 ; [2H]c1ccc2cc(-c3ccc(-c4c5ccccc5c(-c5ccccc5-c5cccc6sc7ccccc7c56)c5ccccc45)cc3)ccc2c1]

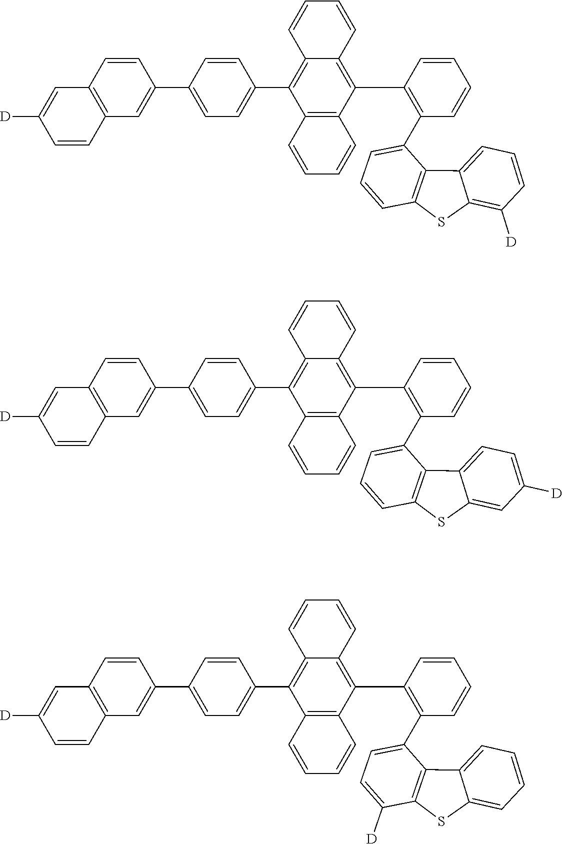 [SMILES: [2H]c1ccc2cc(-c3ccc(-c4c5ccccc5c(-c5ccccc5-c5cccc6sc7c([2H])cccc7c56)c5ccccc45)cc3)ccc2c1 ; [2H]c1ccc2cc(-c3ccc(-c4c5ccccc5c(-c5ccccc5-c5cccc6sc7cc([2H])ccc7c56)c5ccccc45)cc3)ccc2c1 ; [2H]c1ccc2cc(-c3ccc(-c4c5ccccc5c(-c5ccccc5-c5ccc([2H])c6sc7ccccc7c56)c5ccccc45)cc3)ccc2c1]

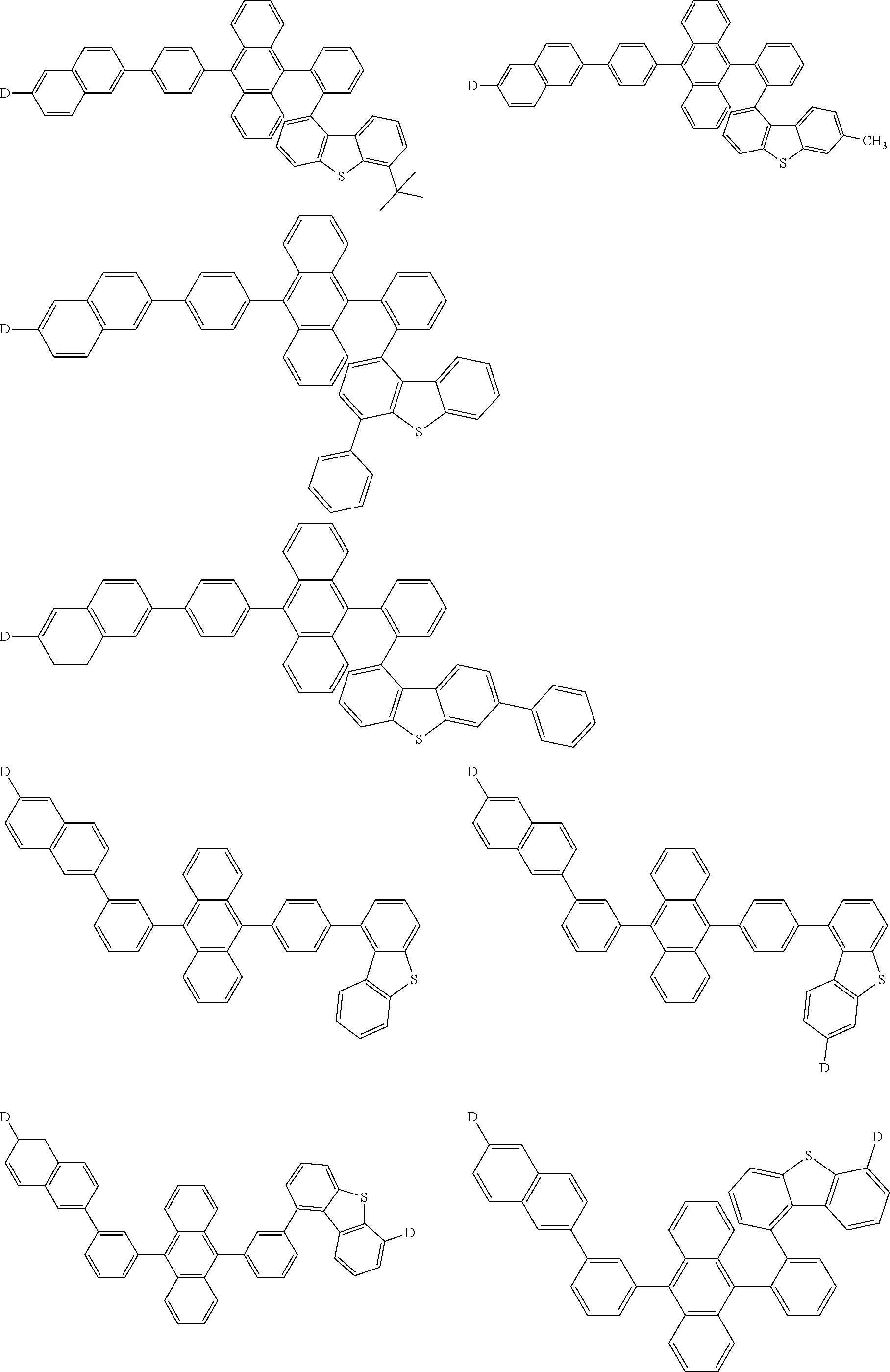

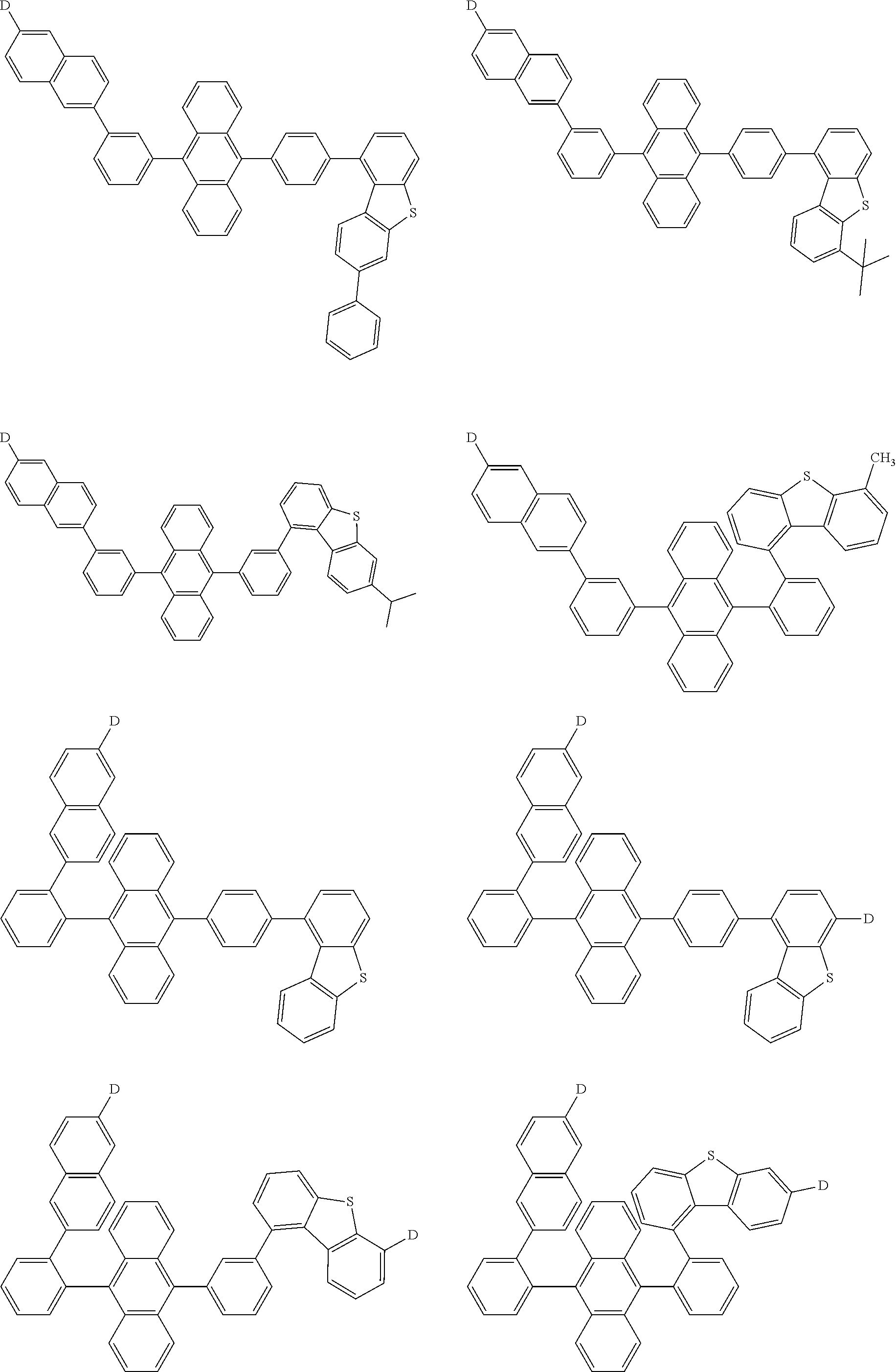

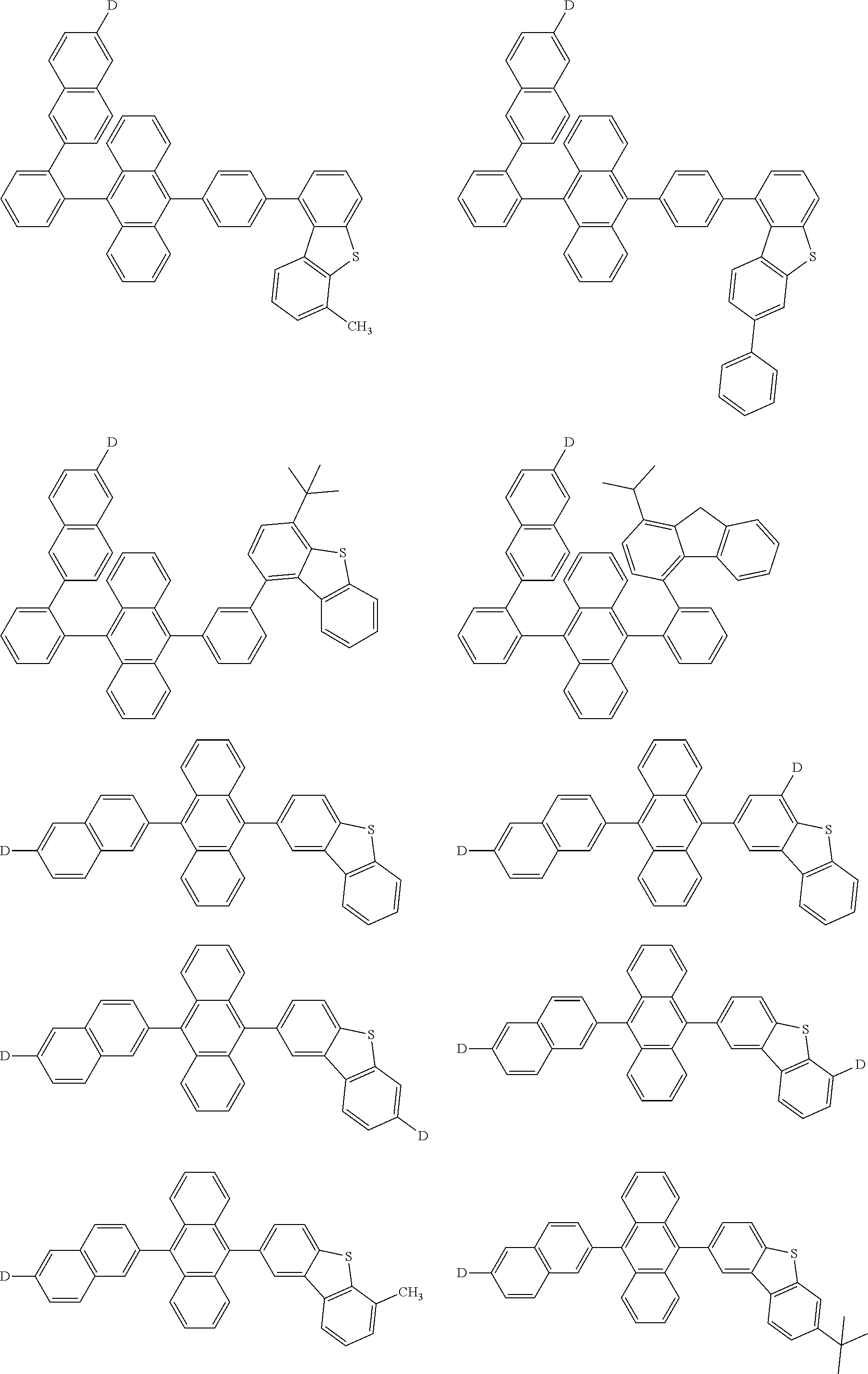

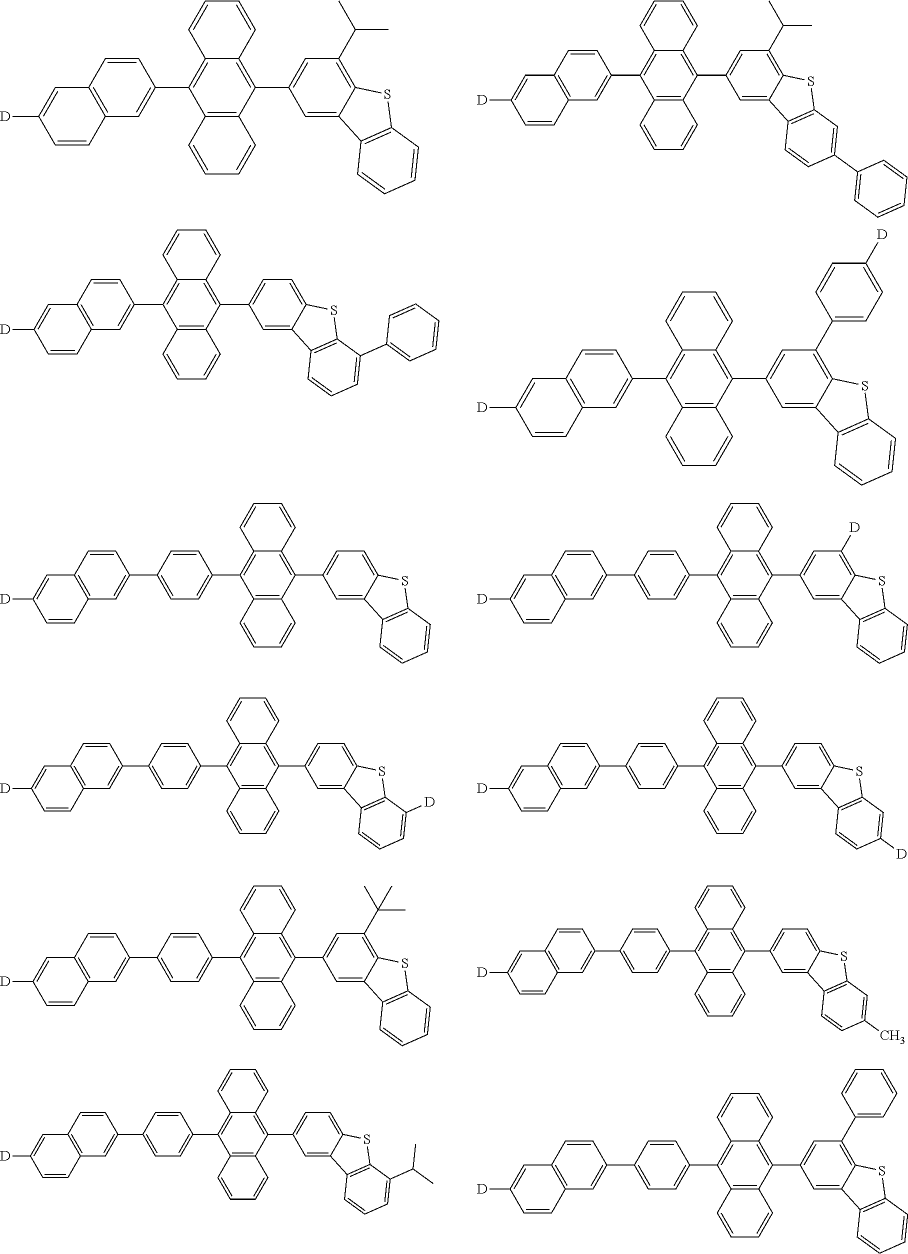

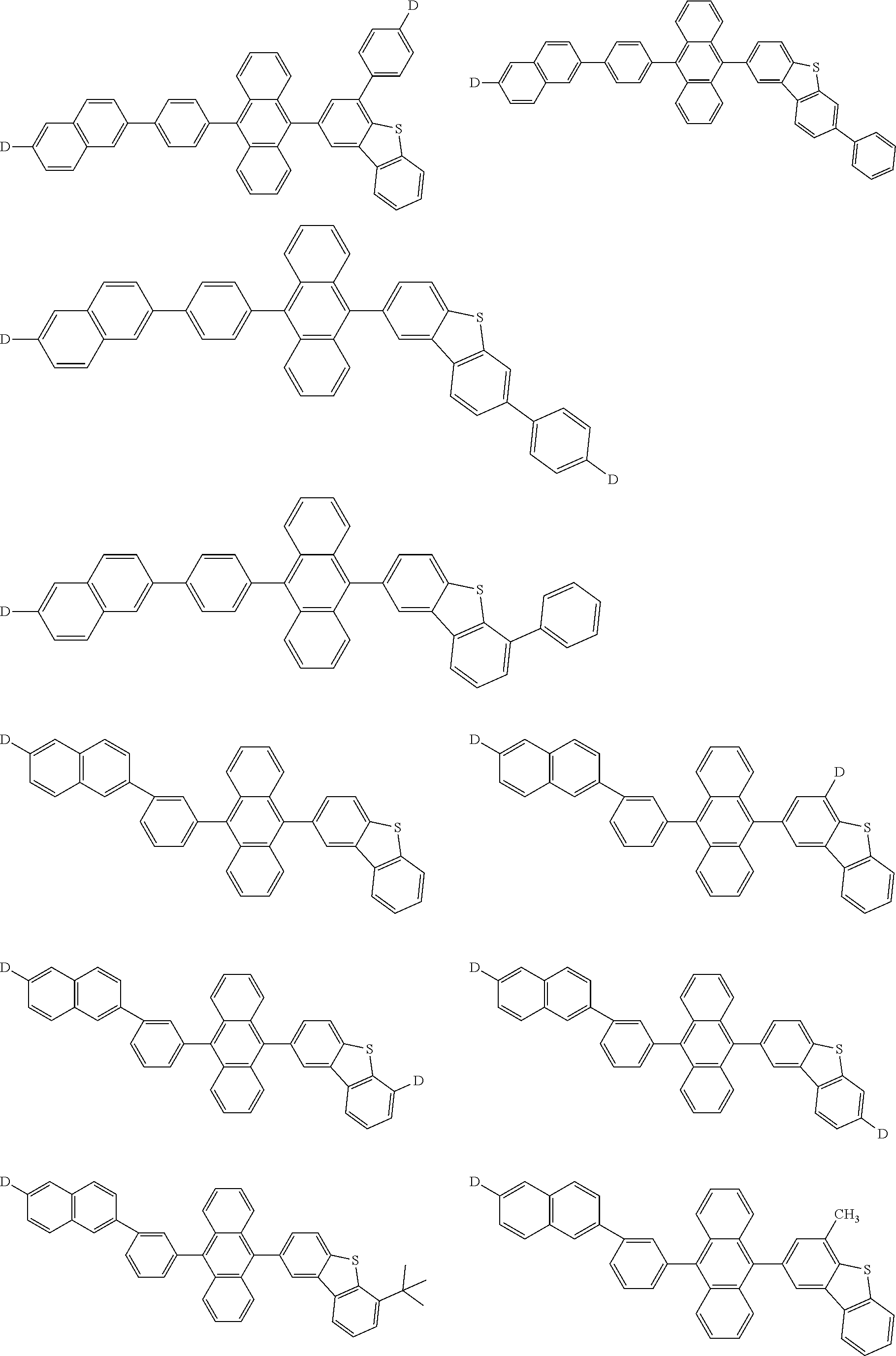

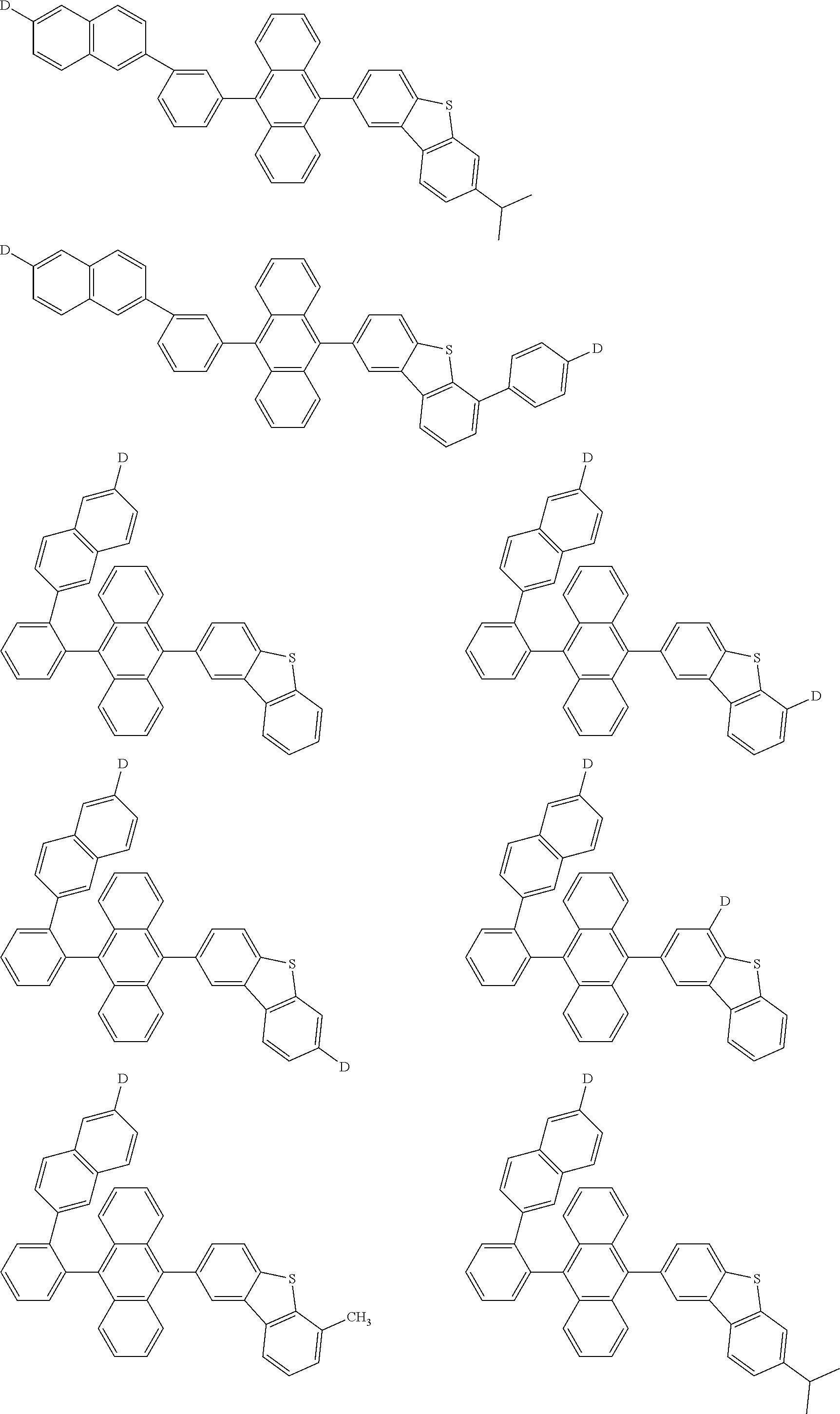

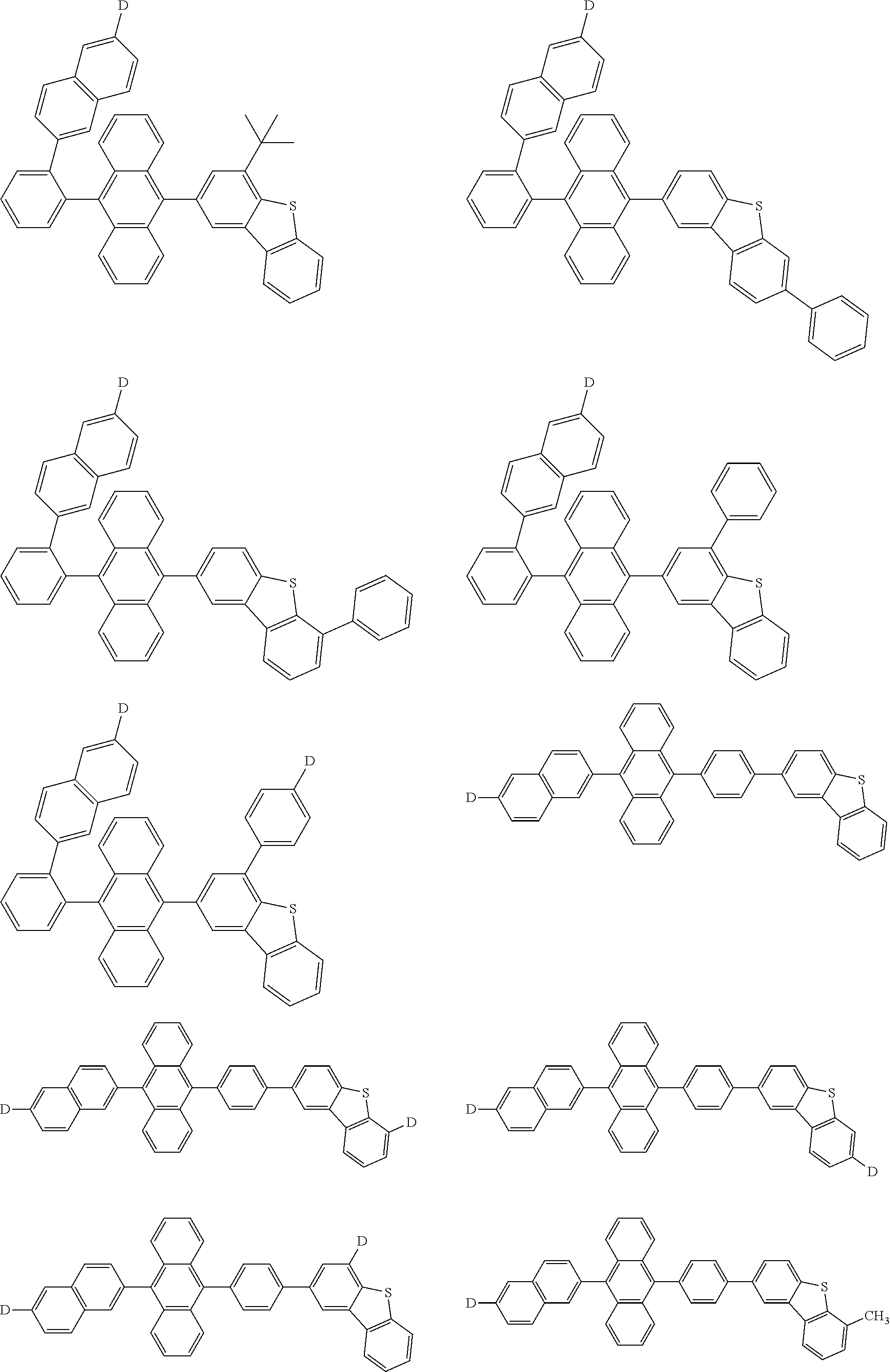

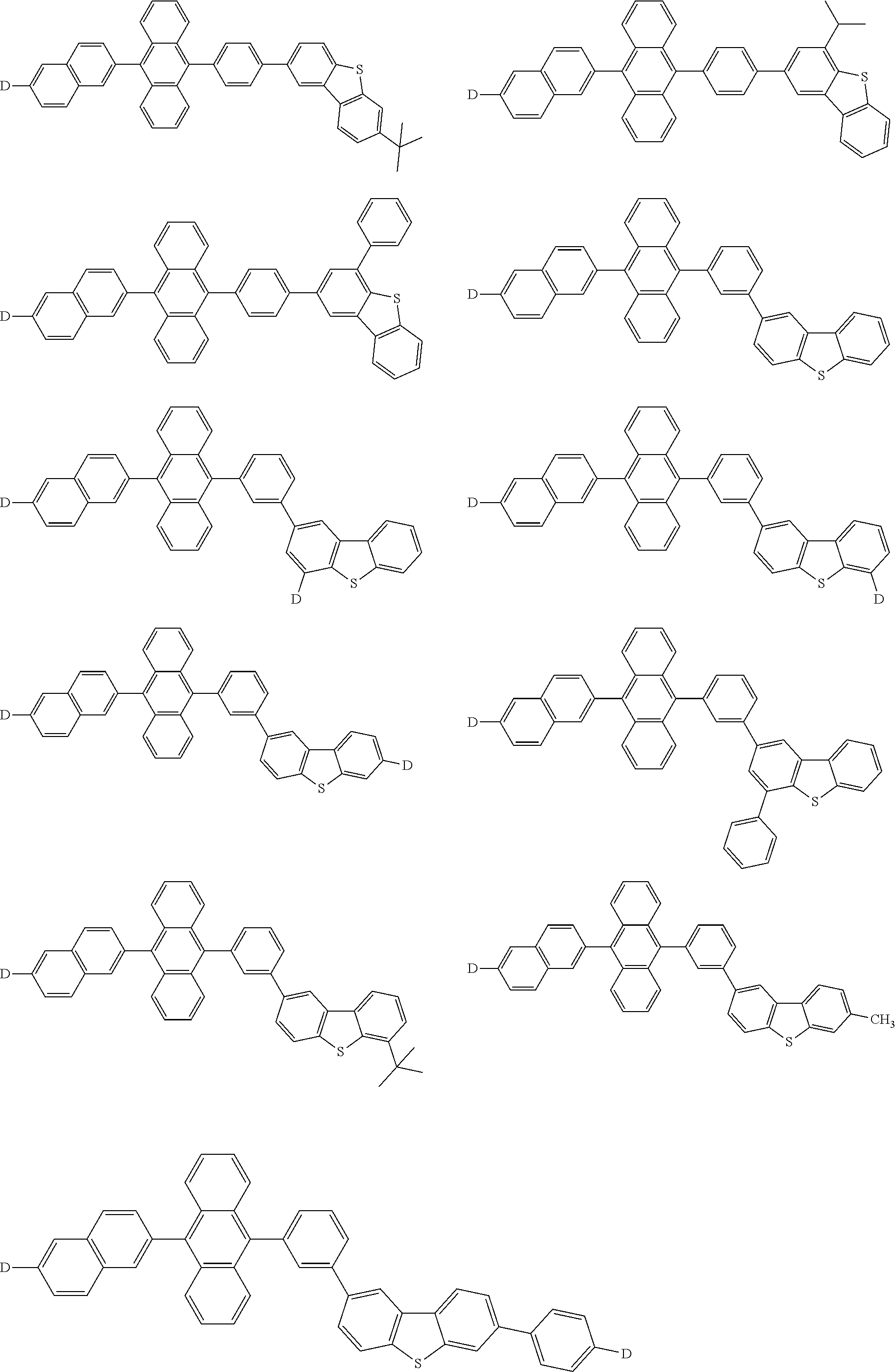

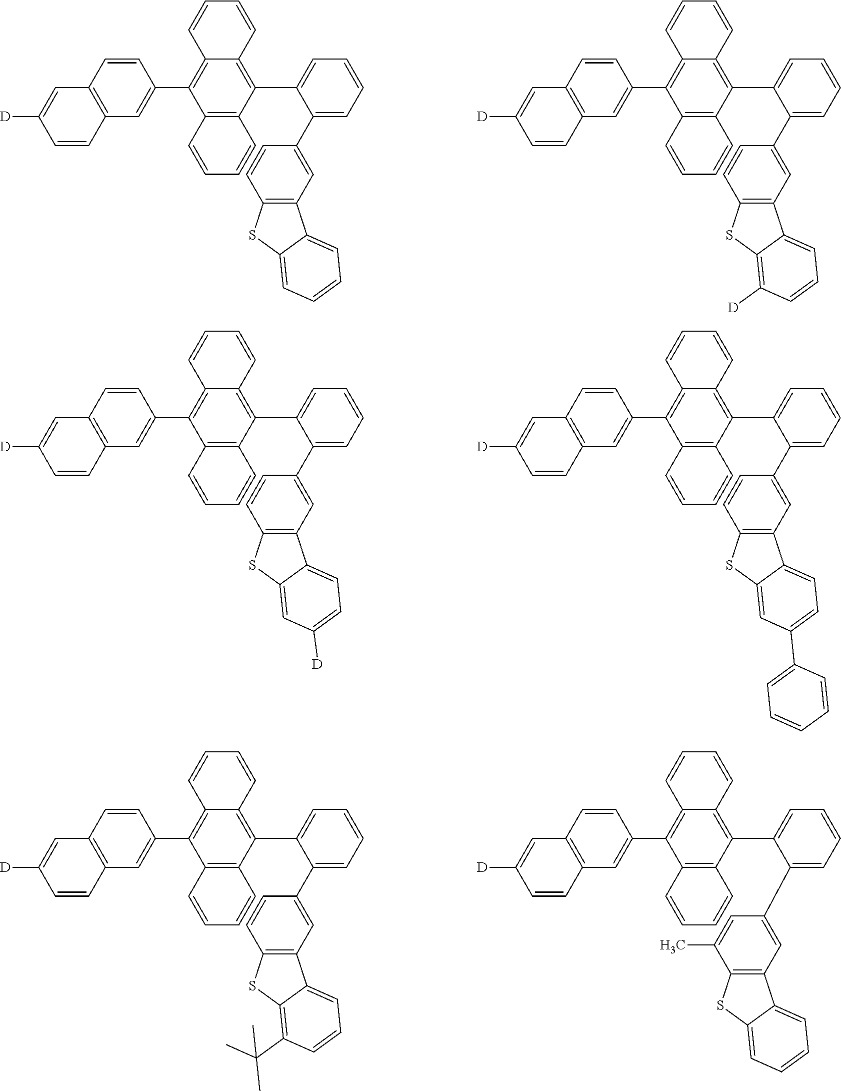

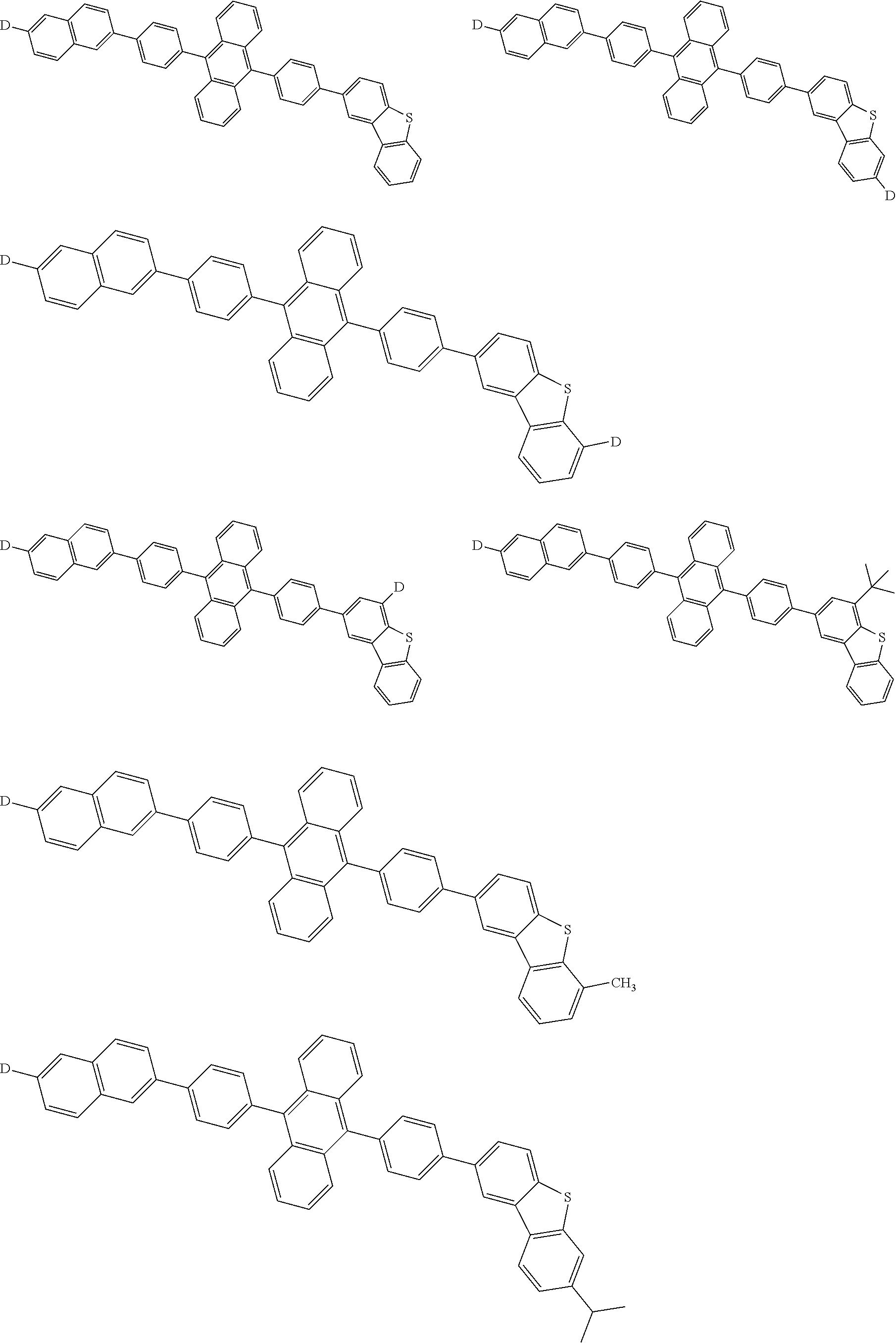

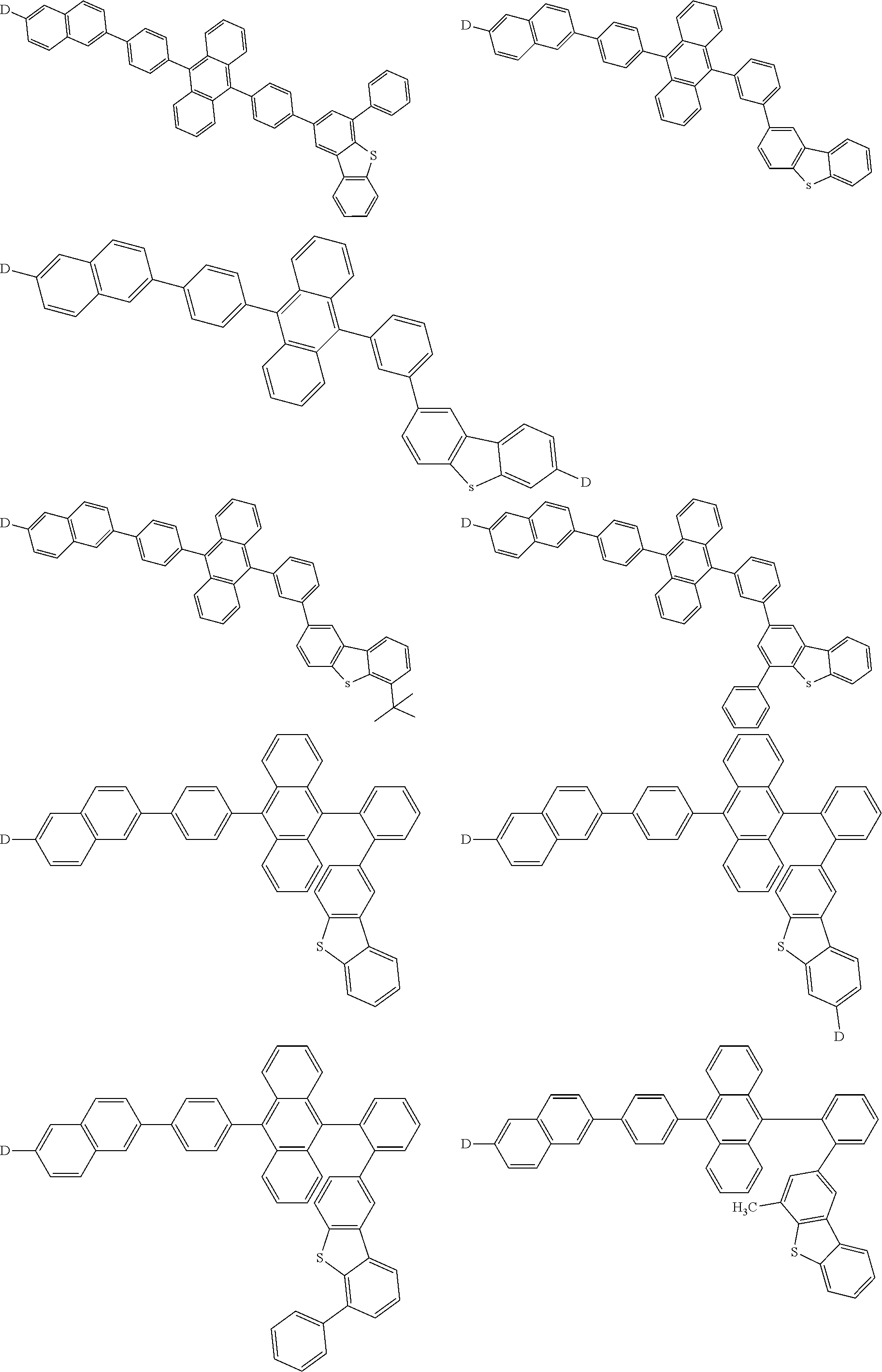

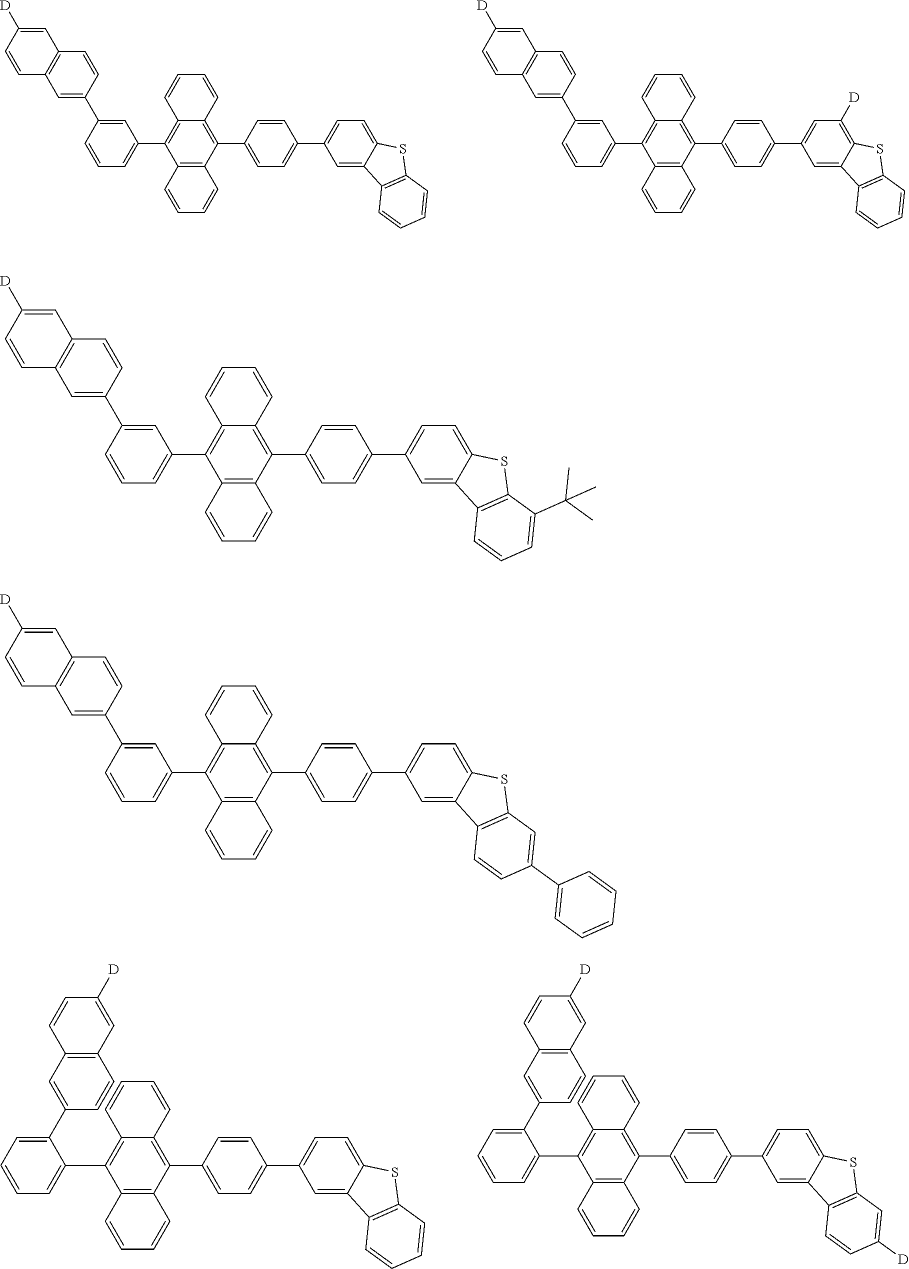

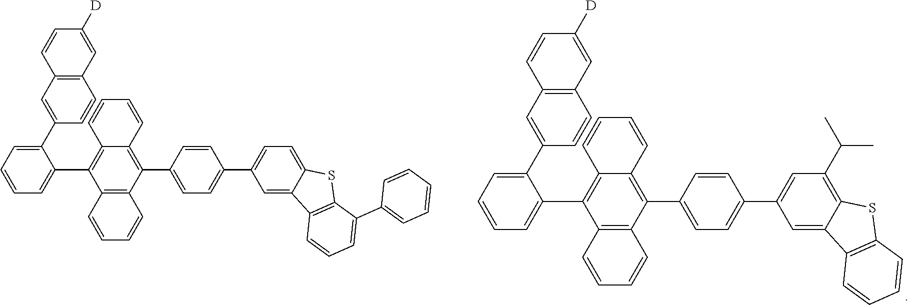 [SMILES: [2H]c1ccc2cc(-c3ccccc3-c3c4ccccc4c(-c4ccc(-c5ccc6sc7c(-c8ccccc8)cccc7c6c5)cc4)c4ccccc34)ccc2c1 ; [2H]c1ccc2cc(-c3ccccc3-c3c4ccccc4c(-c4ccc(-c5cc(C(C)C)c6sc7ccccc7c6c5)cc4)c4ccccc34)ccc2c1]

12. A mixture, comprising a first organic compound and a second organic compound, wherein the first organic compound is the anthracene-based compound according to claim 1, and the second organic compound is at least one material selected from a hole injection material, a hole transport material, an electron transport material, an electron injection material, an electron barrier material, a hole barrier material, a light-emitting guest material, a light-emitting host material, and an organic dye.

13. The mixture according to claim 12, wherein the second organic compound is selected from organic compounds represented by formula (III-1) or formula (III-2): 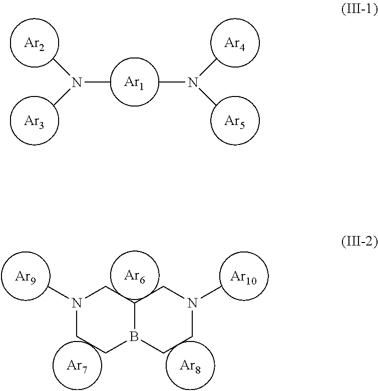
wherein each of Ar₁ to Ar₁₀ is independently selected from a substituted or unsubstituted aromatic group containing 6-40 ring atoms or a substituted or unsubstituted heteroaromatic group containing 5-40 ring atoms, a ring is not formed between any two of Ar₁ to Ar₅, a single ring is formed or multi rings are formed among Ar₁ to Ar₅, a ring is not formed between any two of Ar₆ to Ar₁₀, or a single ring is formed or multi rings are formed among Ar₆ to Ar₁₀.

14. The mixture according to claim 13, wherein Ar₁ is selected from groups represented by formula (D-1), formula (D-2), and formula (D-3); 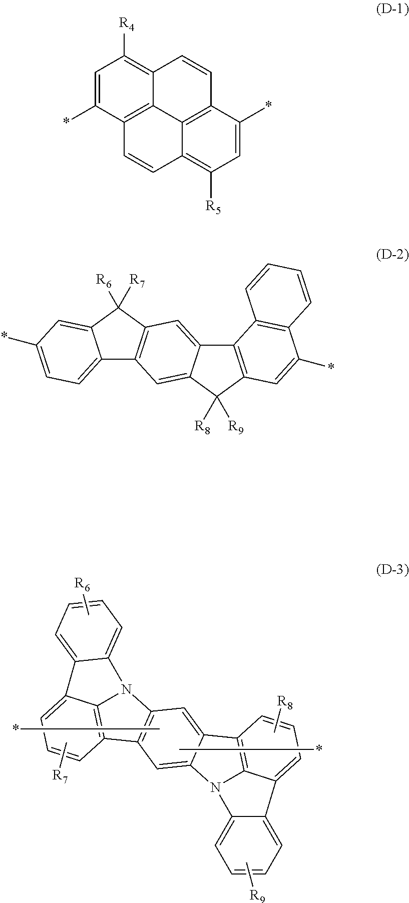
wherein each of R₄ to R₈ is independently selected from —H, -D, a C₁-C₂₀ linear alkyl group, a C₁-C₂₀ alkoxy group, a C₁-C₂₀ thioalkoxy group, a C₃-C₂₀ branched or cyclic alkyl group, a C₃-C₂₀ alkoxy group, a C₃-C₂₀ thioalkoxy group, a silyl group, a C₁-C₂₀ ketone group, a C₂-C₂₀ alkoxycarbonyl group, a C₇-C₂₀ aroxycarbonyl group, a cyano group, a carbamoyl group, a haloformyl group, a formyl group, an isocyano group, an isocyanate group, a thiocyanate ester group, an isothiocyanate ester group, a hydroxyl group, a nitro group, an amino group, —CF₃, —Cl, —Br, —F, —I, a substituted or unsubstituted C₂-C₂₀ alkenyl group, a substituted or unsubstituted aromatic group containing 5-30 ring atoms, a substituted or unsubstituted heteroaromatic group containing 5 to 30 ring atoms, an aryloxy group containing 5-30 ring atoms, a heteroaryloxy group containing 5-30 ring atoms, and combinations of these groups; and
* indicates a binding site.

15. The mixture according to claim 13, wherein the second organic compound represented by the formula (III-1) is selected from following compounds:

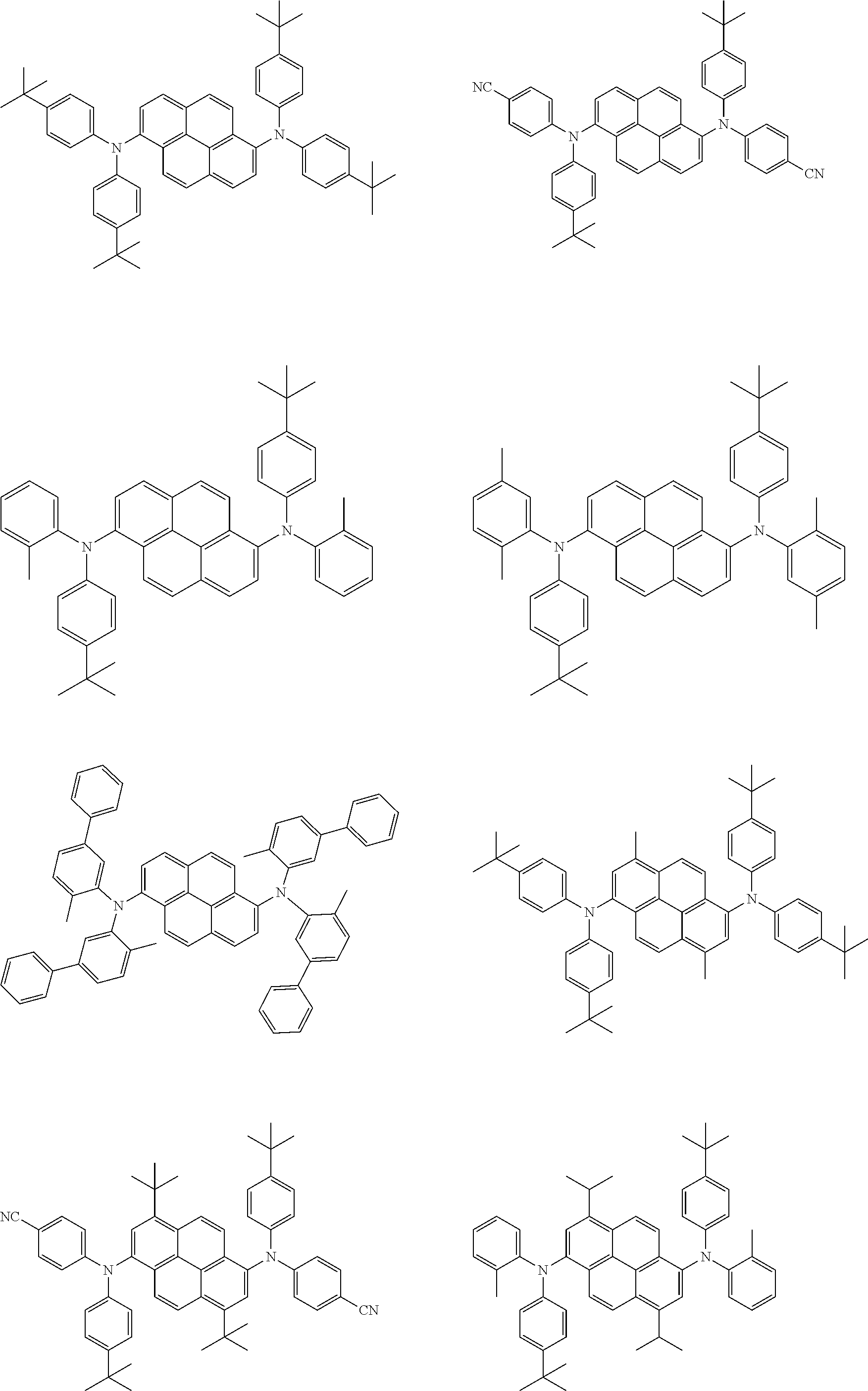

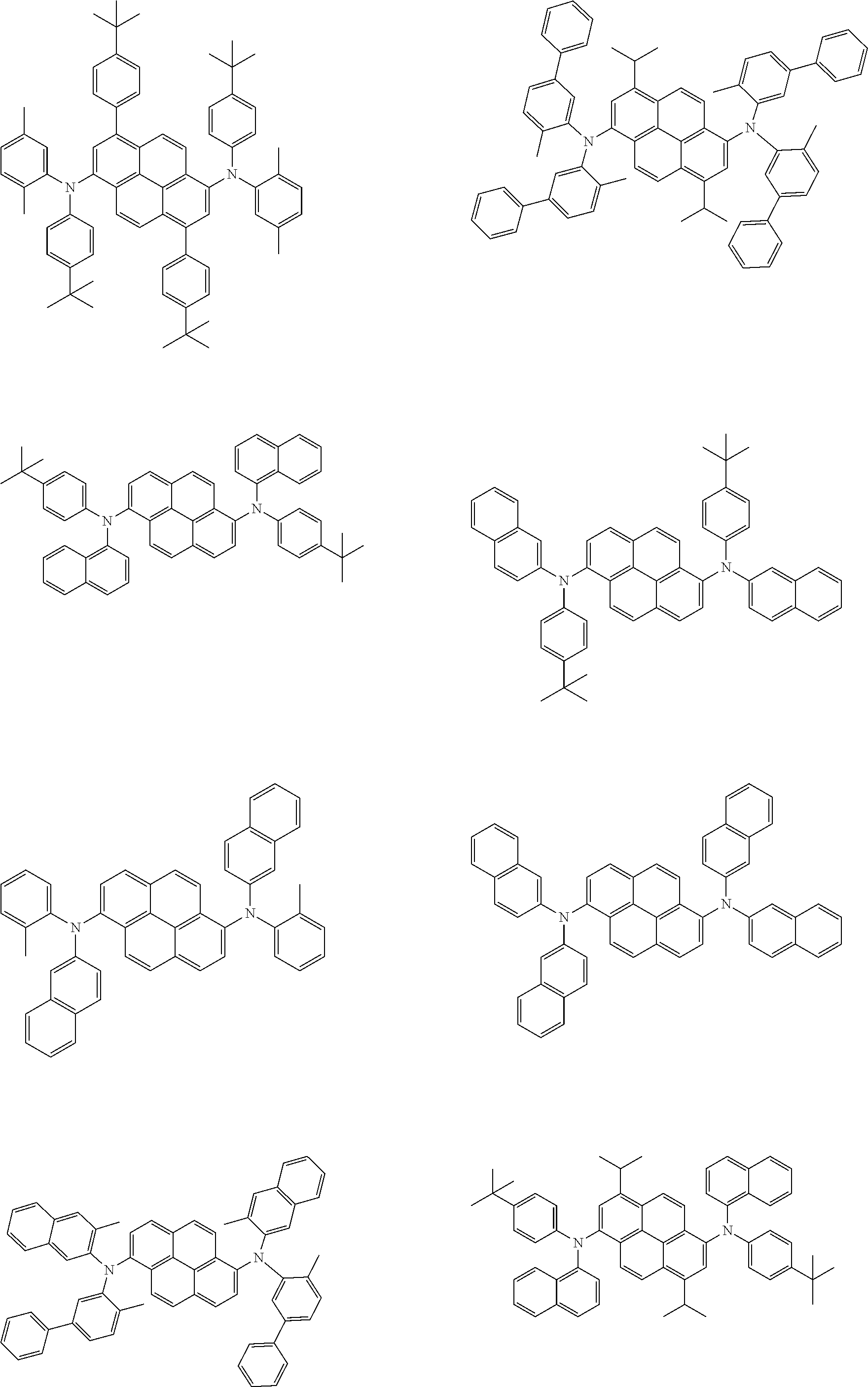

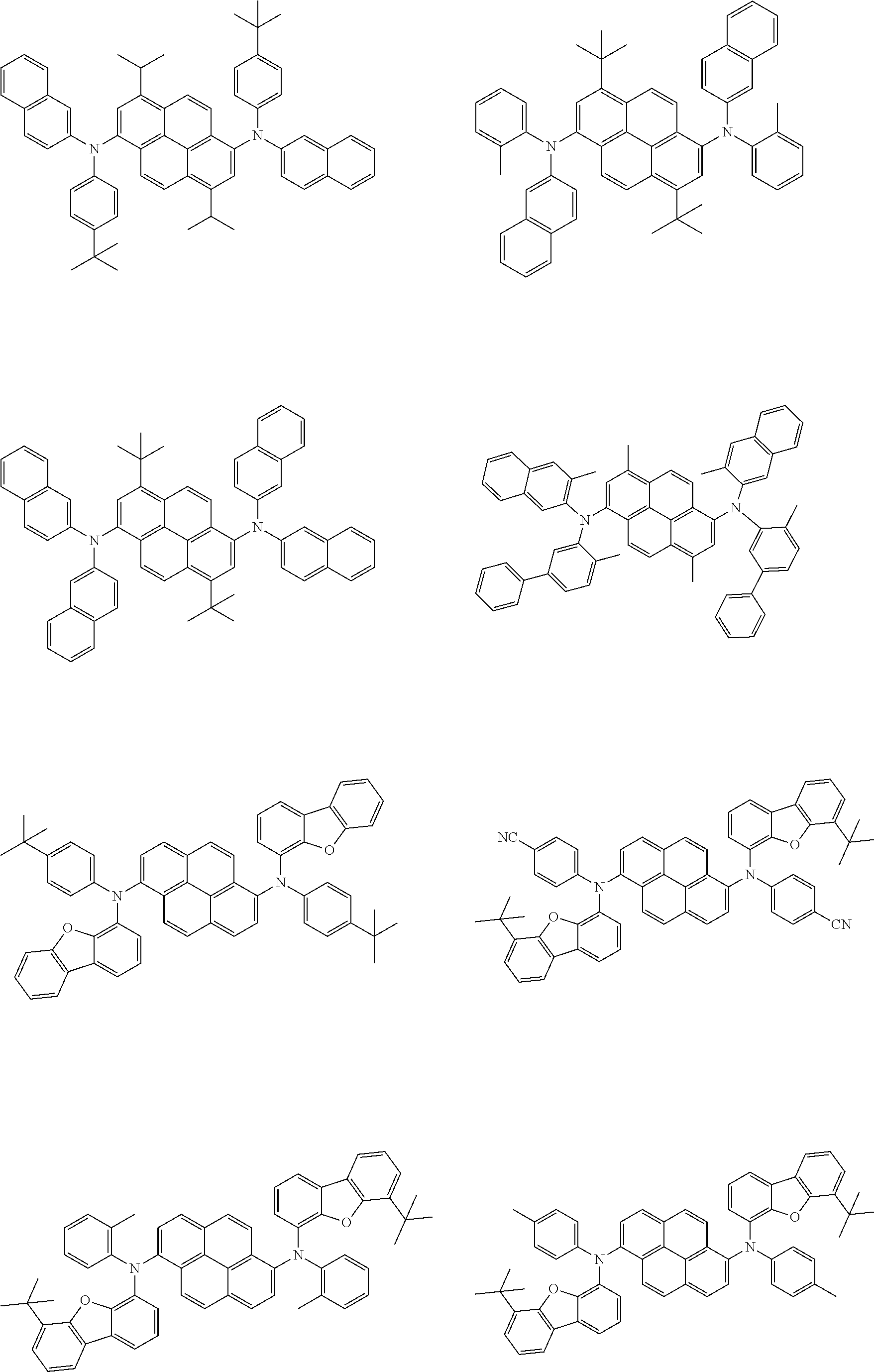

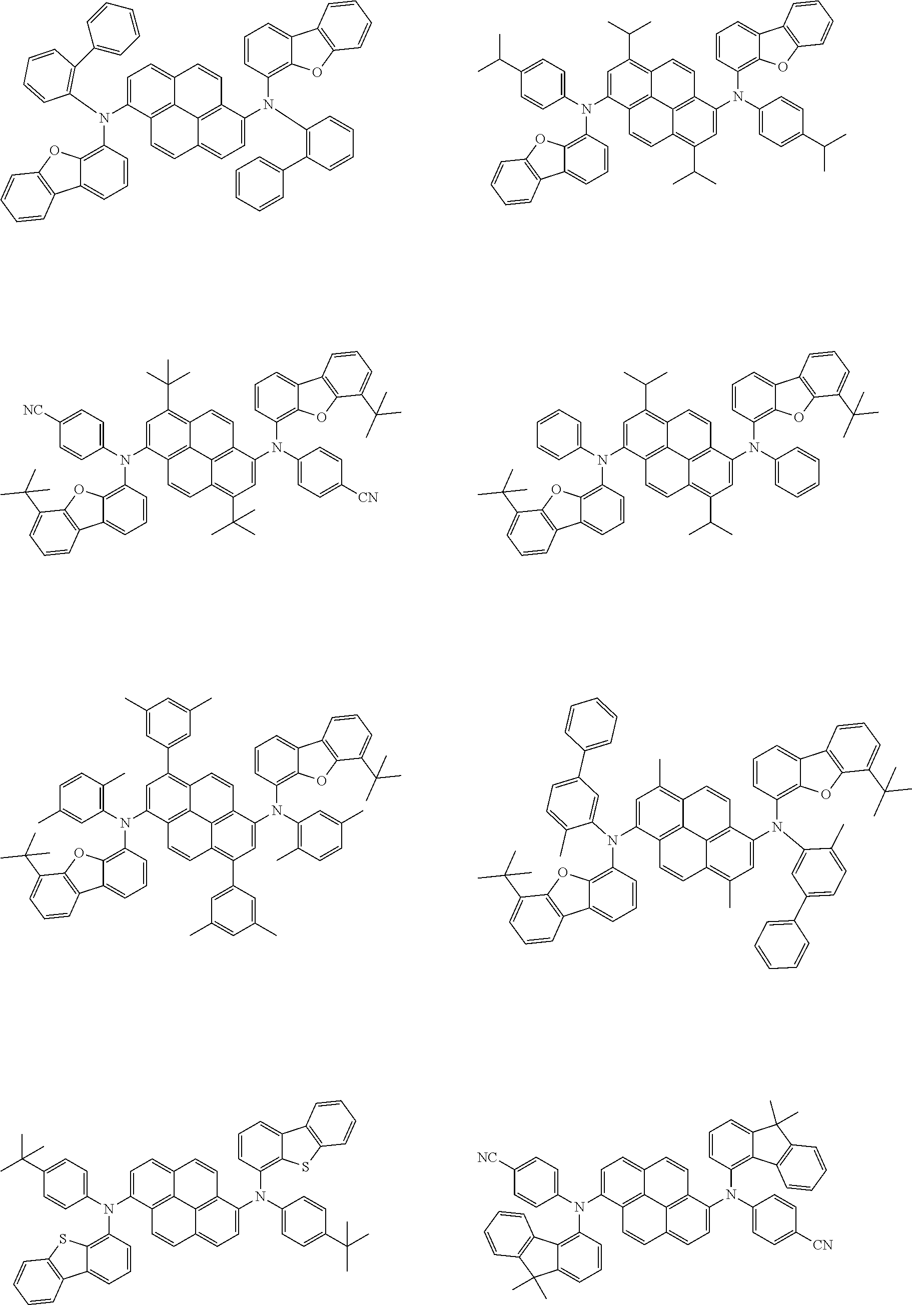

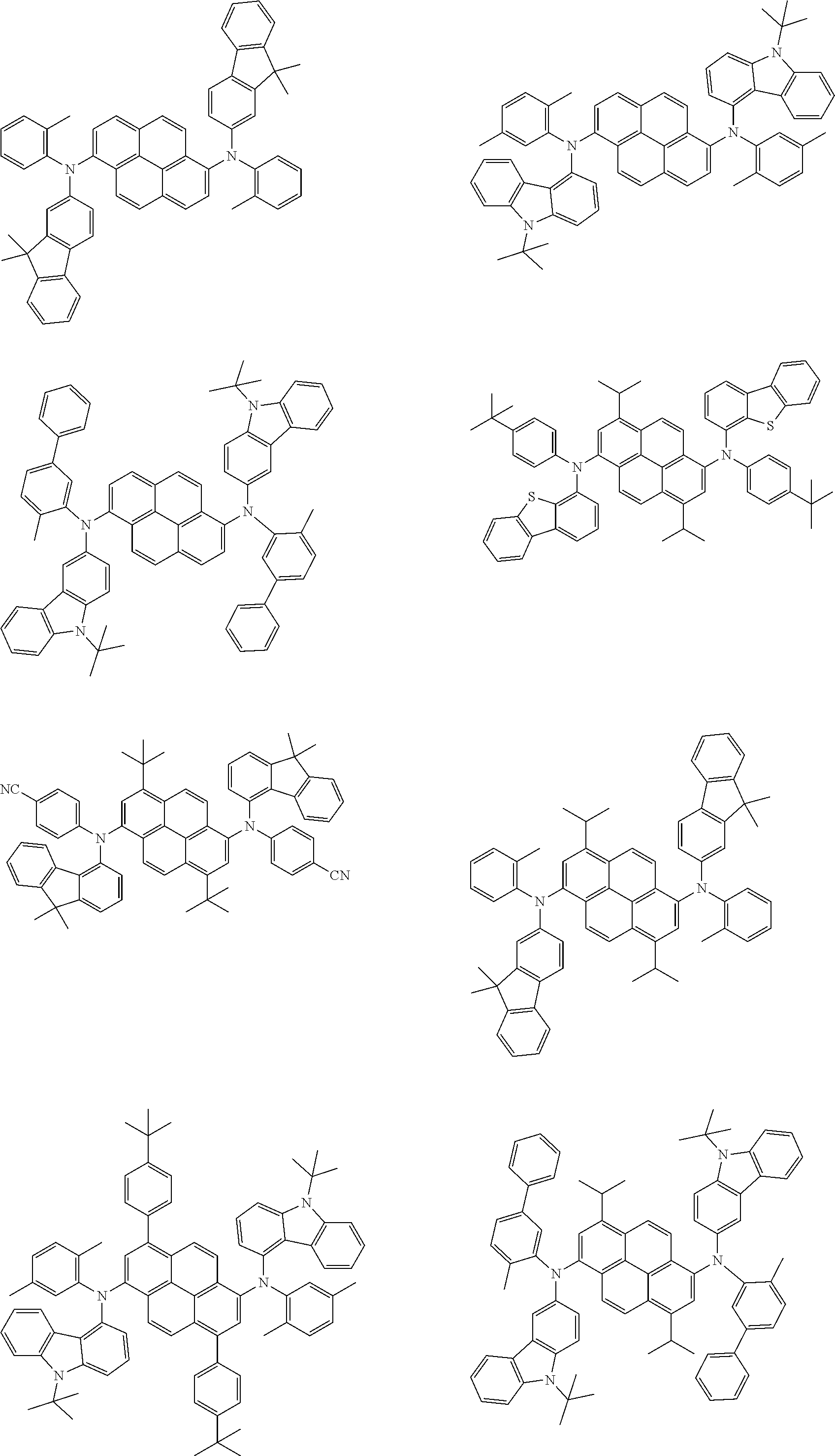

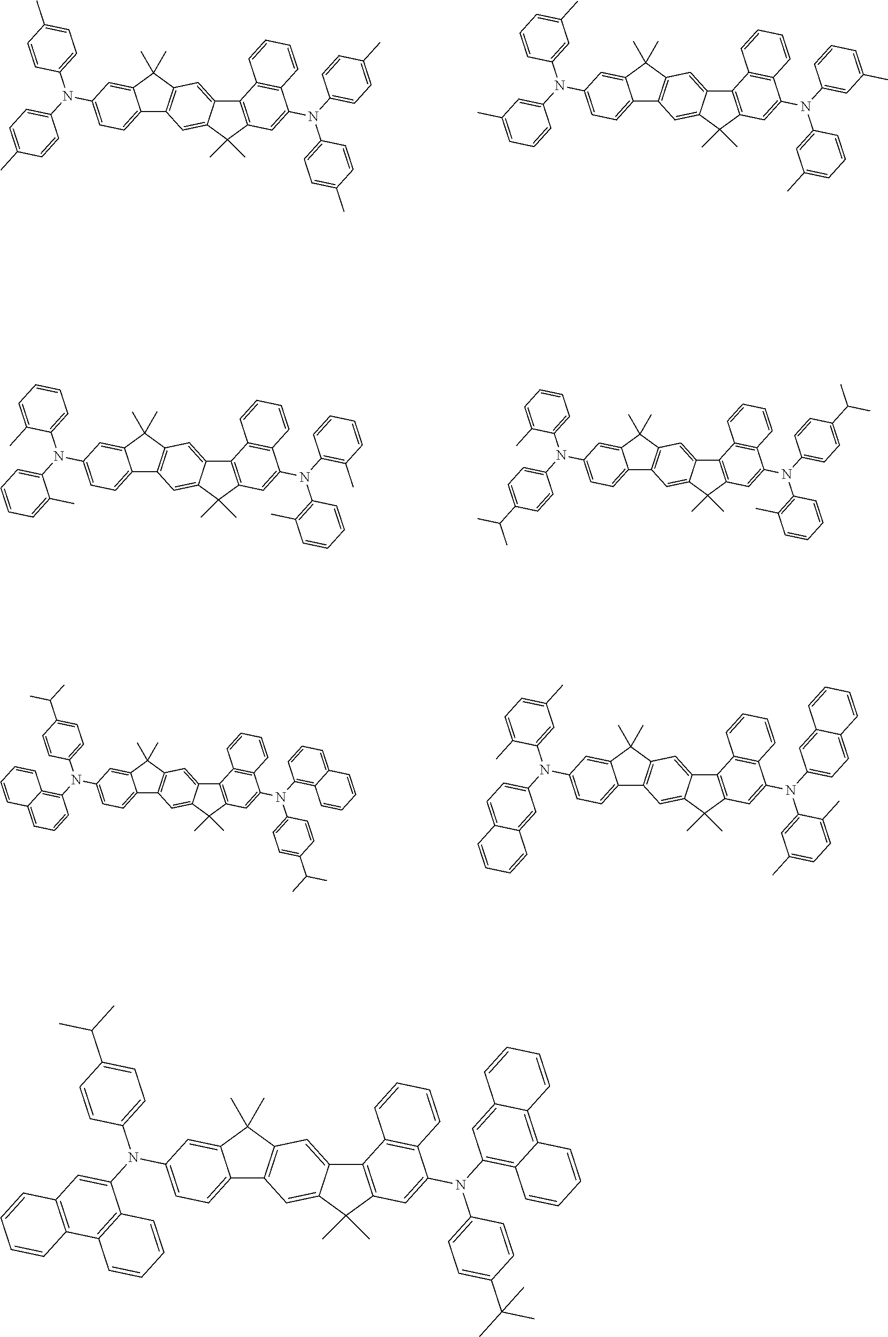

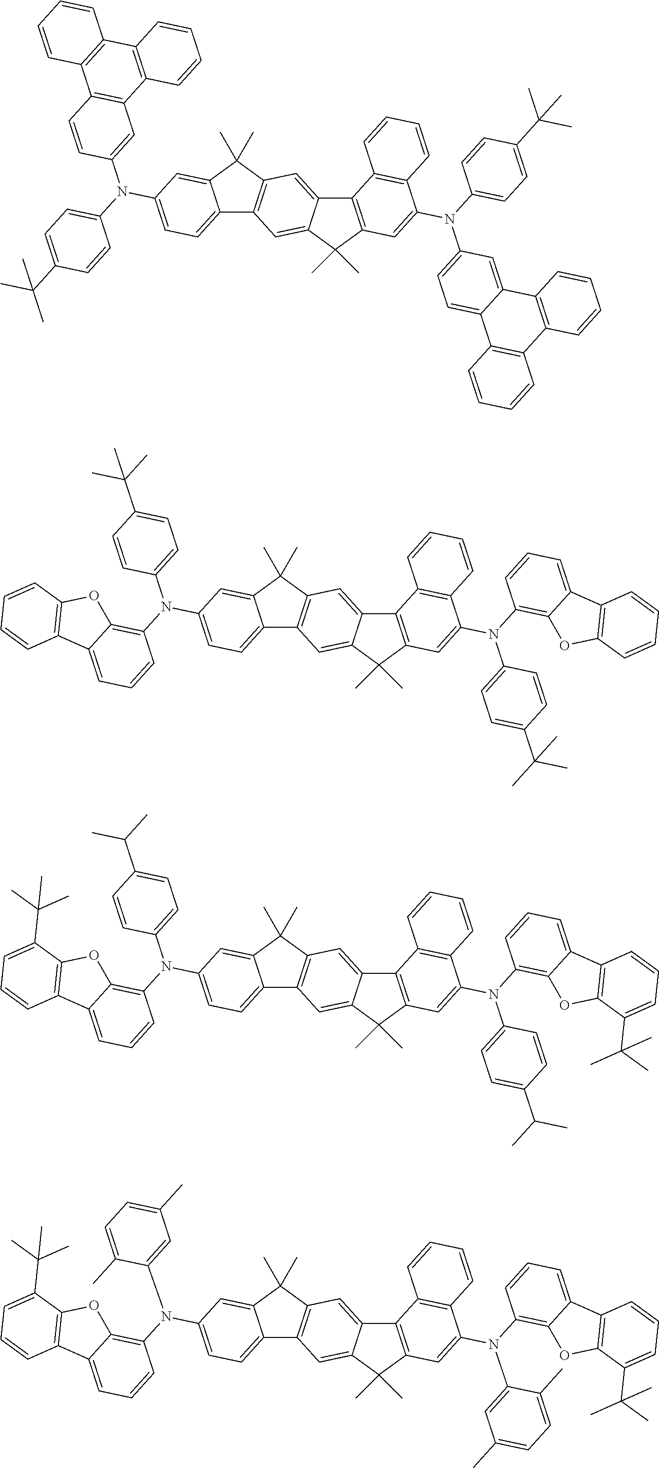

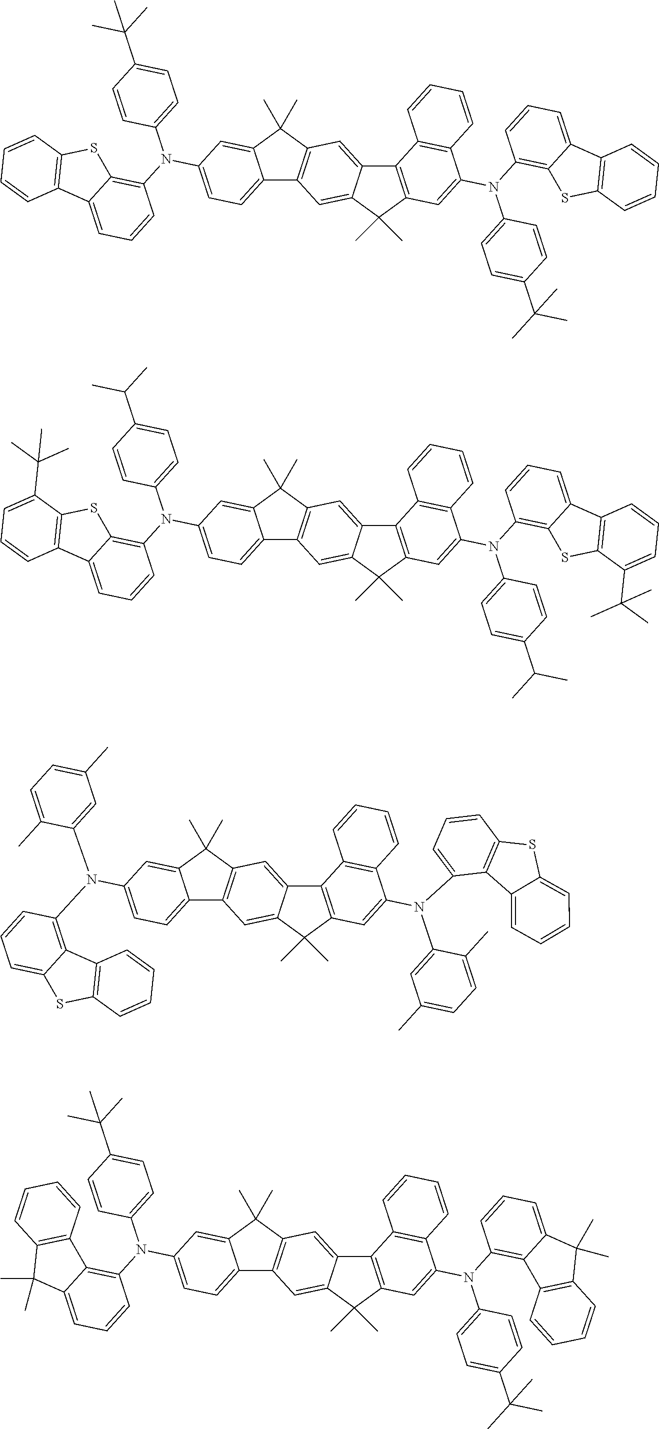

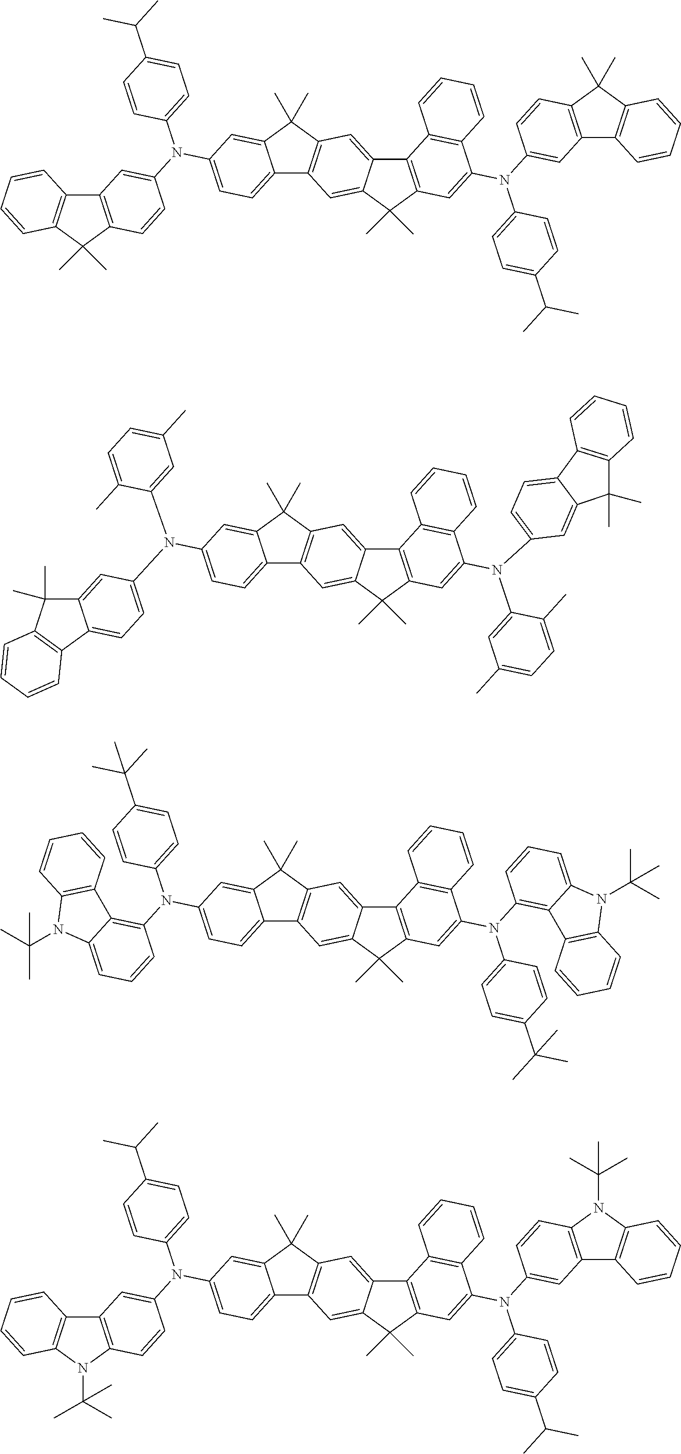

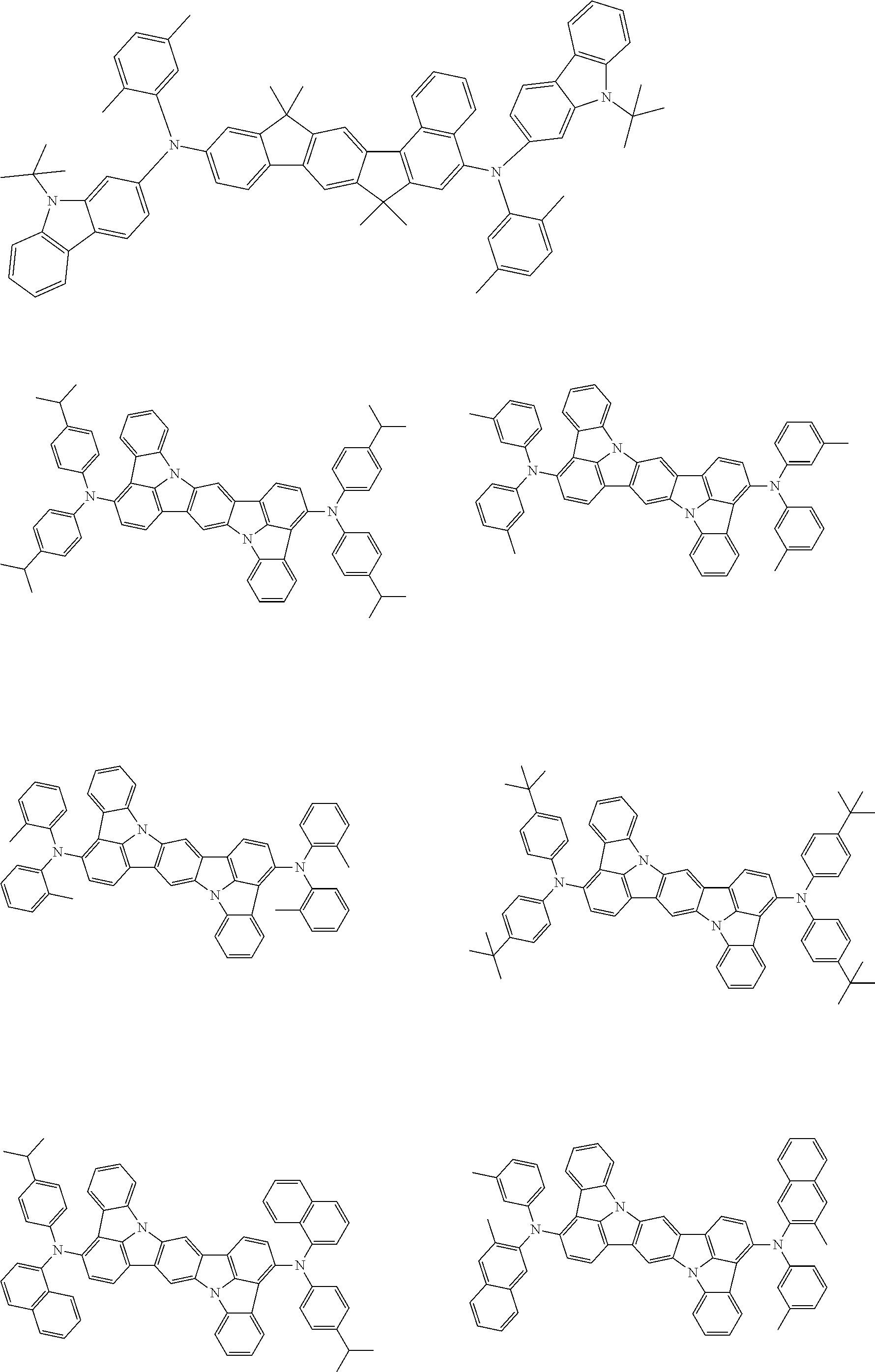

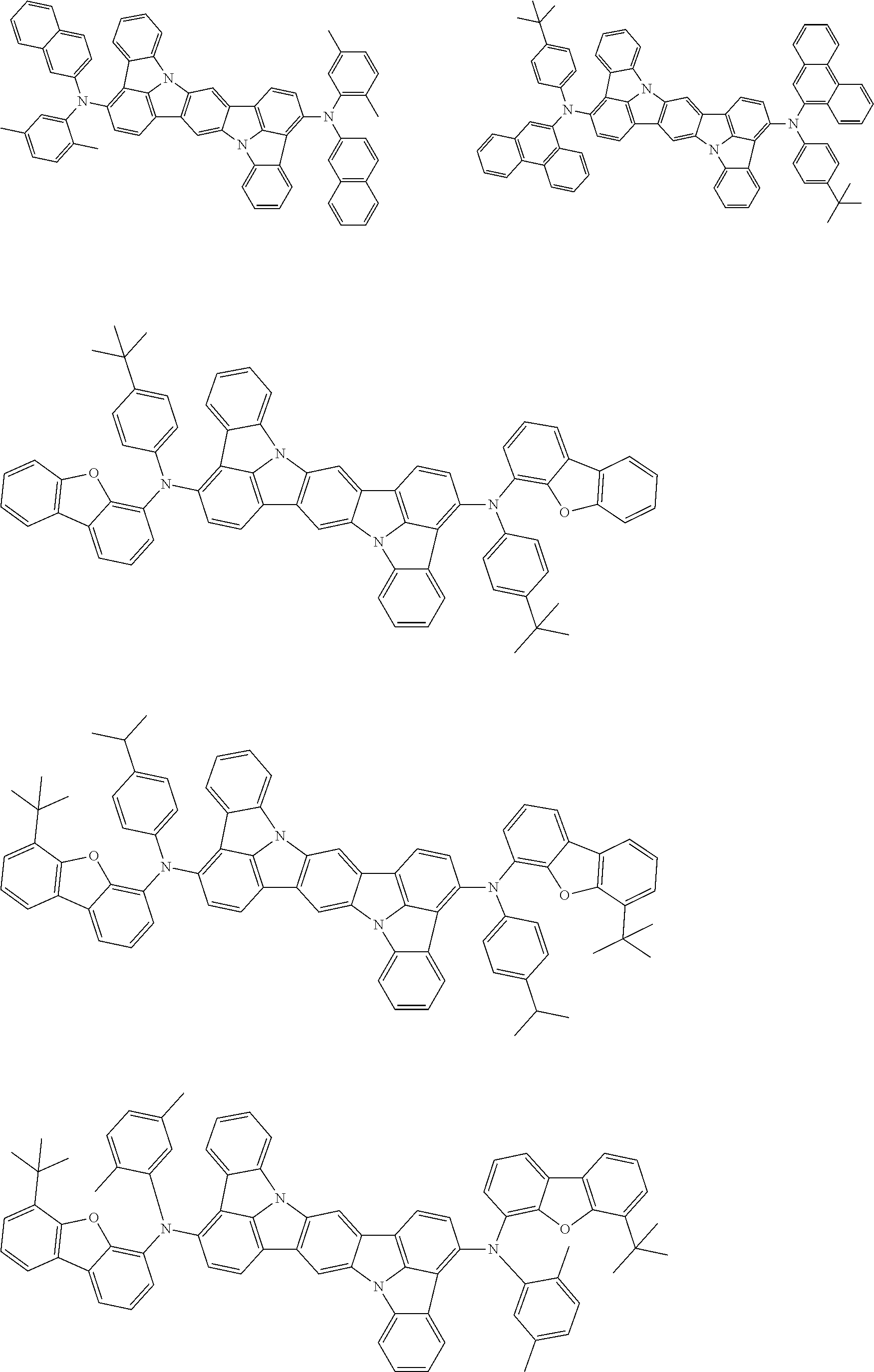

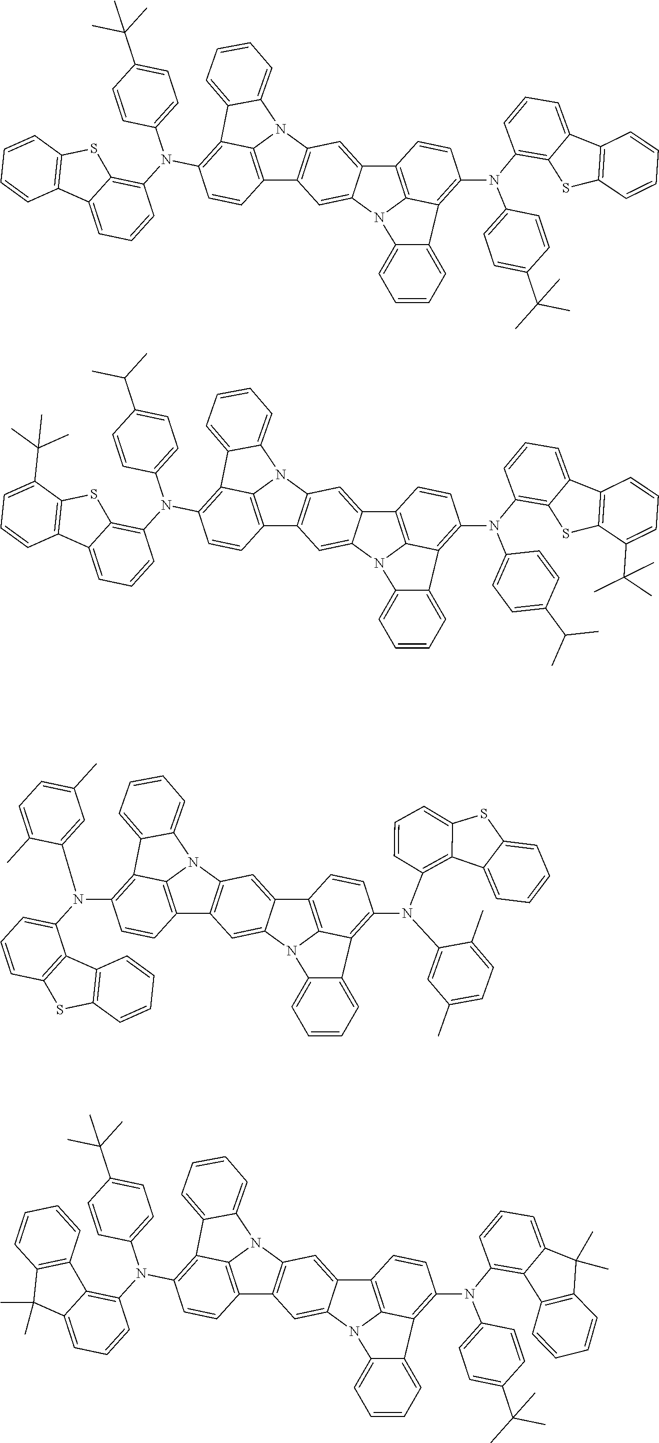

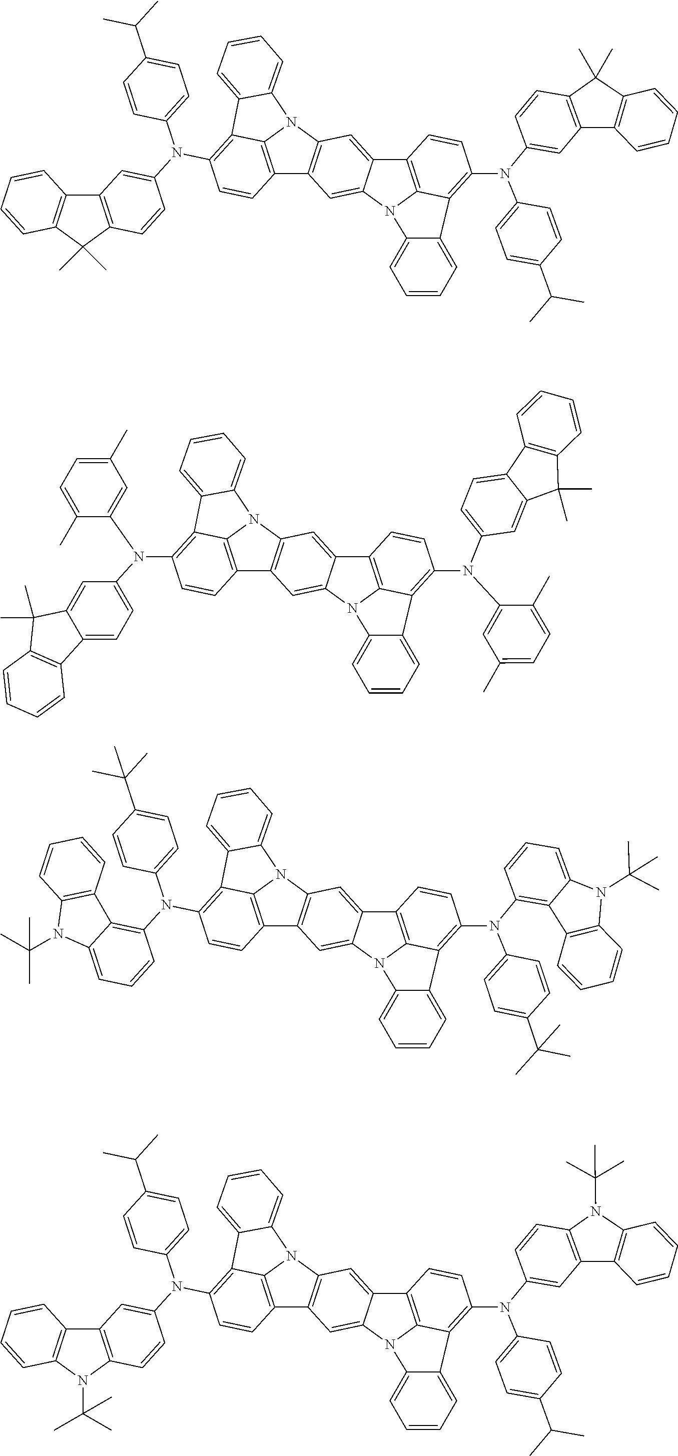

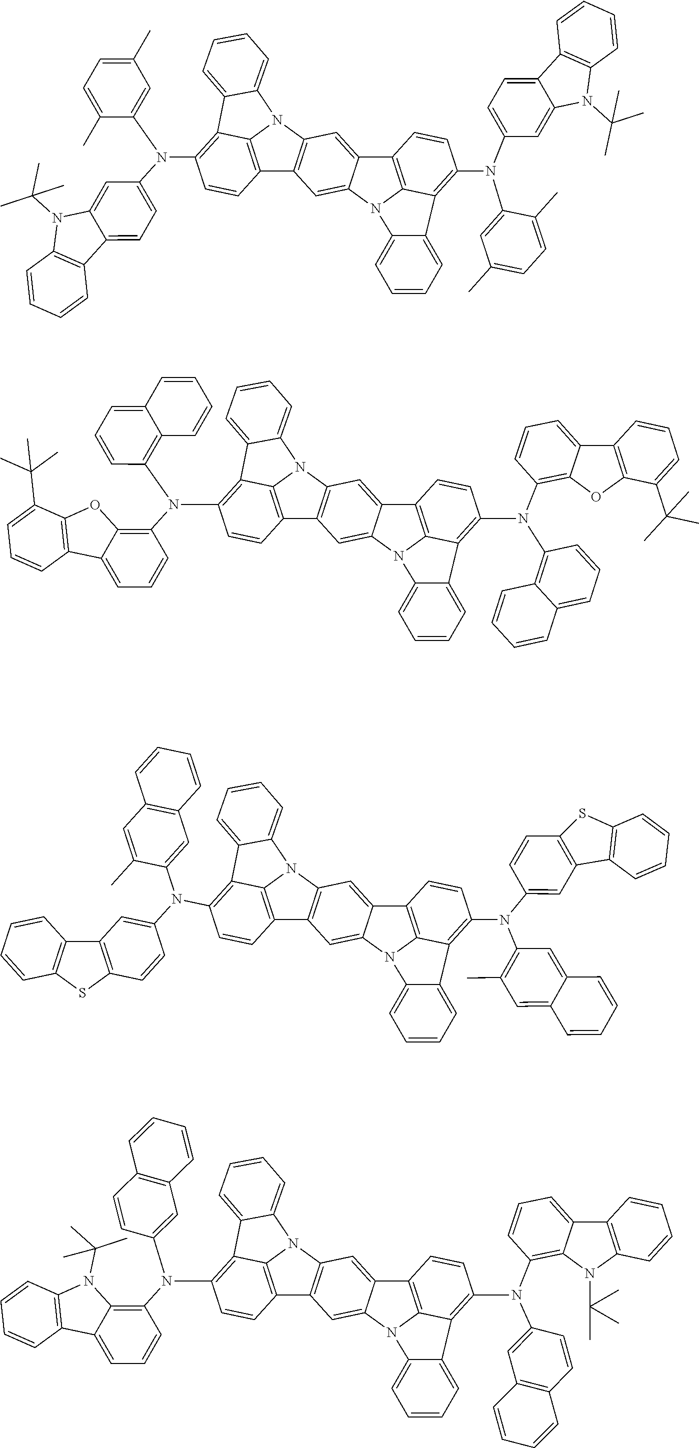

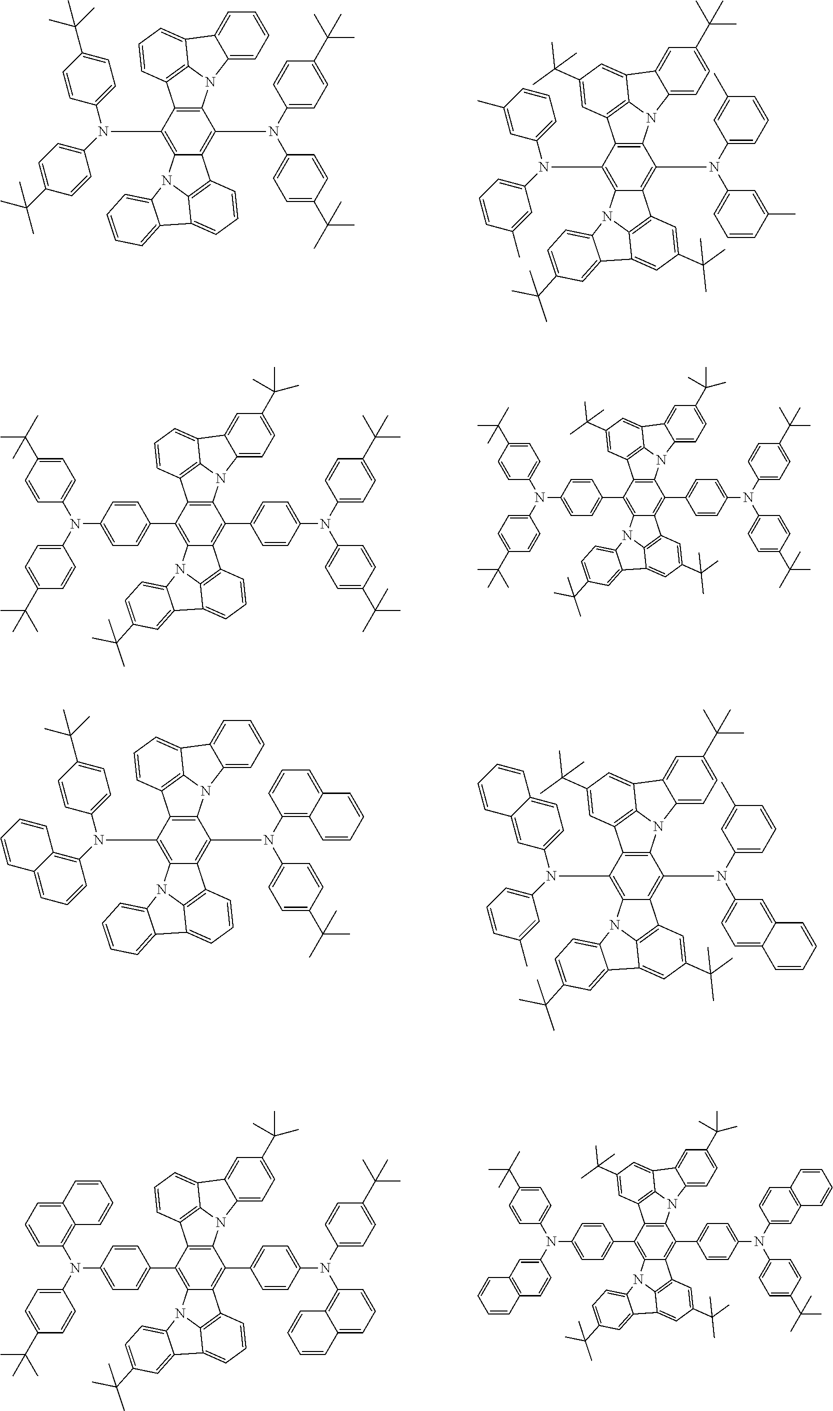

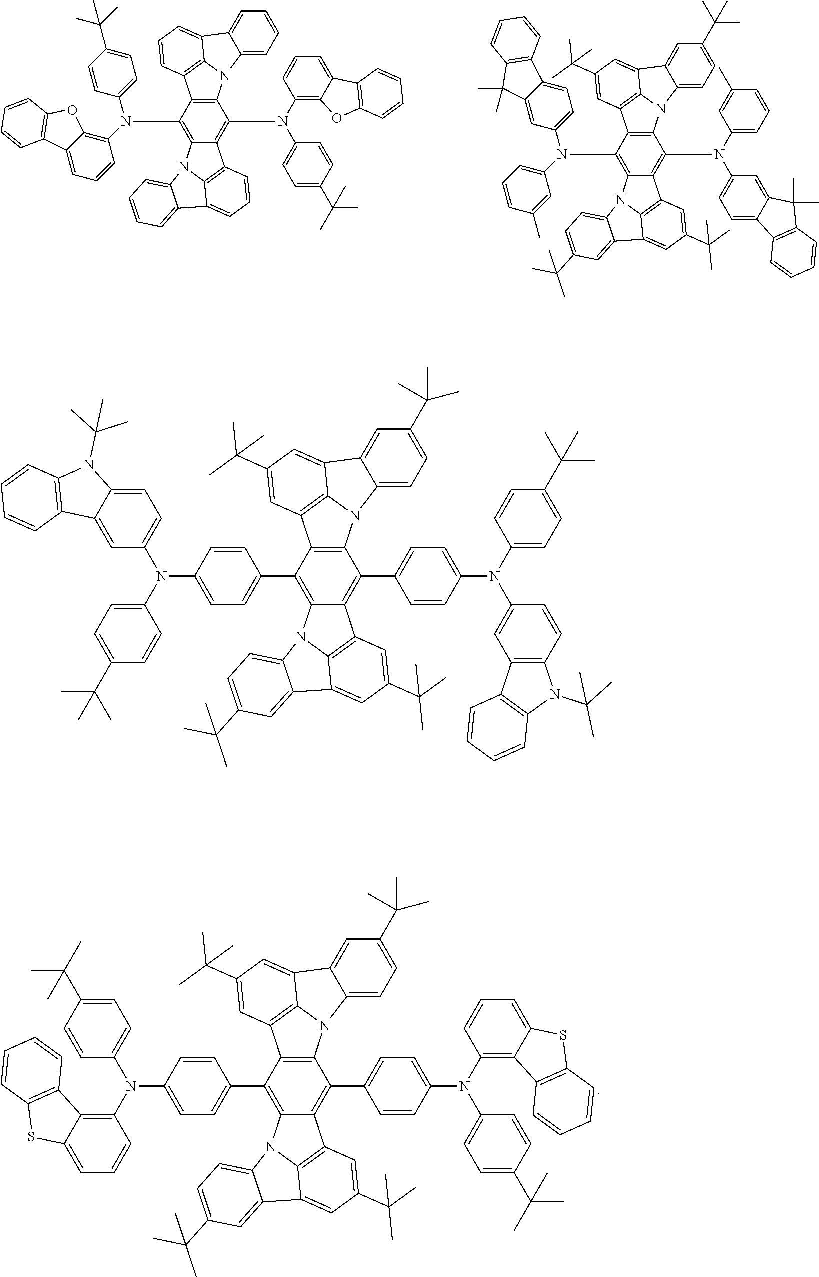

16. The mixture according to claim 13, wherein an organic guest material represented by the formula (III-2) is selected from following compounds:

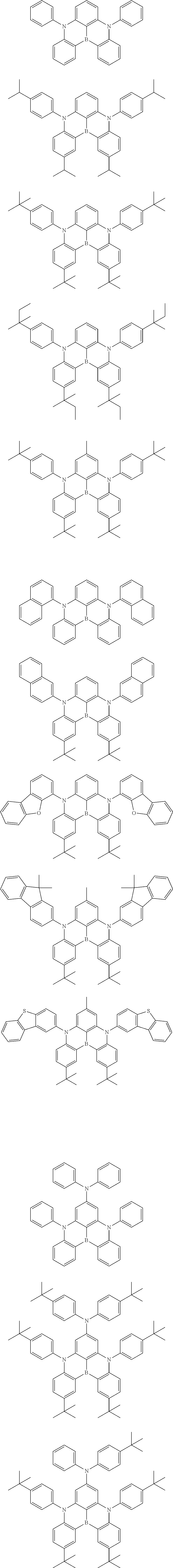

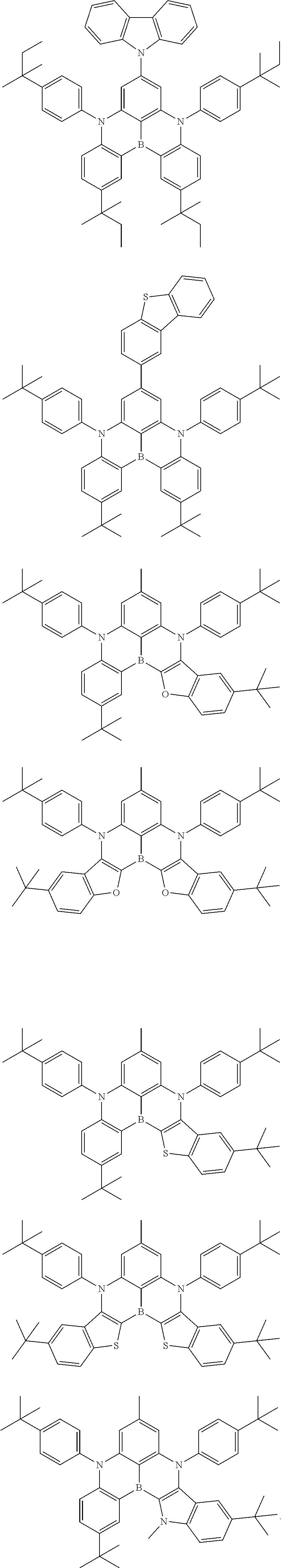

17. An organic electronic device, wherein the organic electronic device comprises a first electrode, a second electrode, and an organic functional layer arranged between the first electrode and the second electrode, and the organic functional layer comprises at least one anthracene-based compound according to claim 1.

18. The organic electronic device according to claim 17, wherein one of R₁, R₂, and R₃ is selected from -D, a methyl group, an ethyl group, a n-propyl group, an isopropyl group, a n-butyl group, a tert-butyl group, and a phenyl group, and the other two are both H.

19. The organic electronic device according to claim 17, wherein the anthracene-based compound is selected from compounds represented by formula (II-1) or formula (II-2): 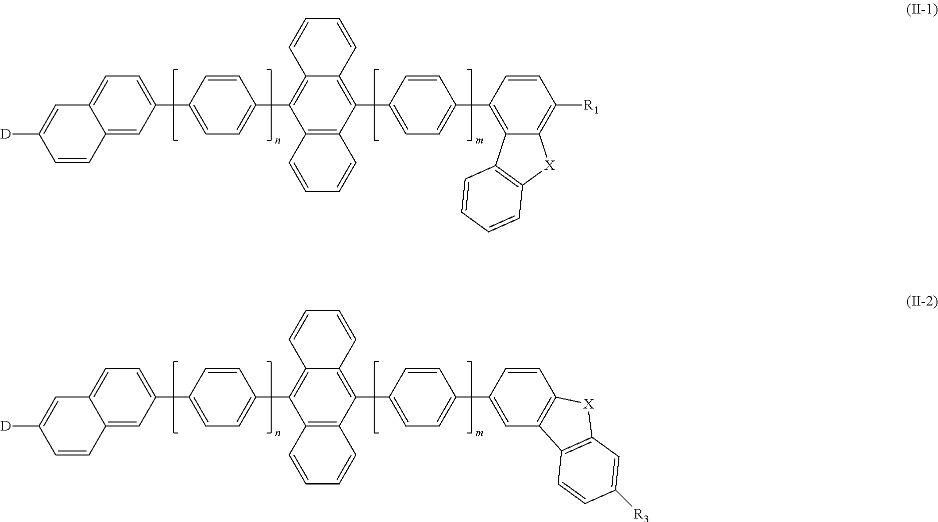
wherein n is selected from 0, 1, and 2, and m is selected from 0, 1, and 2.

20. The organic electronic device according to claim 17, wherein R₁ of the formula (II-1) is selected from —H and -D, R₃ of the formula (II-2) is selected from —H and -D, and m+n=1.
